(19) United States
(12) Patent Application Publication
(10) Pub. No.: US 2026/0096344 A1
(43) Pub. Date: Apr. 2, 2026

(54) LIGHT-EMITTING DEVICE INCLUDING HETEROCYCLIC COMPOUND, ELECTRONIC APPARATUS INCLUDING THE LIGHT-EMITTING DEVICE, ELECTRONIC DEVICE INCLUDING THE LIGHT-EMITTING DEVICE, AND THE HETEROCYCLIC COMPOUND

(71) Applicant: Samsung Display Co., Ltd., Yongin-si (KR)

(72) Inventors: Junghoon Han, Yongin-si (KR); Dojin Kim, Yongin-si (KR); Hyun Shin, Yongin-si (KR); Heechoon Ahn, Yongin-si (KR); Hyunah Um, Yongin-si (KR); Yeseul Lee, Yongin-si (KR)

(21) Appl. No.: 19/344,271

(22) Filed: Sep. 29, 2025

(30) Foreign Application Priority Data
Sep. 30, 2024 (KR) 10-2024-0133251

(51) Int. Cl. H10K 85/60; C07B 59/00; C07D 471/04; C07D 471/14; C07D 519/00; C07F 7/08; C09K 11/02; H10K 50/11; H10K 50/858

(52) U.S. Cl. CPC H10K 85/6572; C07B 59/002; C07D 471/04; C07D 471/14; C07D 519/00; C07F 7/0812; C07F 7/0816; C09K 11/02; H10K 85/654; H10K 50/11; H10K 50/858

(57) ABSTRACT

A light-emitting device, an electronic apparatus including the light-emitting device, an electronic device including the light-emitting device, and the heterocyclic compound represented by Formula 1 are disclosed. The light-emitting device may include a first electrode, a second electrode that faces (e.g., is opposite to) the first electrode, an interlayer provided between the first electrode and the second electrode and including an emission layer, and a heterocyclic compound represented by Formula 1.

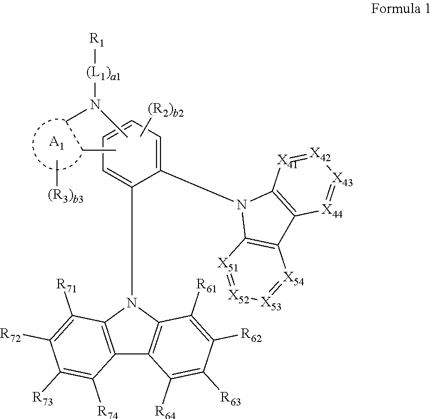

### CROSS-REFERENCE TO RELATED APPLICATION

The present application claims priority to and the benefit of Korean Patent Application No. 10-2024-0133251, filed on Sep. 30, 2024, in the Korean Intellectual Property Office, the entire disclosure of which is incorporated herein by reference.

### BACKGROUND

### 1. Field

One or more embodiments of the present disclosure relate to a light-emitting device including a heterocyclic compound, an electronic apparatus including the light-emitting device, an electronic device including the light-emitting device, and the heterocyclic compound.

### 2. Description of the Related Art

Among light-emitting devices, self-emissive devices (e.g., organic light-emitting devices) are notable for their relatively wide viewing angles, high contrast ratios, short response times, and/or excellent or desirable (suitable) characteristics in terms of luminance, driving voltage, and/or response speed.

In a light-emitting device, a first electrode is arranged on a substrate, followed substantially by a hole transport region, an emission layer, an electron transport region, and a second electrode. Holes provided by the first electrode move toward the emission layer through the hole transport region, while electrons provided by the second electrode move toward the emission layer through the electron transport region. These carriers, namely the holes and electrons, recombine in the emission layer to produce excitons. The excitons may transition and decay from an excited state to a ground state, thereby generating light.

### SUMMARY

One or more aspects of embodiments of present disclosure are directed toward a light-emitting device including a heterocyclic compound, an electronic apparatus including the light-emitting device, an electronic device including the light-emitting device, and the heterocyclic compound.

Additional aspects of embodiments will be set forth in part in the description which follows and, in part, will be apparent from the description, or may be learned by practice of the presented embodiments of the disclosure.

According to one or more embodiments of the present disclosure, a light-emitting device includes
a first electrode,
a second electrode opposite to (e.g., facing) the first electrode,
an interlayer arranged between the first electrode and the second electrode and including an emission layer, and
a heterocyclic compound represented by Formula 1:

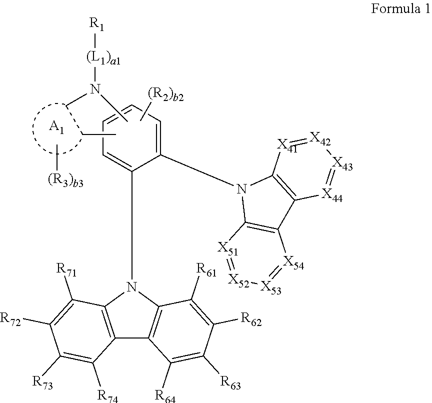

wherein, in Formula 1,
X₄₁ may be N or C(R₄₁), X₄₂ may be N or C(R₄₂), X₄₃ may be N or C(R₄₃), X₄₄ may be N or C(R₄₄), X₅₁ may be N or C(R₅₁), X₅₂ may be N or C(R₅₂), X₅₃ may be N or C(R₅₃), and X₅₄ may be N or C(R₅₄),
at least one selected from among X₄₁ to X₄₄ and X₅₁ to X₅₄ may be N,
ring A₁ may be a C₅-C₆₀ carbocyclic group or a C₁-C₆₀ heterocyclic group,
a1 may be an integer of 0 to 5,
L₁ may be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
-(L₁)ₐ₁-*′ may be a single bond (e.g., a single covalent bond) if (e.g., when) a1 is 0,
b2 may be an integer of 0 to 2,
b3 may be an integer of 0 to 10,
R₁ to R₃, R₄₁ to R₄₄, R₅₁ to R₅₄, R₆₁ to R₆₄, and R₇₁ to R₇₄ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₅-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, a C₇-C₆₀ arylalkyl group unsubstituted or substituted with at least one R₁₀ₐ, —C(Q₁)(Q₂)(Q₃), —Si(Q₁)(Q₂)(Q₃), —B(Q₁)(Q₂), —N(Q₁)(Q₂), —P(Q₁)(Q₂), —C(═O) (Q₁), —S(═O) (Q₁), —S(═O)₂(Q₁), —P(═O)(Q₁)(Q₂), or —P(═S)(Q₁)(Q₂),
neighboring two or more selected from among R₁ to R₃, R₄₁ to R₄₄, R₅₁ to R₅₄, R₆₁ to R₆₄, and R₇₁ to R₇₄ may optionally be bonded together to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ may be
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O) (Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, or a C₁-C₆₀ heteroarylthio group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O) (Q₂₁), —S(═O)₂(Q₂₁), —P(═O)(Q₂₁)(Q₂₂), or any combination thereof, or
—O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O) (Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group, or
a C₃-C₆₀ carbocyclic group, a C₁-C₆₀ heterocyclic group, a C₇-C₆₀ arylalkyl group, or a C₂-C₆₀ heteroarylalkyl group, each being unsubstituted or substituted with group, a biphenyl group, or any combination thereof.

According to one or more embodiments of the present disclosure, an electronic apparatus includes the light-emitting device.

According to one or more embodiments of the present disclosure, an electronic device includes the light-emitting device.

According to one or more embodiments of the present disclosure, provided is the heterocyclic compound represented by Formula 1.

### BRIEF DESCRIPTION OF THE DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects and features of certain embodiments of the present disclosure will be more apparent from the following description taken in conjunction with the accompanying drawings, in which:

FIG. 1 is a schematic cross-sectional view of a structure of a light-emitting device according to one or more embodiments;

FIG. 2 is a schematic cross-sectional view of a structure of an electronic apparatus according to one or more embodiments;

FIG. 3 is a schematic cross-sectional view of a structure of an electronic apparatus according to one or more embodiments;

FIG. 4 is a schematic view of an electronic device including a light-emitting device according to one or more embodiments;

FIG. 5 is a schematic view of the exterior of a vehicle as an electronic device including a light-emitting device according to one or more embodiments; and

FIGS. 6A-6C are each a schematic view of the interior of a vehicle as an electronic device including a light-emitting device according to one or more embodiments of the present disclosure.

### DETAILED DESCRIPTION

Reference will be made in more detail to one or more embodiments, examples of which are illustrated in the accompanying drawings, wherein like reference numerals refer to like elements throughout the accompanying drawings and the written description, and duplicative descriptions thereof may not be provided in the specification for conciseness. In this regard, the subject matter of the present disclosure may be embodied in different forms and should not be construed as being limited to one or more embodiments set forth herein. Rather, these embodiments are provided as examples, by referring to the drawings, to explain the aspects and features of the present disclosure to those skilled in the art.

As used herein, the term “and/or” or “or” includes any and all combinations of one or more of the associated listed items. Throughout the present disclosure, the expressions such as “at least one of,” “one of,” and “selected from,” when preceding a list of elements, modify the entire list of elements and do not modify the individual elements of the list. For example, “at least one of a, b, or c,” “at least one selected from a, b, and c,” or “at least one selected from among a to c”, and/or the like indicates only a, only b, only c, both (e.g., simultaneously) a and b, both (e.g., simultaneously) a and c, both (e.g., simultaneously) b and c, all of a, b, and c, or variations thereof.

The symbol “/” utilized herein may be interpreted as “and” or “or” depending on the situation.

In the present disclosure, it will be understood that the term “comprise(s)/comprising,” “include(s)/including,” or “have/has/having” specifies the presence of stated features, integers, steps, operations, elements, and/or components, but do not preclude the presence or addition of one or more other features, integers, steps, operations, elements, components, and/or groups thereof. Additionally, the terms “comprise(s)/comprising,” “include(s)/including,” “have/has/having” or similar terms include or support the terms “consisting of” and “consisting essentially of,” indicating the presence of stated features, integers, steps, operations, elements, and/or components, without or essentially without the presence of other features, integers, steps, operations, elements, components, and/or groups thereof.

In the context of the present application and unless otherwise defined, the terms “use,” “using,” and “used” may be considered synonymous with the terms “utilize,” “utilizing,” and “utilized,” respectively.

Throughout the present disclosure, if (e.g., when) a component, such as a layer, a film, a region, or a plate, is mentioned to be placed “on” another component, it will be understood that it may be directly on another component or that another component may be therebetween. In one or more embodiments, “directly on” may refer to that there are no additional layers, films, regions, plates, and/or the like between a layer, a film, a region, a plate, and/or the like and the other part. For example, “directly on” may refer to that two layers or two members are arranged without utilizing an additional member, such as an adhesive member, therebetween.

In the present disclosure, although the terms “first,” “second,” and/or the like, may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” if (e.g., when) describing embodiments of the present disclosure refers to “one or more embodiments of the present disclosure”.

As utilized herein, the terms “substantially,” “about,” or similar terms are used as terms of approximation and not as terms of degree and are intended to account for the inherent deviations in measured or calculated values that would be recognized by those of ordinary skill in the art. “About” as used herein, is inclusive of the stated value and refers to as being within an acceptable range of deviation for the particular value as determined by one of ordinary skill in the art, considering the measurement in question and the error associated with measurement of the particular quantity (e.g., the limitations of the measurement system). For example, “about” may refer to as being within one or more standard deviations, or within ±30%, ±20%, ±10%, or ±5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, for example, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in the present disclosure is intended to include all higher numerical limitations subsumed therein. Accordingly, Applicant reserves the right to amend the disclosure, including the claims, to expressly recite any sub-range subsumed within the ranges expressly recited herein.

A light-emitting device (e.g., an organic light-emitting device) according to one or more embodiments may include: a first electrode; a second electrode opposite to (e.g., facing) the first electrode; an interlayer between (e.g., arranged between) the first electrode and the second electrode and including an emission layer; and a heterocyclic compound represented by Formula 1.

Hereinafter, the heterocyclic compound represented by Formula 1 will be described in more detail:

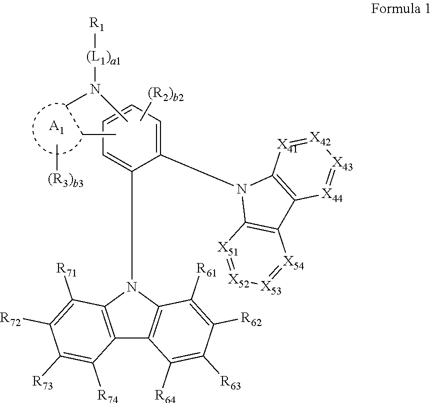

wherein X₄₁ may be N or C(R₄₁), X₄₂ may be N or C(R₄₂), X₄₃ may be N or C(R₄₃), X₄₄ may be N or C(R₄₄), X₅₁ may be N or C(R₅₁), X₅₂ may be N or C(R₅₂), X₅₃ may be N or C(R₅₃), and X₅₄ may be N or C(R₅₄), wherein at least one selected from among X₄₁ to X₄₄ and X₅₁ to X₅₄ may be N.

In one or more embodiments, one or two selected from among X₄₁ to X₄₄ and X₅₁ to X₅₄ may be N.

Ring A₁ in Formula 1 may be a C₅-C₆₀ carbocyclic group or a C₁-C₆₀ heterocyclic group.

In one or more embodiments, ring A₁ may be a benzene group, a naphthalene group, an anthracene group, a phenanthrene group, a triphenylene group, a pyrene group, a chrysene group, an acenaphthylene group, a perylene group, a benzopyrene group, a benzochrysene group, a benzotriphenylene group, a fluoranthene group, a coronene group, a cyclopentadiene group, a 1,2,3,4-tetrahydronaphthalene group, an acridine group, a thiophene group, a furan group, an indole group, a benzoborole group, a benzophosphole group, an indene group, a benzosilole group, a benzogermole group, a benzothiophene group, a benzoselenophene group, a benzofuran group, a benzotellurophene group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzotellurophene group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

For example, ring A₁ may be a benzene group or a naphthalene group.

a1 in Formula 1 may be an integer of 0 to 5.

In one or more embodiments, a1 may be 0 or 1.

L₁ in Formula 1 may be a C₃-C₆₀ carbocyclic group unsubstituted or substituted with at least one R₁₀ₐ or a C₁-C₆₀ heterocyclic group unsubstituted or substituted with at least one R₁₀ₐ.

In one or more embodiments, L₁ may be a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluoren-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each being unsubstituted or substituted with at least one R₁₀ₐ.

In one or more embodiments, L₁ may be a group represented by one (e.g., any one selected from among Formulae 3-1 to 3-28:

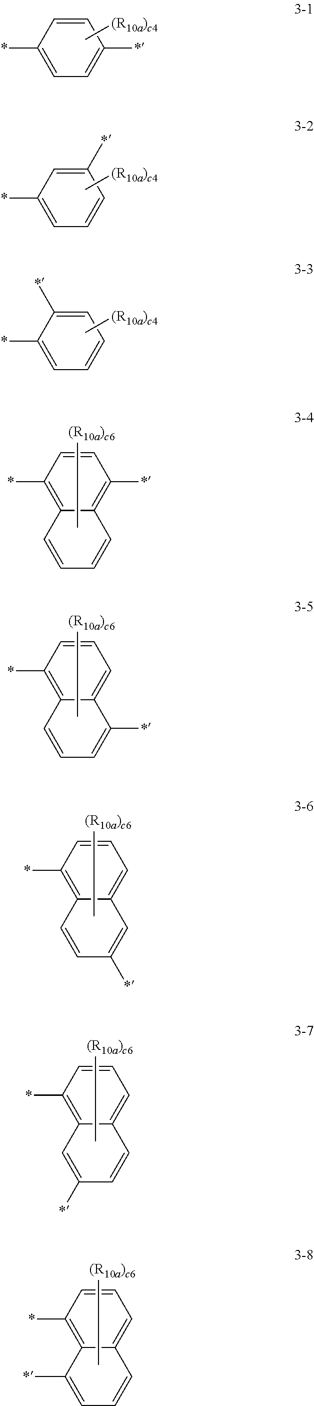

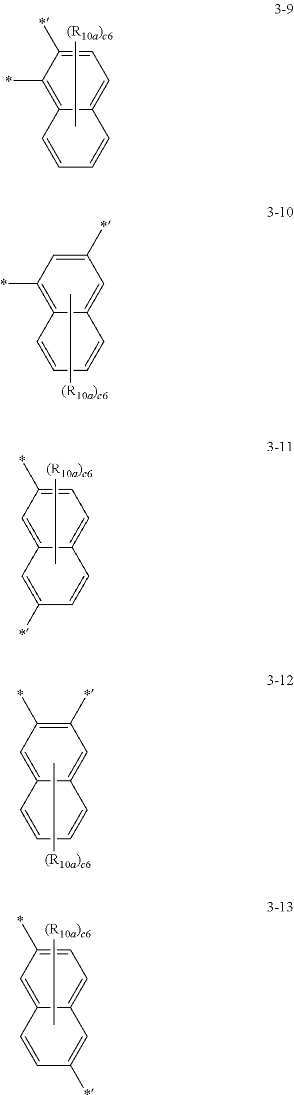

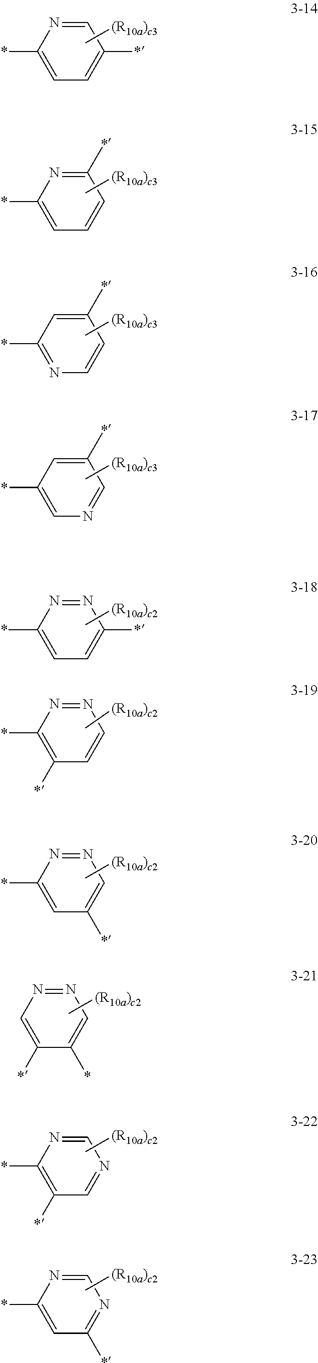

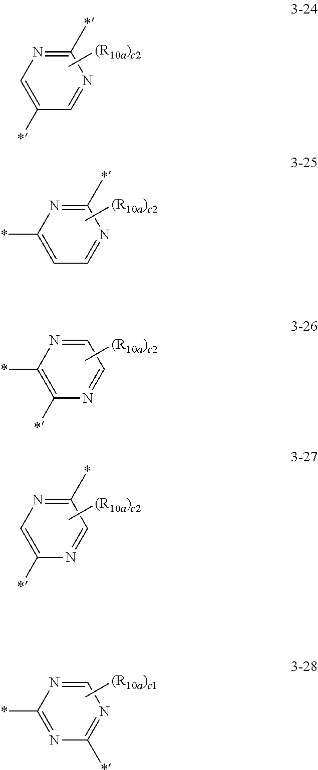

wherein, in Formulae 3-1 to 3-28,
R₁₀ₐ may be the same as defined in one or more embodiments,
c1 may be 0 or 1,
c2 may be an integer of 0 to 2,
c3 may be an integer of 0 to 3,
c4 may be an integer of 0 to 4,
c6 may be an integer of 0 to 6, and
* and *′ each indicate a binding site to a neighboring atom.

In one or more embodiments, L₁ may be a group represented by one (e.g., any one) selected from among Formulae 3-1 to 3-3 and 3-14 to 3-17.
*-(L₁)ₐ₁-*′ may be a single bond (e.g., a single covalent bond) if (e.g., when) a1 in Formula 1 is 0.
b2 in Formula 1 may be an integer of 0 to 2.
b3 in Formula 1 may be an integer of 0 to 10.
R₁ to R₃, R₄₁ to R₄₄, R₅₁ to R₅₄, R₆₁ to R₆₄, and R₇₁ to R₇₄ in Formula 1 may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, a C₇-C₆₀ arylalkyl group unsubstituted or substituted with at least one R₁₀ₐ, —C(Q₁)(Q₂)(Q₃), —Si(Q₁)(Q₂)(Q₃), —B(Q₁)(Q₂), —N(Q₁)(Q₂), —P(Q₁)(Q₂), —C(═O) (Q₁), —S(═O) (Q₁), —S(═O)₂(Q₁), —P(═O)(Q₁)(Q₂), or —P(═S)(Q₁)(Q₂), and

neighboring two or more selected from among R₁ to R₃, R₄₁ to R₄₄, R₅₁ to R₅₄, R₆₁ to R₆₄, and R₇₁ to R₇₄ may optionally be bonded together to form a C₅-C₆₀ carbocyclic group unsubstituted or substituted with at least one R₁₀ₐ or a C₁-C₆₀ heterocyclic group unsubstituted or substituted with at least one R₁₀ₐ.
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O) (Q₁₁), —S(═O)₂ (Q₁₁), —P(═O)(Q₁₁)(Q₁₂), or any combination thereof;
a C₅-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, or a C₁-C₆₀ heteroarylthio group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O) (Q₂₁), —S(═O)₂ (Q₂₁), —P(═O)(Q₂₁)(Q₂₂), or any combination thereof; or
—O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O) (Q₃₁), —S(═O)₂ (Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each being unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, R₁ to R₃, R₄₁ to R₄₄, R₅₁ to R₅₄, R₆₁ to R₆₄, and R₇₁ to R₇₄ may each independently be hydrogen, deuterium, —F, —Cl, —Br, —I, a hydroxyl group, a cyano group, a nitro group, a C₁-C₂₀ alkyl group, or a C₁-C₂₀ alkoxy group;
a C₁-C₂₀ alkyl group or a C₁-C₂₀ alkoxy group, each being unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl (or adamantyl) group, a norbornanyl (or norbornyl) group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl (or adamantyl) group, a norbornany (or norbornyl) I group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a tetraphenylenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each being unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl (or adamantyl) group, a norbornanyl (or norbornyl) group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a achrysenyl group, a tetraphenylenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzosilolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O) (Q₃₁), —S(═O)₂ (Q₃₁), —P(═O)(Q₃₁)(Q₃₂), or any combination thereof; or
C(Q₁)(Q₂)(Q₃), —Si(Q₁)(Q₂)(Q₃), —N(Q₁)(Q₂), —B(Q₁)(Q₂), —C(═O) (Q₁), —S(═O)₂ (Q₁), or —P(═O)(Q₁)(Q₂), and

Q₁ to Q₃ and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each being unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, at least one selected from among R₁ to R₃, R₄₁ to R₄₄, R₅₁ to R₅₄, R₆₁ to R₆₄, and R₇₁ to R₇₄ may be deuterium.

In one or more embodiments, the heterocyclic compound represented by Formula 1 may be represented by Formula 1A:

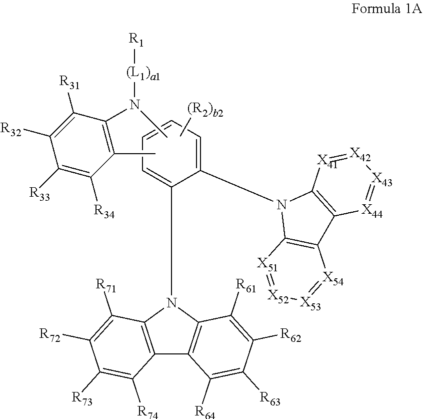

wherein, in Formula 1A,
X₄₁ to X₄₄, X₅₁ to X₅₄, a1, L₁, b2, R₁, R₂, R₄₁ to R₄₄, R₅₁ to R₅₄, R₆₁ to R₆₄, and R₇₁ to R₇₄ may each be the same as defined in one or more embodiments,
R₃₁ to R₃₄ may each be the same as defined with respect to R₃.

In one or more embodiments, the heterocyclic compound represented by Formula 1 may be represented by any of Formulae 1-1 to 1-6:

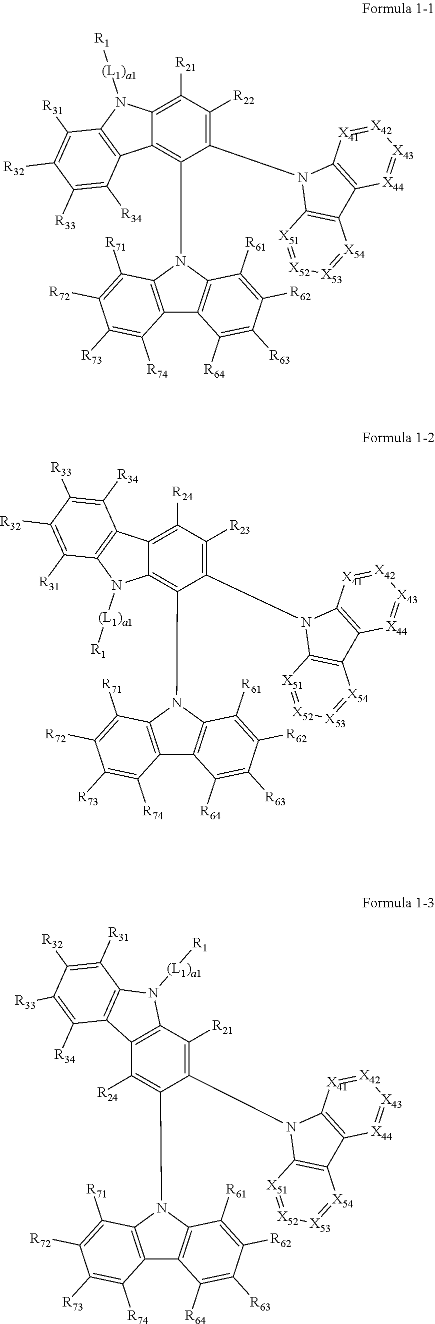

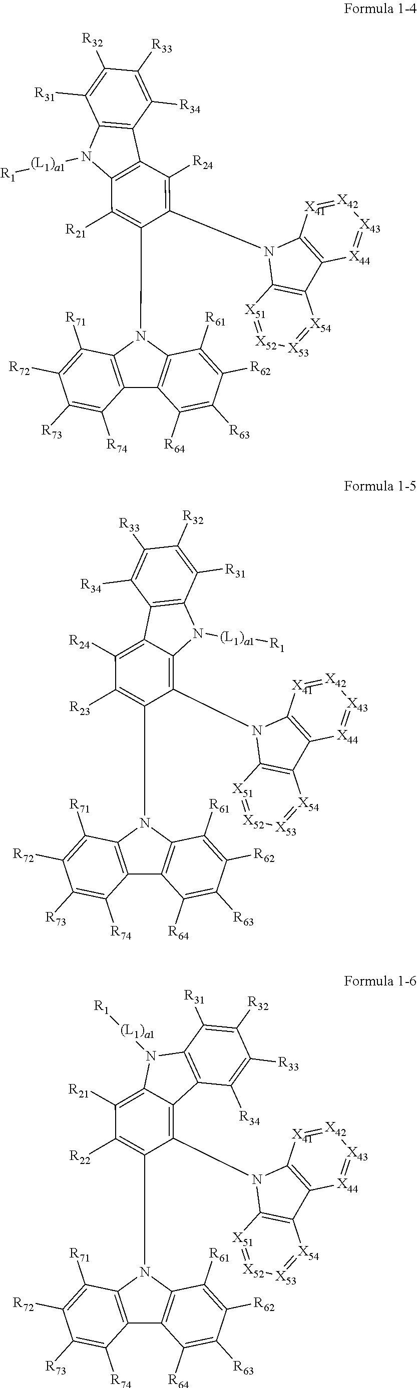

wherein, in Formulae 1-1 to 1-6,
X₄₁ to X₄₄, X₅₁ to X₅₄, a1, L₁, R₁, R₄₁ to R₄₄, R₅₁ to R₅₄, R₆₁ to R₆₄, and R₇₁ to R₇₄ may each be the same as defined in one or more embodiments,
R₂₁ to R₂₄ may each be the same as defined with respect to R₂, and
R₃₁ to R₃₄ may each be the same as defined with respect to R₃.

In one or more embodiments, the heterocyclic compound represented by Formula 1 may be represented by any one selected from among Compounds 1 to 170:

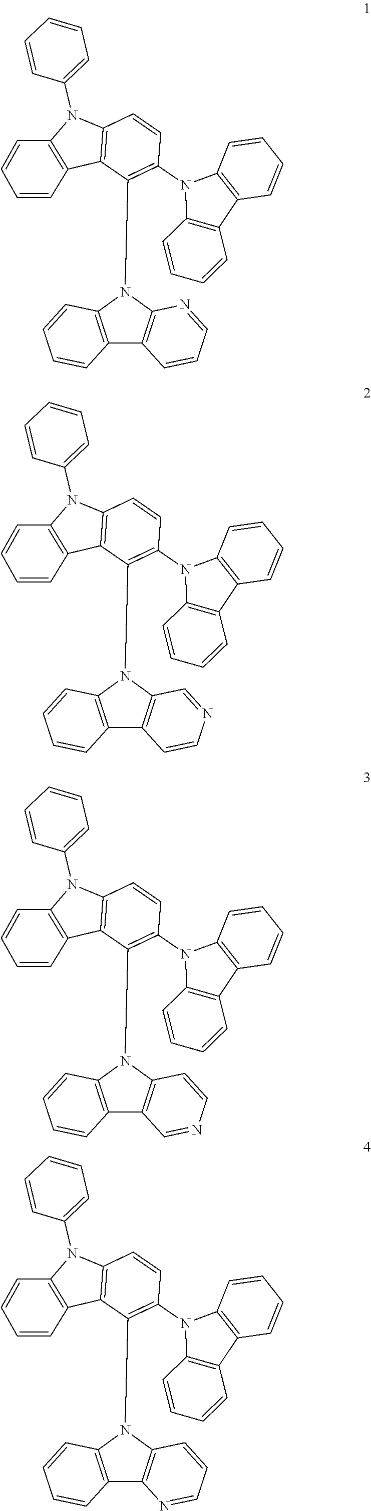

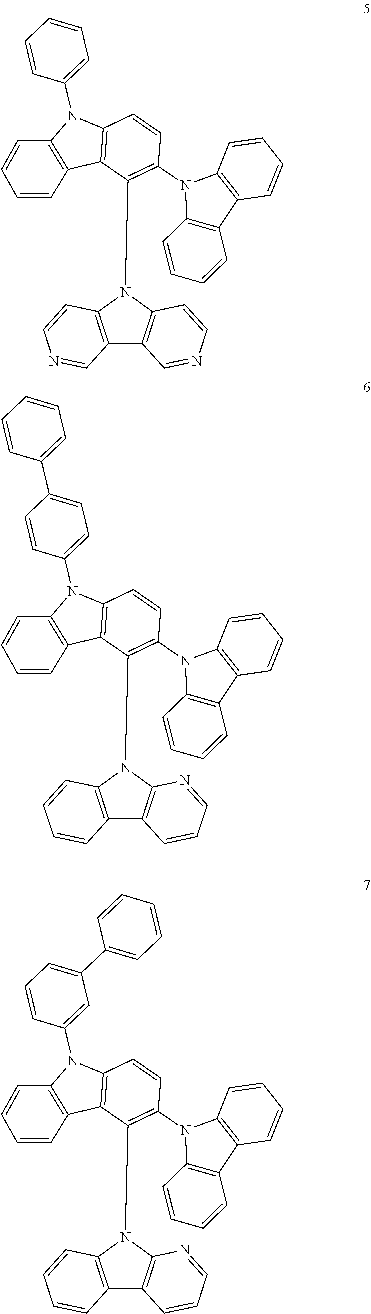

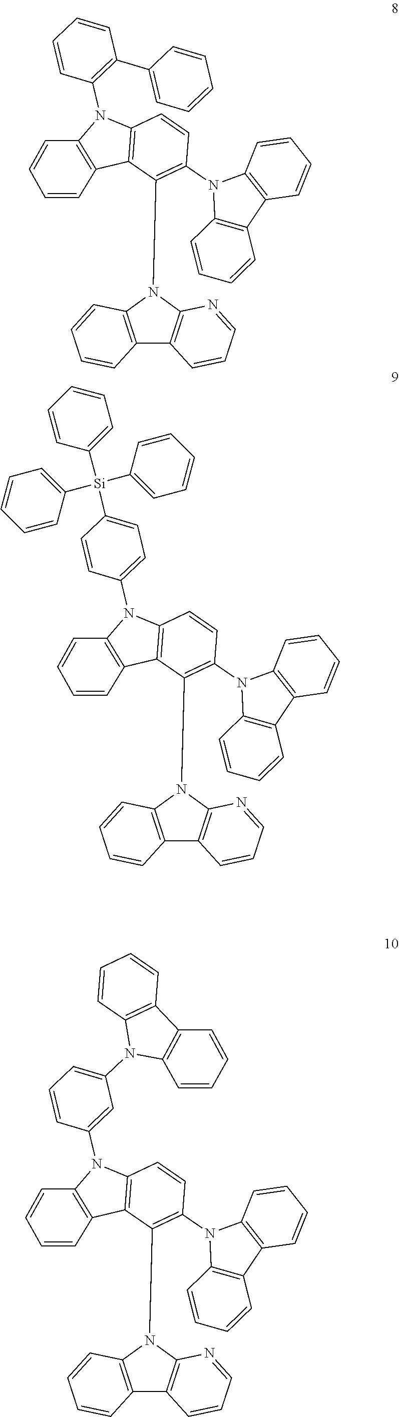

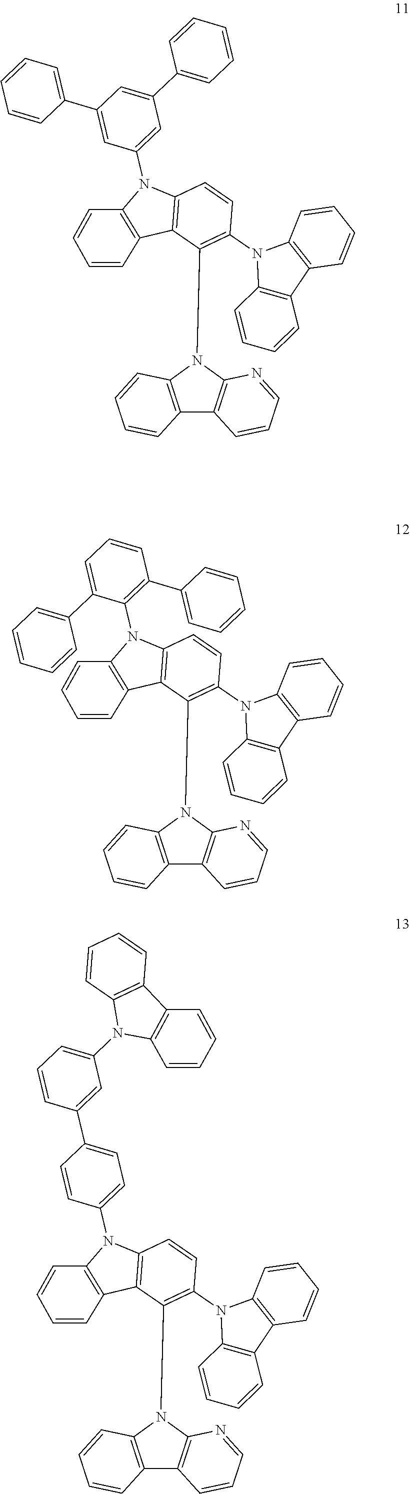 [SMILES: c1ccc(-c2cc(-c3ccccc3)cc(-n3c4ccccc4c4c(-n5c6ccccc6c6cccnc65)c(-n5c6ccccc6c6ccccc65)ccc43)c2)cc1 ; c1ccc(-c2cccc(-c3ccccc3)c2-n2c3ccccc3c3c(-n4c5ccccc5c5cccnc54)c(-n4c5ccccc5c5ccccc54)ccc32)cc1 ; c1cc(-c2ccc(-n3c4ccccc4c4c(-n5c6ccccc6c6cccnc65)c(-n5c6ccccc6c6ccccc65)ccc43)cc2)cc(-n2c3ccccc3c3ccccc32)c1]

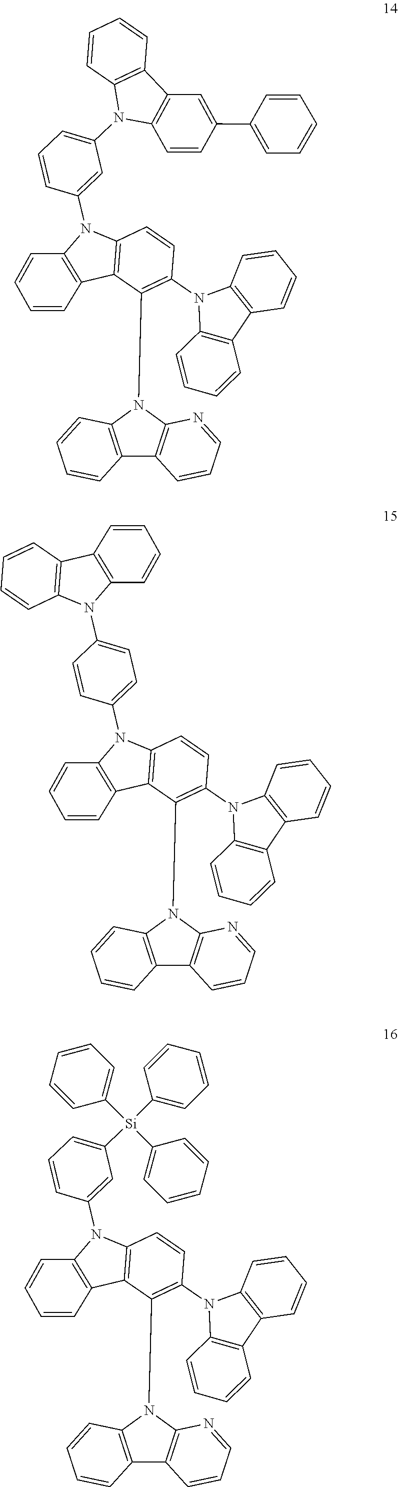

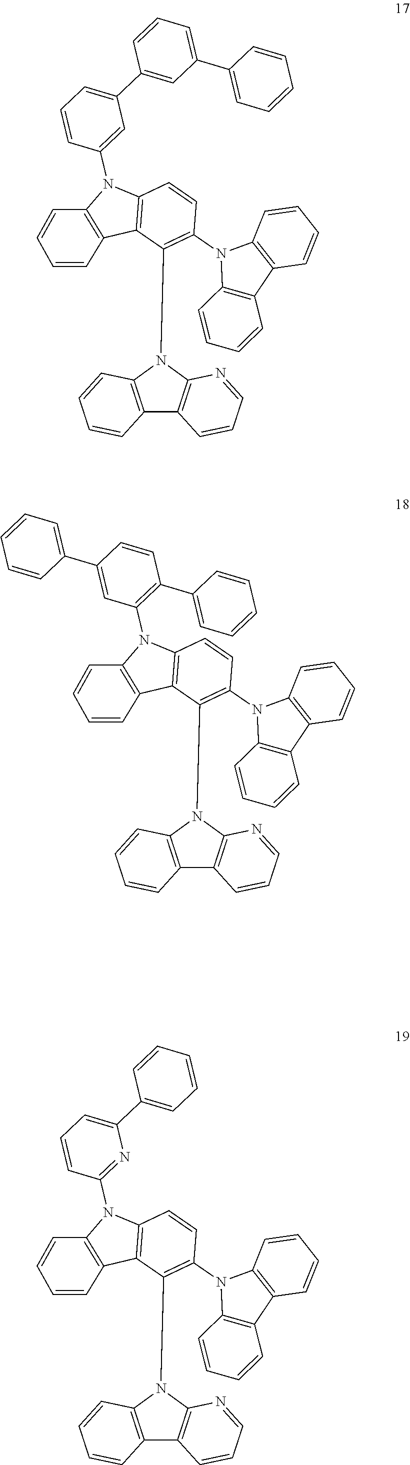 [SMILES: c1ccc(-c2cccc(-c3cccc(-n4c5ccccc5c5c(-n6c7ccccc7c7cccnc76)c(-n6c7ccccc7c7ccccc76)ccc54)c3)c2)cc1 ; c1ccc(-c2ccc(-c3ccccc3)c(-n3c4ccccc4c4c(-n5c6ccccc6c6cccnc65)c(-n5c6ccccc6c6ccccc65)ccc43)c2)cc1 ; c1ccc(-c2cccc(-n3c4ccccc4c4c(-n5c6ccccc6c6cccnc65)c(-n5c6ccccc6c6ccccc65)ccc43)n2)cc1]

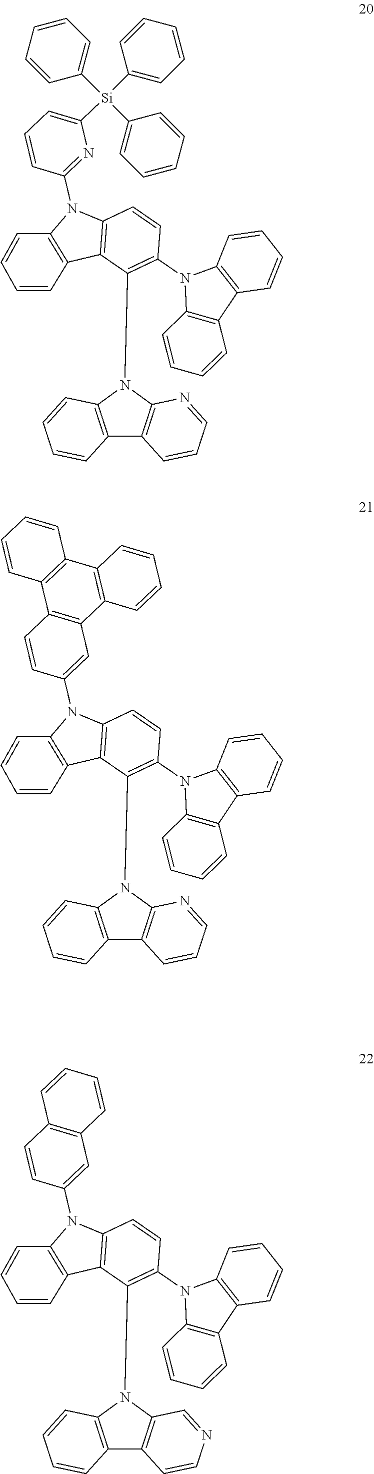 [SMILES: c1ccc([Si](c2ccccc2)(c2ccccc2)c2cccc(-n3c4ccccc4c4c(-n5c6ccccc6c6cccnc65)c(-n5c6ccccc6c6ccccc65)ccc43)n2)cc1 ; c1ccc2c(c1)c1ccccc1c1cc(-n3c4ccccc4c4c(-n5c6ccccc6c6cccnc65)c(-n5c6ccccc6c6ccccc65)ccc43)ccc21 ; c1ccc2cc(-n3c4ccccc4c4c(-n5c6ccccc6c6ccncc65)c(-n5c6ccccc6c6ccccc65)ccc43)ccc2c1]

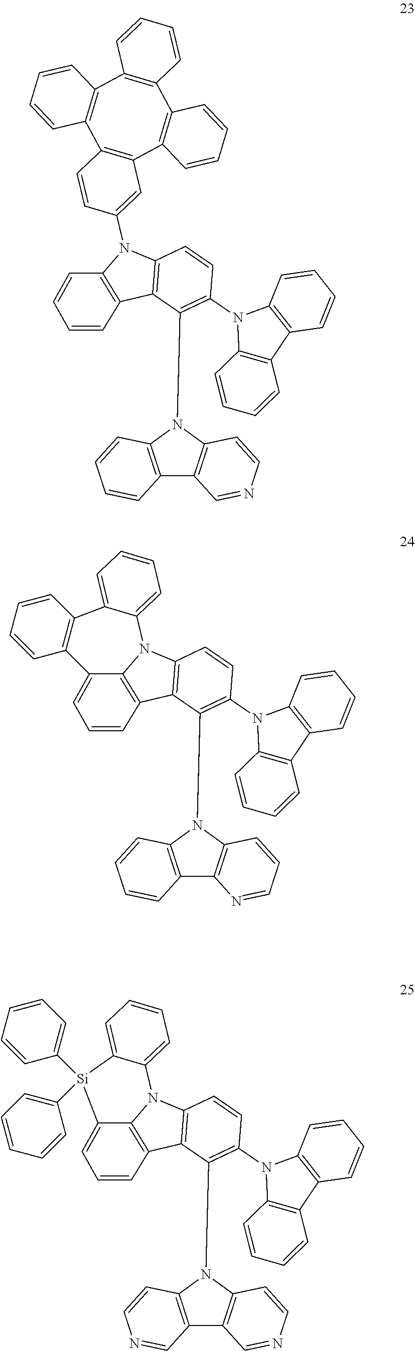 [SMILES: c1ccc2c(c1)-c1ccccc1-c1ccc(-n3c4ccccc4c4c(-n5c6ccccc6c6cnccc65)c(-n5c6ccccc6c6ccccc65)ccc43)cc1-c1ccccc1-2 ; c1ccc2c(c1)-c1ccccc1-n1c3ccc(-n4c5ccccc5c5ccccc54)c(-n4c5ccccc5c5ncccc54)c3c3cccc-2c31 ; c1ccc([Si]2(c3ccccc3)c3ccccc3-n3c4ccc(-n5c6ccccc6c6ccccc65)c(-n5c6ccncc6c6cnccc65)c4c4cccc2c43)cc1]

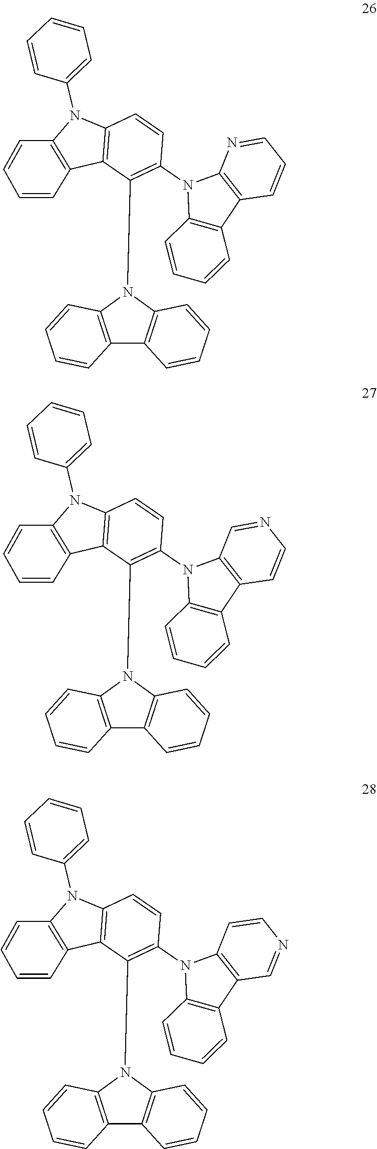

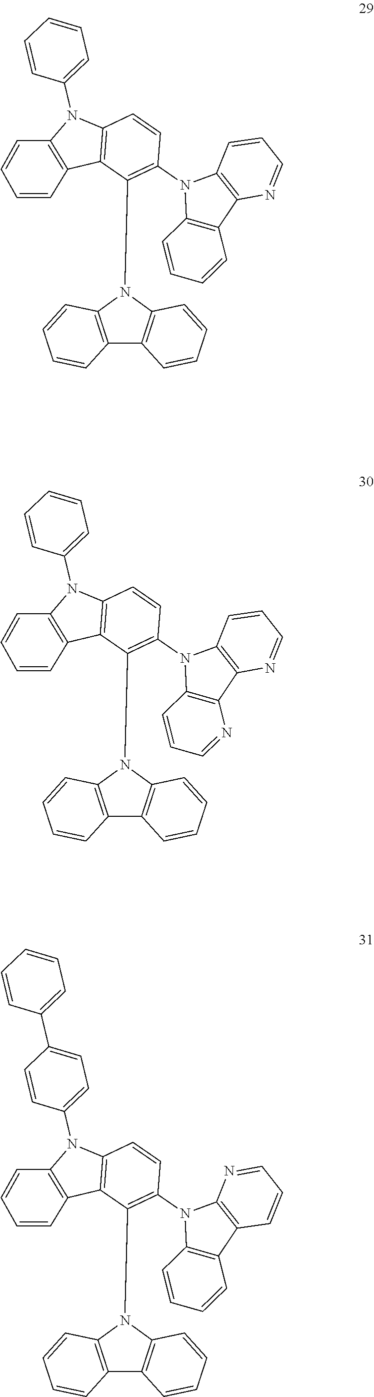 [SMILES: c1ccc(-n2c3ccccc3c3c(-n4c5ccccc5c5ccccc54)c(-n4c5ccccc5c5ncccc54)ccc32)cc1 ; c1ccc(-n2c3ccccc3c3c(-n4c5ccccc5c5ccccc54)c(-n4c5cccnc5c5ncccc54)ccc32)cc1 ; c1ccc(-c2ccc(-n3c4ccccc4c4c(-n5c6ccccc6c6ccccc65)c(-n5c6ccccc6c6cccnc65)ccc43)cc2)cc1]

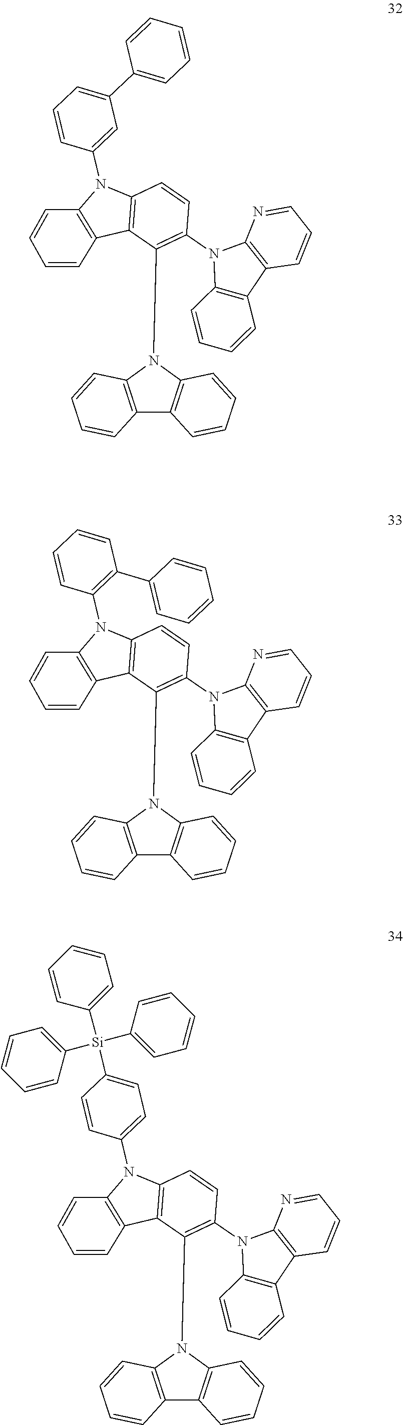 [SMILES: c1ccc(-c2cccc(-n3c4ccccc4c4c(-n5c6ccccc6c6ccccc65)c(-n5c6ccccc6c6cccnc65)ccc43)c2)cc1 ; c1ccc(-c2ccccc2-n2c3ccccc3c3c(-n4c5ccccc5c5ccccc54)c(-n4c5ccccc5c5cccnc54)ccc32)cc1 ; c1ccc([Si](c2ccccc2)(c2ccccc2)c2ccc(-n3c4ccccc4c4c(-n5c6ccccc6c6ccccc65)c(-n5c6ccccc6c6cccnc65)ccc43)cc2)cc1]

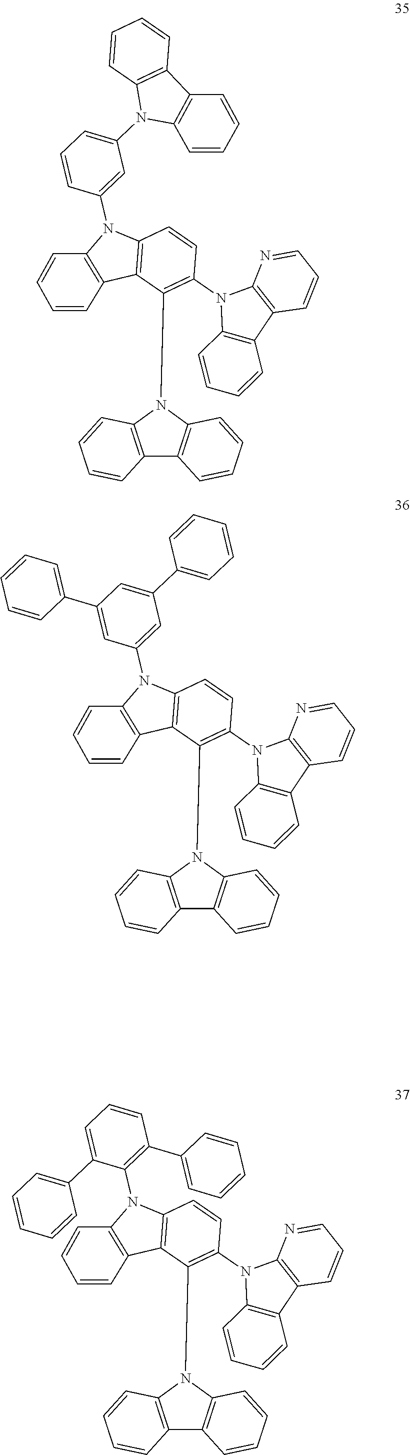

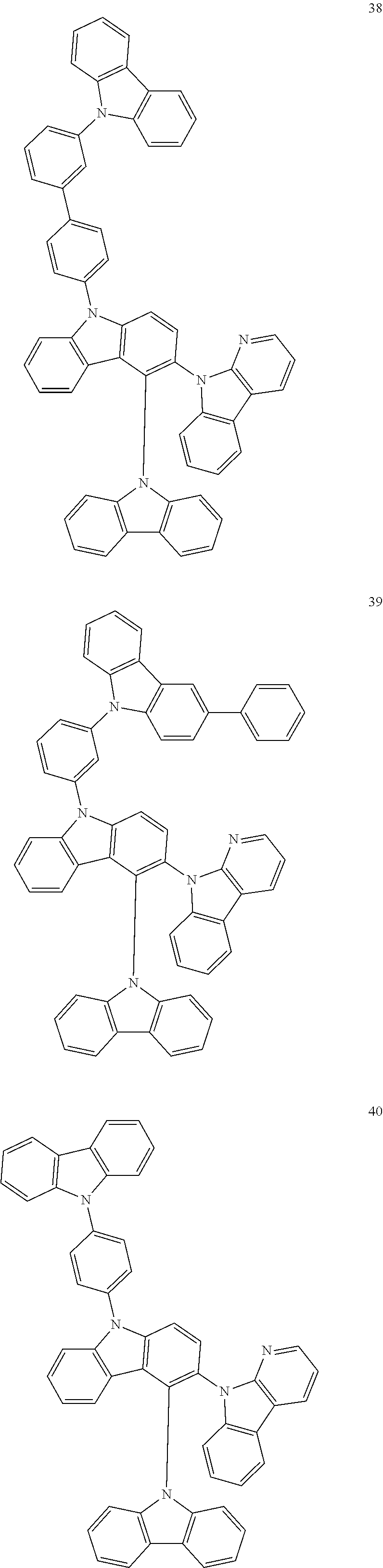 [SMILES: c1cc(-c2ccc(-n3c4ccccc4c4c(-n5c6ccccc6c6ccccc65)c(-n5c6ccccc6c6cccnc65)ccc43)cc2)cc(-n2c3ccccc3c3ccccc32)c1 ; c1ccc(-c2ccc3c(c2)c2ccccc2n3-c2cccc(-n3c4ccccc4c4c(-n5c6ccccc6c6ccccc65)c(-n5c6ccccc6c6cccnc65)ccc43)c2)cc1 ; c1ccc2c(c1)c1ccccc1n2-c1ccc(-n2c3ccccc3c3c(-n4c5ccccc5c5ccccc54)c(-n4c5ccccc5c5cccnc54)ccc32)cc1]

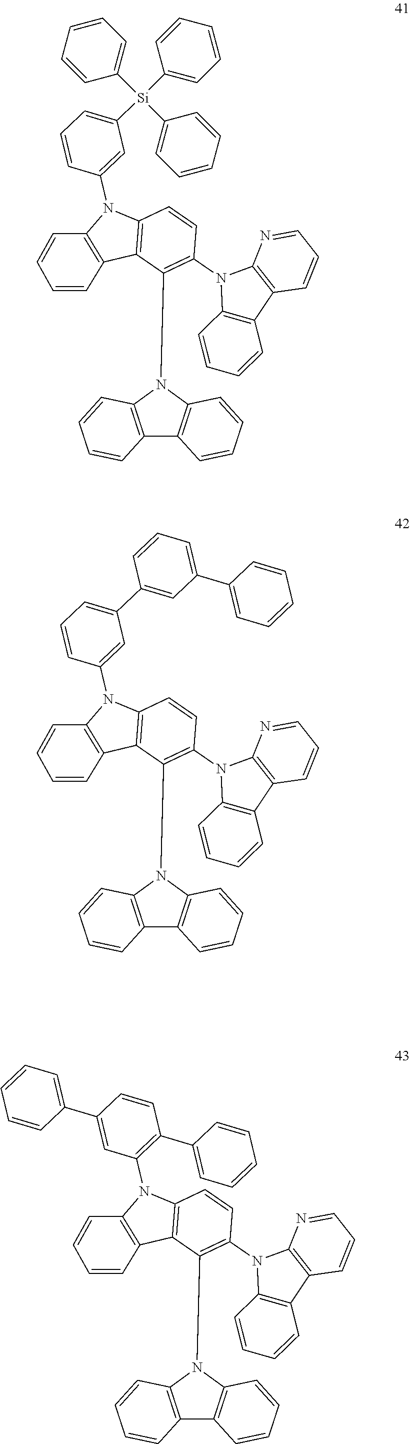 [SMILES: c1ccc([Si](c2ccccc2)(c2ccccc2)c2cccc(-n3c4ccccc4c4c(-n5c6ccccc6c6ccccc65)c(-n5c6ccccc6c6cccnc65)ccc43)c2)cc1 ; c1ccc(-c2cccc(-c3cccc(-n4c5ccccc5c5c(-n6c7ccccc7c7ccccc76)c(-n6c7ccccc7c7cccnc76)ccc54)c3)c2)cc1 ; c1ccc(-c2ccc(-c3ccccc3)c(-n3c4ccccc4c4c(-n5c6ccccc6c6ccccc65)c(-n5c6ccccc6c6cccnc65)ccc43)c2)cc1]

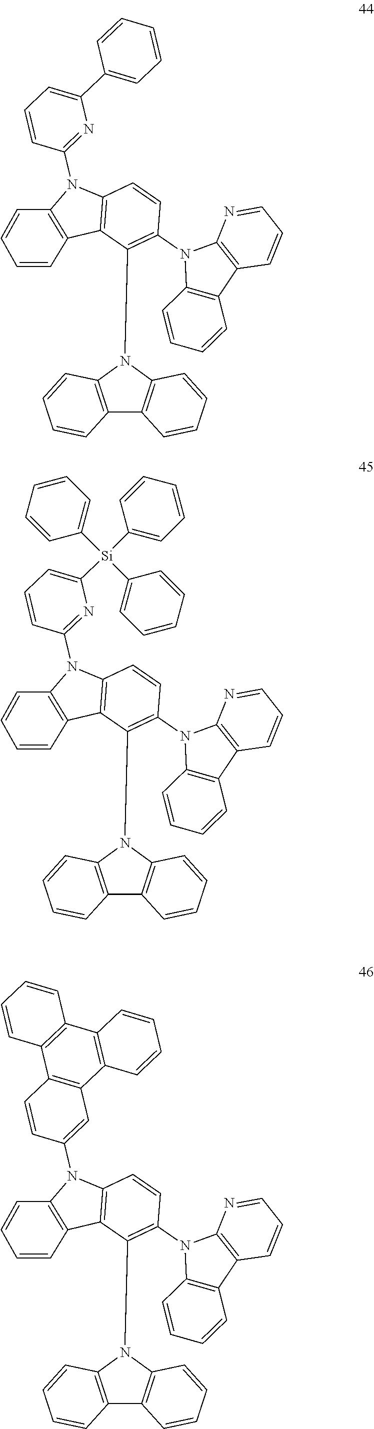

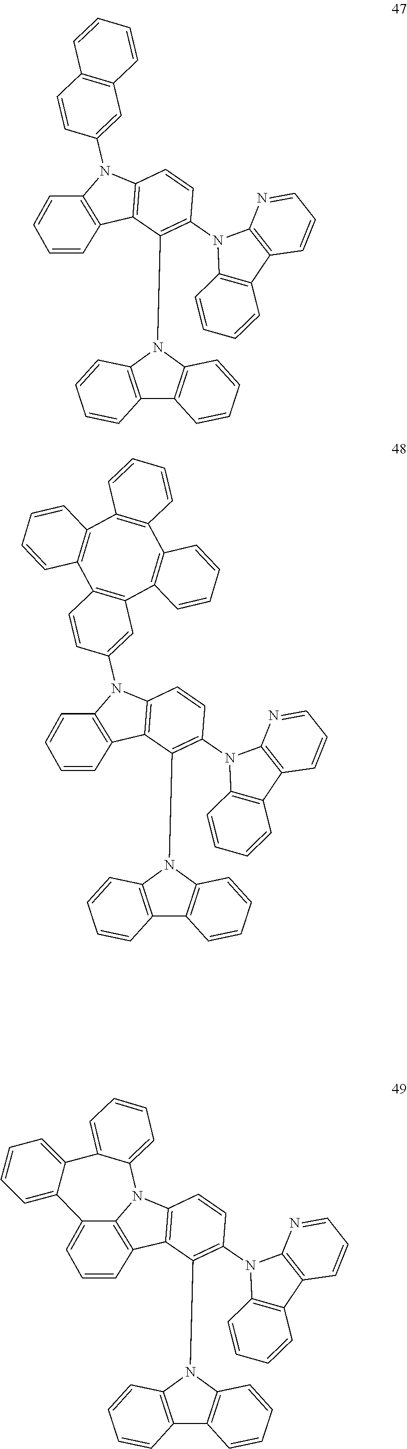

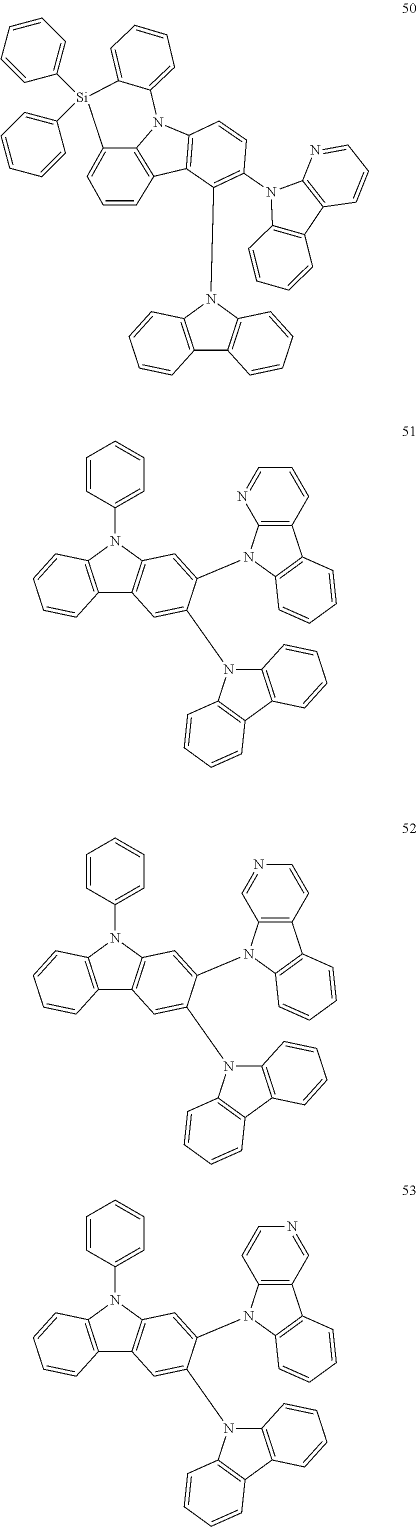 [SMILES: c1ccc([Si]2(c3ccccc3)c3ccccc3-n3c4ccc(-n5c6ccccc6c6cccnc65)c(-n5c6ccccc6c6ccccc65)c4c4cccc2c43)cc1 ; c1ccc(-n2c3ccccc3c3cc(-n4c5ccccc5c5ccccc54)c(-n4c5ccccc5c5cccnc54)cc32)cc1 ; c1ccc(-n2c3ccccc3c3cc(-n4c5ccccc5c5ccccc54)c(-n4c5ccccc5c5ccncc54)cc32)cc1 ; c1ccc(-n2c3ccccc3c3cc(-n4c5ccccc5c5ccccc54)c(-n4c5ccccc5c5cnccc54)cc32)cc1]

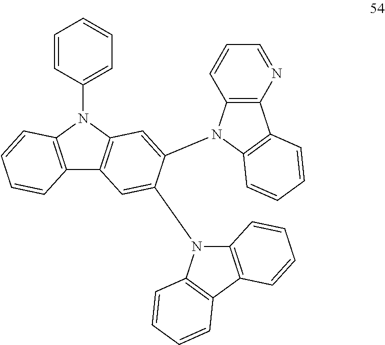 [SMILES: c1ccc(-n2c3ccccc3c3cc(-n4c5ccccc5c5ccccc54)c(-n4c5ccccc5c5ncccc54)cc32)cc1]

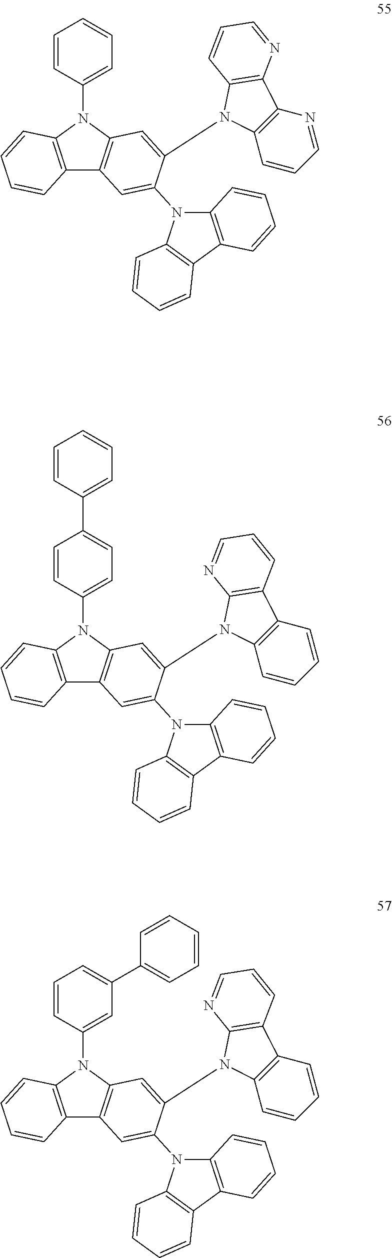 [SMILES: c1ccc(-n2c3ccccc3c3cc(-n4c5ccccc5c5ccccc54)c(-n4c5cccnc5c5ncccc54)cc32)cc1 ; c1ccc(-c2ccc(-n3c4ccccc4c4cc(-n5c6ccccc6c6ccccc65)c(-n5c6ccccc6c6cccnc65)cc43)cc2)cc1 ; c1ccc(-c2cccc(-n3c4ccccc4c4cc(-n5c6ccccc6c6ccccc65)c(-n5c6ccccc6c6cccnc65)cc43)c2)cc1]

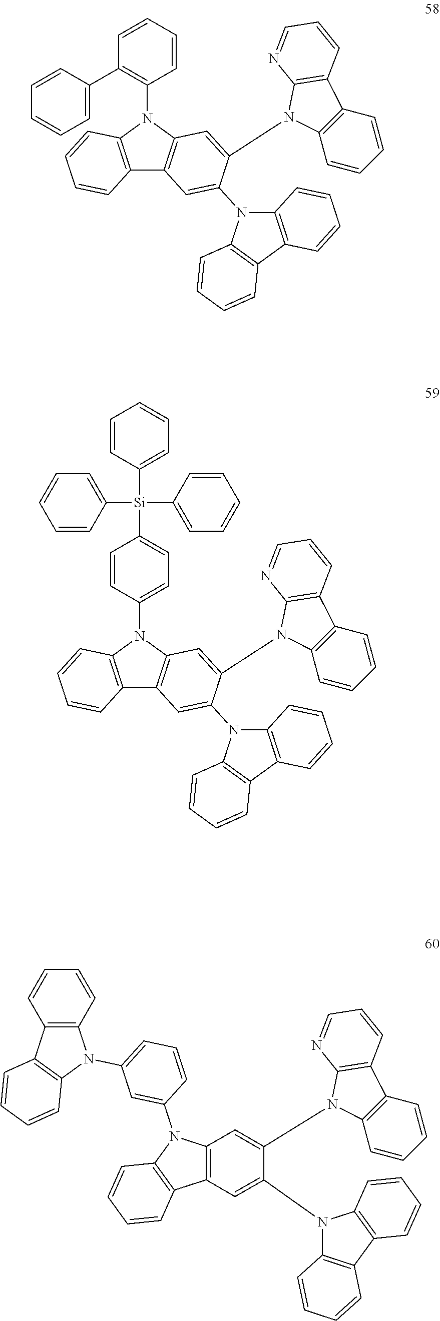 [SMILES: c1ccc(-c2ccccc2-n2c3ccccc3c3cc(-n4c5ccccc5c5ccccc54)c(-n4c5ccccc5c5cccnc54)cc32)cc1 ; c1ccc([Si](c2ccccc2)(c2ccccc2)c2ccc(-n3c4ccccc4c4cc(-n5c6ccccc6c6ccccc65)c(-n5c6ccccc6c6cccnc65)cc43)cc2)cc1 ; c1cc(-n2c3ccccc3c3ccccc32)cc(-n2c3ccccc3c3cc(-n4c5ccccc5c5ccccc54)c(-n4c5ccccc5c5cccnc54)cc32)c1]

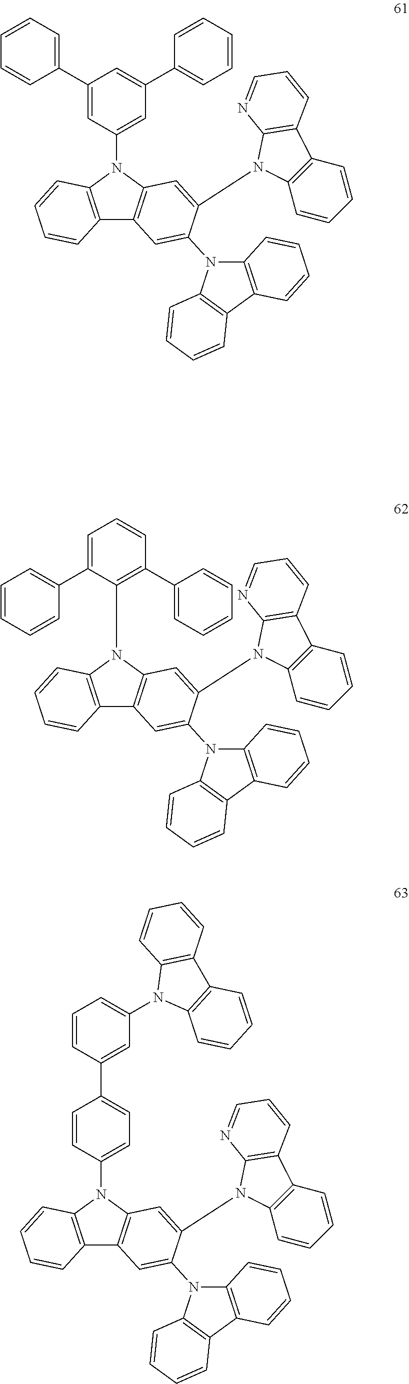 [SMILES: c1ccc(-c2cc(-c3ccccc3)cc(-n3c4ccccc4c4cc(-n5c6ccccc6c6ccccc65)c(-n5c6ccccc6c6cccnc65)cc43)c2)cc1 ; c1ccc(-c2cccc(-c3ccccc3)c2-n2c3ccccc3c3cc(-n4c5ccccc5c5ccccc54)c(-n4c5ccccc5c5cccnc54)cc32)cc1 ; c1cc(-c2ccc(-n3c4ccccc4c4cc(-n5c6ccccc6c6ccccc65)c(-n5c6ccccc6c6cccnc65)cc43)cc2)cc(-n2c3ccccc3c3ccccc32)c1]

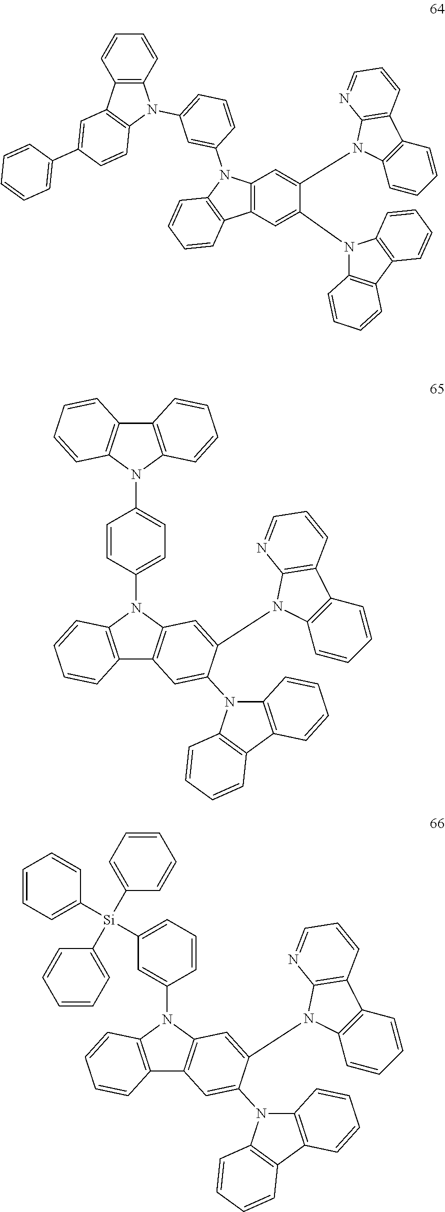 [SMILES: c1ccc(-c2ccc3c(c2)c2ccccc2n3-c2cccc(-n3c4ccccc4c4cc(-n5c6ccccc6c6ccccc65)c(-n5c6ccccc6c6cccnc65)cc43)c2)cc1 ; c1ccc2c(c1)c1ccccc1n2-c1ccc(-n2c3ccccc3c3cc(-n4c5ccccc5c5ccccc54)c(-n4c5ccccc5c5cccnc54)cc32)cc1 ; c1ccc([Si](c2ccccc2)(c2ccccc2)c2cccc(-n3c4ccccc4c4cc(-n5c6ccccc6c6ccccc65)c(-n5c6ccccc6c6cccnc65)cc43)c2)cc1]

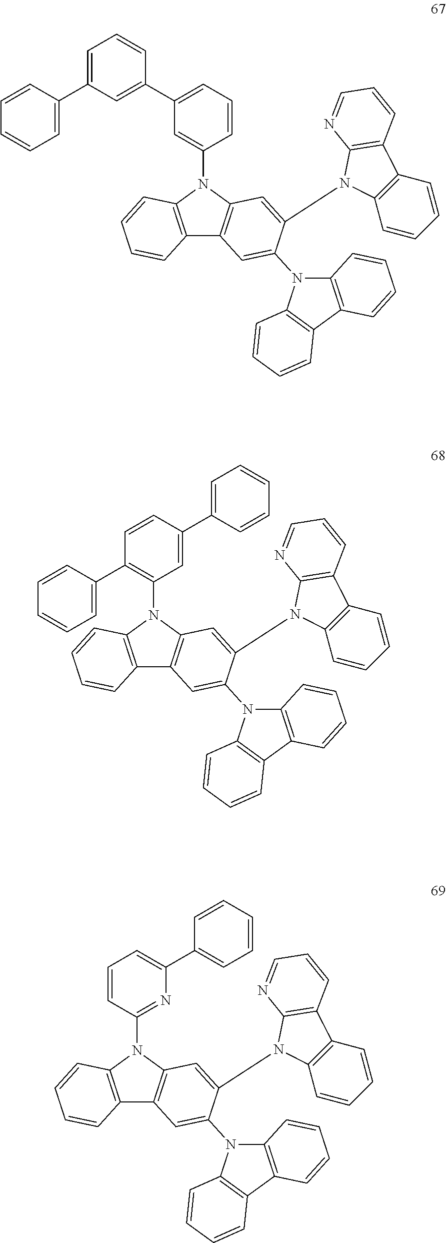 [SMILES: c1ccc(-c2cccc(-c3cccc(-n4c5ccccc5c5cc(-n6c7ccccc7c7ccccc76)c(-n6c7ccccc7c7cccnc76)cc54)c3)c2)cc1 ; c1ccc(-c2ccc(-c3ccccc3)c(-n3c4ccccc4c4cc(-n5c6ccccc6c6ccccc65)c(-n5c6ccccc6c6cccnc65)cc43)c2)cc1 ; c1ccc(-c2cccc(-n3c4ccccc4c4cc(-n5c6ccccc6c6ccccc65)c(-n5c6ccccc6c6cccnc65)cc43)n2)cc1]

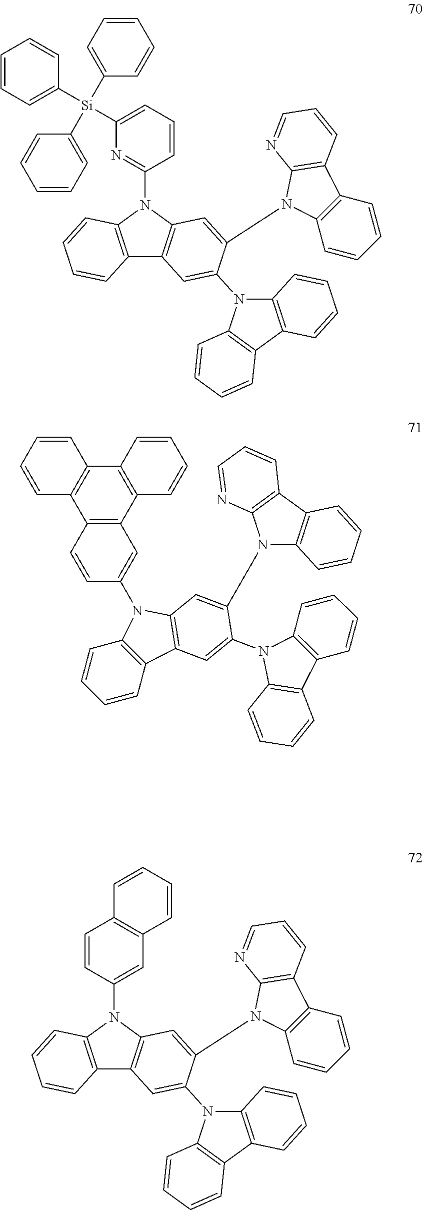 [SMILES: c1ccc([Si](c2ccccc2)(c2ccccc2)c2cccc(-n3c4ccccc4c4cc(-n5c6ccccc6c6ccccc65)c(-n5c6ccccc6c6cccnc65)cc43)n2)cc1 ; c1ccc2c(c1)c1ccccc1c1cc(-n3c4ccccc4c4cc(-n5c6ccccc6c6ccccc65)c(-n5c6ccccc6c6cccnc65)cc43)ccc21 ; c1ccc2cc(-n3c4ccccc4c4cc(-n5c6ccccc6c6ccccc65)c(-n5c6ccccc6c6cccnc65)cc43)ccc2c1]

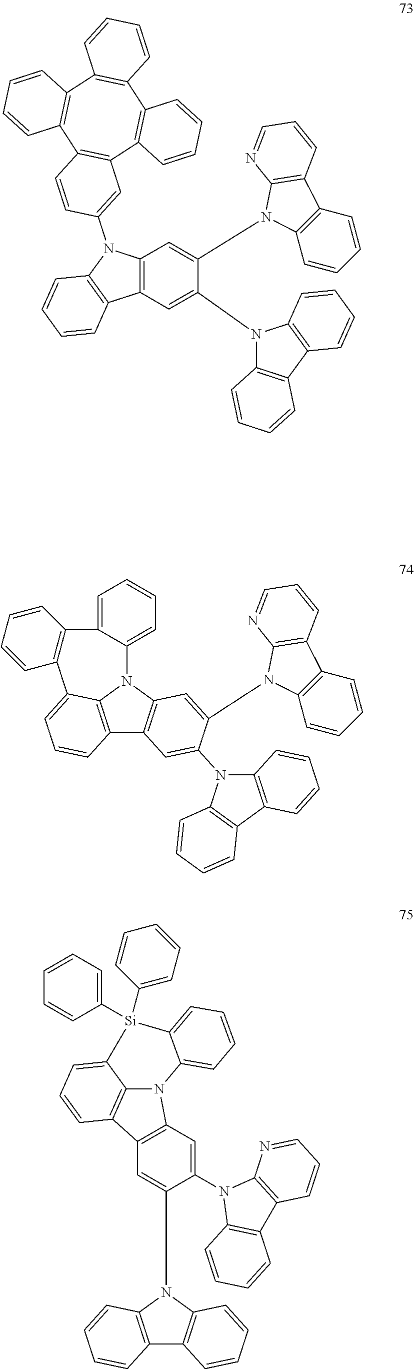 [SMILES: c1ccc2c(c1)-c1ccccc1-c1ccc(-n3c4ccccc4c4cc(-n5c6ccccc6c6ccccc65)c(-n5c6ccccc6c6cccnc65)cc43)cc1-c1ccccc1-2 ; c1ccc2c(c1)-c1ccccc1-n1c3cc(-n4c5ccccc5c5cccnc54)c(-n4c5ccccc5c5ccccc54)cc3c3cccc-2c31 ; c1ccc([Si]2(c3ccccc3)c3ccccc3-n3c4cc(-n5c6ccccc6c6cccnc65)c(-n5c6ccccc6c6ccccc65)cc4c4cccc2c43)cc1]

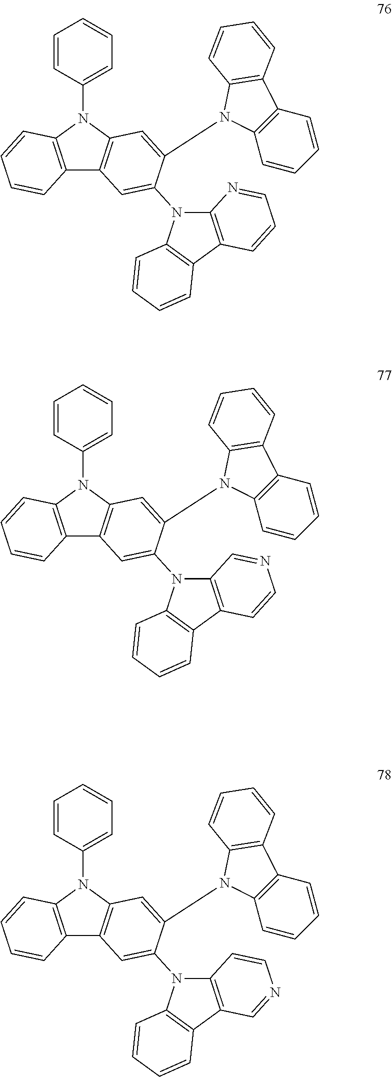 [SMILES: c1ccc(-n2c3ccccc3c3cc(-n4c5ccccc5c5cccnc54)c(-n4c5ccccc5c5ccccc54)cc32)cc1 ; c1ccc(-n2c3ccccc3c3cc(-n4c5ccccc5c5ccncc54)c(-n4c5ccccc5c5ccccc54)cc32)cc1 ; c1ccc(-n2c3ccccc3c3cc(-n4c5ccccc5c5cnccc54)c(-n4c5ccccc5c5ccccc54)cc32)cc1]

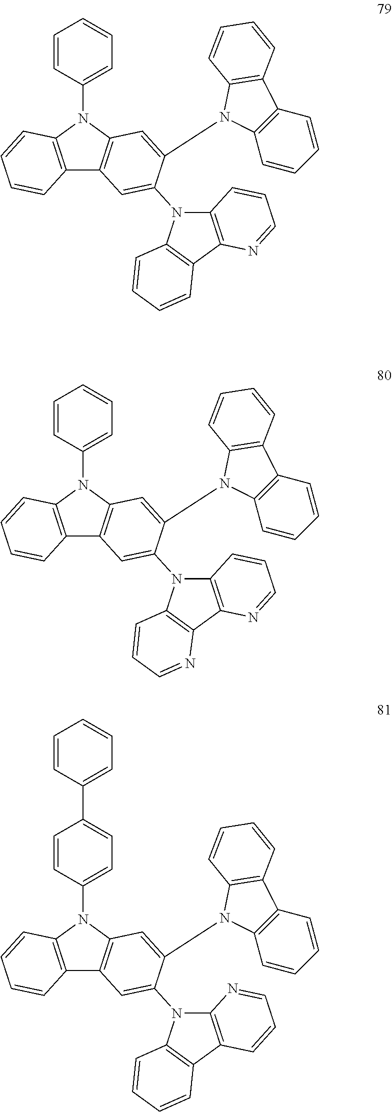 [SMILES: c1ccc(-n2c3ccccc3c3cc(-n4c5ccccc5c5ncccc54)c(-n4c5ccccc5c5ccccc54)cc32)cc1 ; c1ccc(-n2c3ccccc3c3cc(-n4c5cccnc5c5ncccc54)c(-n4c5ccccc5c5ccccc54)cc32)cc1 ; c1ccc(-c2ccc(-n3c4ccccc4c4cc(-n5c6ccccc6c6cccnc65)c(-n5c6ccccc6c6ccccc65)cc43)cc2)cc1]

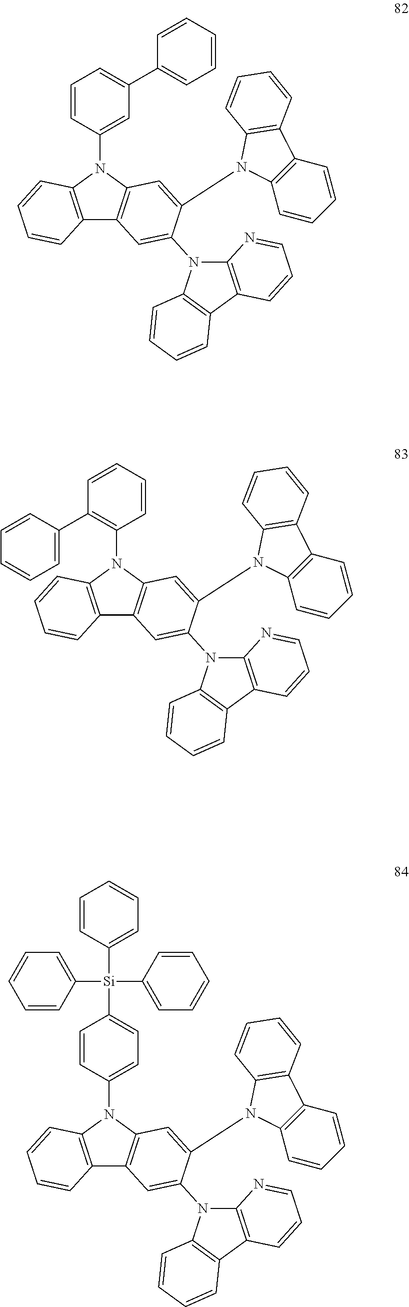 [SMILES: c1ccc(-c2cccc(-n3c4ccccc4c4cc(-n5c6ccccc6c6cccnc65)c(-n5c6ccccc6c6ccccc65)cc43)c2)cc1 ; c1ccc(-c2ccccc2-n2c3ccccc3c3cc(-n4c5ccccc5c5cccnc54)c(-n4c5ccccc5c5ccccc54)cc32)cc1 ; c1ccc([Si](c2ccccc2)(c2ccccc2)c2ccc(-n3c4ccccc4c4cc(-n5c6ccccc6c6cccnc65)c(-n5c6ccccc6c6ccccc65)cc43)cc2)cc1]

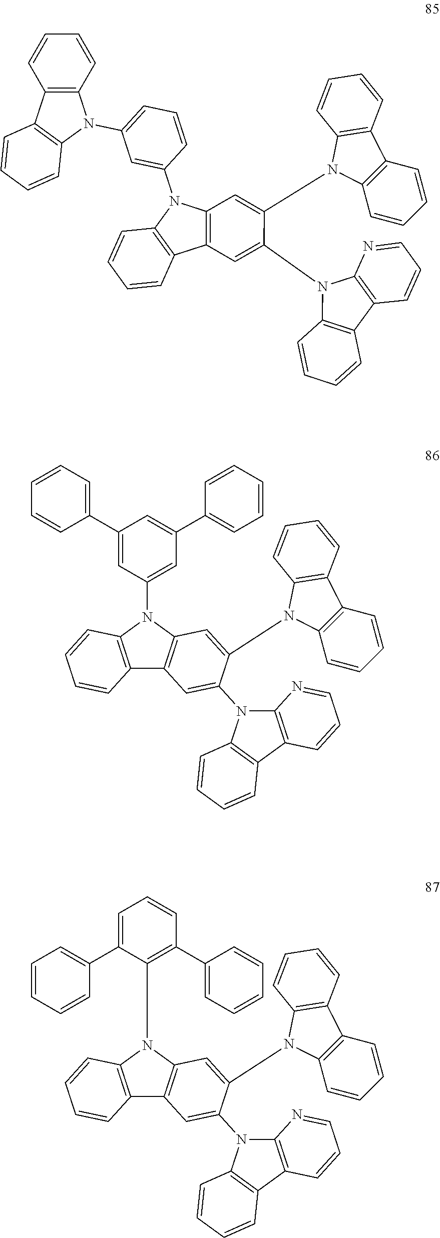 [SMILES: c1cc(-n2c3ccccc3c3ccccc32)cc(-n2c3ccccc3c3cc(-n4c5ccccc5c5cccnc54)c(-n4c5ccccc5c5ccccc54)cc32)c1 ; c1ccc(-c2cc(-c3ccccc3)cc(-n3c4ccccc4c4cc(-n5c6ccccc6c6cccnc65)c(-n5c6ccccc6c6ccccc65)cc43)c2)cc1 ; c1ccc(-c2cccc(-c3ccccc3)c2-n2c3ccccc3c3cc(-n4c5ccccc5c5cccnc54)c(-n4c5ccccc5c5ccccc54)cc32)cc1]

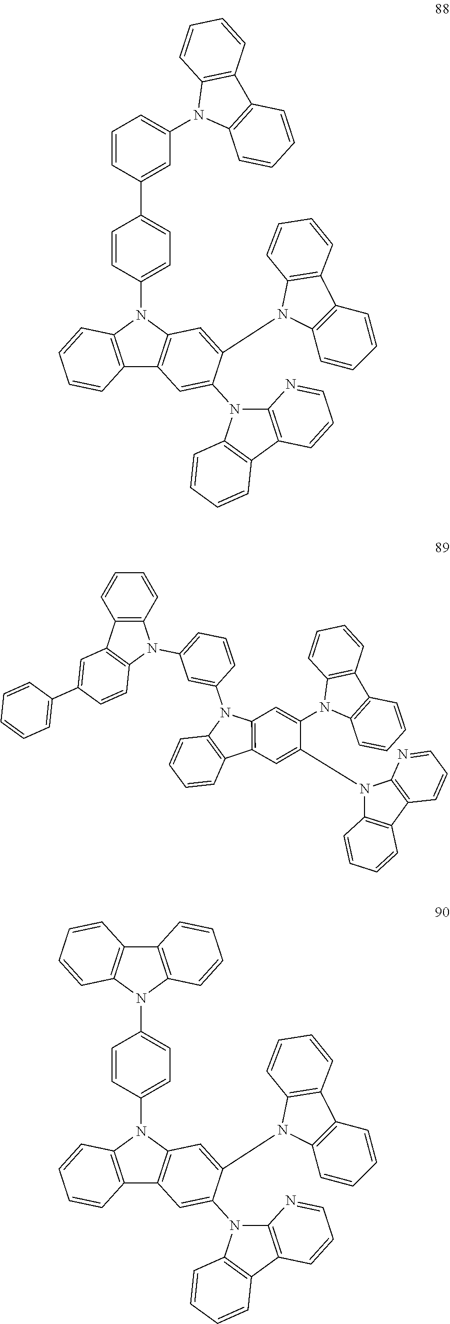 [SMILES: c1cc(-c2ccc(-n3c4ccccc4c4cc(-n5c6ccccc6c6cccnc65)c(-n5c6ccccc6c6ccccc65)cc43)cc2)cc(-n2c3ccccc3c3ccccc32)c1 ; c1ccc(-c2ccc3c(c2)c2ccccc2n3-c2cccc(-n3c4ccccc4c4cc(-n5c6ccccc6c6cccnc65)c(-n5c6ccccc6c6ccccc65)cc43)c2)cc1 ; c1ccc2c(c1)c1ccccc1n2-c1ccc(-n2c3ccccc3c3cc(-n4c5ccccc5c5cccnc54)c(-n4c5ccccc5c5ccccc54)cc32)cc1]

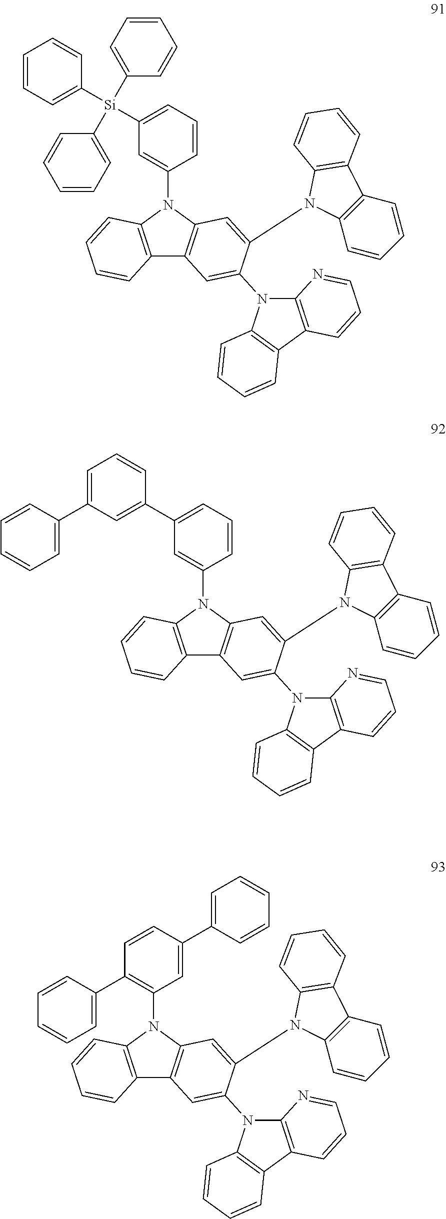 [SMILES: c1ccc([Si](c2ccccc2)(c2ccccc2)c2cccc(-n3c4ccccc4c4cc(-n5c6ccccc6c6cccnc65)c(-n5c6ccccc6c6ccccc65)cc43)c2)cc1 ; c1ccc(-c2cccc(-c3cccc(-n4c5ccccc5c5cc(-n6c7ccccc7c7cccnc76)c(-n6c7ccccc7c7ccccc76)cc54)c3)c2)cc1 ; c1ccc(-c2ccc(-c3ccccc3)c(-n3c4ccccc4c4cc(-n5c6ccccc6c6cccnc65)c(-n5c6ccccc6c6ccccc65)cc43)c2)cc1]

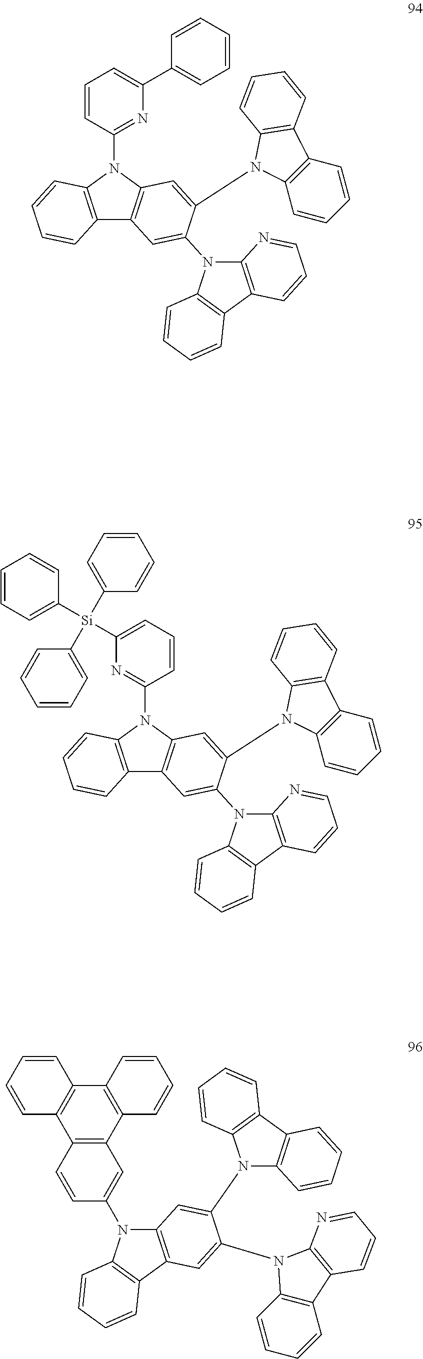 [SMILES: c1ccc(-c2cccc(-n3c4ccccc4c4cc(-n5c6ccccc6c6cccnc65)c(-n5c6ccccc6c6ccccc65)cc43)n2)cc1 ; c1ccc([Si](c2ccccc2)(c2ccccc2)c2cccc(-n3c4ccccc4c4cc(-n5c6ccccc6c6cccnc65)c(-n5c6ccccc6c6ccccc65)cc43)n2)cc1 ; c1ccc2c(c1)c1ccccc1c1cc(-n3c4ccccc4c4cc(-n5c6ccccc6c6cccnc65)c(-n5c6ccccc6c6ccccc65)cc43)ccc21]

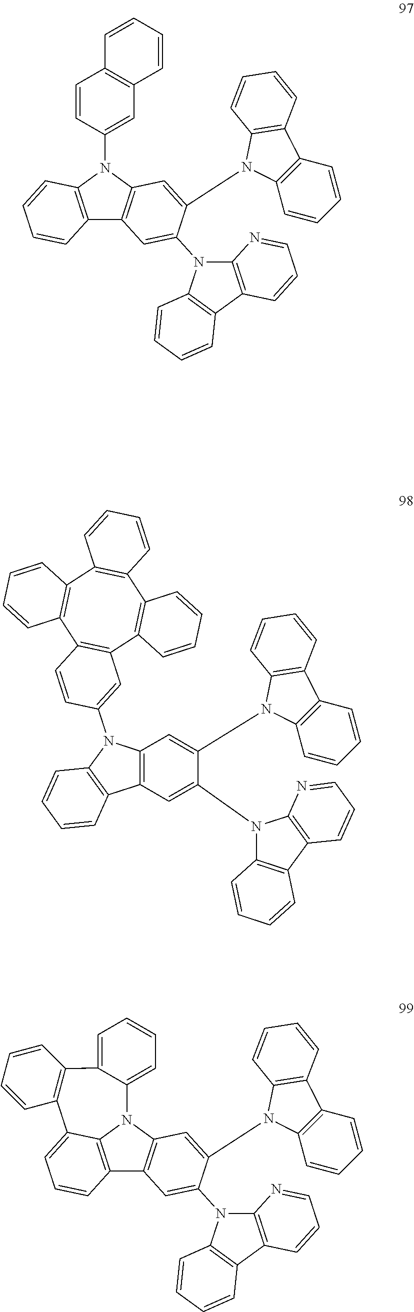 [SMILES: c1ccc2cc(-n3c4ccccc4c4cc(-n5c6ccccc6c6cccnc65)c(-n5c6ccccc6c6ccccc65)cc43)ccc2c1 ; c1ccc2c(c1)-c1ccccc1-c1ccc(-n3c4ccccc4c4cc(-n5c6ccccc6c6cccnc65)c(-n5c6ccccc6c6ccccc65)cc43)cc1-c1ccccc1-2 ; c1ccc2c(c1)-c1ccccc1-n1c3cc(-n4c5ccccc5c5ccccc54)c(-n4c5ccccc5c5cccnc54)cc3c3cccc-2c31]

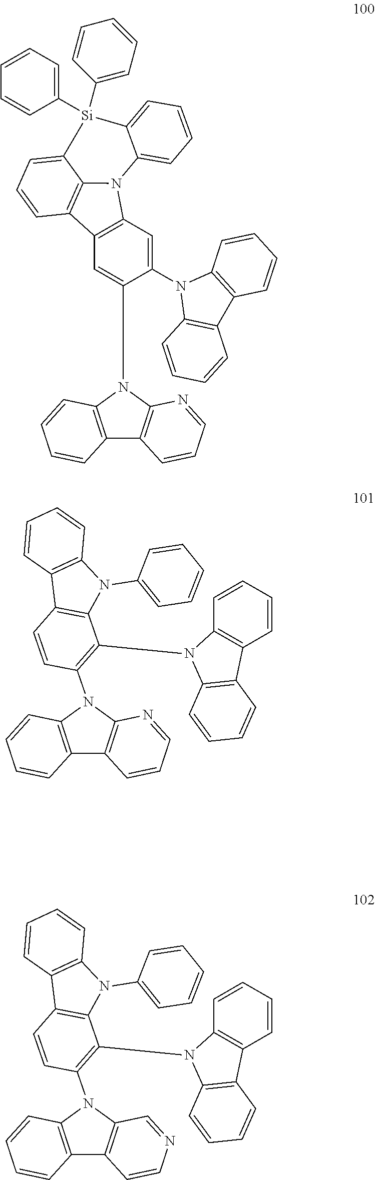 [SMILES: c1ccc([Si]2(c3ccccc3)c3ccccc3-n3c4cc(-n5c6ccccc6c6ccccc65)c(-n5c6ccccc6c6cccnc65)cc4c4cccc2c43)cc1 ; c1ccc(-n2c3ccccc3c3ccc(-n4c5ccccc5c5cccnc54)c(-n4c5ccccc5c5ccccc54)c32)cc1 ; c1ccc(-n2c3ccccc3c3ccc(-n4c5ccccc5c5ccncc54)c(-n4c5ccccc5c5ccccc54)c32)cc1]

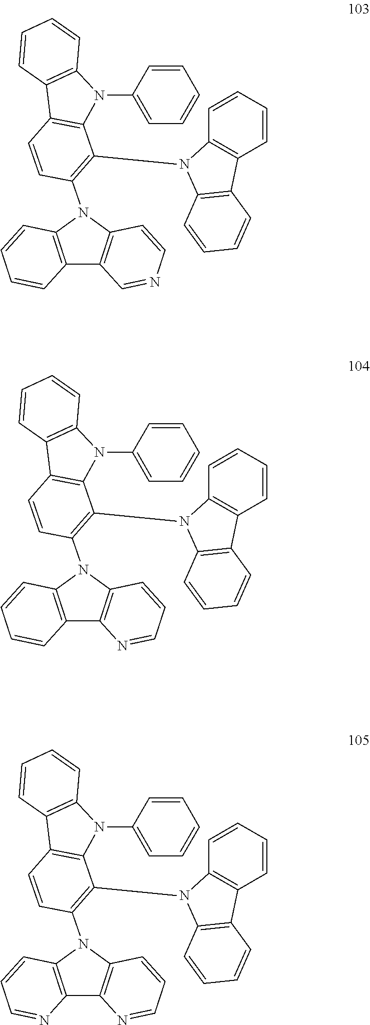 [SMILES: c1ccc(-n2c3ccccc3c3ccc(-n4c5ccccc5c5cnccc54)c(-n4c5ccccc5c5ccccc54)c32)cc1 ; c1ccc(-n2c3ccccc3c3ccc(-n4c5ccccc5c5ncccc54)c(-n4c5ccccc5c5ccccc54)c32)cc1 ; c1ccc(-n2c3ccccc3c3ccc(-n4c5cccnc5c5ncccc54)c(-n4c5ccccc5c5ccccc54)c32)cc1]

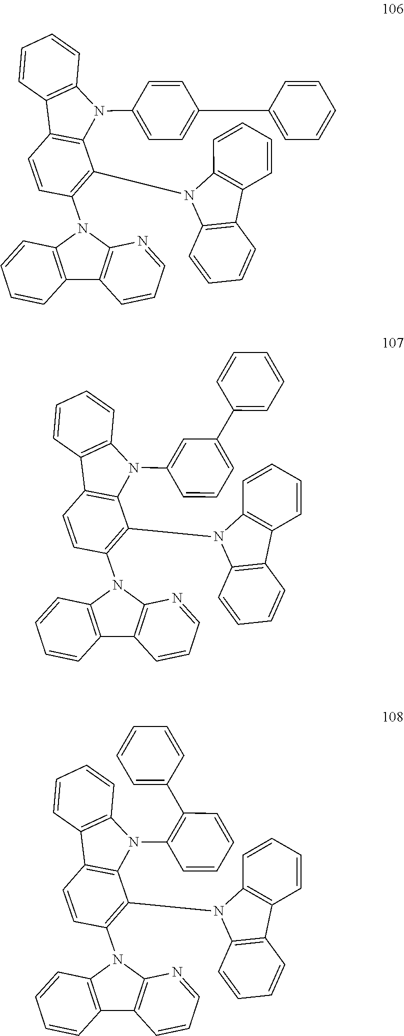 [SMILES: c1ccc(-c2ccc(-n3c4ccccc4c4ccc(-n5c6ccccc6c6cccnc65)c(-n5c6ccccc6c6ccccc65)c43)cc2)cc1 ; c1ccc(-c2cccc(-n3c4ccccc4c4ccc(-n5c6ccccc6c6cccnc65)c(-n5c6ccccc6c6ccccc65)c43)c2)cc1 ; c1ccc(-c2ccccc2-n2c3ccccc3c3ccc(-n4c5ccccc5c5cccnc54)c(-n4c5ccccc5c5ccccc54)c32)cc1]

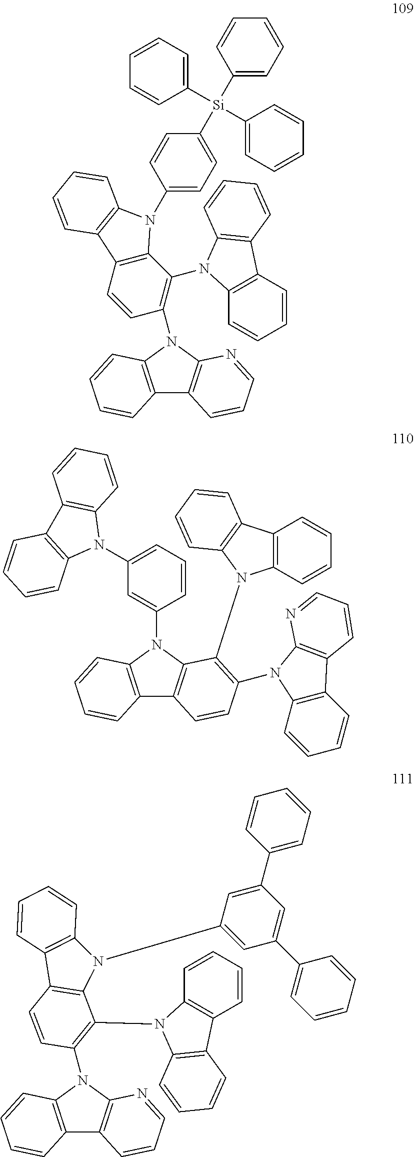

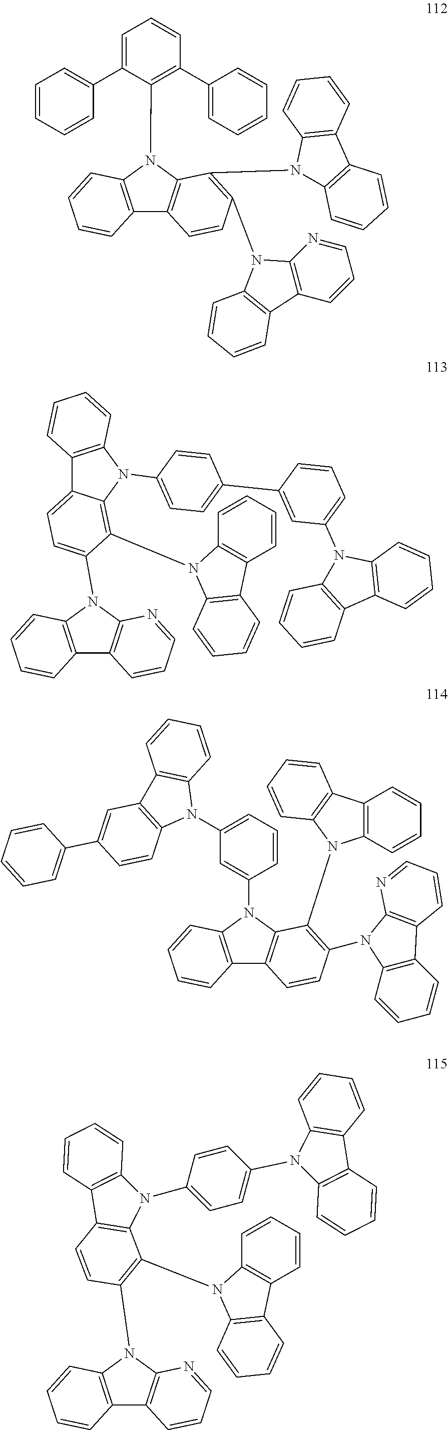

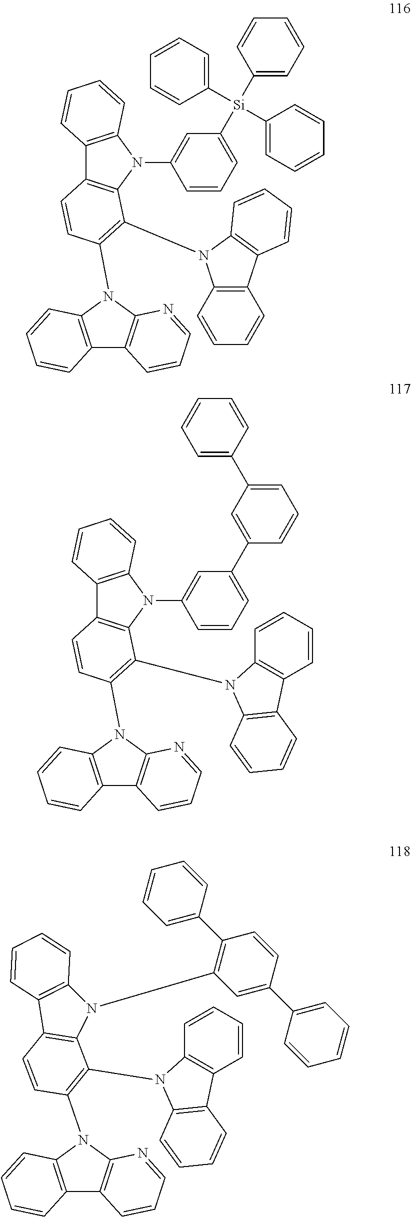

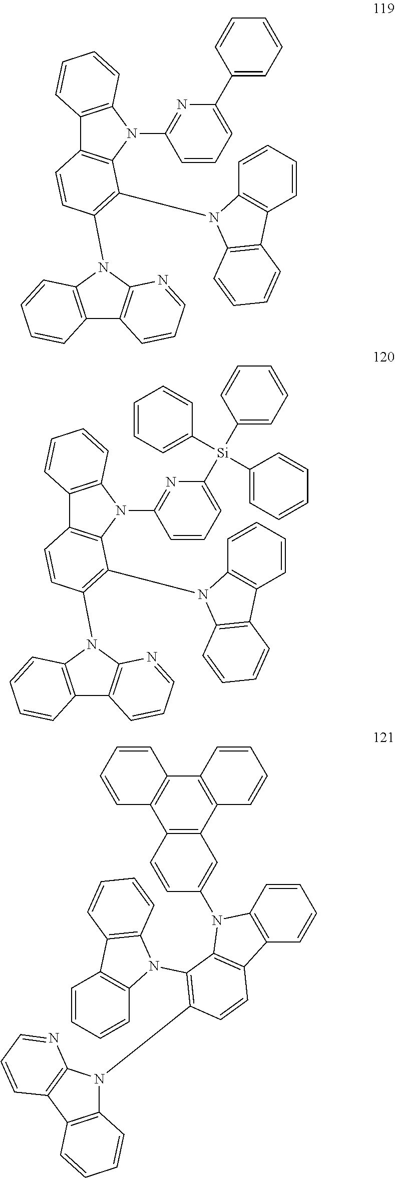

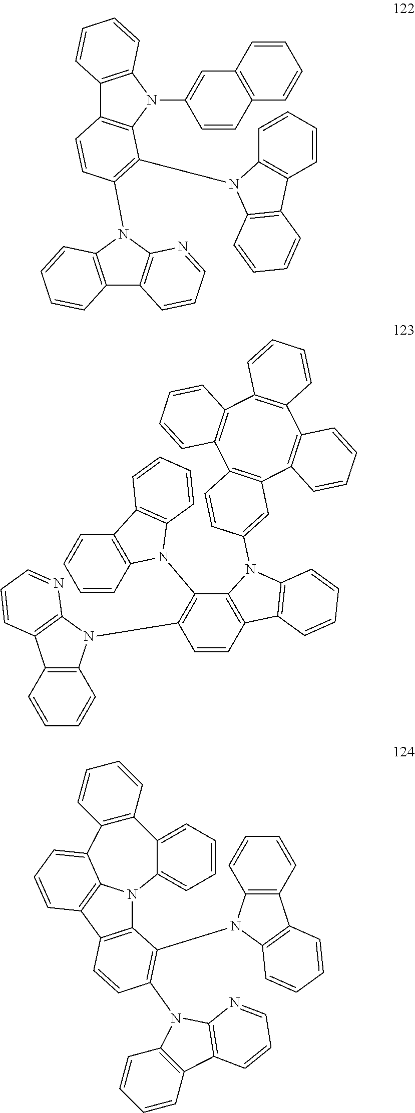

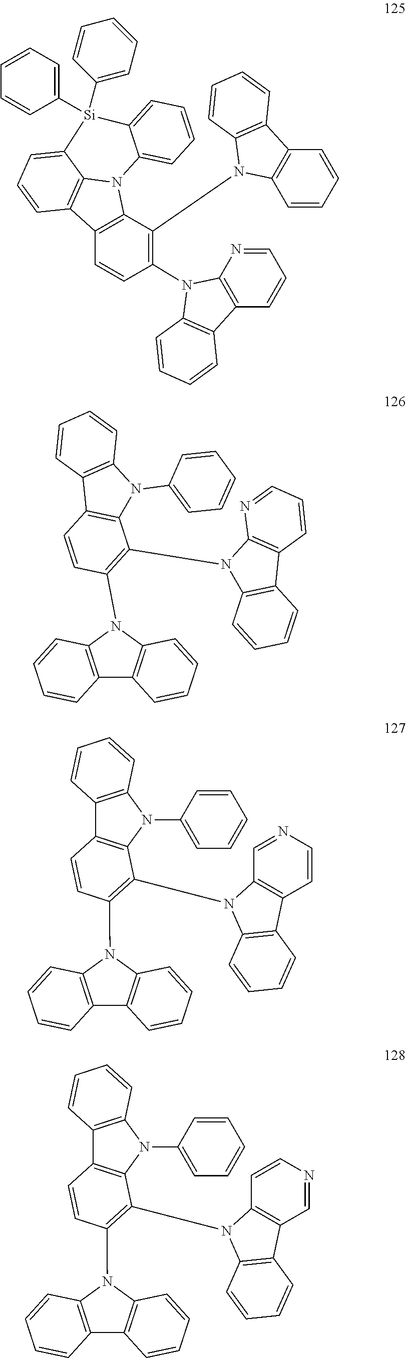

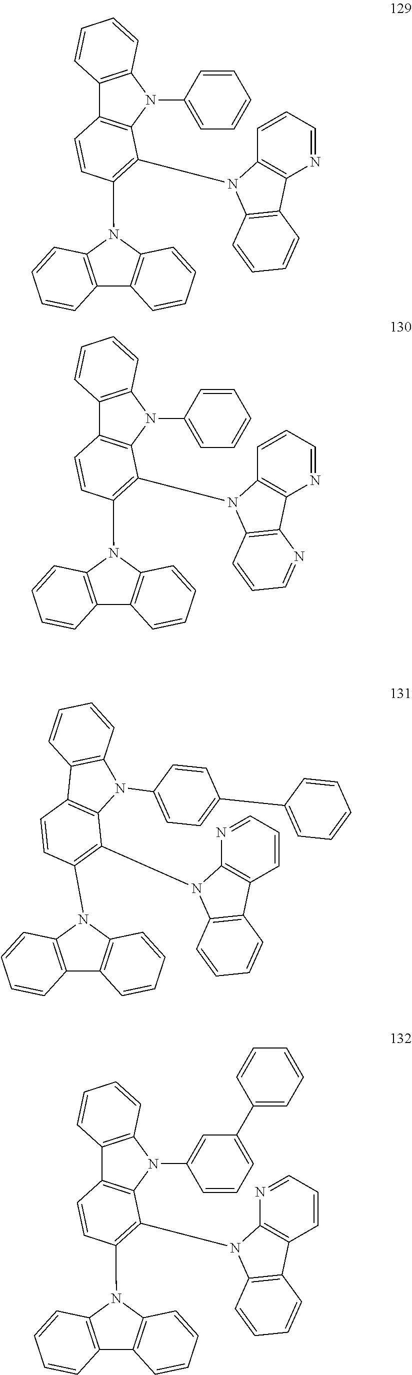

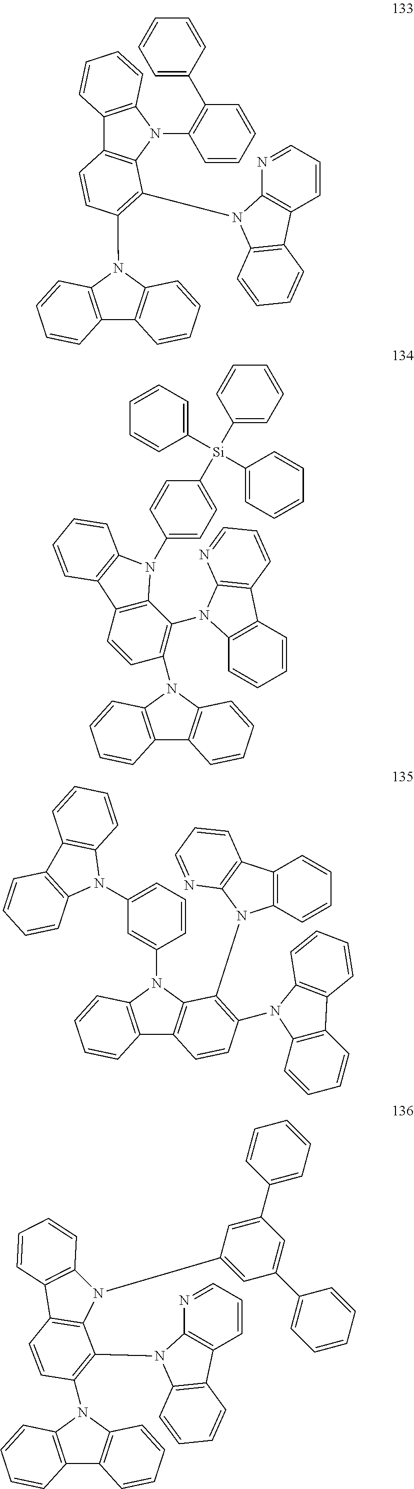

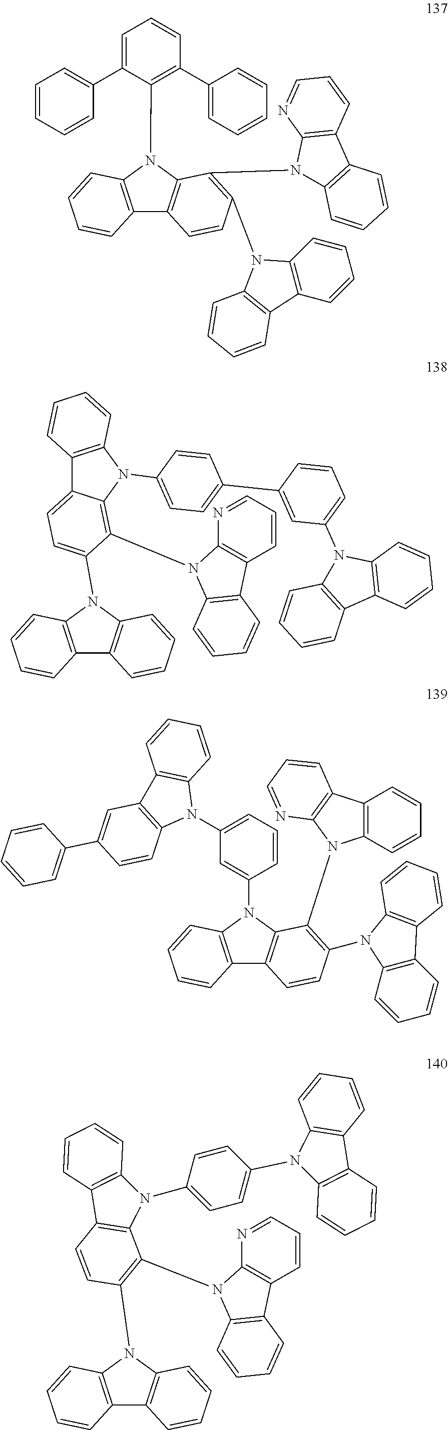

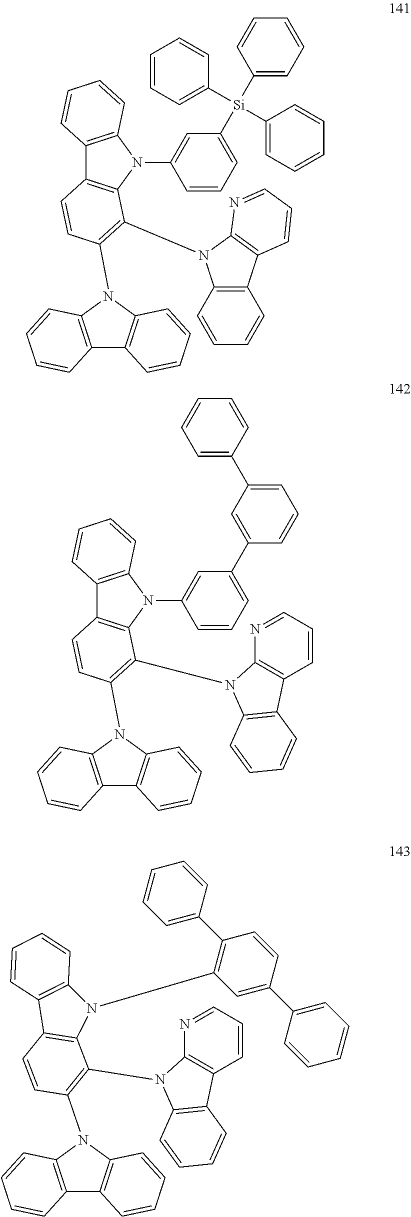

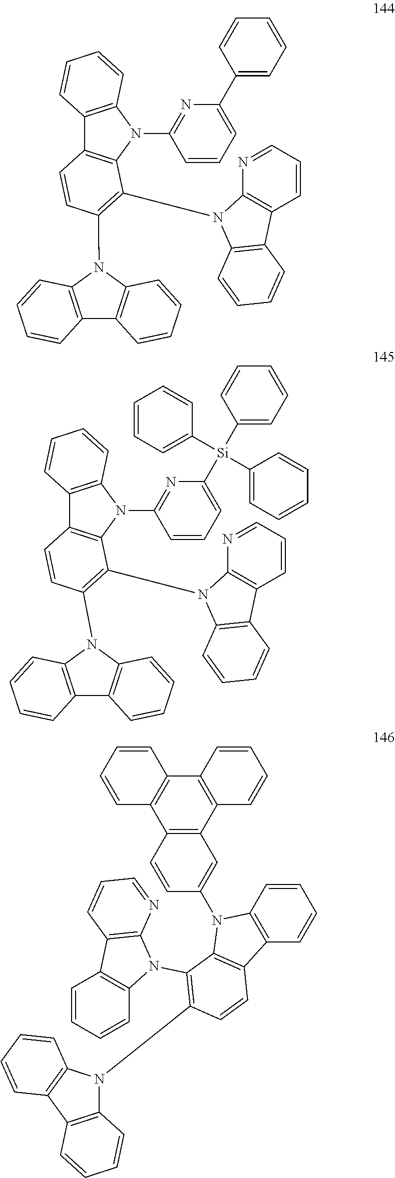

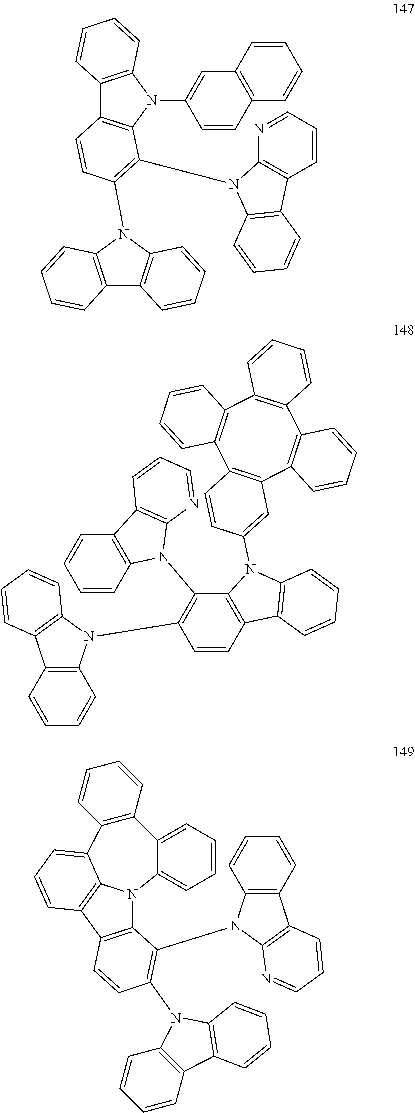

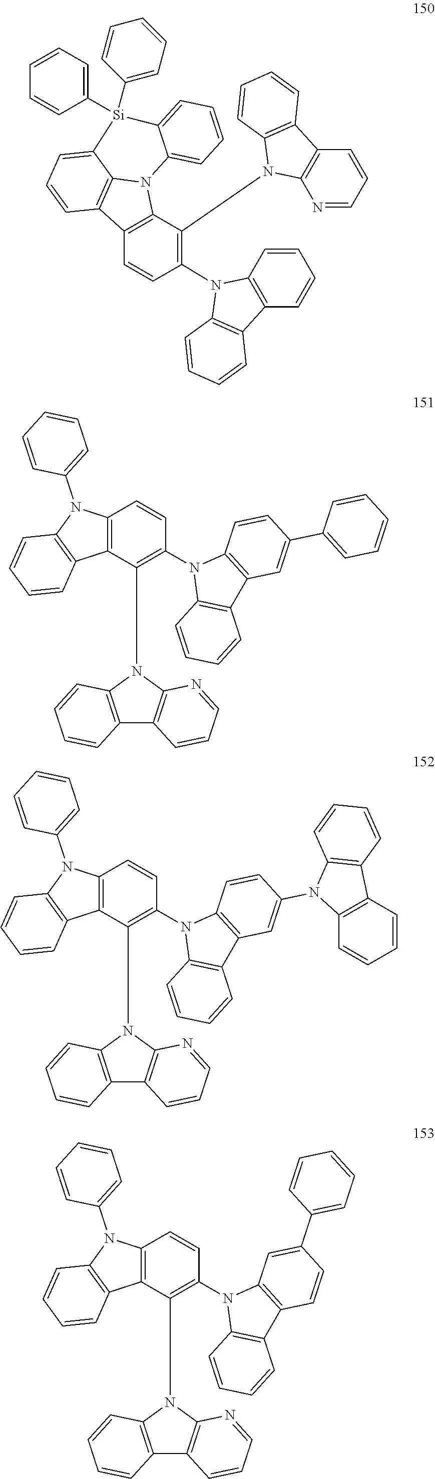

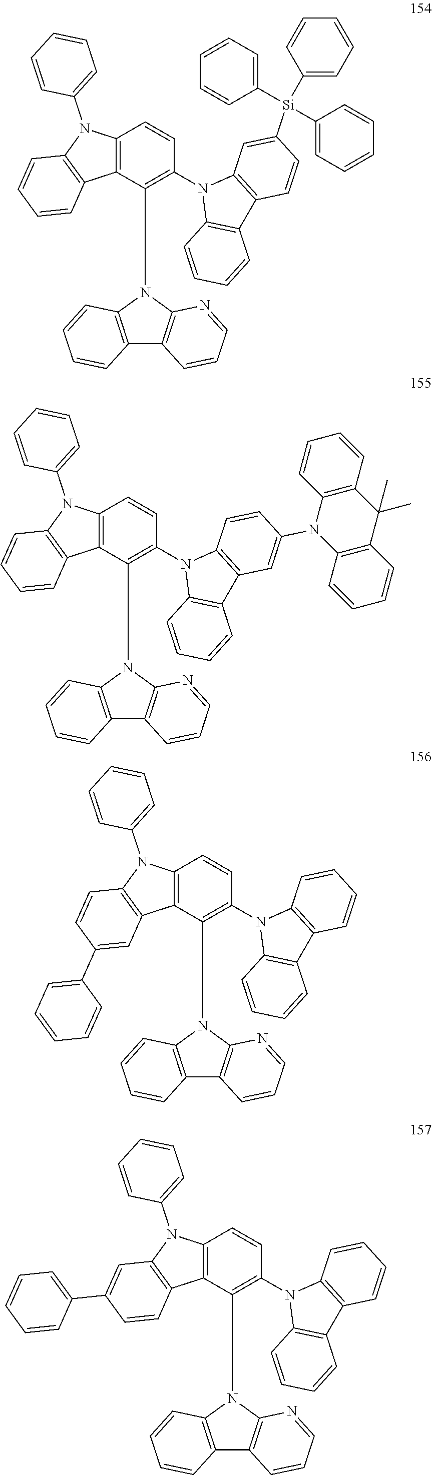

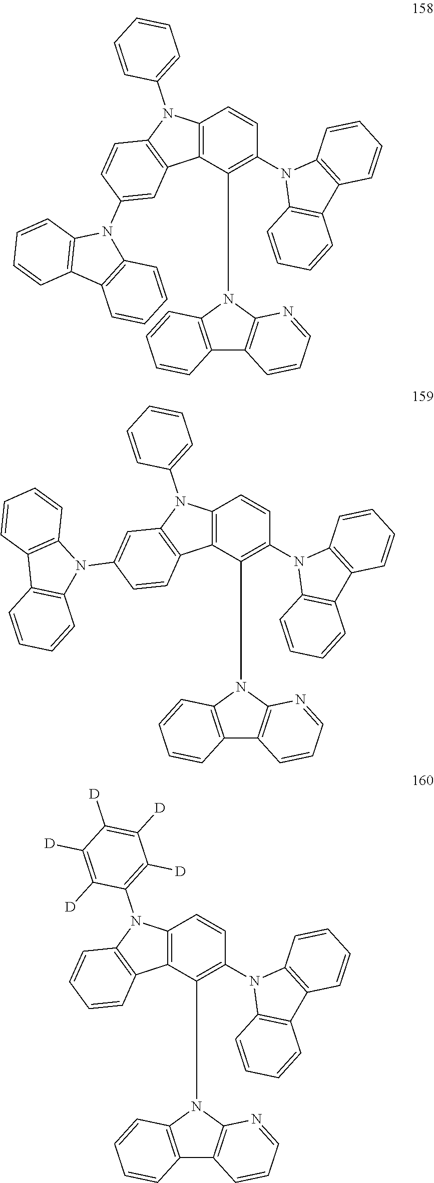

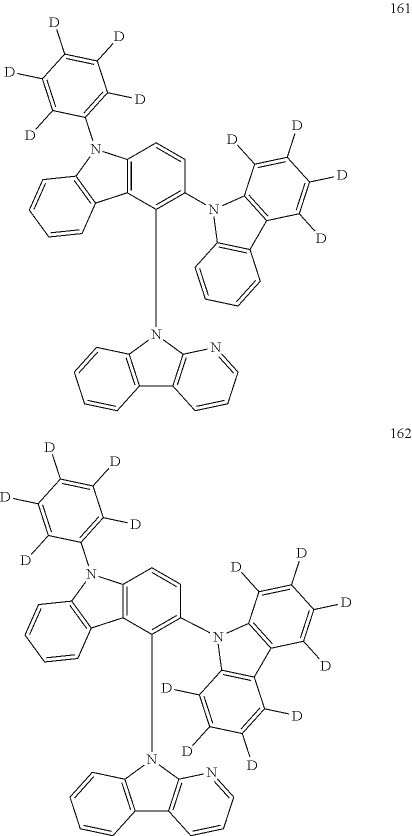 [SMILES: [2H]c1c([2H])c([2H])c(-n2c3ccccc3c3c(-n4c5ccccc5c5cccnc54)c(-n4c5ccccc5c5c([2H])c([2H])c([2H])c([2H])c54)ccc32)c([2H])c1[2H] ; [2H]c1c([2H])c([2H])c(-n2c3ccccc3c3c(-n4c5ccccc5c5cccnc54)c(-n4c5c([2H])c([2H])c([2H])c([2H])c5c5c([2H])c([2H])c([2H])c([2H])c54)ccc32)c([2H])c1[2H]]

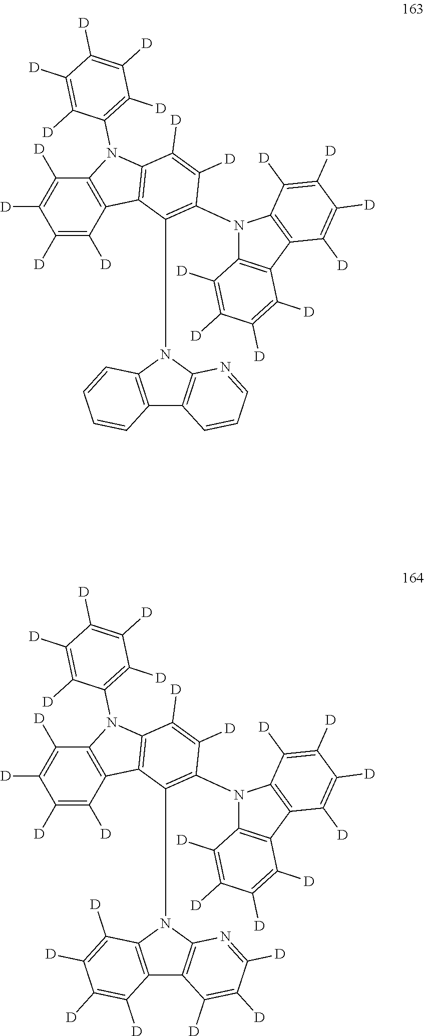 [SMILES: [2H]c1c([2H])c([2H])c(-n2c3c([2H])c([2H])c([2H])c([2H])c3c3c(-n4c5ccccc5c5cccnc54)c(-n4c5c([2H])c([2H])c([2H])c([2H])c5c5c([2H])c([2H])c([2H])c([2H])c54)c([2H])c([2H])c32)c([2H])c1[2H] ; [2H]c1nc2c(c([2H])c1[2H])c1c([2H])c([2H])c([2H])c([2H])c1n2-c1c(-n2c3c([2H])c([2H])c([2H])c([2H])c3c3c([2H])c([2H])c([2H])c([2H])c32)c([2H])c([2H])c2c1c1c([2H])c([2H])c([2H])c([2H])c1n2-c1c([2H])c([2H])c([2H])c([2H])c1[2H]]

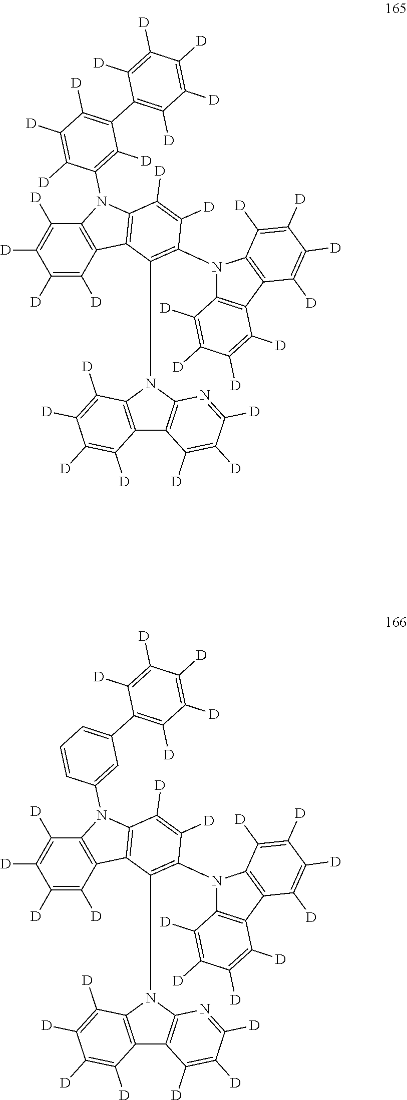 [SMILES: [2H]c1nc2c(c([2H])c1[2H])c1c([2H])c([2H])c([2H])c([2H])c1n2-c1c(-n2c3c([2H])c([2H])c([2H])c([2H])c3c3c([2H])c([2H])c([2H])c([2H])c32)c([2H])c([2H])c2c1c1c([2H])c([2H])c([2H])c([2H])c1n2-c1c([2H])c([2H])c([2H])c(-c2c([2H])c([2H])c([2H])c([2H])c2[2H])c1[2H] ; [2H]c1nc2c(c([2H])c1[2H])c1c([2H])c([2H])c([2H])c([2H])c1n2-c1c(-n2c3c([2H])c([2H])c([2H])c([2H])c3c3c([2H])c([2H])c([2H])c([2H])c32)c([2H])c([2H])c2c1c1c([2H])c([2H])c([2H])c([2H])c1n2-c1cccc(-c2c([2H])c([2H])c([2H])c([2H])c2[2H])c1]

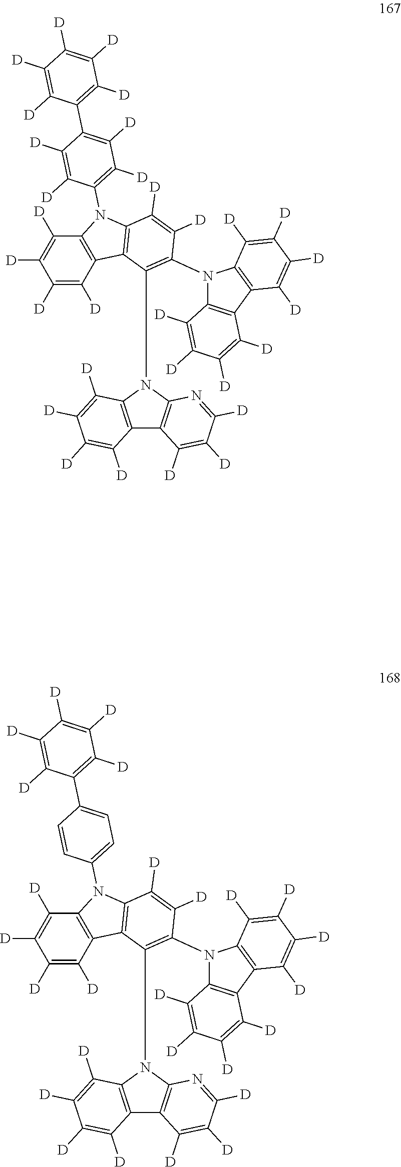 [SMILES: [2H]c1nc2c(c([2H])c1[2H])c1c([2H])c([2H])c([2H])c([2H])c1n2-c1c(-n2c3c([2H])c([2H])c([2H])c([2H])c3c3c([2H])c([2H])c([2H])c([2H])c32)c([2H])c([2H])c2c1c1c([2H])c([2H])c([2H])c([2H])c1n2-c1c([2H])c([2H])c(-c2c([2H])c([2H])c([2H])c([2H])c2[2H])c([2H])c1[2H] ; [2H]c1nc2c(c([2H])c1[2H])c1c([2H])c([2H])c([2H])c([2H])c1n2-c1c(-n2c3c([2H])c([2H])c([2H])c([2H])c3c3c([2H])c([2H])c([2H])c([2H])c32)c([2H])c([2H])c2c1c1c([2H])c([2H])c([2H])c([2H])c1n2-c1ccc(-c2c([2H])c([2H])c([2H])c([2H])c2[2H])cc1]

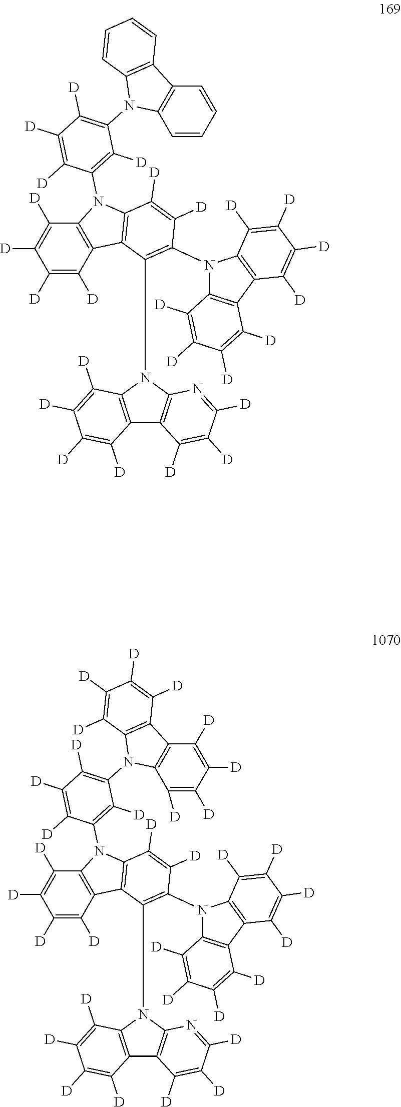

The heterocyclic compound represented by Formula 1 according to one or more embodiments may have inhibited or reduced intermolecular interactions due to its structural characteristic where two N-containing heterocycles exist at ortho positions to each other. Therefore, if (e.g., when) the heterocyclic compound is used in an emission layer, the color purity and lifespan may be improved or enhanced.

In one or more embodiments, the heterocyclic compound may include at least one heterocycle containing two or more N atoms, making hole transport characteristics relatively easy to control. As a result, if (e.g., when) the heterocyclic compound is used in the emission layer, the recombination region of the emission layer may be improved (or enhanced) or optimized, leading to improved or enhanced luminescence efficiency and lifespan.

Therefore, a light-emitting device including the heterocyclic compound may have excellent or suitable driving voltage, efficiency, and lifespan characteristics.

According to one or more embodiments, the heterocyclic compound represented by Formula 1 may have a highest occupied molecular orbital (HOMO) energy level of about-5.8 eV or higher.

According to one or more embodiments, the heterocyclic compound represented by Formula 1 may have a triplet excited state (T1) energy level of about 2.6 eV or higher.

The synthesis methods of the heterocyclic compound represented by Formula 1 may be recognizable by one of ordinary skill in the art by referring to Synthesis Examples and/or Examples provided herein.

At least one heterocyclic compound represented by Formula 1 may be used in a light-emitting device (for example, an organic light-emitting device). Accordingly, provided is a light-emitting device including: a first electrode; a second electrode opposite to (e.g., facing) the first electrode; and an interlayer arranged between the first electrode and the second electrode and including an emission layer, wherein the interlayer includes the heterocyclic compound represented by Formula 1.

In one or more embodiments,
the first electrode of the light-emitting device may be an anode,
the second electrode of the light-emitting device may be a cathode,
the interlayer may further include a hole transport region between the first electrode and the emission layer and an electron transport region between the emission layer and the second electrode,
the hole transport region may include a hole injection layer, a hole transport layer, an emission auxiliary layer, an electron-blocking layer, or a (e.g., any suitable) combination thereof, and
the electron transport region may include a buffer layer, a hole-blocking layer, an electron control layer, an electron transport layer, an electron injection layer, or a (e.g., any suitable) combination thereof.

The hole transport region may include a single layer or two or more layers, and the electron transport region may include a single layer or two or more layers.

In one or more embodiments, the heterocyclic compound may be included between the first electrode and the second electrode of the light-emitting device. Accordingly, the heterocyclic compound may be included in the interlayer of the light-emitting device, for example, in the emission layer of the interlayer.

In one or more embodiments, the emission layer of the interlayer of the light-emitting device may include a dopant and a host, and the heterocyclic compound may be included in the host. For example, the heterocyclic compound may act or serve as a host. The emission layer may be to emit red light, green light, blue light, and/or white light. For example, the emission layer may be to emit blue light. The blue light may have a maximum emission wavelength (e.g., the wavelength of maximum emission intensity) in a range of, for example, about 400 nm to about 490 nm. The blue light may be to emit light of, for example, about 430 nm to about 480 nm.

In one or more embodiments, the emission layer of the interlayer of the light-emitting device may include a dopant and a host, and the heterocyclic compound may be included in the host, and the dopant may be to emit blue light. For example, the dopant may include a transition metal and ligand(s) in the number of m, and m may be an integer of 1 to 6. The ligand(s) in the number of m may be identical to or different from each other, at least one selected from among the ligand(s) in the number of m may be bonded to the transition metal via a carbon-transition metal bond, and the carbon-transition metal bond may be a coordinate bond. For example, at least one selected from among the ligand(s) in the number of m may be a carbene ligand (e.g., Ir(pmp)₃ and/or the like). The transition metal may be, for example, iridium, platinum, osmium, palladium, rhodium, gold, and/or the like. More details on the emission layer and the dopant may be as described in one or more embodiments.

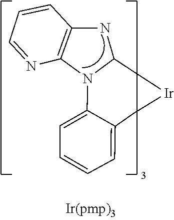

In one or more embodiments, the light-emitting device may include a capping layer outside the first electrode or outside the second electrode.

In one or more embodiments, the light-emitting device may further include at least one selected from among a first capping layer outside the first electrode and a second capping layer outside the second electrode, and at least one selected from among the first capping layer and the second capping layer may include the heterocyclic compound represented by Formula 1. More details on the first capping layer and/or the second capping layer may be referred to the descriptions provided in one or more embodiments.

In one or more embodiments, the light-emitting device may include:
a first capping layer arranged outside the first electrode and including the heterocyclic compound represented by Formula 1;
a second capping layer arranged outside the second electrode and including the heterocyclic compound represented by Formula 1; or
the first capping layer and the second capping layer.

The expression “(an interlayer and/or a capping layer) includes at least one heterocyclic compound” as used herein may include a case in which “(an interlayer and/or a capping layer) includes substantially identical heterocyclic compounds represented by Formula 1” and a case in which “(an interlayer and/or a capping layer) includes two or more different heterocyclic compounds represented by Formula 1.”

For example, the interlayer and/or the capping layer may include Compound 1 only as the heterocyclic compound. In one or more embodiments, Compound 1 may be in the emission layer of the light-emitting device. In one or more embodiments, the interlayer may include, as the heterocyclic compound, Compound 1 and Compound 2. In one or more embodiments, Compound 1 and Compound 2 may be in substantially the same layer (for example, both (e.g., simultaneously) Compound 1 and Compound 2 may be in the emission layer), or may be in different layers (for example, Compound 1 may be in the emission layer, and Compound 2 may be in the electron transport region).

The term “interlayer” as used herein refers to a single layer and/or all of multiple layers between the first electrode and the second electrode of the light-emitting device.

One or more embodiments of the present disclosure provide an electronic apparatus including the light-emitting device. The electronic device may further include a thin-film transistor. For example, the electronic apparatus may further include a thin-film transistor including a source electrode and a drain electrode, wherein the first electrode of the light-emitting device may be electrically connected to the source electrode or the drain electrode. In one or more embodiments, the electronic apparatus may further include a color filter, a color conversion layer, a touch screen layer, a polarizing layer, or a (e.g., any suitable) combination thereof. More details on the 1 electronic apparatus may be referred to the descriptions provided in one or more embodiments.

One or more embodiments of the present disclosure provide an electronic device including the light-emitting device. The electronic device may be one selected from among a flat panel display, a curved display, a computer monitor, a medical monitor, a television, a billboard, an indoor light, an outdoor light, a light for signal, a head-up display, a fully transparent display, a partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, and a signboard. More details on the electronic device may be as described in one or more embodiments.

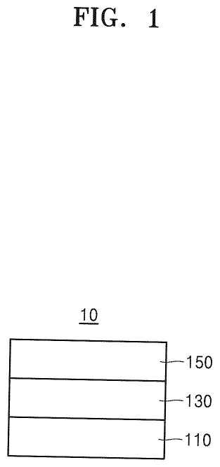

### DESCRIPTION OF FIG. 1

FIG. 1 is a schematic cross-sectional view of a light-emitting device 10 according to one or more embodiments. The light-emitting device 10 may include a first electrode 110, an interlayer 130, and a second electrode 150.

Hereinafter, a structure of the light-emitting device 10 according to one or more embodiments and a method of manufacturing the light-emitting device 10 are described with reference to FIG. 1.

### First Electrode 110

In FIG. 1, a substrate may be additionally arranged under the first electrode 110 or on the second electrode 150. As the substrate, a glass substrate and/or a plastic substrate may be used. In one or more embodiments, the substrate may be a flexible substrate and may include plastics with excellent or suitable heat resistance and durability, such as polyimide, polyethylene terephthalate (PET), polycarbonate, polyethylene naphthalate, polyarylate (PAR), polyetherimide, or a (e.g., any suitable) combination thereof.

The first electrode 110 may be formed or provided by, for example, depositing and/or sputtering a material to form or provide the first electrode 110 on the substrate. If (e.g., when) the first electrode 110 is an anode, a material to form or provide the first electrode 110 may be a high-work function material that facilitates injection of holes.

The first electrode 110 may be a reflective electrode, a transflective electrode, or a transmissive electrode. If (e.g., when) the first electrode 110 is a transmissive electrode, a material to form or provide the first electrode 110 may include indium tin oxide (ITO), indium zinc oxide (IZO), tin oxide (e.g., SnO₂), zinc oxide (e.g., ZnO), or a (e.g., any suitable) combination thereof. In one or more embodiments, if (e.g., when) the first electrode 110 is a transflective electrode or a reflective electrode, a material to form or provide the first electrode 110 may include magnesium (Mg), silver (Ag), aluminum (Al), aluminum-lithium (Al—Li), calcium (Ca), magnesium-indium (Mg—In), magnesium-silver (Mg—Ag), or a (e.g., any suitable) combination thereof.

The first electrode 110 may have a single-layer structure including (e.g., consisting of) a single layer structure or a multilayer structure including a plurality of layers. In one or more embodiments, the first electrode 110 may have a three-layer structure of ITO/Ag/ITO.

### Interlayer 130

The interlayer 130 may be above the first electrode 110. The interlayer 130 may include the emission layer.

The interlayer 130 may further include a hole transport region between the first electrode 110 and the emission layer and an electron transport region between the emission layer and the second electrode 150.

The interlayer 130 may further include, in addition to one or more suitable organic materials, a metal-containing compound, such as an organometallic compound, an inorganic material, such as quantum dots, and/or the like.

In one or more embodiments, the interlayer 130 may include, i) two or more emitting units sequentially stacked between the first electrode 110 and the second electrode 150, and ii) a charge generation layer between adjacent emitting units among the two or more emitting units. If (e.g., when) the interlayer 130 includes the emitting units and a charge generation layer as described in one or more embodiments, the light-emitting device 10 may be a tandem light-emitting device.

### Hole Transport Region in Interlayer 130

The hole transport region may have: i) a single-layer structure including (e.g., consisting of) a single layer including (e.g., consisting of) a single material, ii) a single-layer structure including (e.g., consisting of) a single layer including (e.g., consisting of) a plurality of materials that are different from each other, or iii) a multilayer structure including a plurality of layers including a plurality of materials that are different from each other.

The hole transport region may include a hole injection layer, a hole transport layer, an emission auxiliary layer, an electron-blocking layer, or a (e.g., any suitable) combination thereof.

For example, the hole transport region may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, wherein the layers in each structure are sequentially stacked from the first electrode 110.

The hole transport region may include a compound represented by Formula 201, a compound represented by Formula 202, or a (e.g., any suitable) combination thereof:

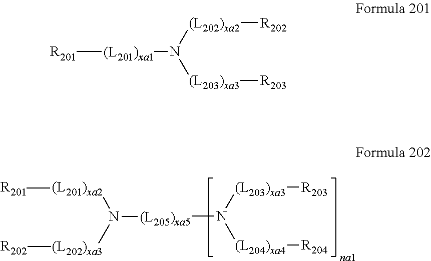

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer of 0 to 5,
xa5 may be an integer of 1 to 10,
R₂₀₁ to R₂₀₄ and Q₂₀₁ may each independently be a C₃-Coo carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond (e.g., a single covalent bond), a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group (for example, a carbazole group) that is unsubstituted or substituted with at least one R₁₀ₐ (for example, Compound HT16),
R₂₀₃ and R₂₀₄ may optionally be linked to each other via a single bond (e.g., a single covalent bond), a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer of 1 to 4.

In one or more embodiments, each of Formulae 201 and 202 may include at least one selected from among the groups represented by Formulae CY201 to CY217:

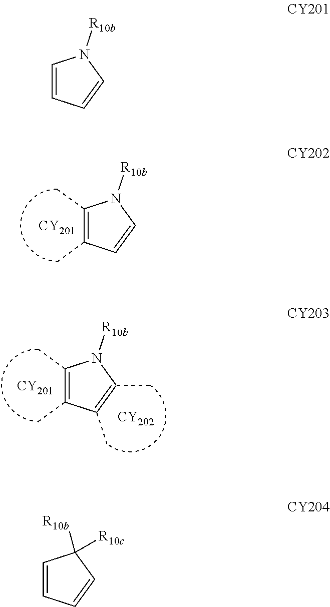

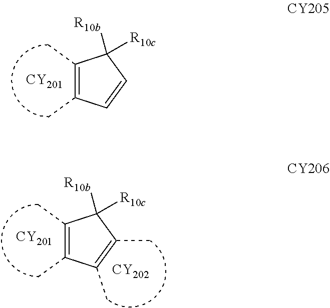

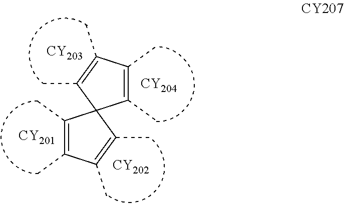

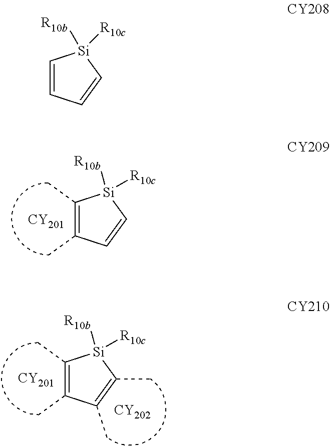

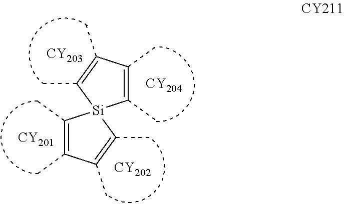

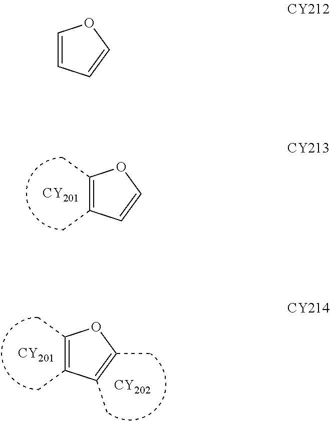

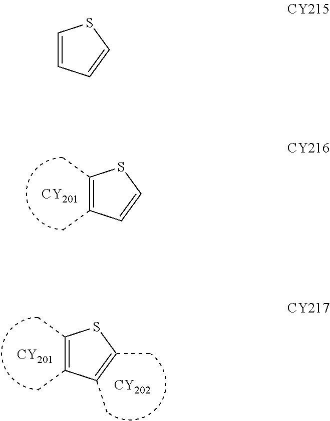

wherein, in Formulae CY201 to CY217, R_{10b} and R_{10c} may each be the same as defined with respect to R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, in Formulae CY201 to CY217, ring CY₂₀₁ to ring CY₂₀₄ may each independently be a benzene group, a naphthalene group, a phenanthrene group, or an anthracene group.

In one or more embodiments, each of Formulae 201 and 202 may include at least one selected from among the groups represented by Formulae CY201 to CY203.

In one or more embodiments, Formula 201 may include at least one selected from among the groups represented by Formulae CY201 to CY203 and at least one selected from among the groups represented by Formulae CY204 to CY217.

In one or more embodiments, in Formula 201, xa1 may be 1, R₂₀₁ may be a group represented by one selected from among Formulae CY201 to CY203, xa2 may be 0, and R₂₀₂ may be a group represented by one selected from among Formulae CY204 to CY207.

In one or more embodiments, each of Formulae 201 and 202 may not include (e.g., may exclude) groups represented by Formulae CY201 to CY203.

In one or more embodiments, each of Formulae 201 and 202 may not include (e.g., may exclude) groups represented by Formulae CY201 to CY203 and may include at least one selected from among the groups represented by Formulae CY204 to CY217.

In one or more embodiments, each of Formulae 201 and 202 may not include (e.g., may exclude) groups represented by Formulae CY201 to CY217.

In one or more embodiments, the hole transport region may include one (e.g., at least one) selected from among Compounds HT1 to HT46, m-MTDATA, TDATA, 2-TNATA, NPB (NPD), β-NPB, TPD, Spiro-TPD, Spiro-NPB, methylated NPB, TAPC, HMTPD, CzSi, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or a (e.g., any suitable) combination thereof:

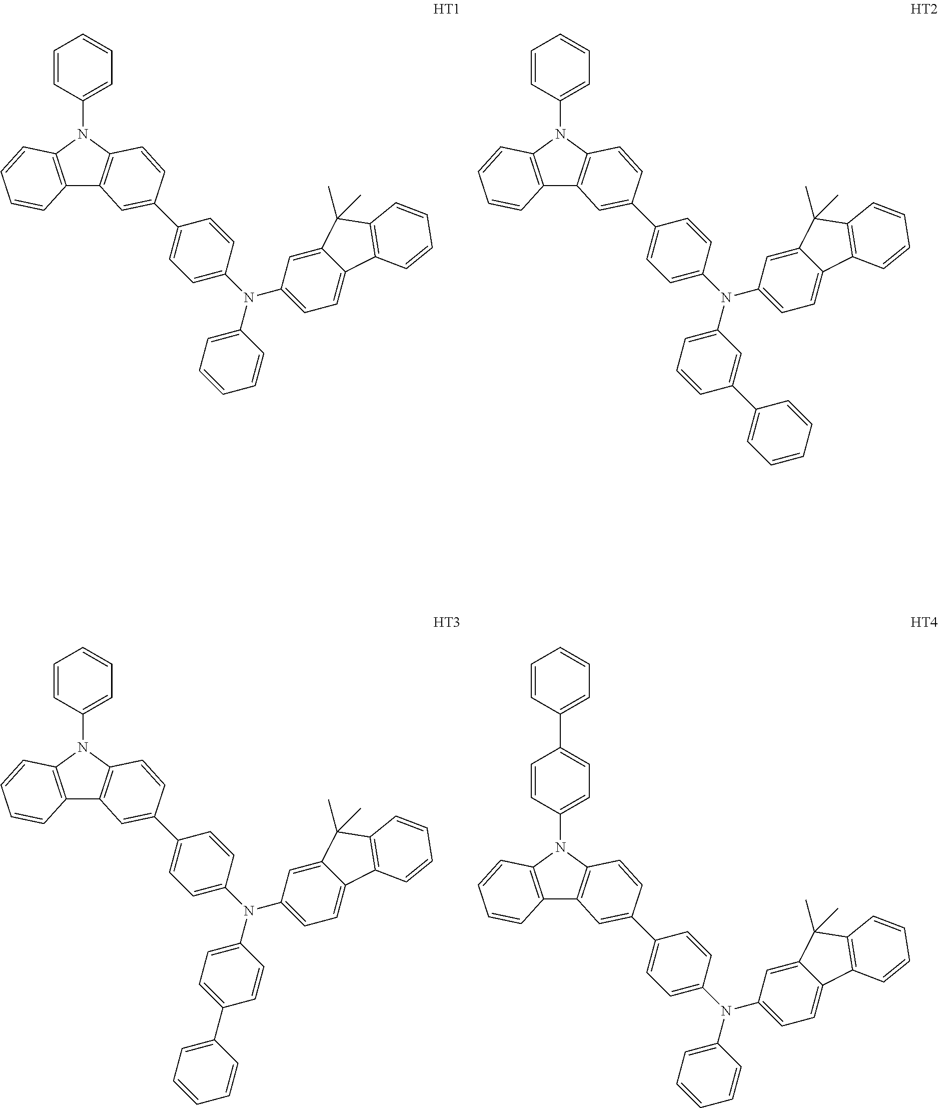 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

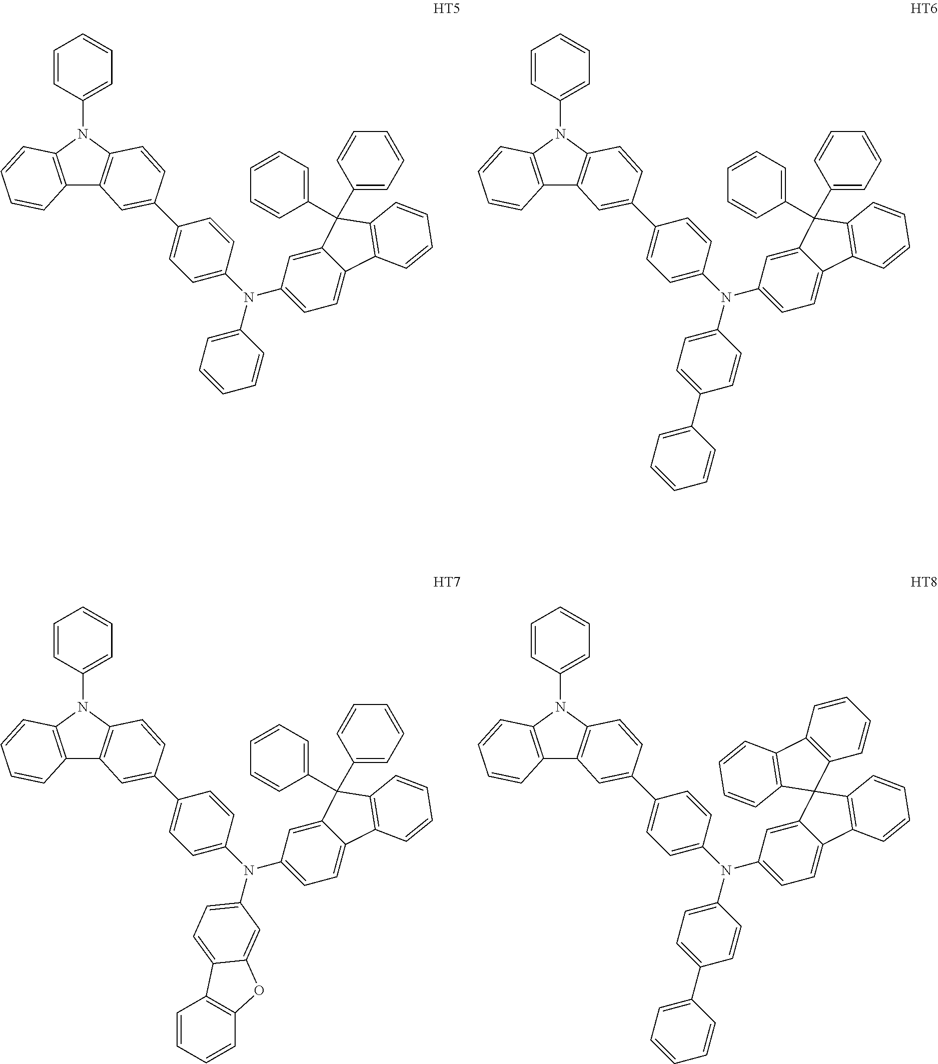 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

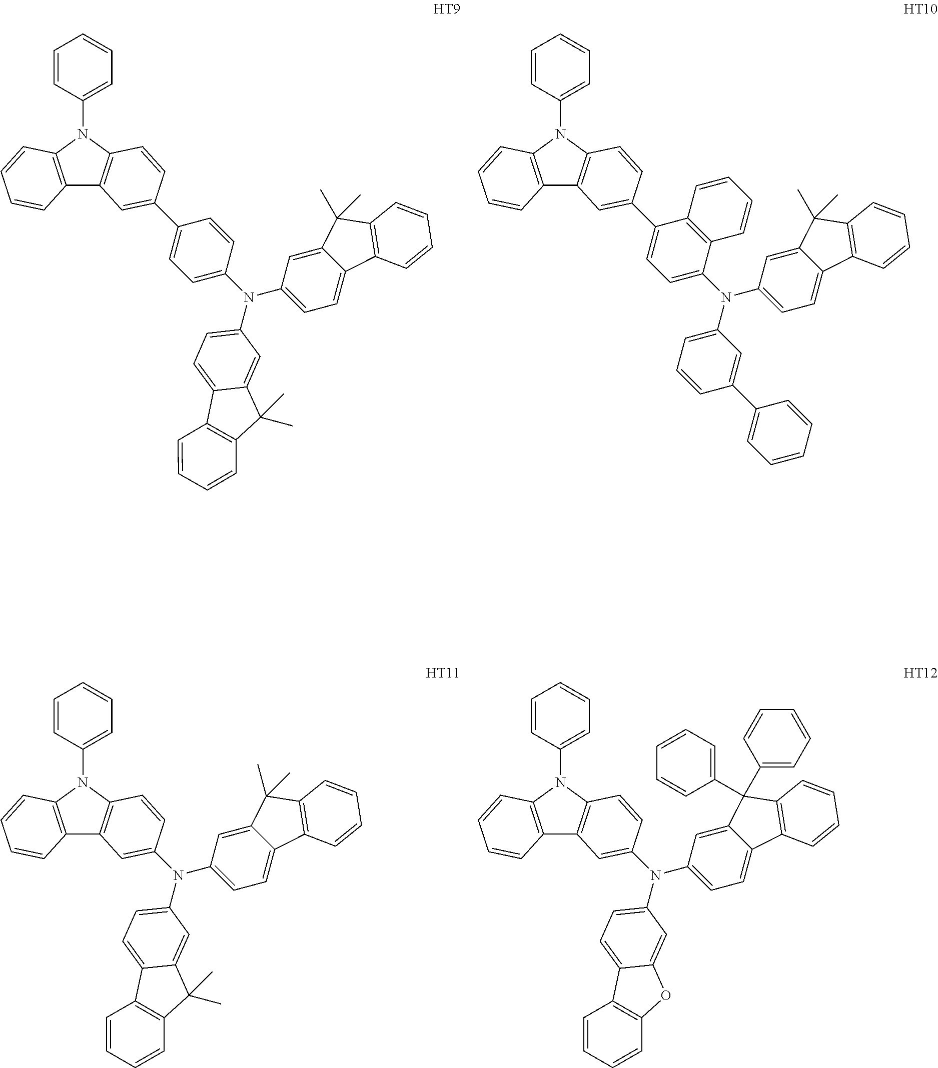 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

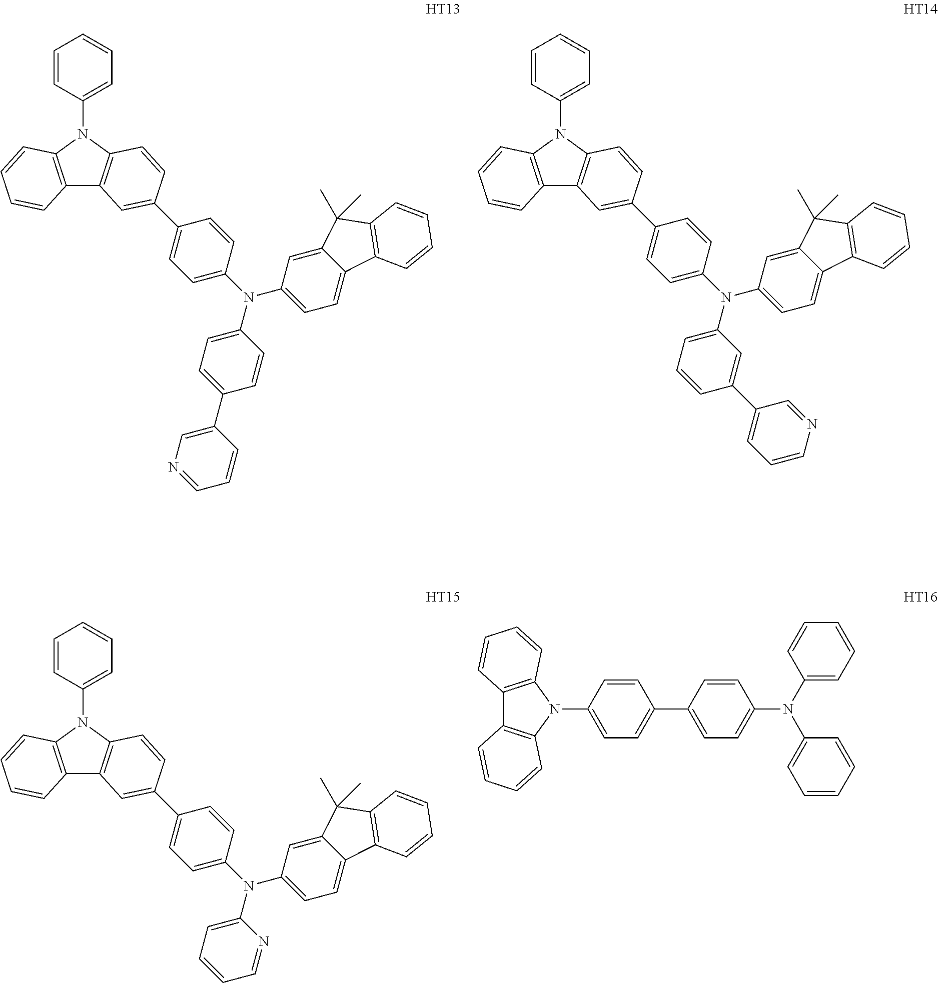 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

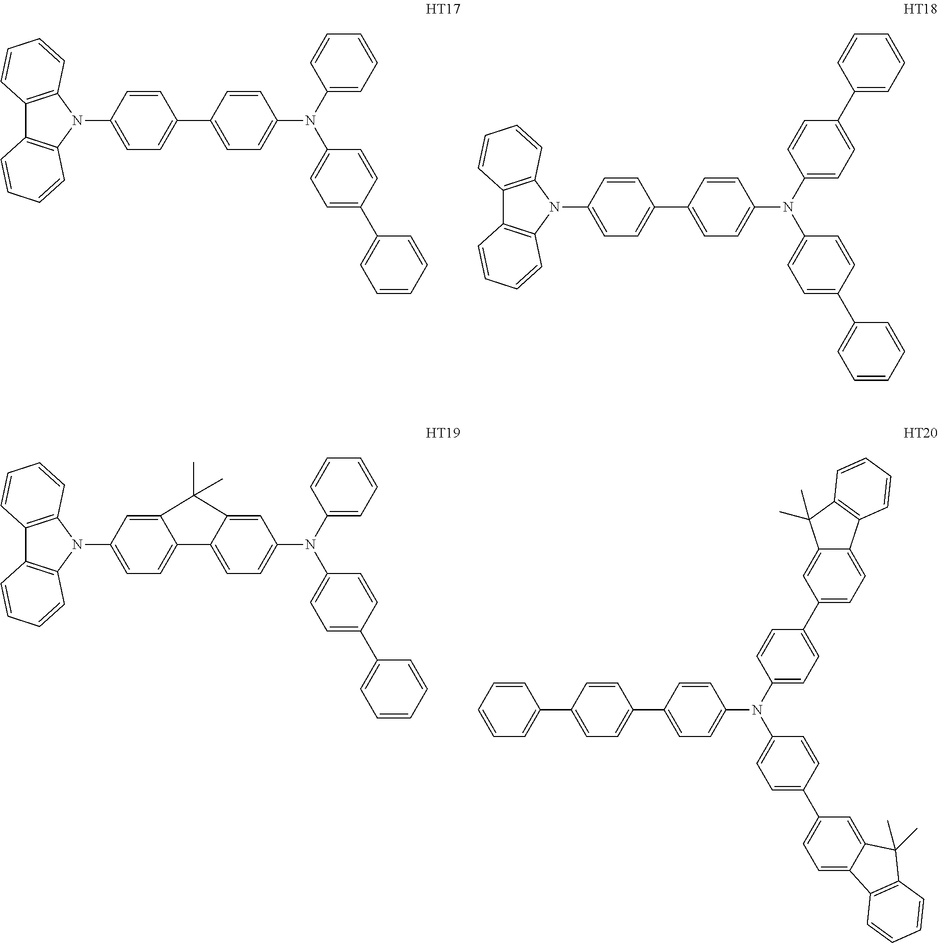 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

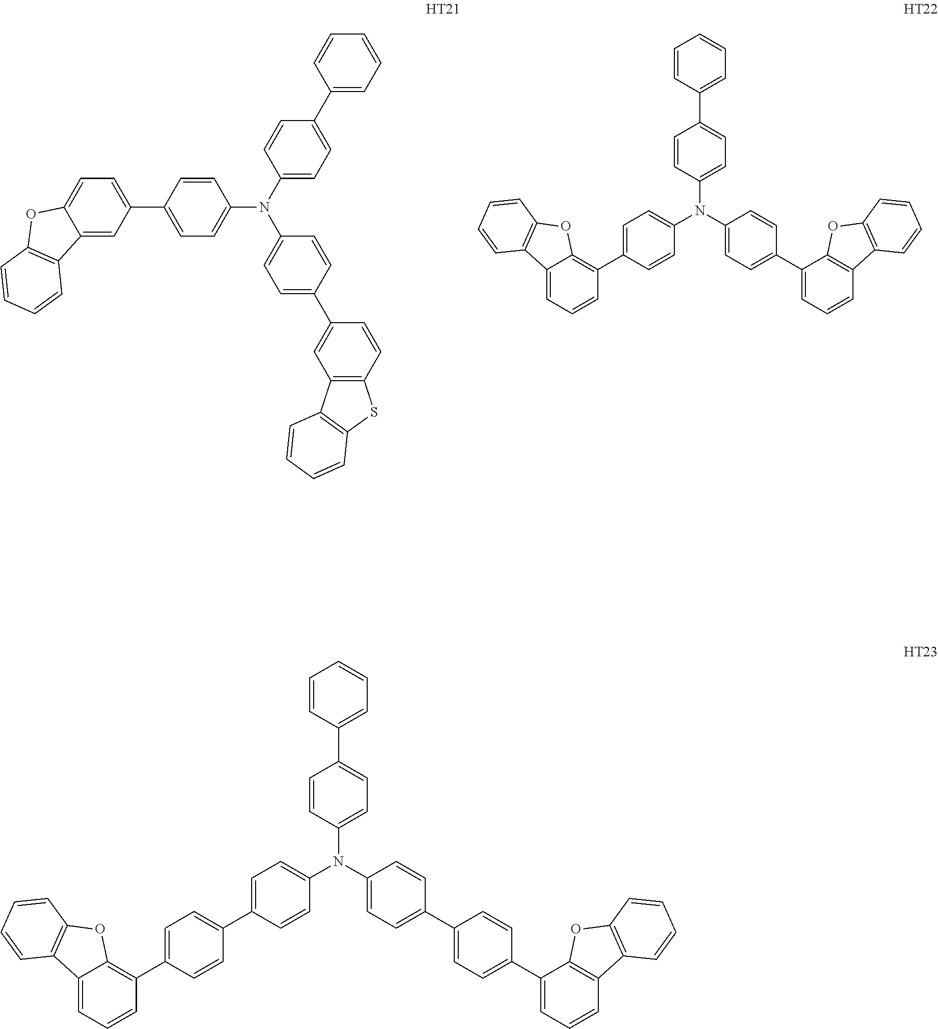 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

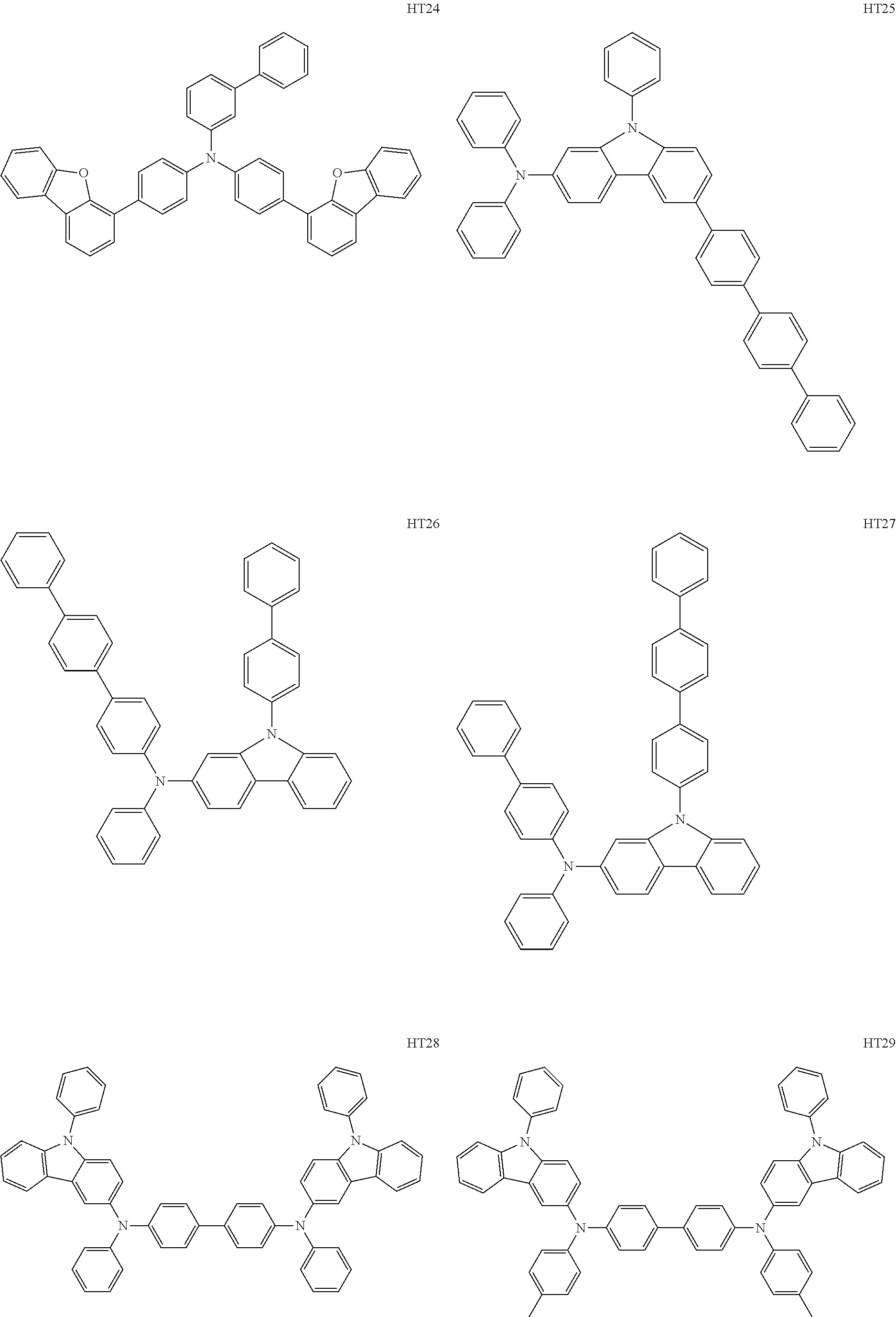

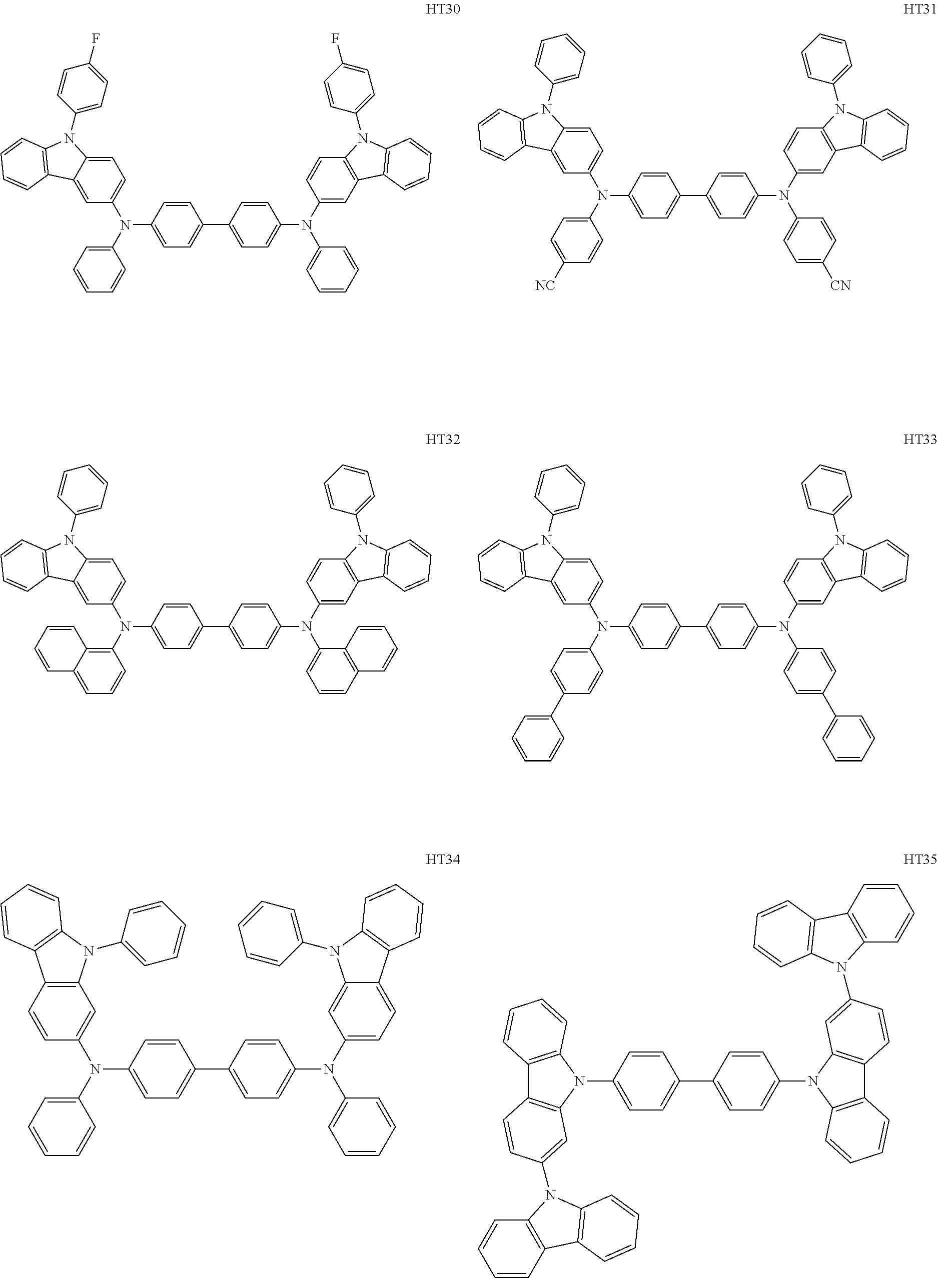

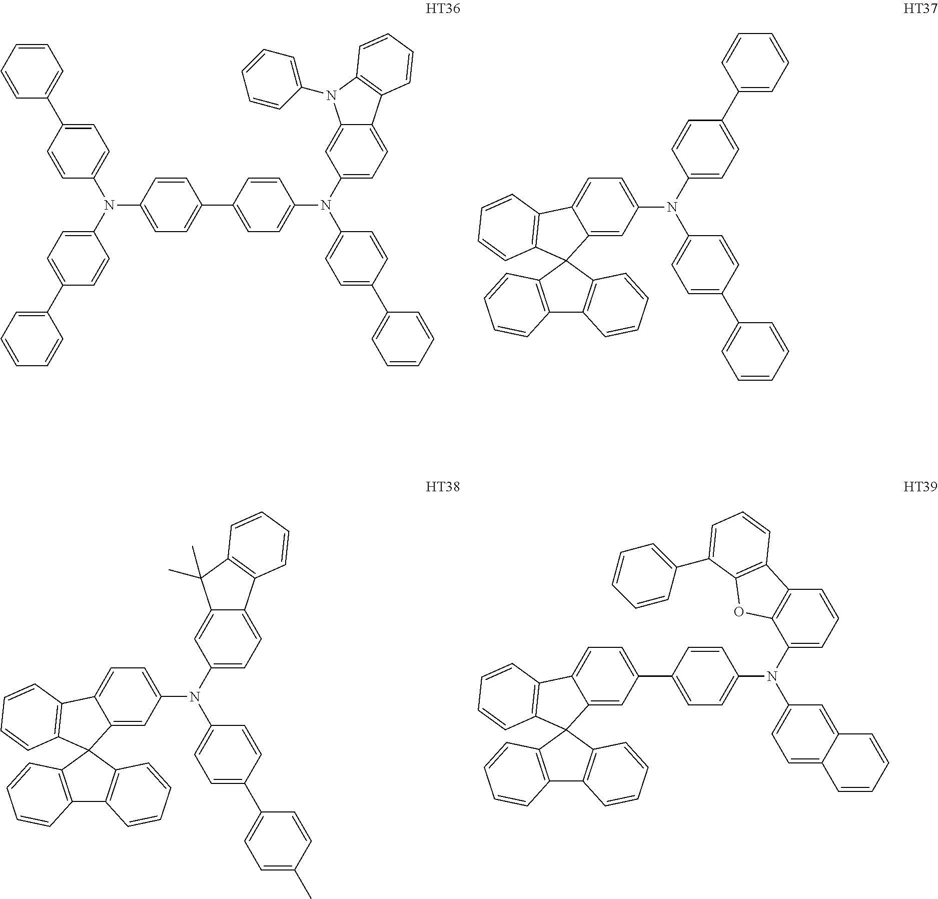 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

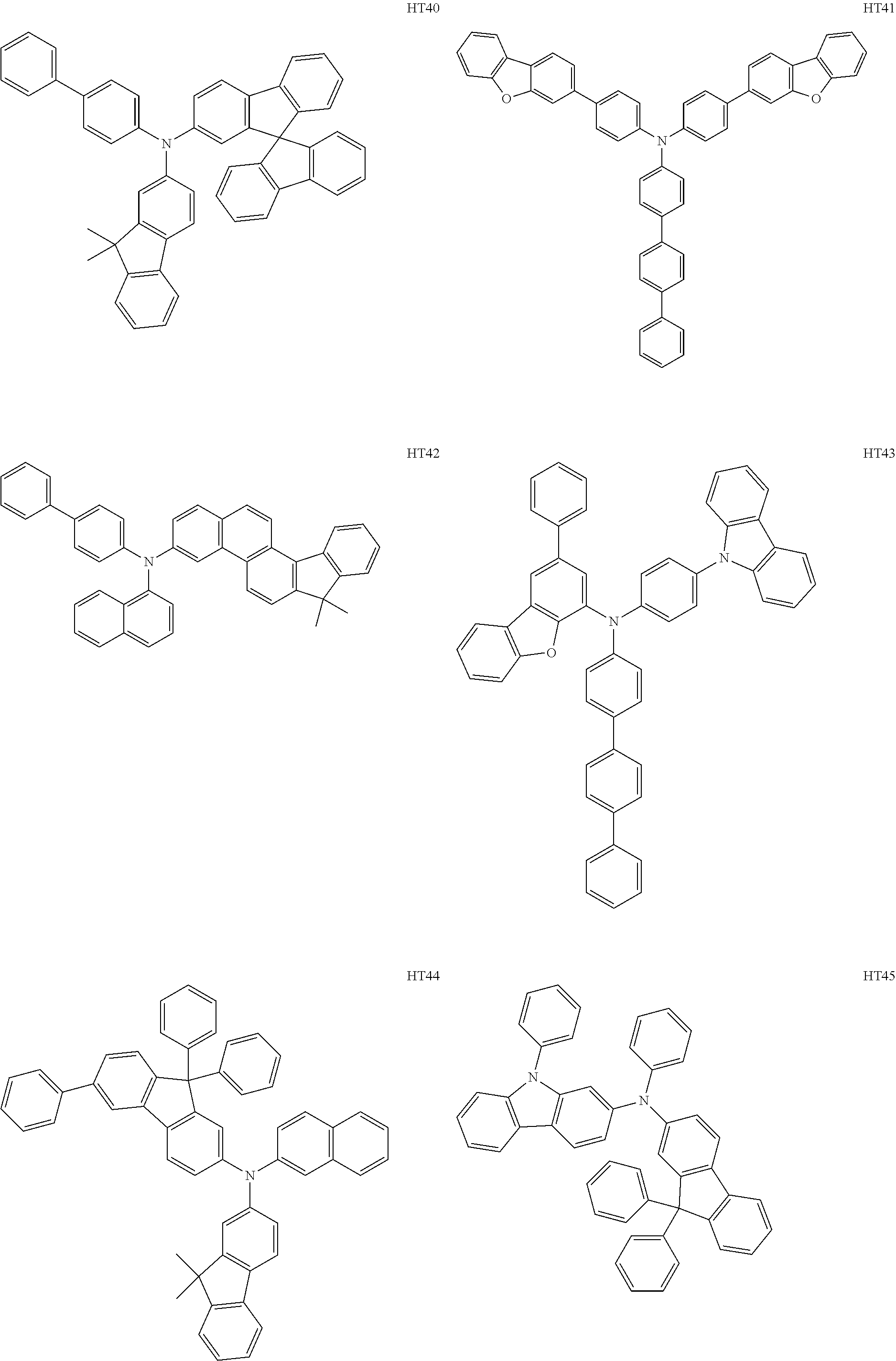

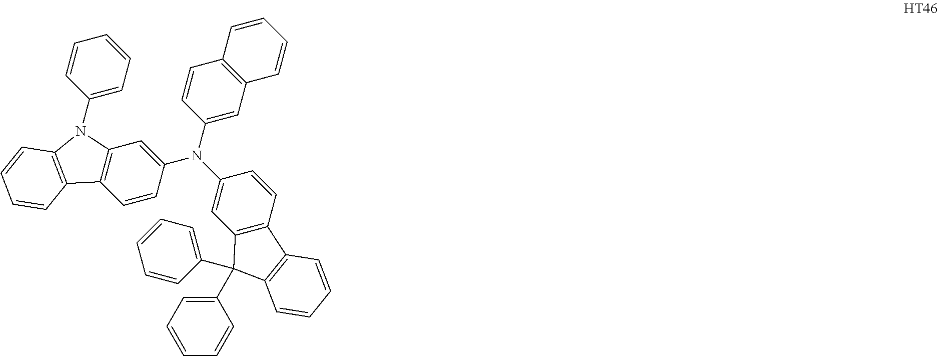 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

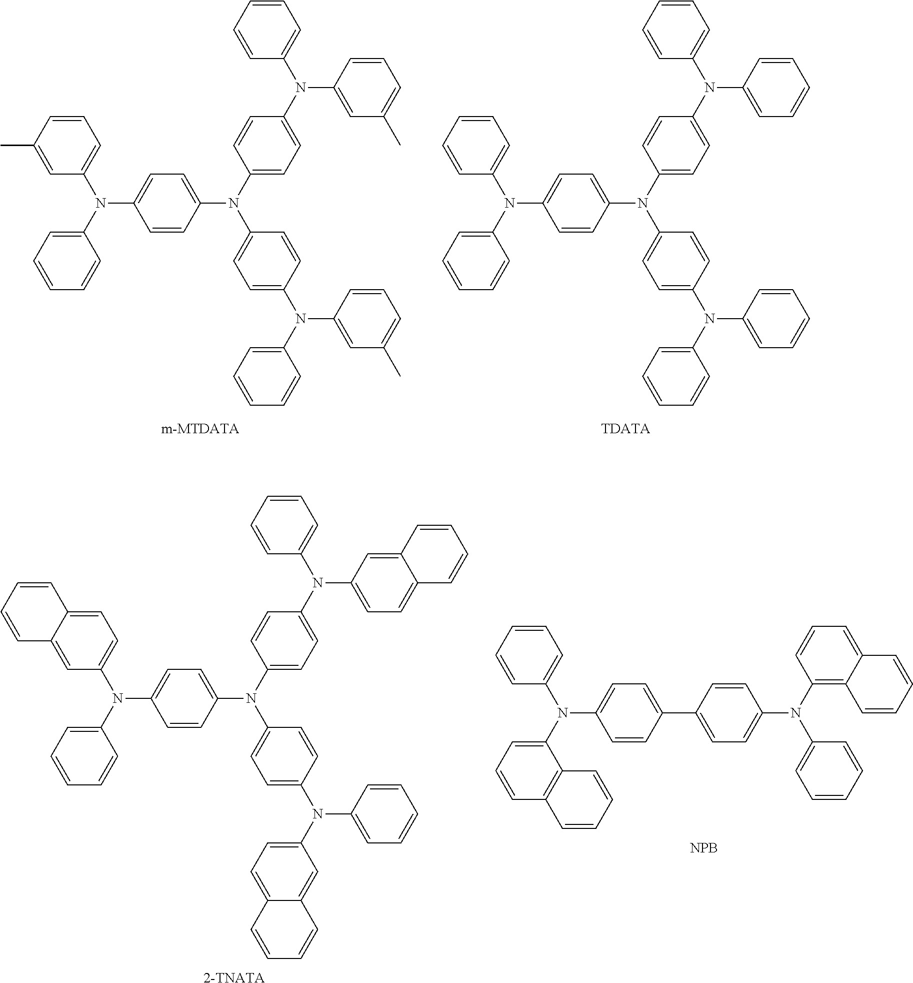

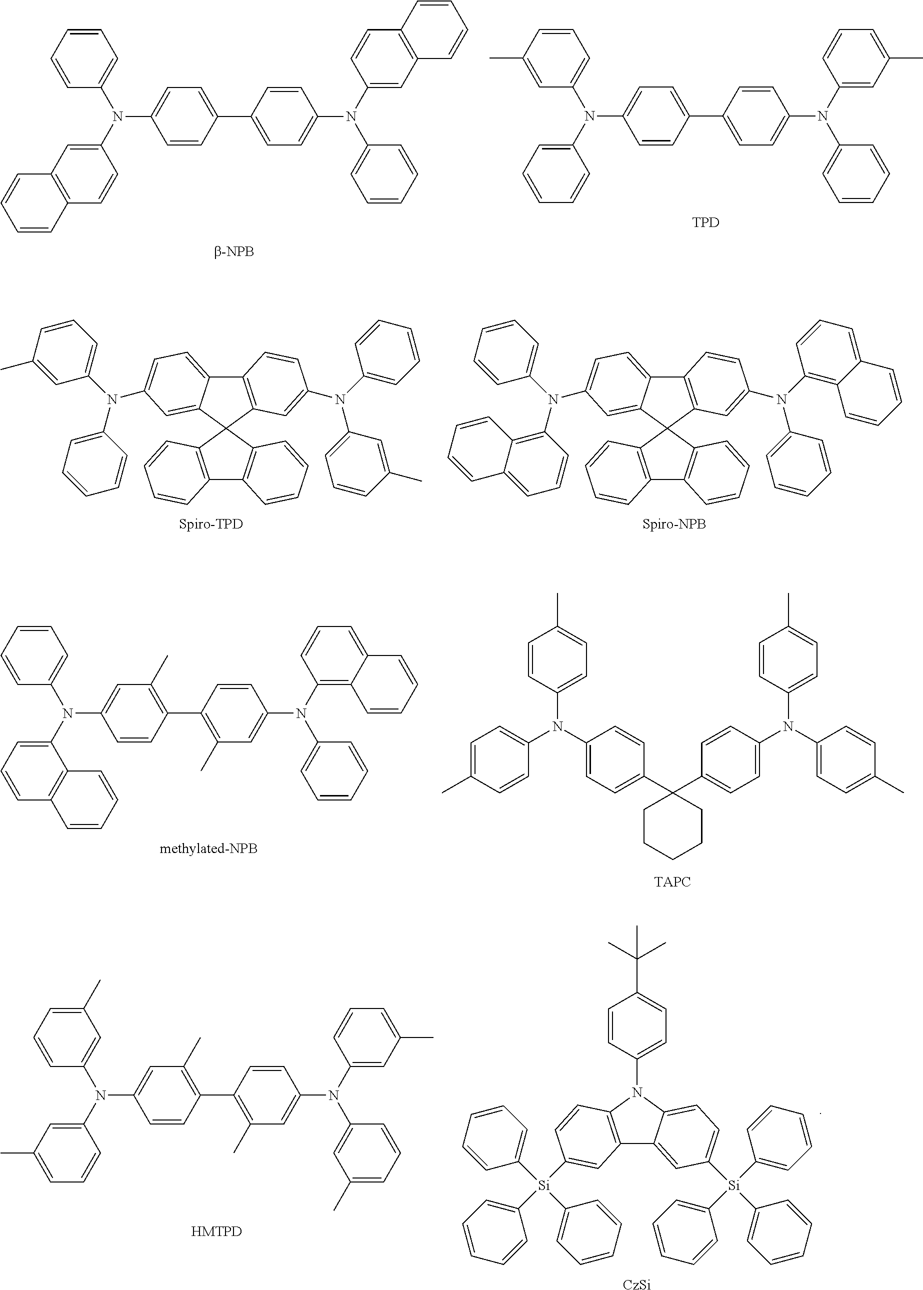

The thickness of the hole transport region may be about 50 Å to about 10,000 Å, for example, about 100 Å to about 4,000 Å. If (e.g., when) the hole transport region includes a hole injection layer, a hole transport layer, or a (e.g., any suitable) combination thereof, the thickness of the hole injection layer may be about 100 Å to about 9,000 Å, for example, about 100 Å to about 1,000 Å, and the thickness of the hole transport layer may be about 50 Å to about 2,000 Å, for example, about 100 Å to about 1,500 Å. If (e.g., when) the thicknesses of the hole transport region, the hole injection layer, and the hole transport layer are within the foregoing ranges, satisfactory or desired hole transporting characteristics may be obtained without a substantial increase in driving voltage.

The emission auxiliary layer may increase or enhance luminescence efficiency by compensating for an optical resonance distance according to the wavelength of light emitted by the emission layer, and the electron-blocking layer may block the leakage of electrons (or reduce a degree or occurrence of the leakage of electrons) from the emission layer to the hole transport region. Materials that may be included in the hole transport region may be included in the emission auxiliary layer and the electron-blocking layer.

p-Dopant

The hole transport region may further include, in addition to the materials as described in one or more embodiments, a charge-generation material for the improvement or enhancement of conductive (e.g., electrically conductive) properties. The charge-generation material may be uniformly (e.g., substantially uniformly) or non-uniformly (e.g., substantially non-uniformly) dispersed in the hole transport region (for example, in the form of a single layer including (e.g., consisting of) a charge-generation material).

The charge-generation material may be, for example, a p-dopant.

For example, the lowest unoccupied molecular orbital (LUMO) energy of the p-dopant may be less than or equal to about-3.5 eV.

In one or more embodiments, the p-dopant may include a quinone derivative, a cyano group-containing compound, a compound including an element EL1 and an element EL2, or a (e.g., any suitable) combination thereof.

Non-limiting examples of the quinone derivative may be TCNQ and F4-TCNQ.

Non-limiting examples of the cyano group-containing compound may be HAT-CN and a compound represented by Formula 221.

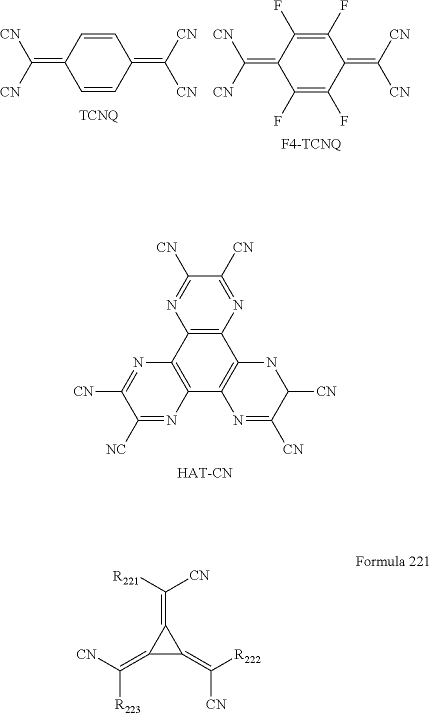

In Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one selected from among R₂₂₁ to R₂₂₃ may each independently be a C₃-C₆₀ carbocyclic group or a C₁-C₆₀ heterocyclic group, each being unsubstituted or substituted with: a cyano group; —F; —Cl; —Br; —I; a C₁-C₂₀ alkyl group unsubstituted or substituted with a cyano group, —F, —Cl, —Br, —I, or any combination thereof; or any combination thereof.

In the compound including the element EL1 and the element EL2, the element EL1 may be a metal, a metalloid, or a (e.g., any suitable) combination thereof, and the element EL2 may be a non-metal, a metalloid, or a (e.g., any suitable) combination thereof.

Non-limiting examples of the metal may be an alkali metal (for example, lithium (Li), sodium (Na), potassium (K), rubidium (Rb), cesium (Cs), and/or the like); alkaline earth metal (for example, beryllium (Be), magnesium (Mg), calcium (Ca), strontium (Sr), barium (Ba), and/or the like); transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); post-transition metal (for example, zinc (Zn), indium (In), tin (Sn), and/or the like); and/or lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like).

Non-limiting examples of the metalloid may be silicon (Si), antimony (Sb), and/or tellurium (Te).

Non-limiting examples of the non-metal may be oxygen (O) and/or halogen (for example, F, Cl, Br, I, and/or the like).

Non-limiting examples of the compound including the element EL1 and the element EL2 may be a metal oxide, a metal halide (for example, a metal fluoride, a metal chloride, a metal bromide, a metal iodide, and/or the like.), a metalloid halide (for example, a metalloid fluoride, a metalloid chloride, a metalloid bromide, a metalloid iodide, and/or the like.), a metal telluride, or a (e.g., any suitable) combination thereof.

Non-limiting examples of the metal oxide may be a tungsten oxide (for example, WO, W₂O₃, WO₂, WO₃, W₂O₅, and/or the like), a vanadium oxide (for example, VO, V₂O₃, VO₂, V₂O₅, and/or the like), a molybdenum oxide (for example, MoO, MO₂O₃, MoO₂, MoO₃, Mo₂O₅, AND/OR THE LIKE), AND/OR A RHENIUM OXIDE (FOR example, ReO₃ and/or the like).

Non-limiting examples of the metal halide may be an alkali metal halide, an alkaline earth metal halide, a transition metal halide, a post-transition metal halide, and/or a lanthanide metal halide.

Non-limiting examples of the alkali metal halide may be LiF, NaF, KF, RbF, CsF, LiCI, NaCl, KCl, RbCI, CsCl, LiBr, NaBr, KBr, RbBr, CsBr, LiI, NaI, KI, RbI, and/or CsI.

Non-limiting examples of the alkaline earth metal halide may be BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, Bel₂, Mgl₂, Cal₂, Srl₂, and/or BaI₂.

Non-limiting examples of the transition metal halide may be a titanium halide (for example, TiF₄, TiCl₄, TiBr₄, Til₄, and/or the like), a zirconium halide (for example, ZrF₄, ZrCl₄, ZrBr₄, Zrl₄, and/or the like), a hafnium halide (for example, HfF₄, HfCI₄, HfBr₄, Hfl₄, and/or the like), a vanadium halide (for example, VF₃, VCI₃, VBr₃, Vl₃, and/or the like), a niobium halide (for example, NbF₃, NbCl₃, NbBr₃, Nbl₃, and/or the like), a tantalum halide (for example, TaF₃, TaCl₃, TaBr₃, Tal₃, and/or the like), a chromium halide (for example, CrF₃, CrCl₃, CrBr₃, Crl₃, and/or the like), a molybdenum halide (for example, MoF₃, MoCl₃, MoBr₃, MoI₃, and/or the like), a tungsten halide (for example, WF₃, WCl₃, WBr₃, Wl₃, and/or the like), a manganese halide (for example, MnF₂, MnCl₂, MnBr₂, Mnl₂, and/or the like), a technetium halide (for example, TcF₂, TcCl₂, TcBr₂, TcI₂, and/or the like), a rhenium halide (for example, ReF₂, ReCl₂, ReBr₂, ReI₂, and/or the like), an Iron (II) halide (for example, FeF₂, FeCl₂, FeBr₂, Fel₂, and/or the like), a ruthenium halide (for example, RuF₂, RuCl₂, RuBr₂, Rul₂, and/or the like), an osmium halide (for example, OsF₂, OsCl₂, OsBr₂, Osl₂, and/or the like), a cobalt halide (for example, CoF₂, CoCl₂, CoBr₂, Col₂, and/or the like), a rhodium halide (for example, RhF₂, RhCl₂, RhBr₂, Rhl₂, and/or the like), an iridium halide (for example, IrF₂, IrCl₂, IrBr₂, IrI₂, and/or the like), a nickel halide (for example, NiF₂, NiCl₂, NiBr₂, NiI₂, and/or the like), a palladium halide (for example, PdF₂, PdCl₂, PdBr₂, PdI₂, and/or the like), a platinum halide (for example, PtF₂, PtCl₂, PtBr₂, PtI₂, and/or the like), a Copper(I) halide (for example, CuF, CuCl, CuBr, CuI, and/or the like), a silver halide (for example, AgF, AgCl, AgBr, AgI, and/or the like), and a gold halide (for example, AuF, AuCl, AuBr, AuI, and/or the like).

Non-limiting examples of the post-transition metal halide may be a zinc halide (for example, ZnF₂, ZnCl₂, ZnBr₂, ZnI₂, and/or the like), an indium halide (for example, InI₃ and/or the like), and a tin halide (for example, SnI₂ and/or the like).

Non-limiting examples of the lanthanide metal halide may be YbF, YbF₂, YbF₃, SmF₃, YbCI, YbCl₂, YbCl₃ SmCl₃, YbBr, YbBr₂, YbBr₃, SmBr₃, YbI, YbI₂, YbI₃, and/or SmI₃.

Non-limiting examples of the metalloid halide may be an antimony halide (for example, SbCl₅ and/or the like).

Non-limiting examples of the metal telluride may be an alkali metal telluride (for example, Li₂Te, Na₂Te, K₂Te, Rb₂Te, Cs₂Te, and/or the like), an alkaline earth metal telluride (for example, BeTe, MgTe, CaTe, SrTe, BaTe, and/or the like), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), a post-transition metal telluride (for example, ZnTe and/or the like), and/or a lanthanide metal telluride (for example, LaTe, CeTe, PrTe, NdTe, PmTe, EuTe, GdTe, TbTe, DyTe, HoTe, ErTe, TmTe, YbTe, LuTe, and/or the like).

### Emission Layer in Interlayer 130

If (e.g., when) the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact each other or are separated from each other, to emit white light. In one or more embodiments, the emission layer may include two or more materials of a red light-emitting material, a green light-emitting material, and a blue light-emitting material, in which the two or more materials are mixed with each other in a single layer, to emit white light. For example, the emission layer may be to emit blue light.

In one or more embodiments, the emission layer may include the heterocyclic compound represented by Formula 1 as described in one or more embodiments.

The emission layer may include a host and a dopant.

In one or more embodiments, the dopant may include the heterocyclic compound represented by Formula 1 as described in one or more embodiments. In one or more embodiments, the dopant may further include a phosphorescent dopant, a fluorescent dopant, or a (e.g., any suitable) combination thereof, in addition to the heterocyclic compound represented by Formula 1. In addition to the heterocyclic compound represented by Formula 1, the phosphorescent dopant, the fluorescent dopant, and/or the like that may be further included in the emission layer are each as described in more detail herein.

The amount of the dopant in the emission layer may be from about 0.01 part by weight to about 15 parts by weight based on 100 parts by weight of the host.

In one or more embodiments, the emission layer may include a quantum dot.

In one or more embodiments, the emission layer may include a delayed fluorescence material. The delayed fluorescence material may act or serve as a host or a dopant in the emission layer.

A thickness of the emission layer may be in a range of about 100 Å to about 1,000 Å, for example, about 200 Å to about 600 Å. If (e.g., when) the thickness of the emission layer is within the foregoing ranges, excellent or suitable light-emission characteristics may be obtained without a substantial increase in driving voltage.

### Host

In one or more embodiments, the host may be a heterocyclic compound represented by Formula 1.

In one or more embodiments, the host may include, for example, a carbazole-containing compound, an anthracene-containing compound, or a (e.g., any suitable) combination thereof.

In one or more embodiments, the host may include a compound represented by Formula 301:

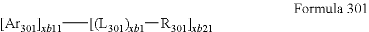

wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer of 0 to 5,
R₃₀₁ may be hydrogen, deuterium, —F, —Cl, —Br, —I, a hydroxyl group, a cyano group, a nitro group, an amidino group, a hydrazino group, a hydrazono group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O) (Q₃₀₁), —S(═O) (Q₃₀₁), —S(═O)₂ (Q₃₀₁), or —P(═O)(Q₃₀₁)(Q₃₀₂), or —P(═S)(Q₃₀₁)(Q₃₀₂),
xb21 may be an integer of 1 to 5, and
Q₃₀₁ to Q₃₀₃ may each be the same as defined with respect to Q₁.

In one or more embodiments, if (e.g., when) xb11 in Formula 301 is 2 or greater, two or more selected from among Ar₃₀₁ (s) may be linked to each other via a single bond (e.g., a single covalent bond).

In one or more embodiments, the host may include a compound represented by Formula 301-1, a compound represented by Formula 301-2, or a (e.g., any suitable) combination thereof:

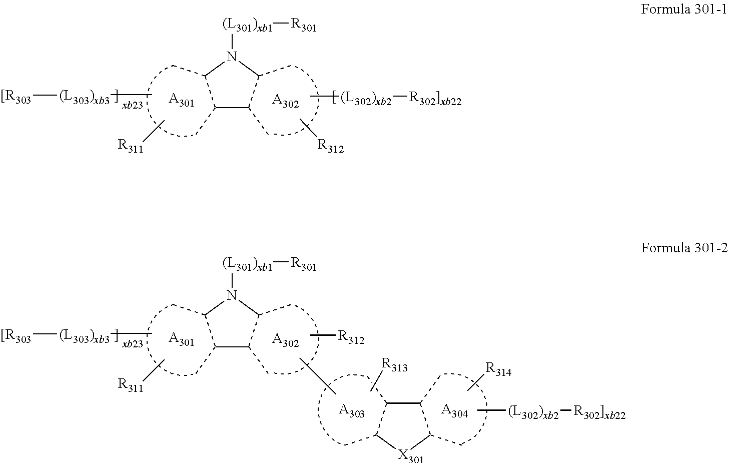

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄) (R₃₀₅), or Si(R₃₀₄)(R₃₀₅),
xb22 and xb23 may each independently be 0, 1, or 2,
L₃₀₁, xb1, and R₃₀₁ may each be the same as defined in one or more embodiments,
L₃₀₂ to L₃₀₄ may each independently be the same as defined with respect to L₃₀₁,
xb2 to xb4 may each independently be the same as defined with respect to xb1, and
R₃₀₂ to R₃₀₅ and R₃₁₁ to R₃₁₄ may each be the same as defined with respect to R₃₀₁.

In one or more embodiments, the host may include an alkaline earth metal complex, a post-transition metal complex, or a (e.g., any suitable) combination thereof. In one or more embodiments, the host may include a Be complex (for example, Compound H55), an Mg complex, a Zn complex, or a (e.g., any suitable) combination thereof.

In one or more embodiments, the host may include: one (e.g., may include at least one or be any one) selected from among Compounds H1 to H128; 9,10-di(2-naphthyl) anthracene (ADN); 2-methyl-9,10-bis(naphthalen-2-yl) anthracene (MADN); 9,10-di-(2-naphthyl)-2-t-butyl-anthracene (TBADN); 4,4′-bis(N-carbazolyl)-1,1′-biphenyl (CBP); 1,3-di(carbazol-9-yl)benzene (mCP); 1,3,5-tri (carbazol-9-yl)benzene (TCP); or a (e.g., any suitable) combination thereof:

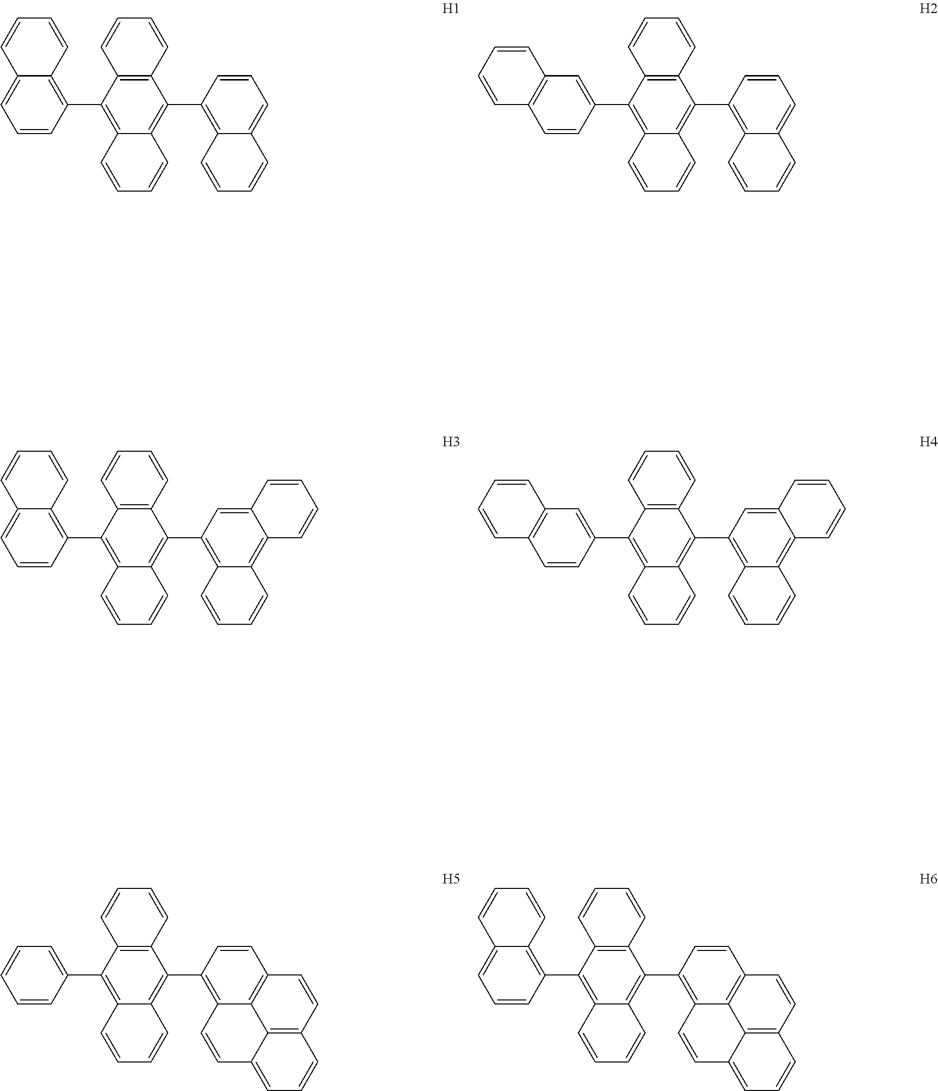

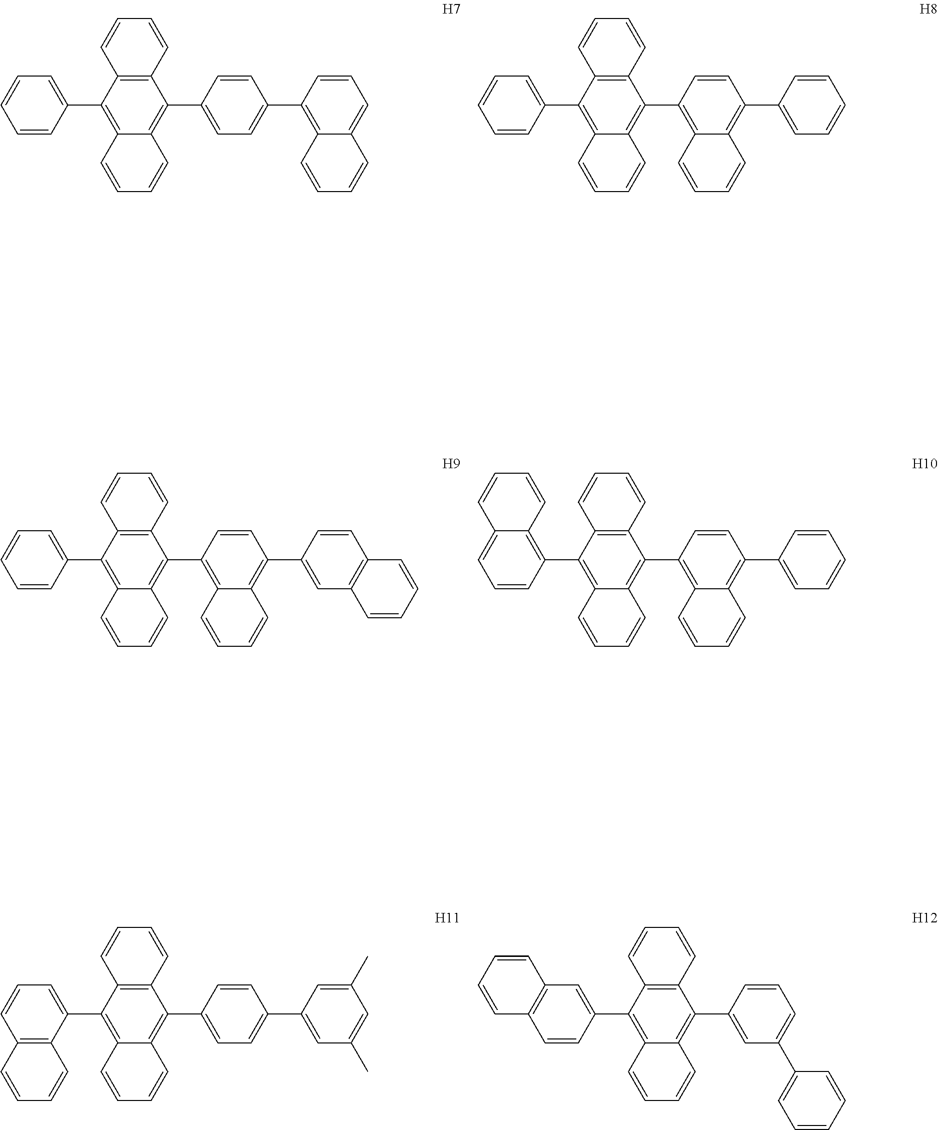

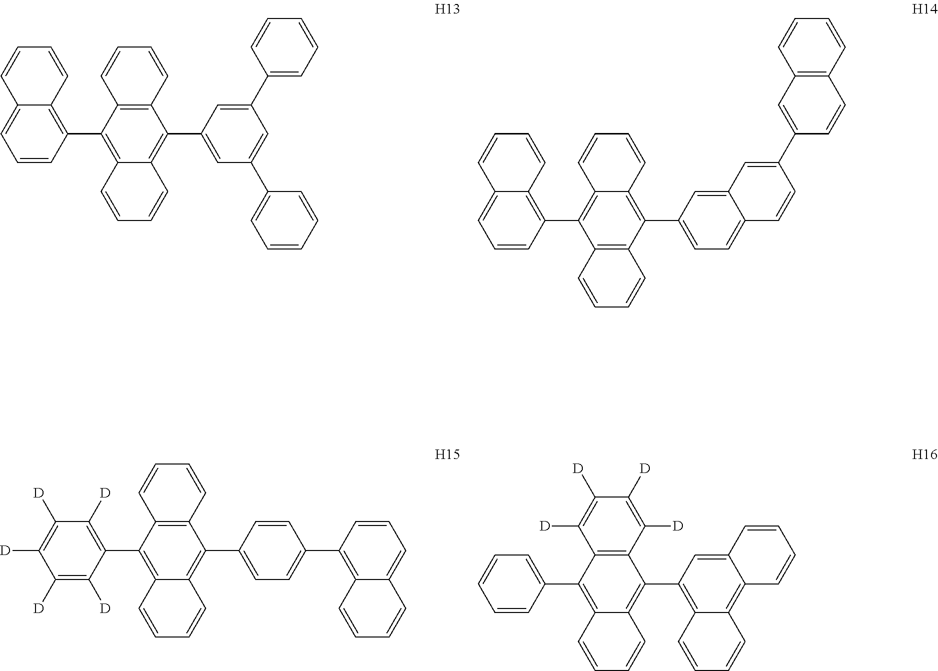

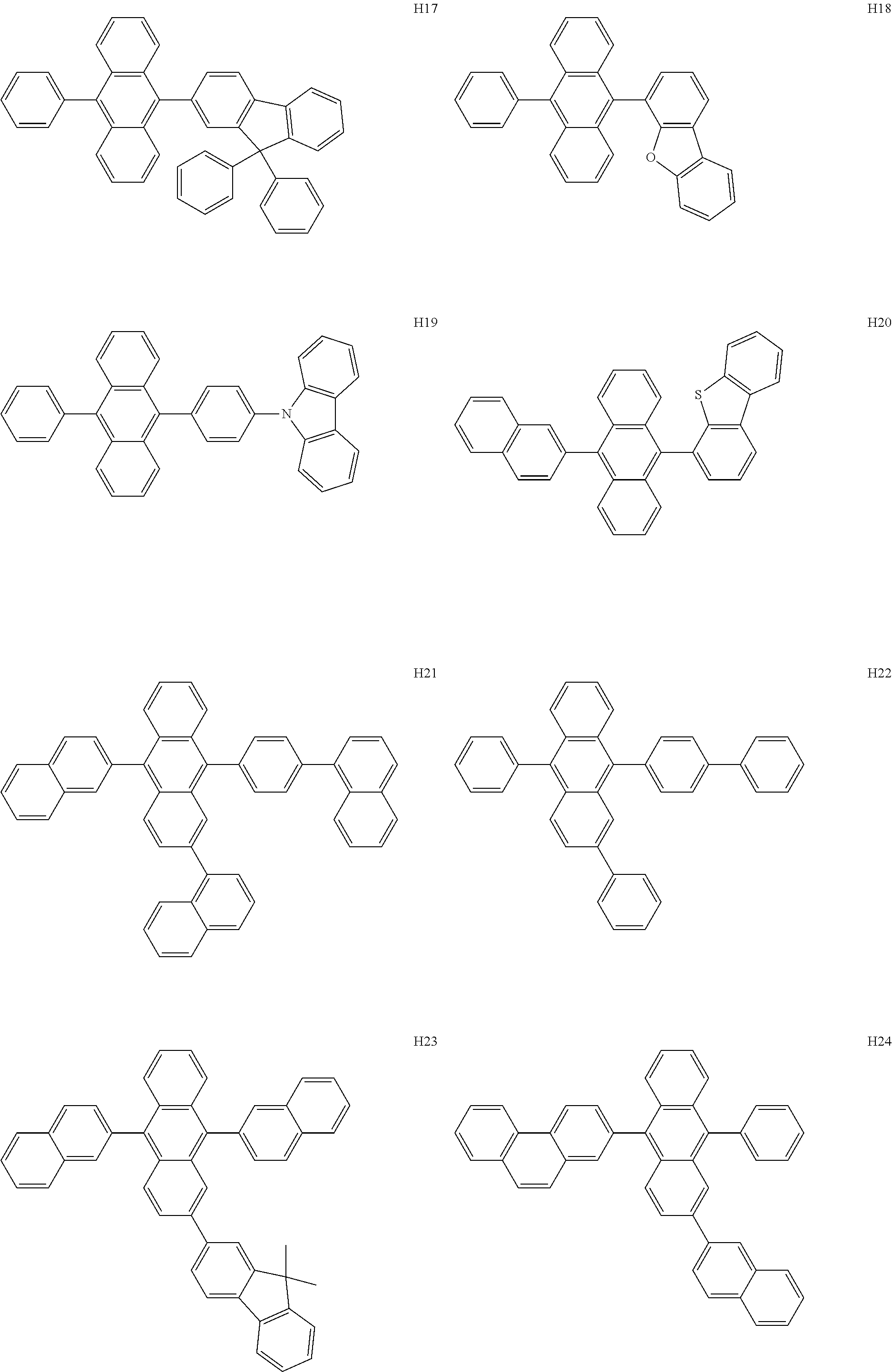

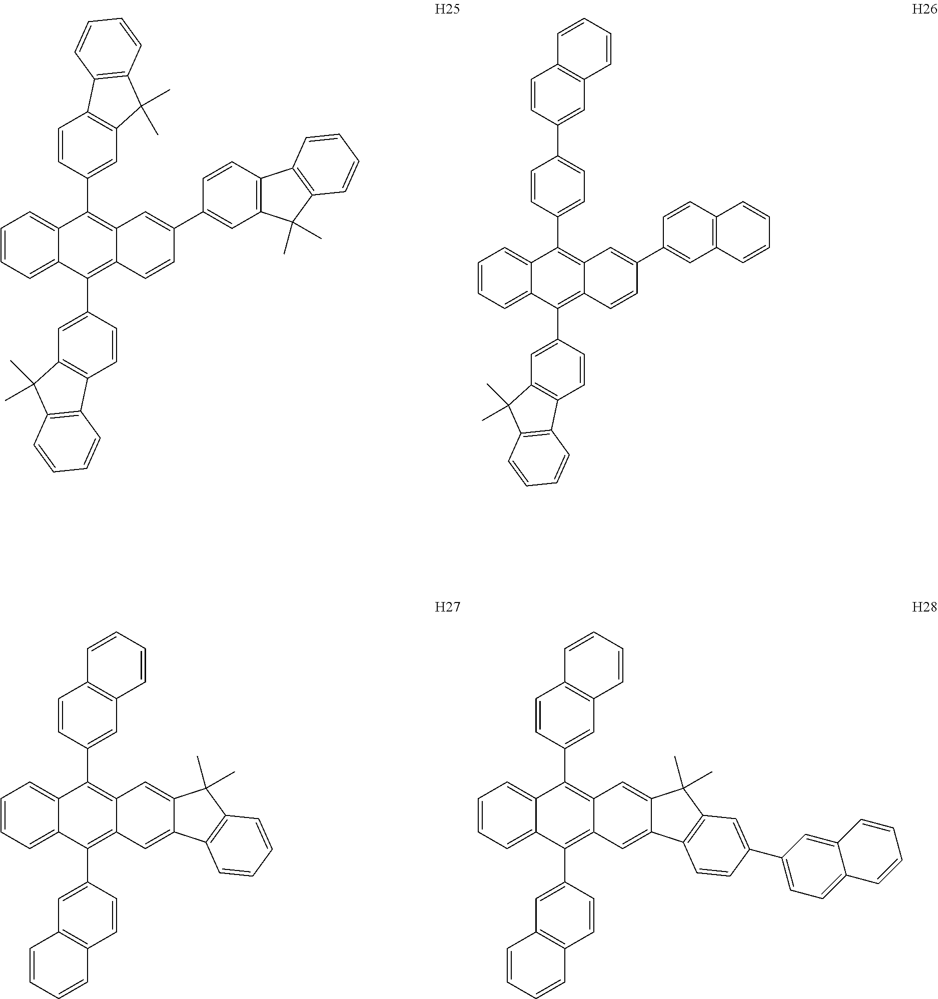

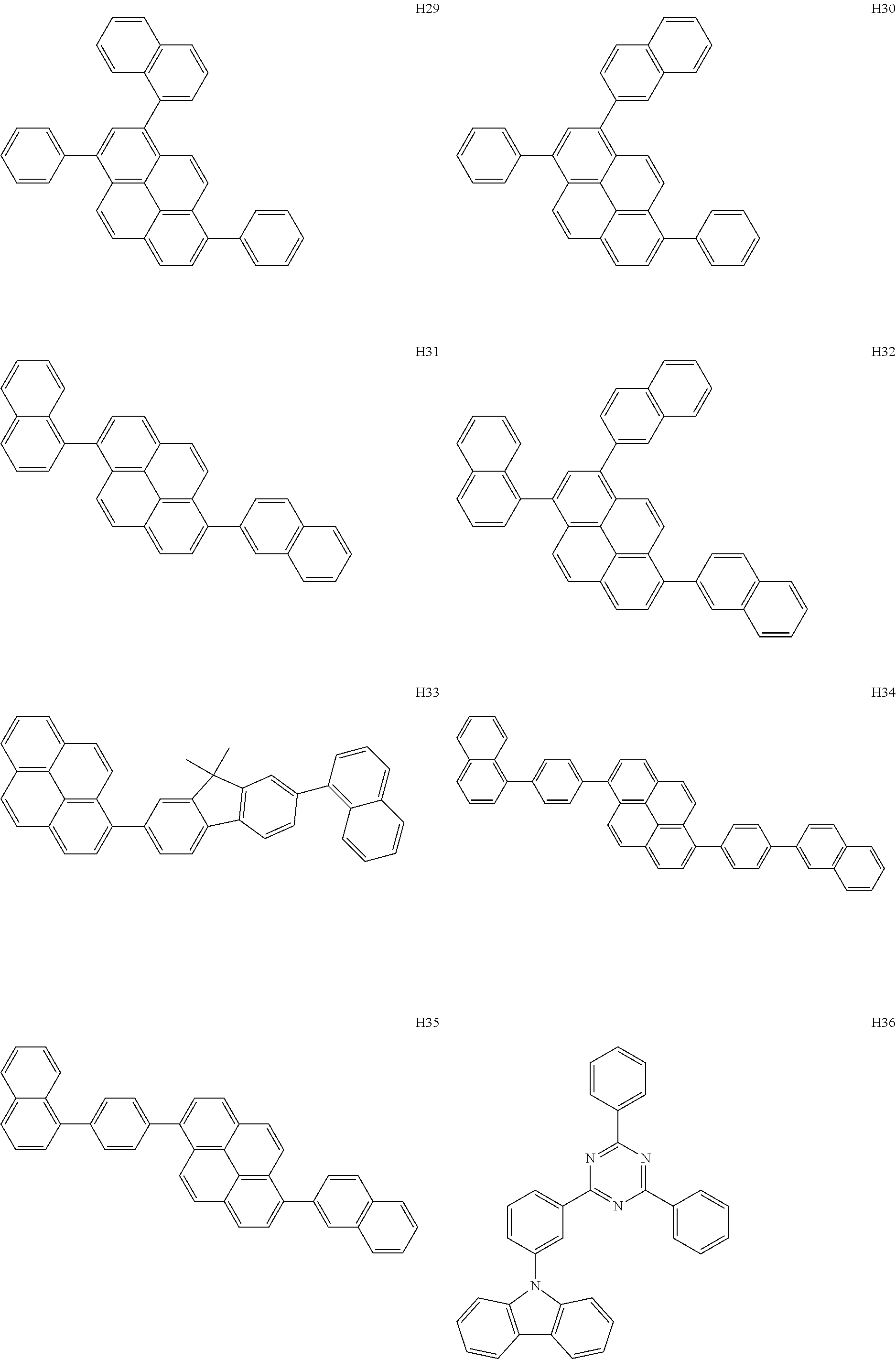

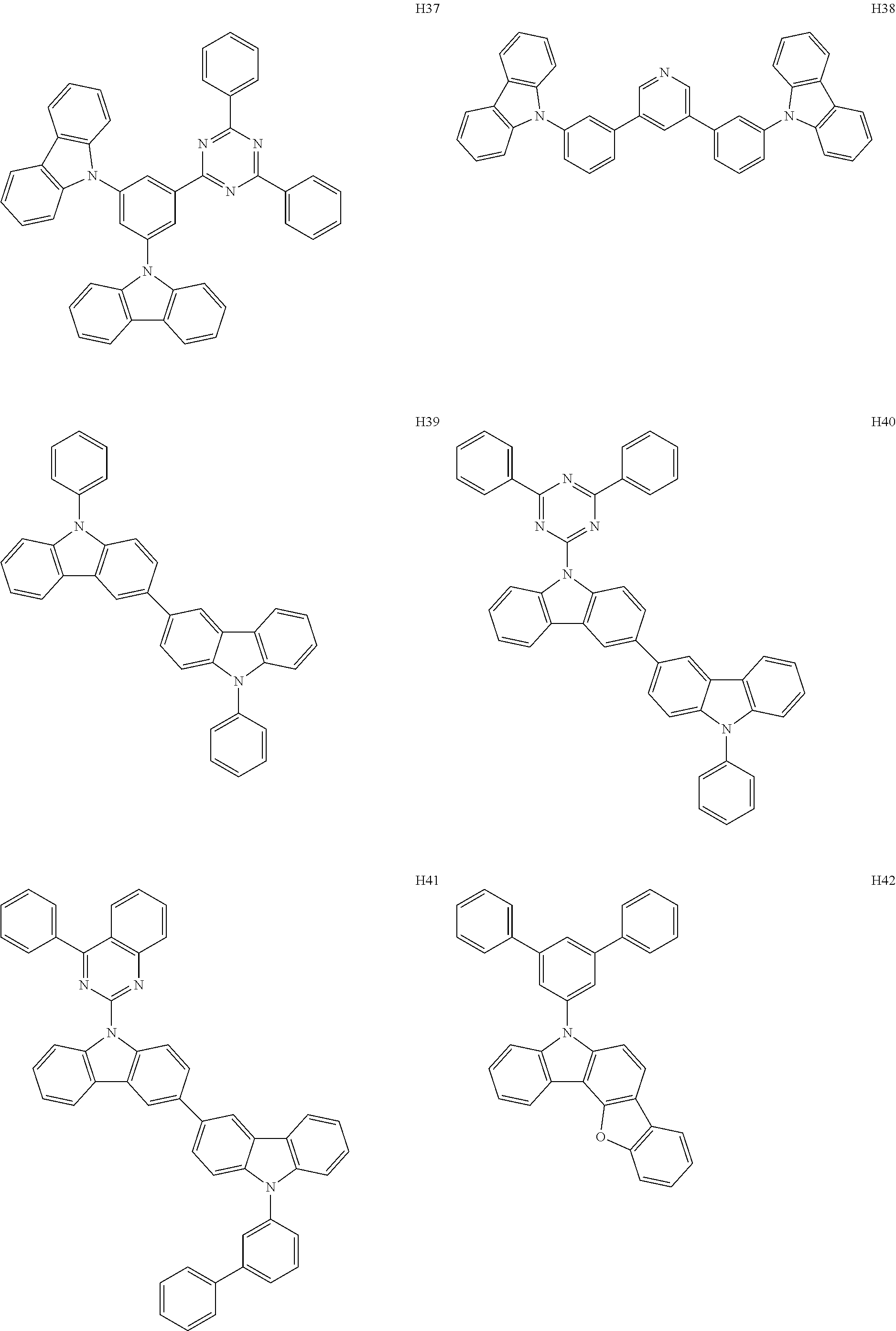 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

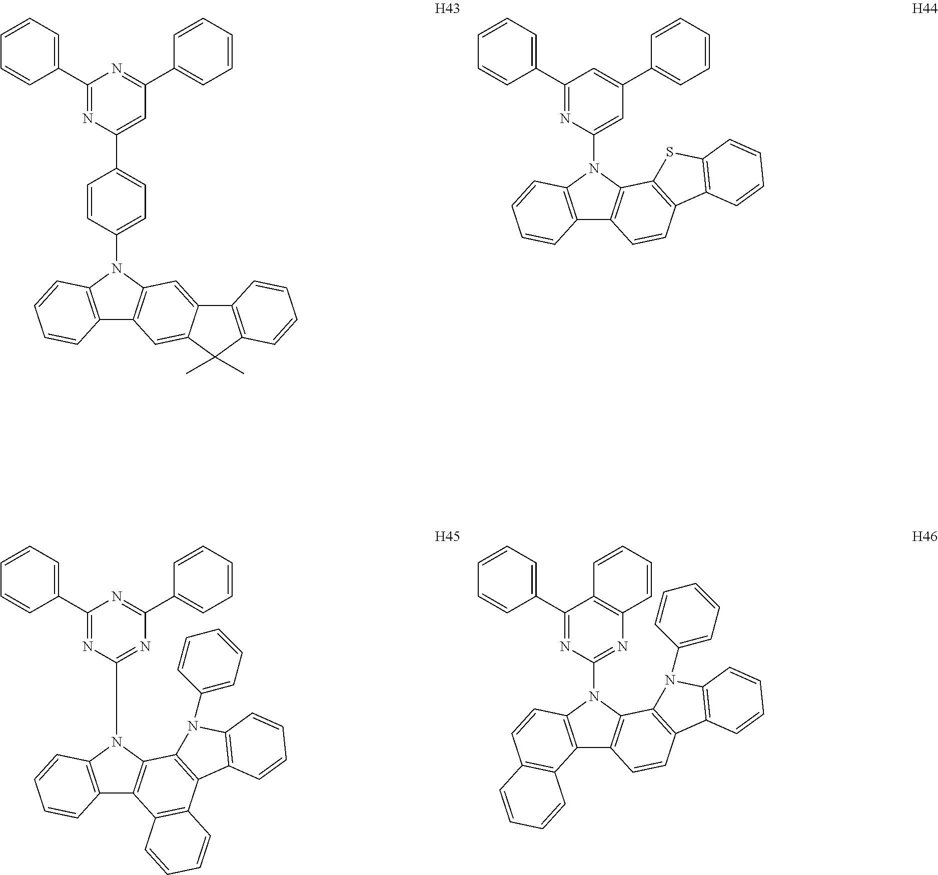 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

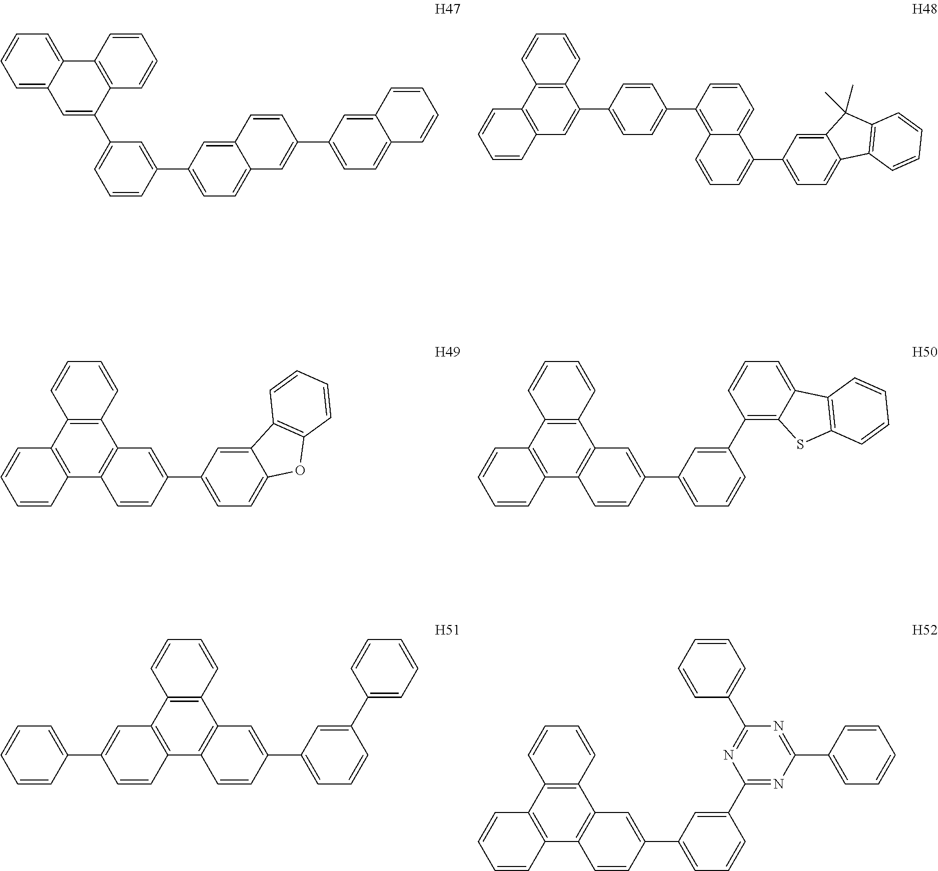

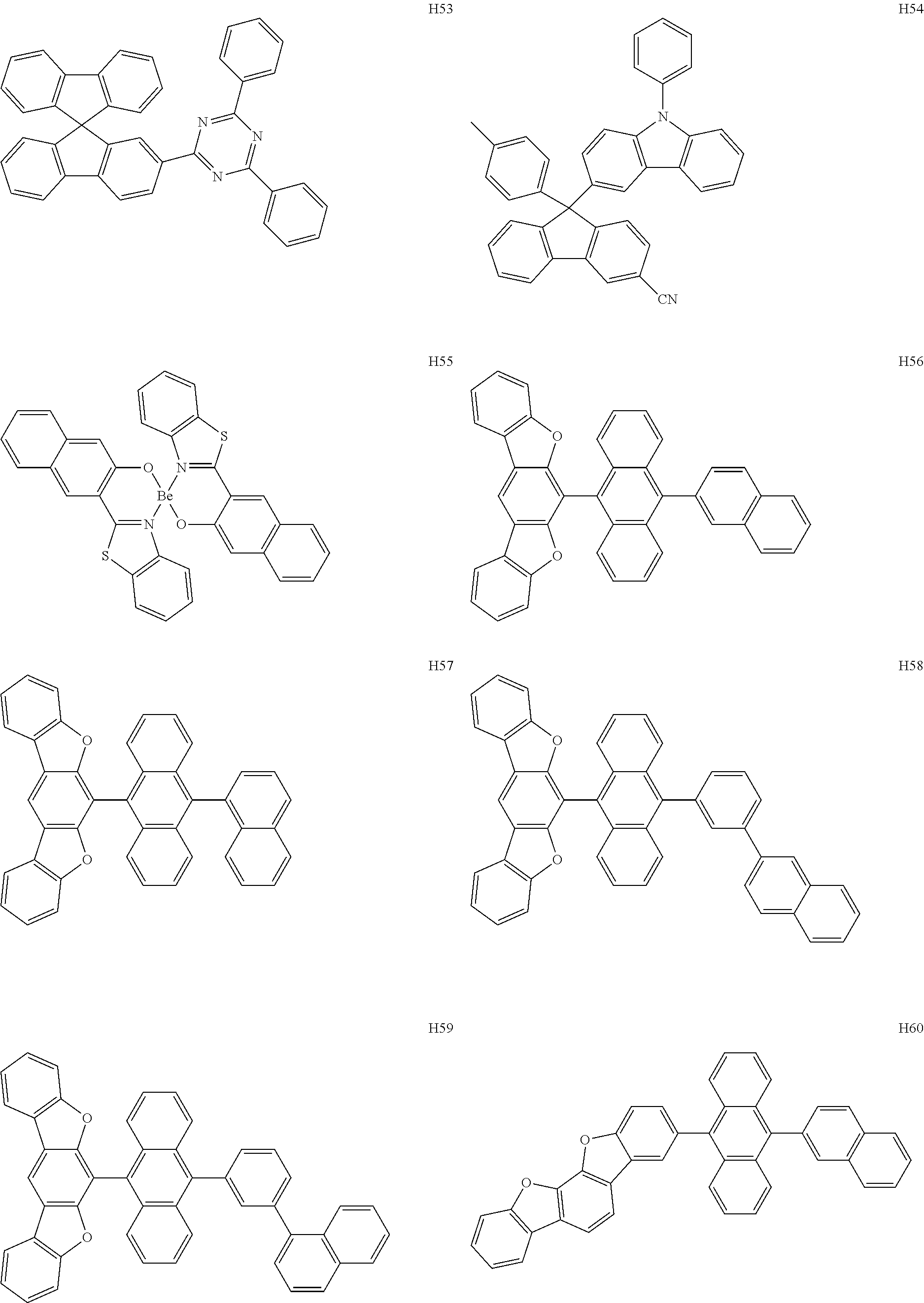

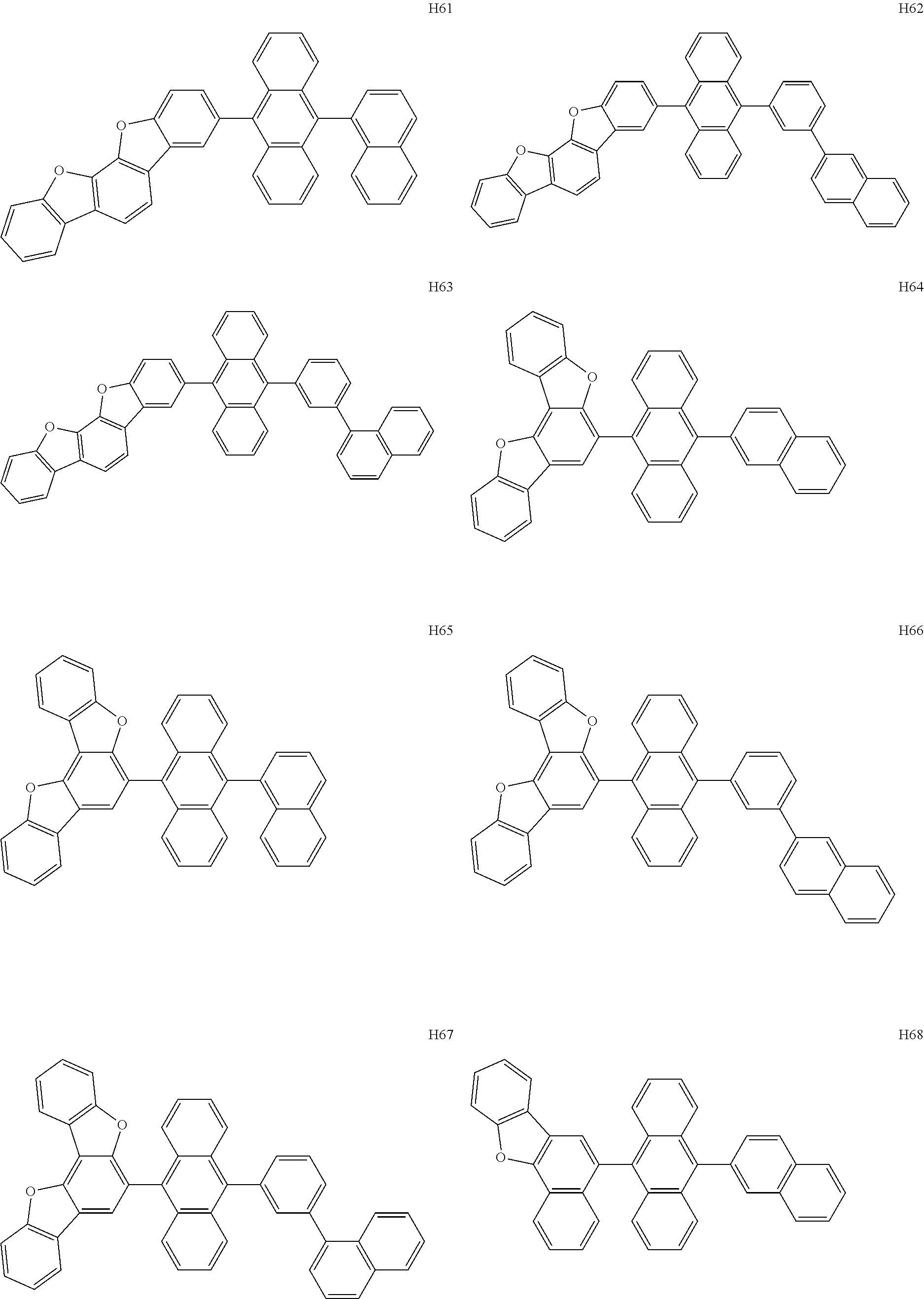

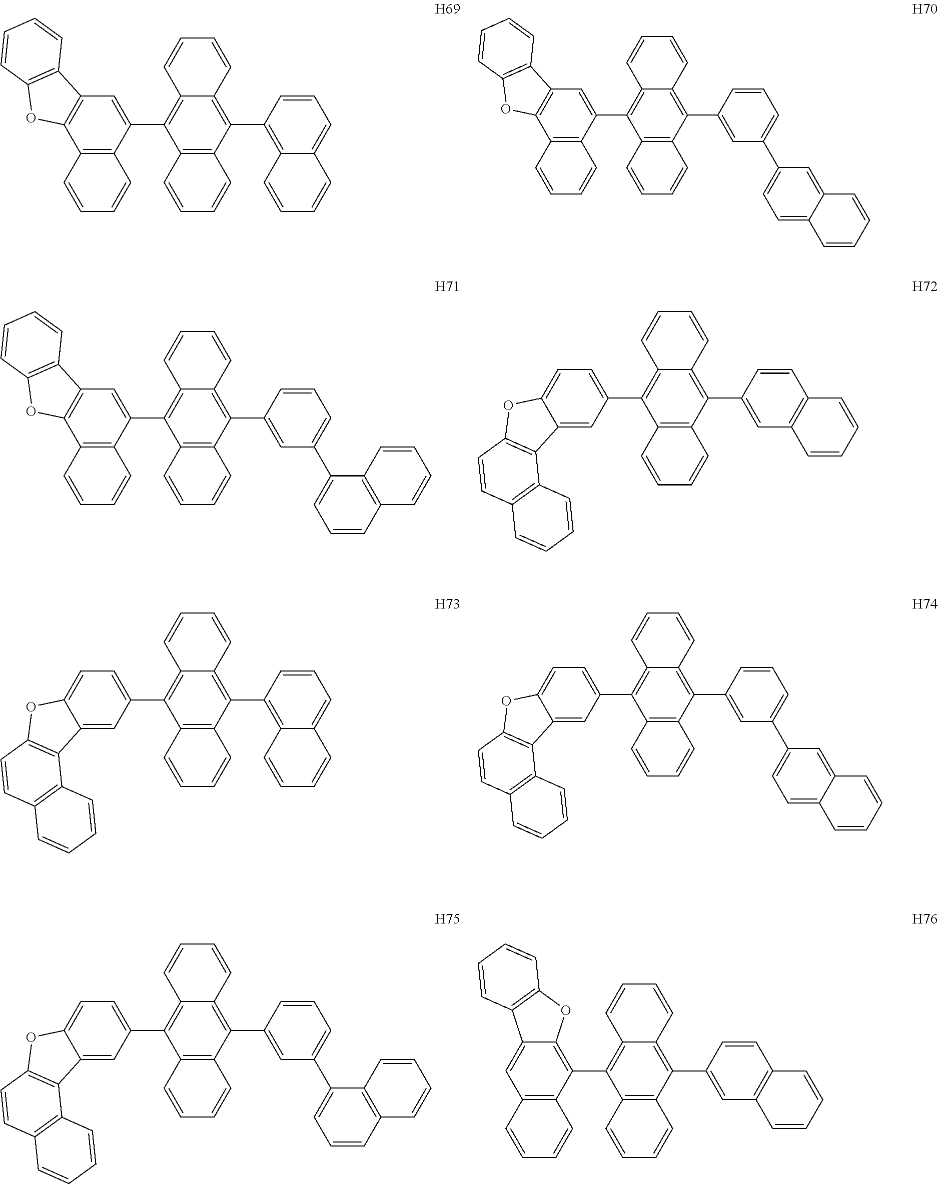

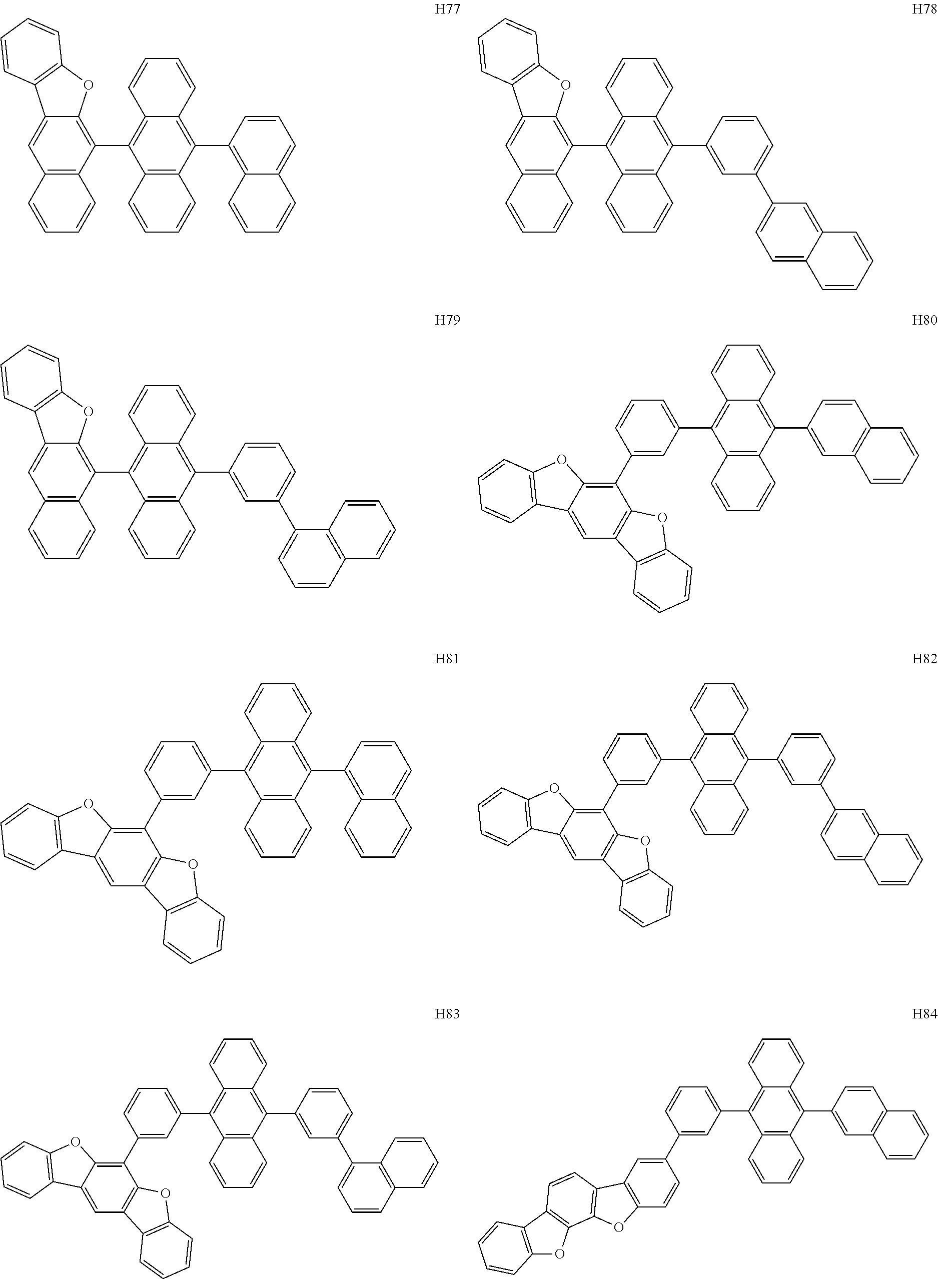

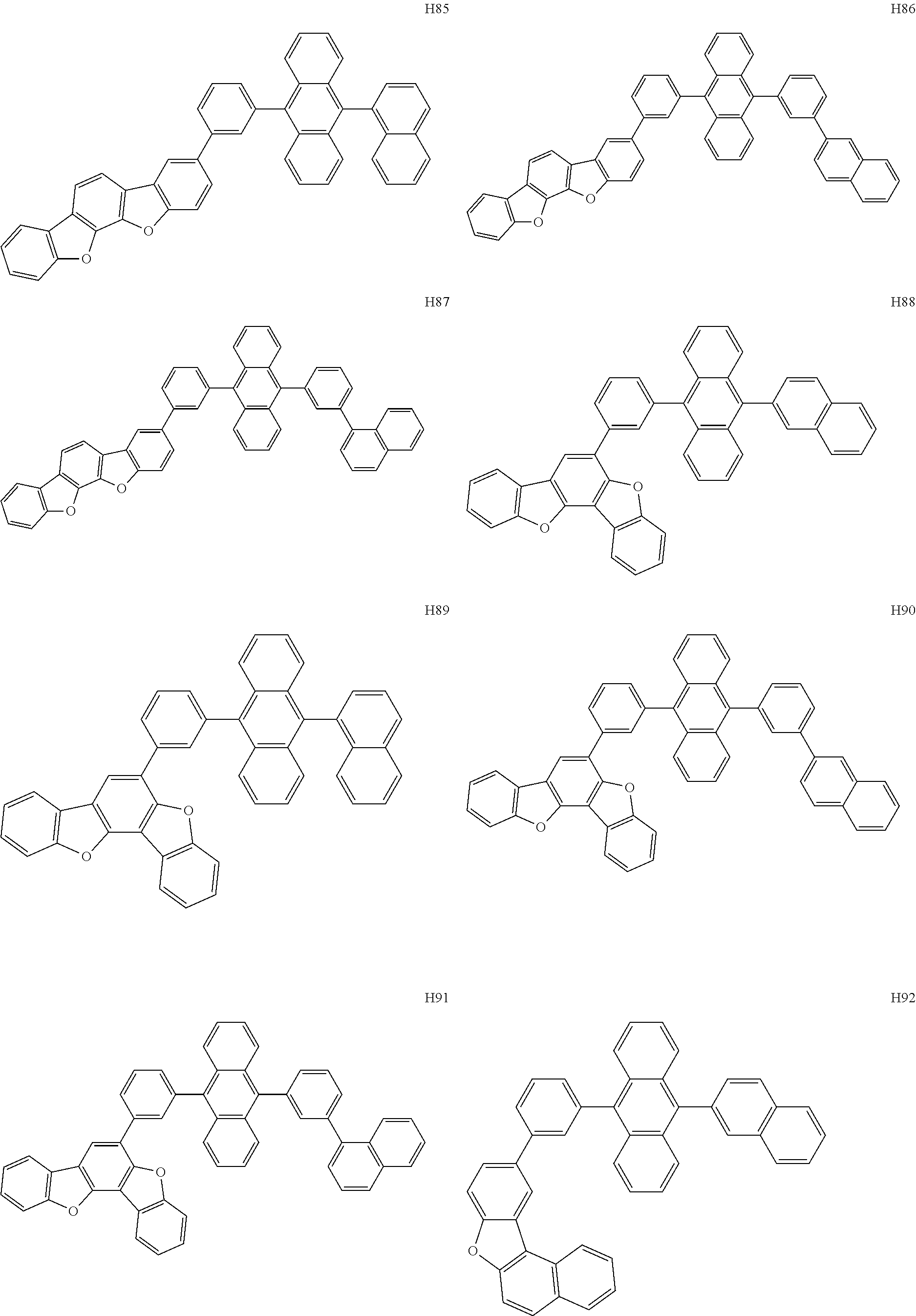

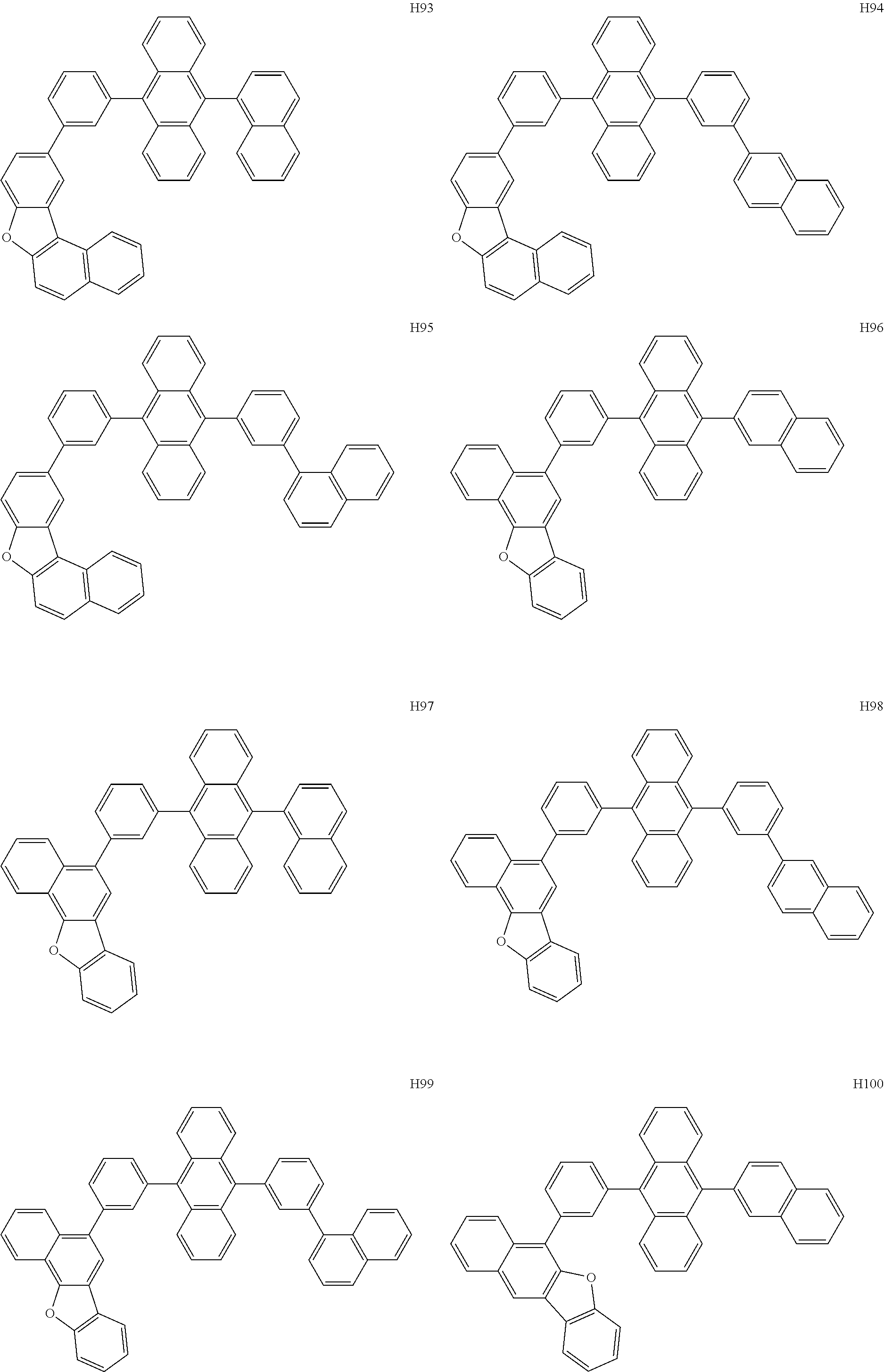

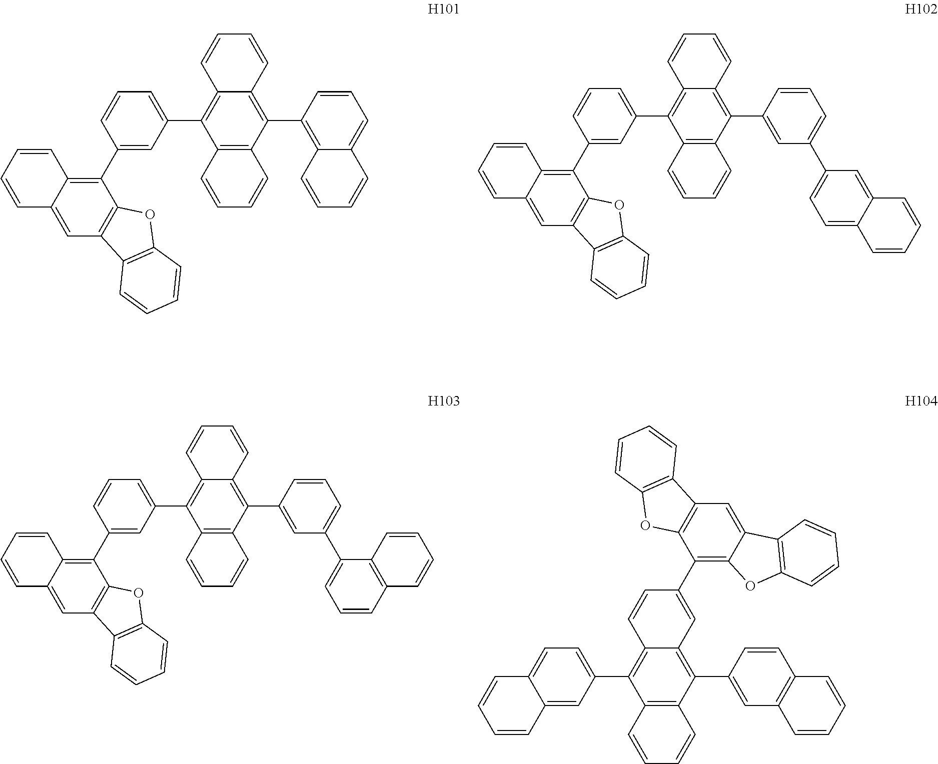 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

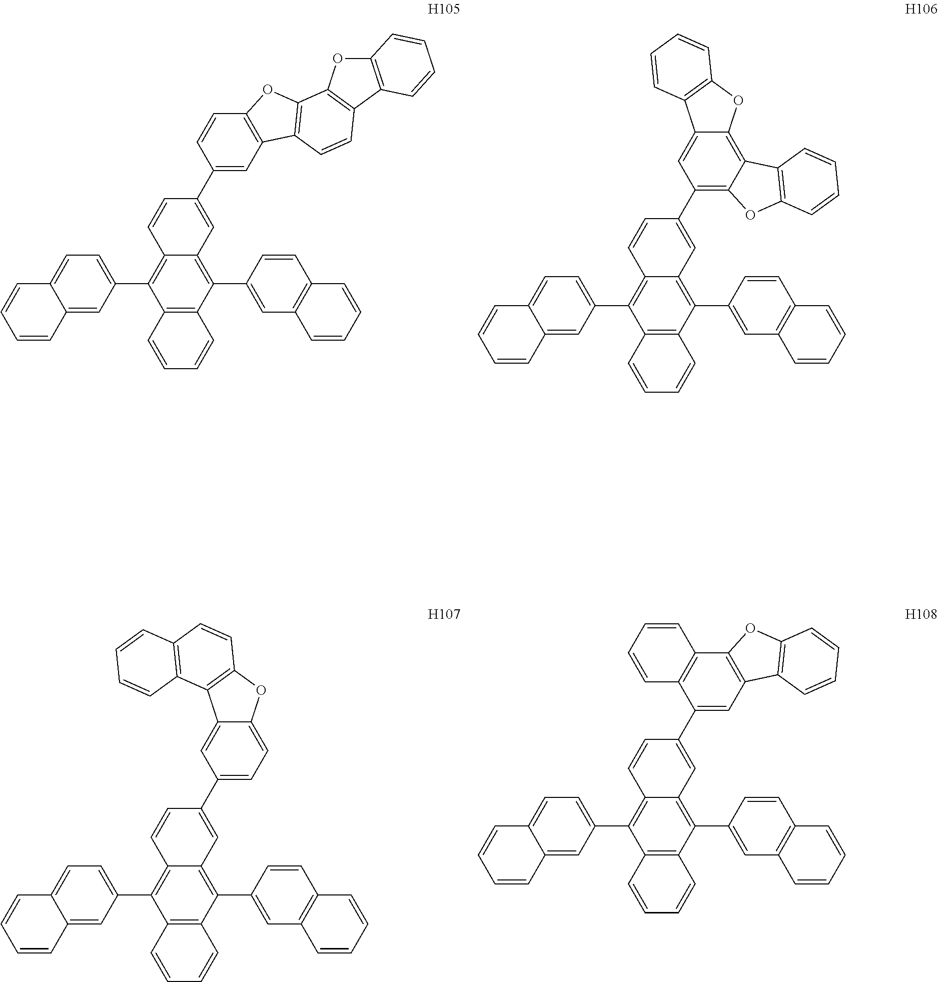 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

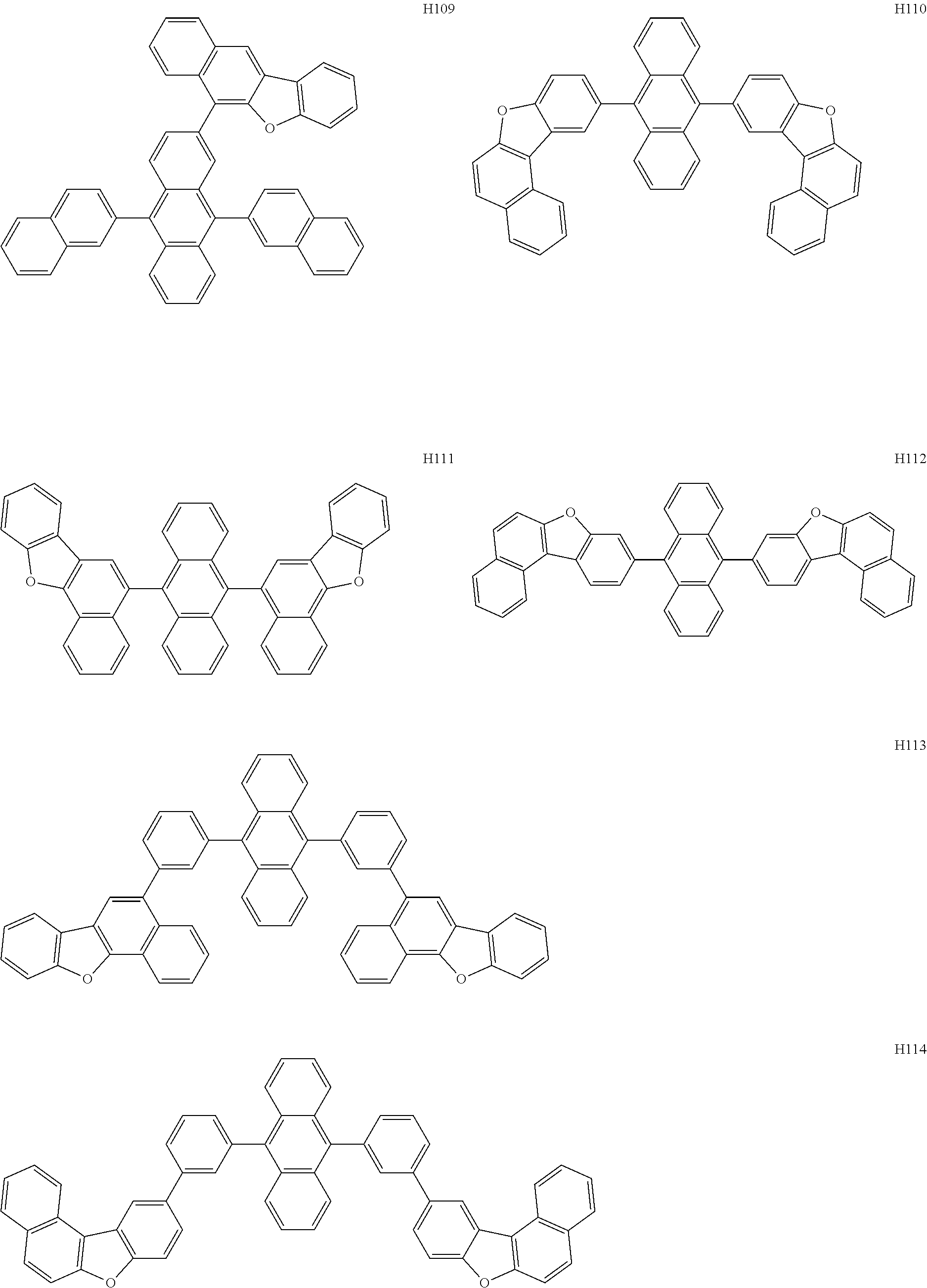

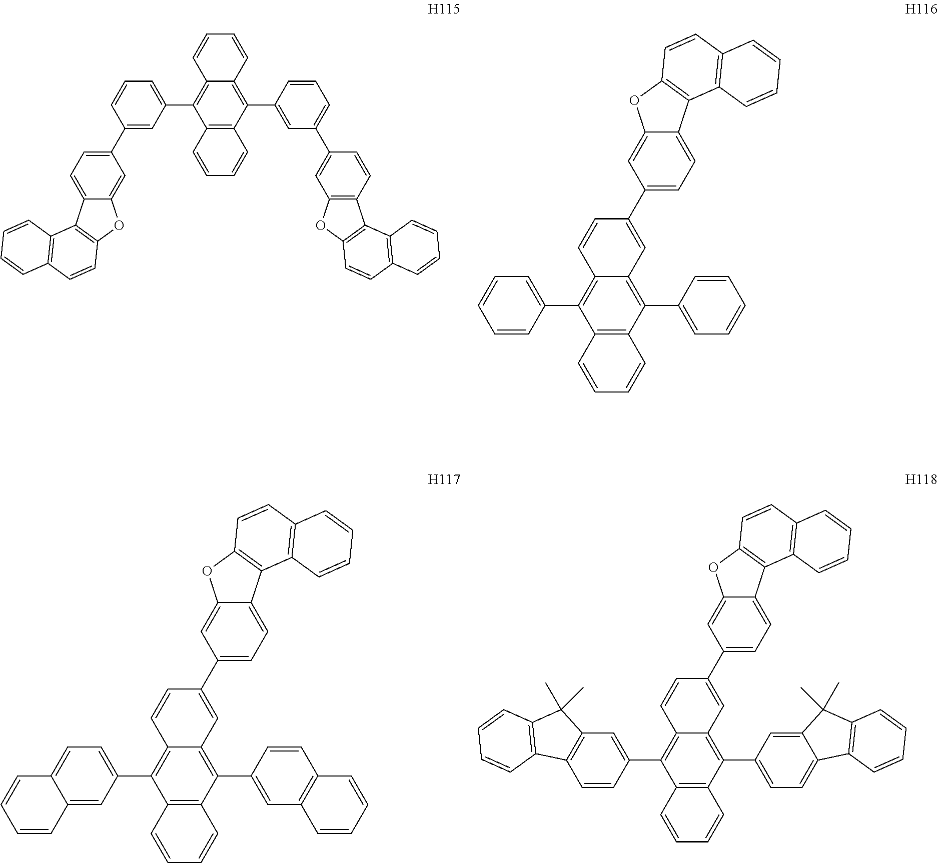 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

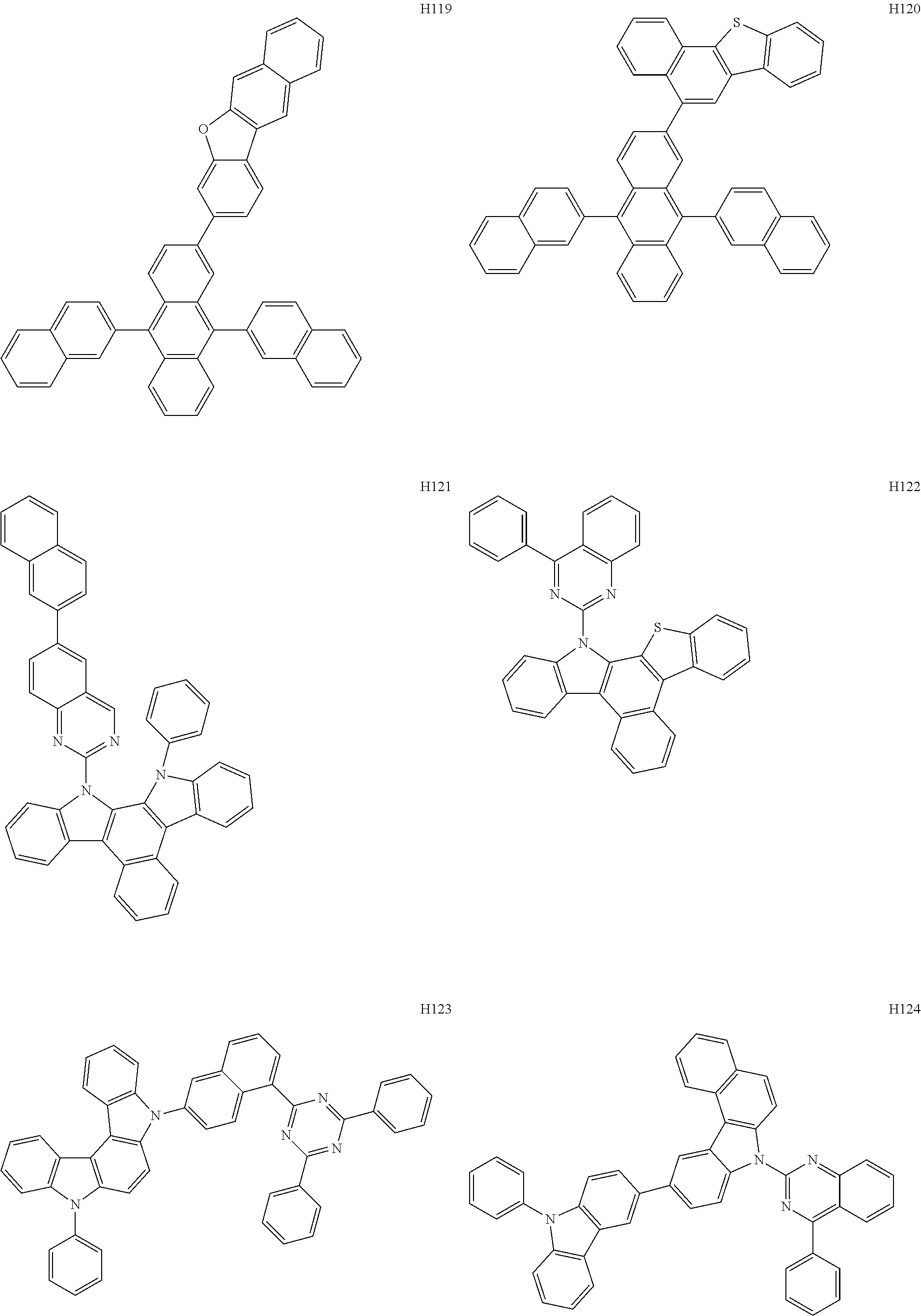

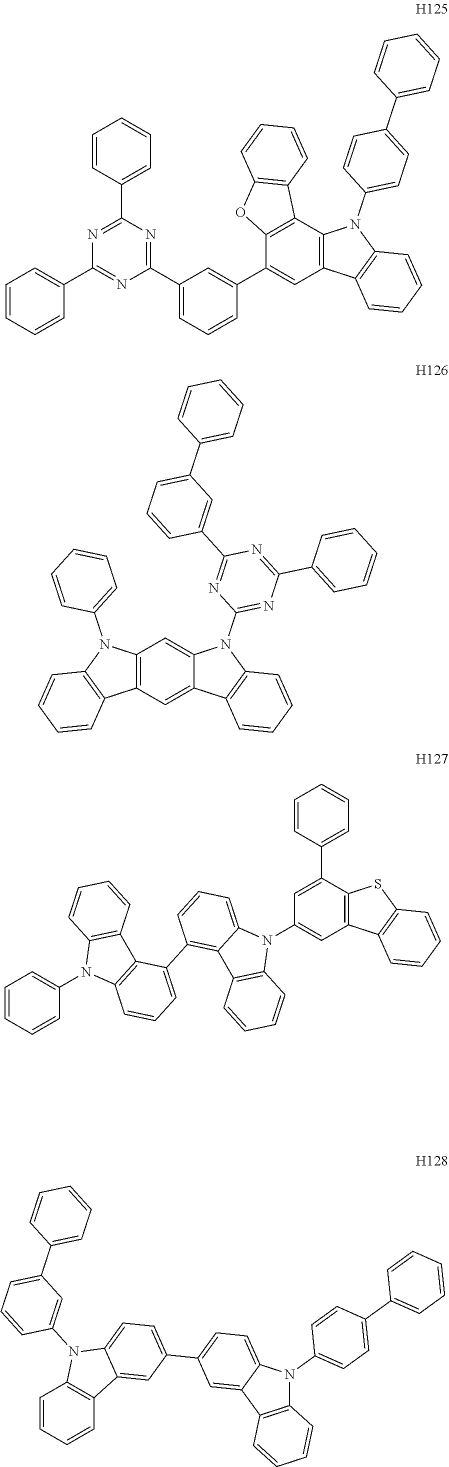 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

In one or more embodiments, the host may include a first host compound and a second host compound.

In one or more embodiments, the first host compound may be a hole transporting host.

In one or more embodiments, the second host compound may be an electron transporting host.

In one or more embodiments, the term “hole transporting host” as used herein refers to a compound including a hole transporting moiety.

In one or more embodiments, the term “electron transporting host” as used herein refers to not only a compound including an electron transporting moiety, but also a compound having bipolar properties.

The terms “hole-transporting host” and “electron-transporting host” may each be understood according to the relative difference between the hole mobility and electron mobility in the hole transporting host and the electron transporting host. For example, even if (e.g., when) the electron transporting host does not include an electron transporting moiety, a bipolar compound that exhibits relatively higher electron mobility than the hole transporting host may be also understood as the electron transporting host.

In one or more embodiments, the hole transporting host may be represented by one selected from among Formulae 311-1 to 311-6, and the electron transporting host may be represented by one (e.g., any one) selected from among Formulae 312-1 to 312-4, 313, and 313A:

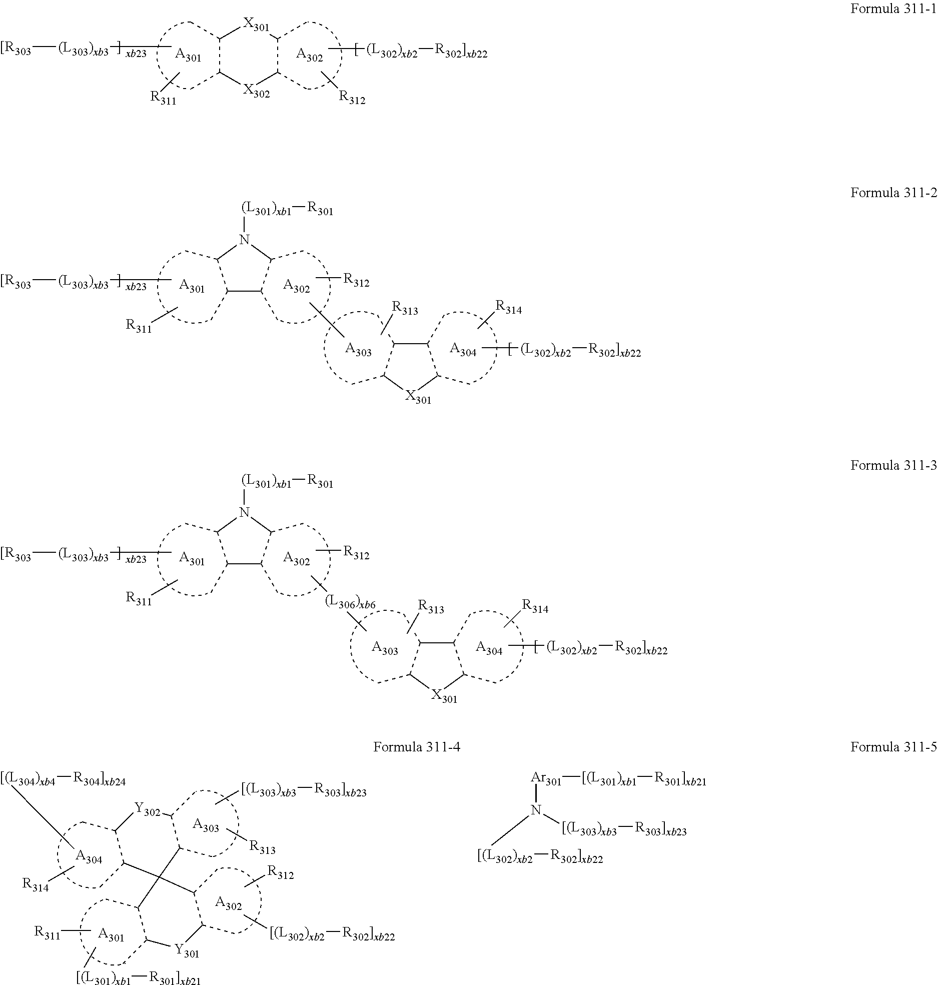

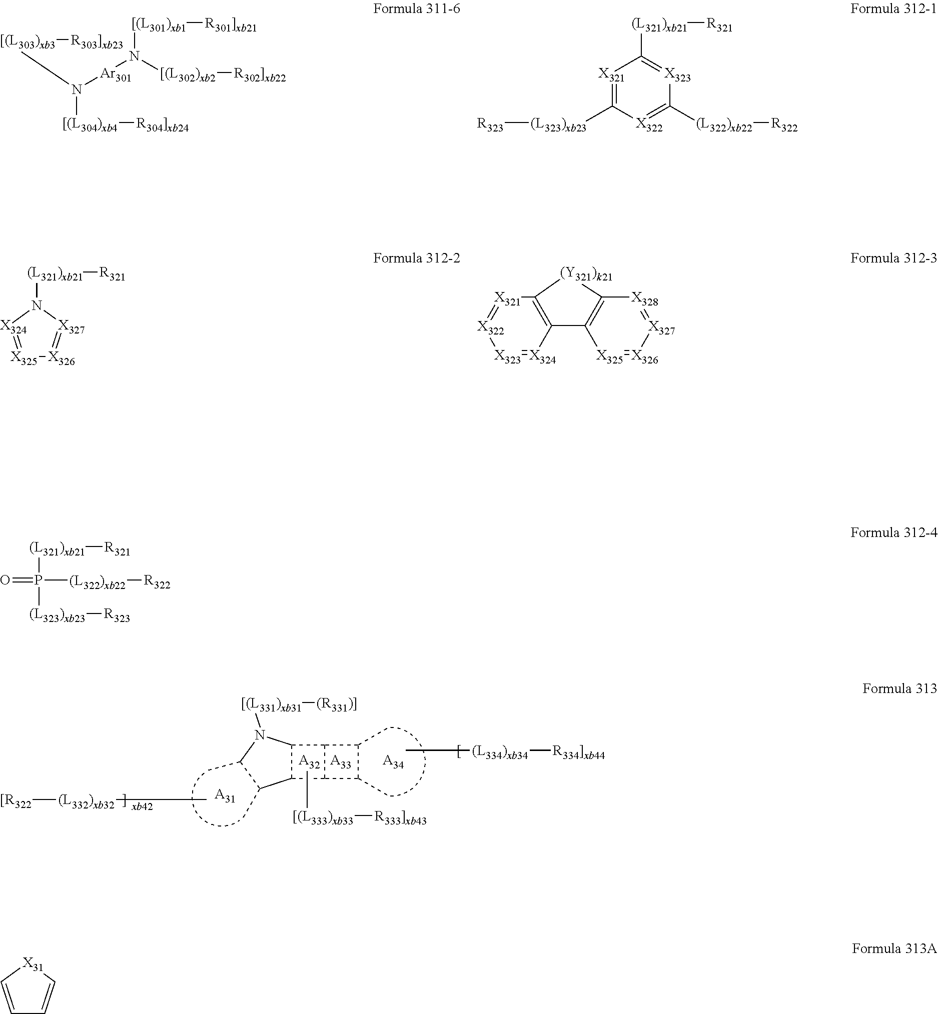

wherein, in Formulae 311-1 to 311-6, 312-1 to 312-4, 313, and 313A,
Ar₃₀₁ may be a C₃-C₆₀ carbocyclic group unsubstituted or substituted with at least one R₁₀ₐ or a C₁-C₆₀ heterocyclic group unsubstituted or substituted with at least one R₁₀ₐ,
A₃₀₁ to A₃₀₄ may each independently be a C₃-C₆₀ carbocyclic group or a C₁-C₆₀ heterocyclic group,
X₃₀₁ may be O, S, N[(L₃₀₄)_{xb4}-R₃₀₄], C[(L₃₀₄)_{xb4}-R₃₀₄][(L₃₀₅)_{xb5}—R₃₀₅], or Si[(L₃₀₄)_{xb4}-R₃₀₄][(L₃₀₅)_{xb5}-R₃₀₅],
X₃₀₂, Y₃₀₁, and Y₃₀₂ may each independently be a single bond (e.g., a single covalent bond), O, S, N[(L₃₀₅)_{xb5}-R₃₀₅], C[(L₃₀₄)ₓ₆₄-R₃₀₄][(L₃₀₅)_{xb5}-R₃₀₅], Si[(L₃₀₄)_{xb4}-R₃₀₄][(L₃₀₅)_{xb5}-R₃₀₅], or S(═O)₂,
xb1 to xb5 may each be 0, 1, 2, 3, 4, or 5,
xb6 may be 1, 2, 3, 4, or 5,
X₃₂₁ to X₃₂₈ may each independently be N or C[(L₃₂₄)_{xb24}-R₃₂₄],
Y₃₂₁ may be *—O—*′, *—S—*′, *—N[(L₃₂₅)_{xb25}-R₃₂₅]—*′, *—C[(L₃₂₅)_{xb25}-R₃₂₅][(L₃₂₆)_{xb26}-R₃₂₆]—*′, *—C[(L₃₂₅)_{xb25}-R₃₂₅]═C[(L₃₂₆)_{xb26}-R₃₂₆]—*′, *—C[(L₃₂₅)_{xb25}-R₃₂₅]=N—*′, or *—N═C[(L₃₂₆)_{xb26}-R₃₂₆]—*′,
k21 may be 0, 1, or 2, wherein Y₃₂₁ is not present if (e.g., when) k21 is 0,
xb21 to xb26 may each independently be 0, 1, 2, 3, 4, or 5,
A₃₁, A₃₂, and A₃₄ may each independently be a C₃-C₆₀ carbocyclic group or a C₁-C₃₀ heterocyclic group,
A₃₃ may be a group represented by Formula 313A,
X₃₁ may be N[(L₃₃₅)_{xb35}-(R₃₃₅)], O, S, Se, C[(L₃₃₅)_{xb35}-(R₃₃₅)][(L₃₃₆)_{xb36}-(R₃₃₆)], or Si[(L₃₃₅)_{xb35}-(R₃₃₅)][(L₃₃₆)_{xb36}-(R₃₃₆)],
xb31 to xb36 may each independently be 0, 1, 2, 3, 4, or 5,
xb42 to xb44 may each independently be 0, 1, 2, 3, 4, 5, 6, 7, 8, 9, or 10,
L₃₀₁ to L₃₀₆, L₃₂₁ to L₃₂₆, and L₃₃₁ to L₃₃₆ may each independently be a single bond (e.g., a single covalent bond), a C₁-C₂₀ alkylene group unsubstituted or substituted with at least one R₁₀ₐ, a C₁-C₂₀ alkenylene group unsubstituted or substituted with at least one R₁₀ₐ, a C₁-C₂₀ alkynylene group unsubstituted or substituted with at least one R₁₀ₐ, a C₃-C₁₀ cycloalkylene group unsubstituted or substituted with at least one R₁₀ₐ, a C₁-C₁₀ heterocycloalkylene group unsubstituted or substituted with at least one R₁₀ₐ, a C₃-C₁₀ cycloalkenylene group unsubstituted or substituted with at least one R₁₀ₐ, a C₁-C₁₀ heterocycloalkenylene group unsubstituted or substituted with at least one R₁₀ₐ, a C₆-C₆₀ arylene group unsubstituted or substituted with at least one R₁₀ₐ, a C₁-C₆₀ heteroarylene group unsubstituted or substituted with at least one R₁₀ₐ, a divalent non-aromatic condensed polycyclic group unsubstituted or substituted with at least one R₁₀ₐ, or a divalent non-aromatic condensed heteropolycyclic group unsubstituted or substituted with at least one R₁₀ₐ,
R₃₀₁ to R₃₀₅, R₃₁₁ to R₃₁₄, R₃₂₁ to R₃₂₄, and R₃₃₁ to R₃₃₆ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O) (Q₁), —S(═O) (Q₁), —S(═O)₂ (Q₁), —P(═O)(Q₁)(Q₂), or —P(═S)(Q₁)(Q₂),
neighboring two or more selected from among R₃₂₁ to R₃₂₄ may optionally be bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₅-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O) (Q₁₁), —S(═O)₂ (Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroaryl alkyl group, a C₁-C₆₀ heteroaryloxy group, or a C₁-C₆₀ heteroarylthio group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroaryl alkyl group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O) (Q₂₁), —S(═O)₂ (Q₂₁), —P(═O)(Q₂₁)(Q₂₂), or any combination thereof; or
—O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O) (Q₃₁), —S(═O)₂ (Q₃₁), or —P(═O)(Q₃₁)(Q₃₂), and
Q₁₁ to Q₁₃, Q₂₁ to Q₂₃, Q₃₁ to Q₃₃, Q₄₁ to Q₄₃, Q₃₀₁ to Q₃₀₃, Q₃₂₁ to Q₃₂₃, and Q₃₃₁ to Q₃₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each being unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, the first host compound and the second host compound may form an exciplex.

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or a (e.g., any suitable) combination thereof.

The phosphorescent dopant may be electrically neutral.

In one or more embodiments, the phosphorescent dopant may include an organometallic compound represented by Formula 401:

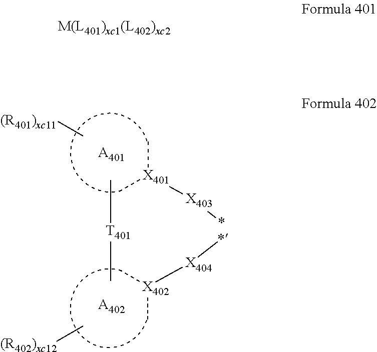

wherein, in Formulae 401 and 402,
M may be a transition metal (e.g., Ir, Pt, Pd, Os, Ti, Au, Hf, Eu, Tb, Rh, Re, or Tm),
L₄₀₁ may be a ligand represented by Formula 402, and xc1 may be 1, 2, or 3, wherein, if (e.g., when) xc1 is 2 or greater, two or more selected from among L₄₀₁ (s) may be identical to or different from each other,
L₄₀₂ may be an organic ligand, and xc2 may be 0, 1, 2, 3, or 4, wherein, if (e.g., when) xc2 is 2 or greater, two or more selected from among L₄₀₂ (s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond (e.g., a single covalent bond), *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *=C=*′,
X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each be the same as defined with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O) (Q₄₀₁), —S(═O)₂ (Q₄₀₁), or —P(═O)(Q₄₀₁)(Q₄₀₂),
Q₄₀₁ to Q₄₀₃ may each be the same as defined with respect to Q₁,
xc11 and xc12 may each independently be an integer of 0 to 10, and
* and *′ in Formula 402 each indicates a binding site to M in Formula 401.

In one or more embodiments, in Formula 402, i) X₄₀₁ may be nitrogen, and X₄₀₂ may be carbon, or ii) each of X₄₀₁ and X₄₀₂ may be nitrogen.

In one or more embodiments, if (e.g., when) xc1 in Formula 401 is 2 or greater, two ring A₄₀₁ (s) in two or more selected from among L₄₀₁ (s) may optionally be linked to each other via T₄₀₂, which is a linking group, or two ring A₄₀₂ (s) may optionally be linked to each other via T₄₀₃, which is a linking group (see Compounds PD1 to PD4 and PD7). T₄₀₂ and T₄₀₃ may each be the same as defined with respect to T₄₀₁.

L₄₀₂ in Formula 401 may be an organic ligand. In one or more embodiments, L₄₀₂ may include a halogen group, a diketone group (for example, an acetylacetonate group), a carboxylic acid group (for example, a picolinate group), —C(═O), an isonitrile group, a —CN group, a phosphorus-containing group (for example, a phosphine group, a phosphite group, and/or the like), or a (e.g., any suitable) combination thereof.

The phosphorescent dopant may include, for example, one (e.g., may include at least one or be any one) selected from among Compounds PD1 to PD39 or a (e.g., any suitable) combination thereof:

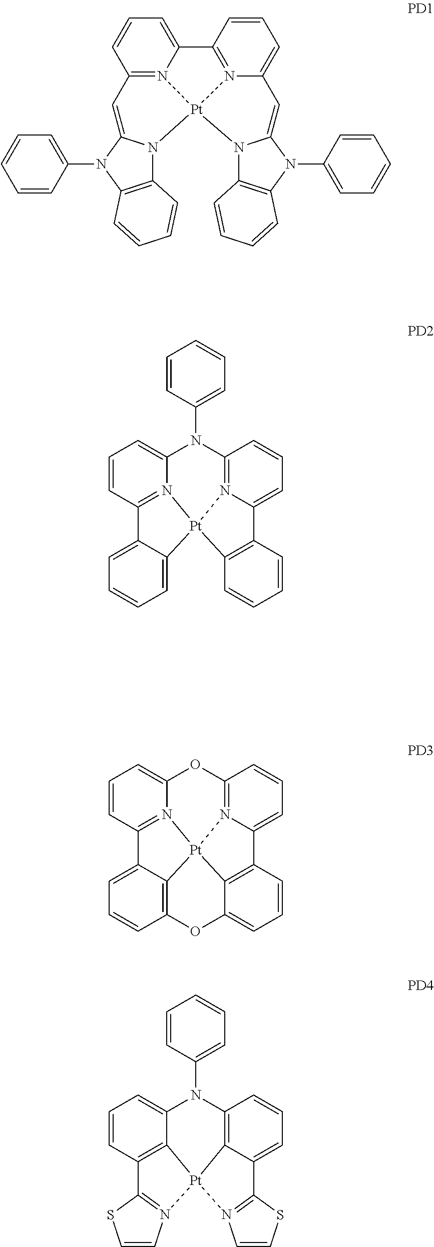

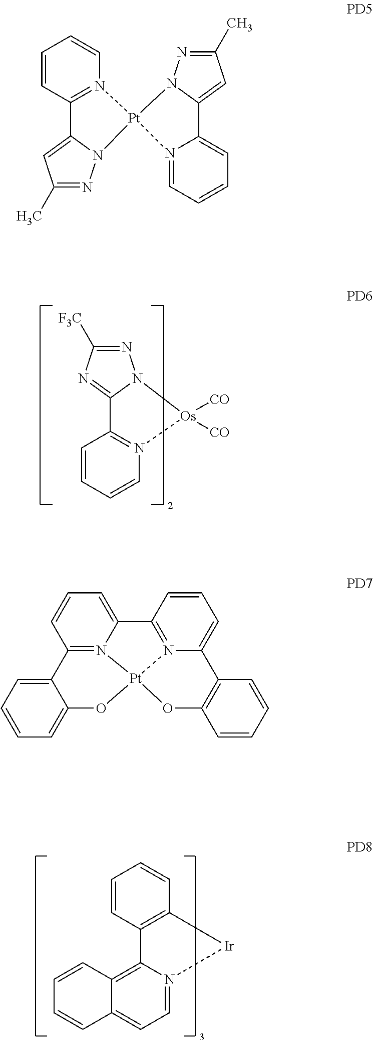

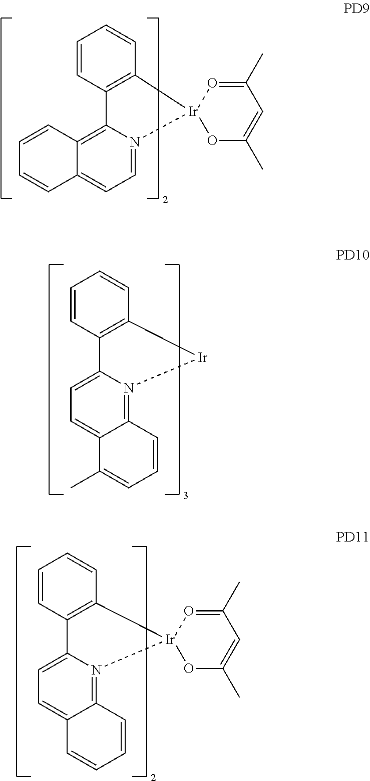

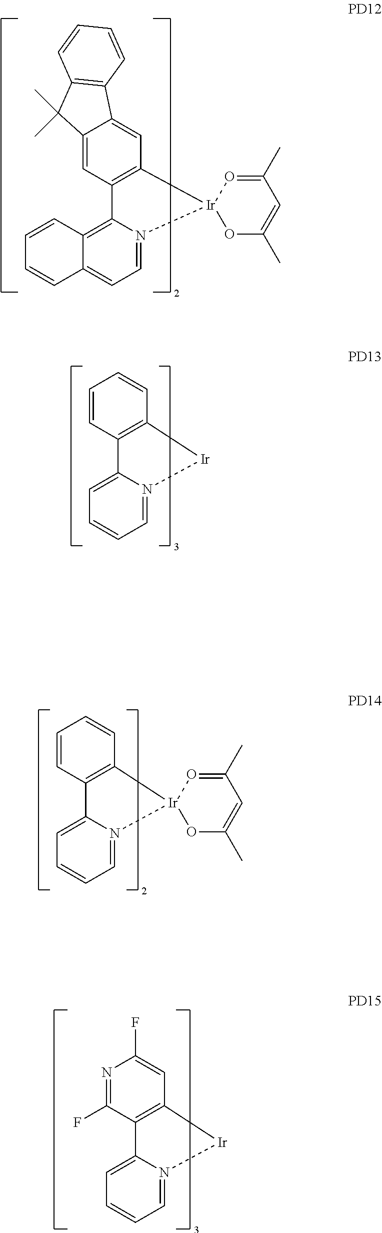

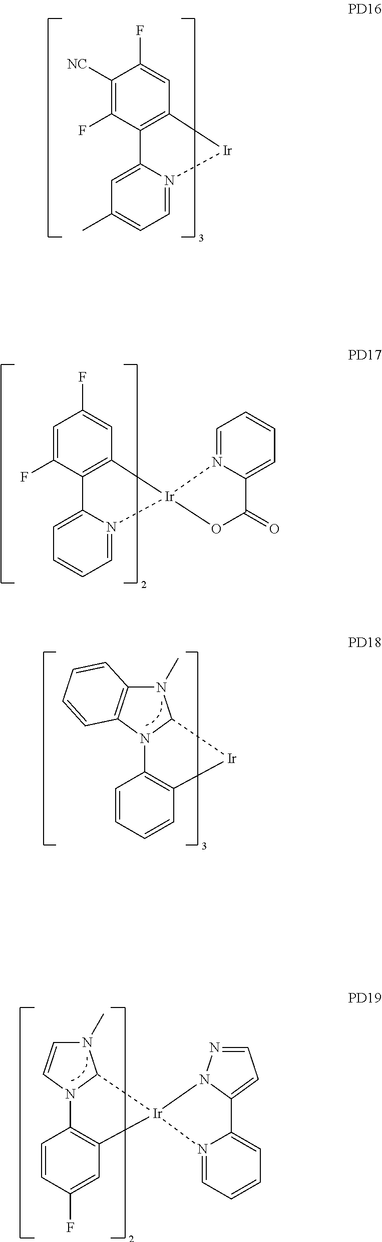

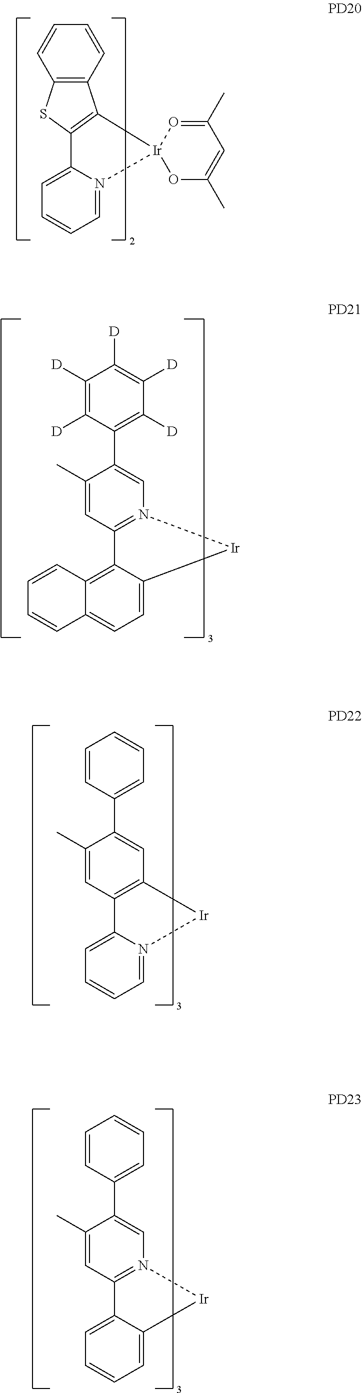

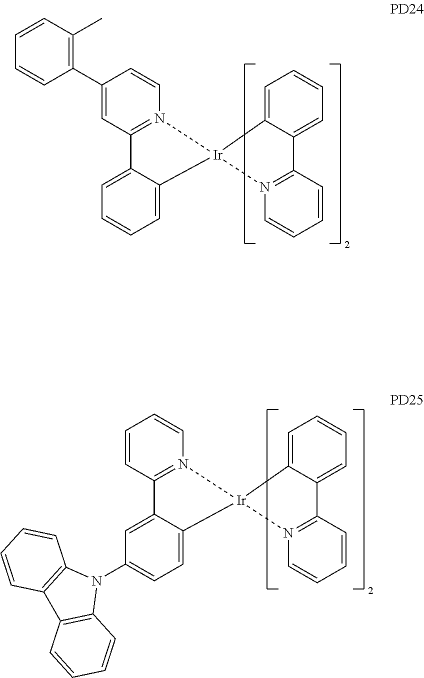

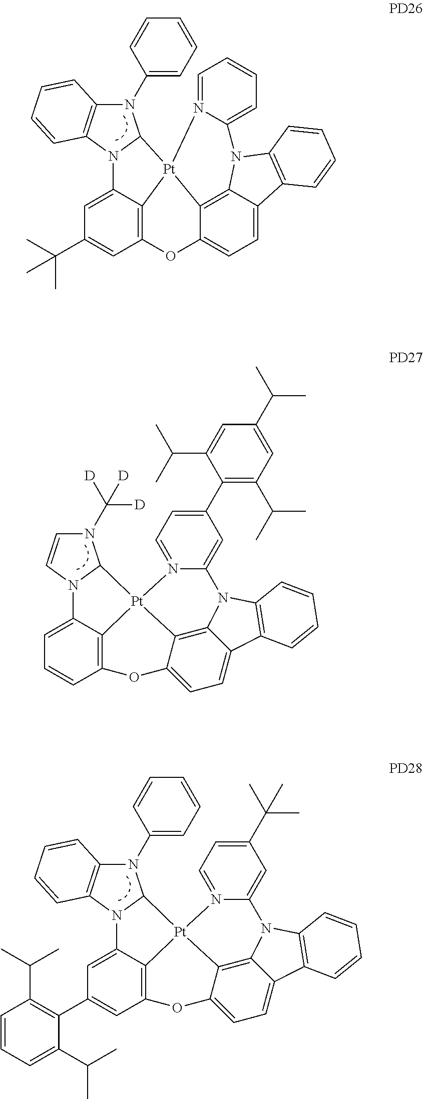

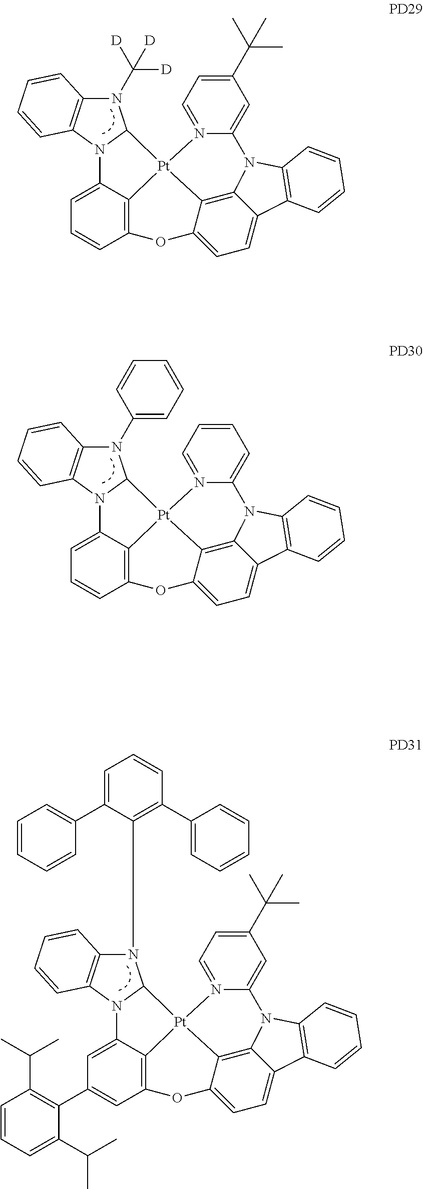

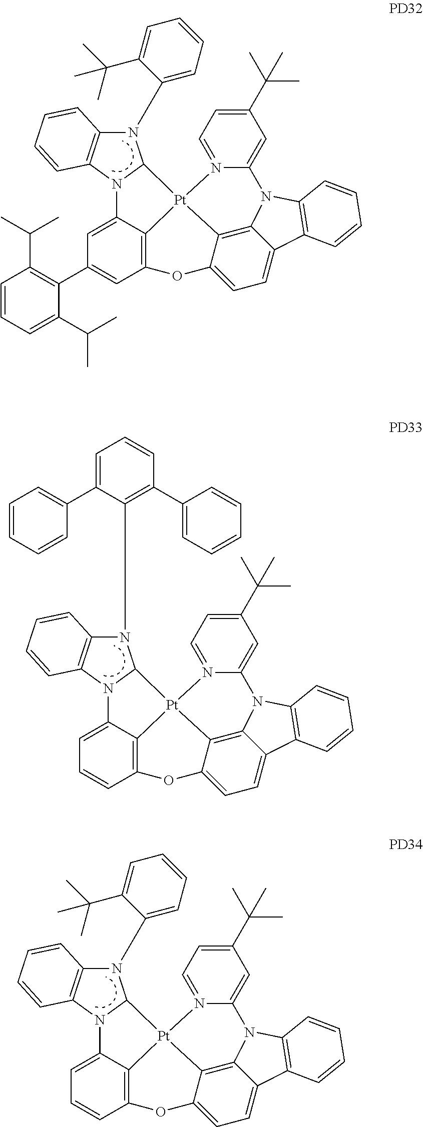

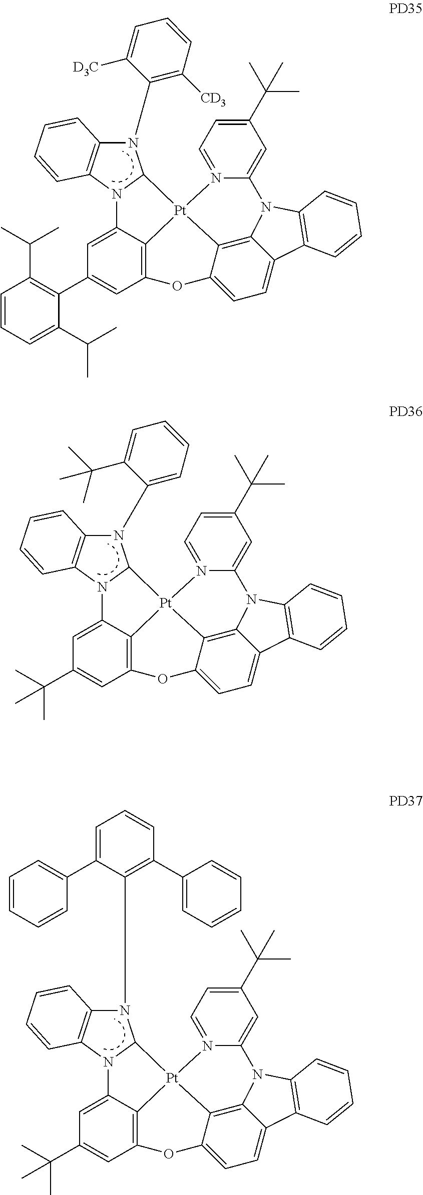

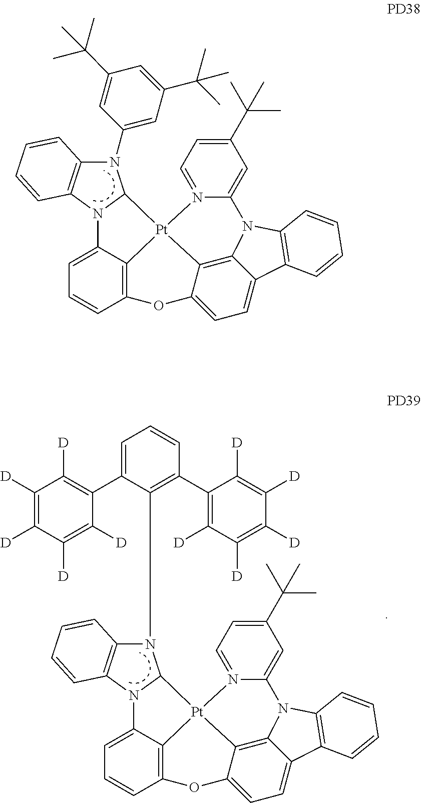

### Fluorescent Dopant

The fluorescent dopant may include an amine group-containing compound, a styryl group-containing compound, or a (e.g., any suitable) combination thereof.

For example, the fluorescent dopant may include a compound represented by Formula 501:

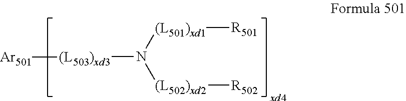

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In one or more embodiments, Ar₅₀₁ in Formula 501 may be a condensed cyclic group (for example, an anthracene group, a chrysene group, a pyrene group, and/or the like) in which three or more monocyclic groups are condensed together.

In one or more embodiments, xd4 in Formula 501 may be 2.

In one or more embodiments, the fluorescent dopant may include: one (e.g., may include at least one or be any one) selected from among Compounds FD1 to FD36; DPVBi; DPAVBi; or a (e.g., any suitable) combination thereof:

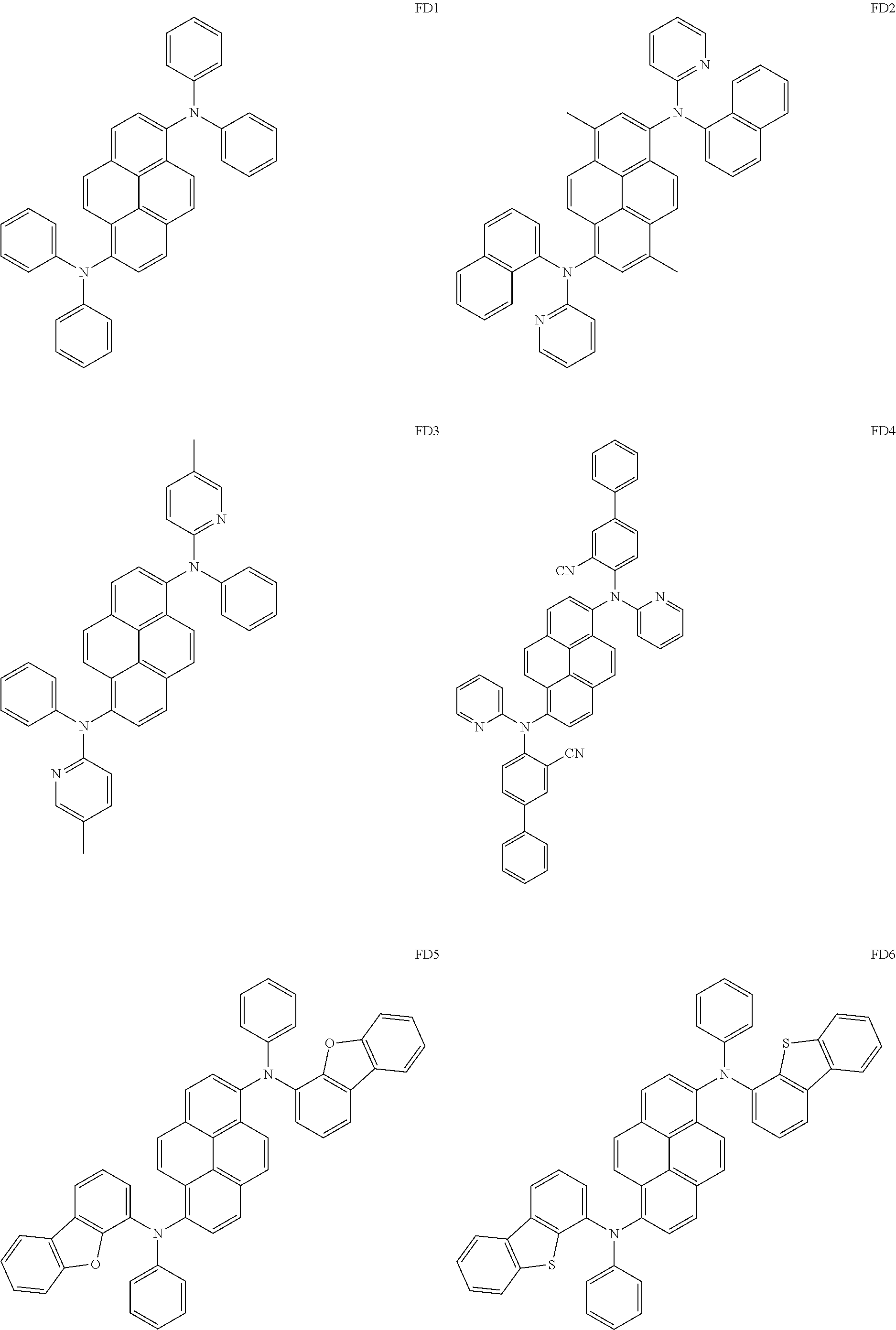

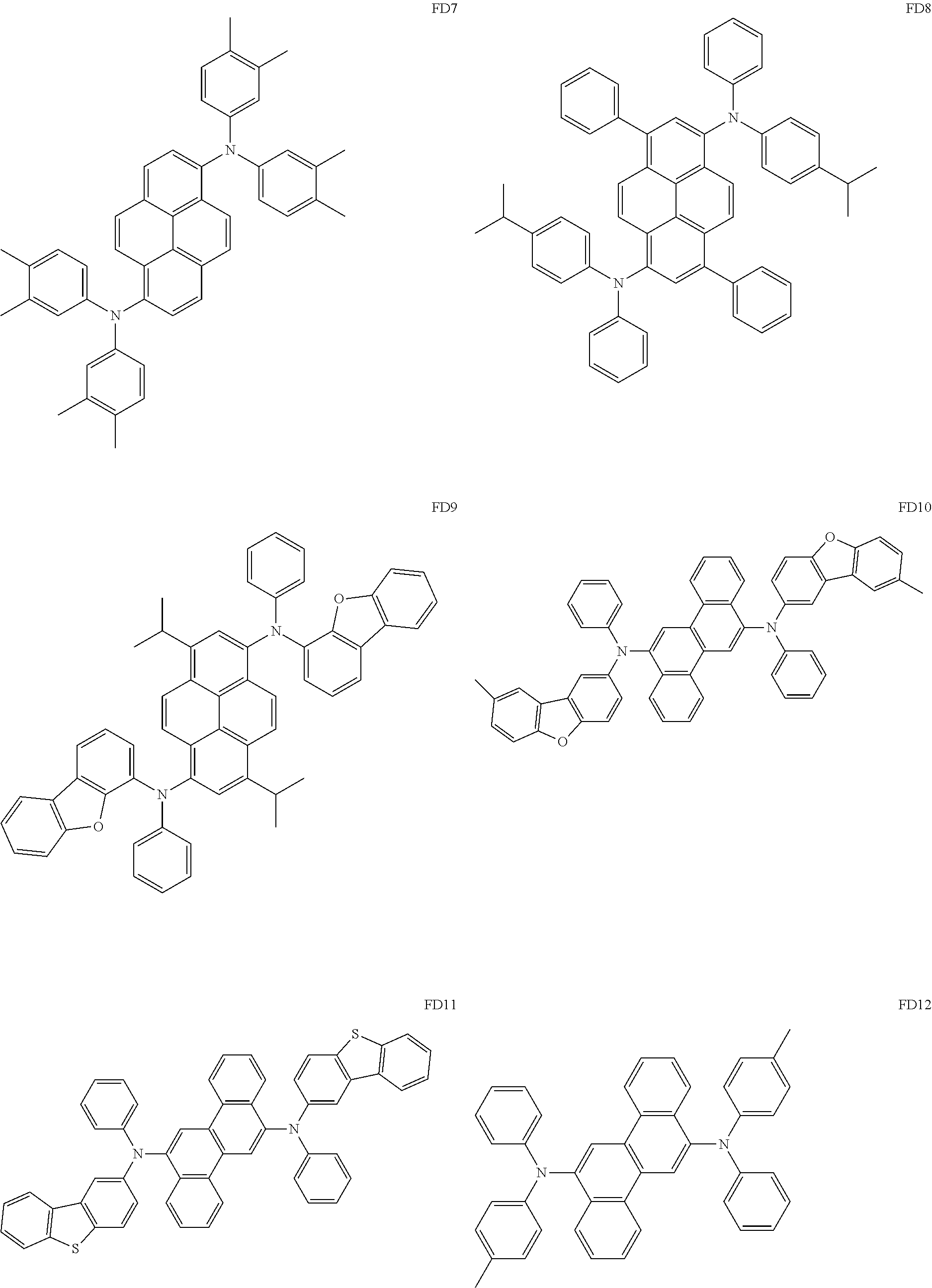

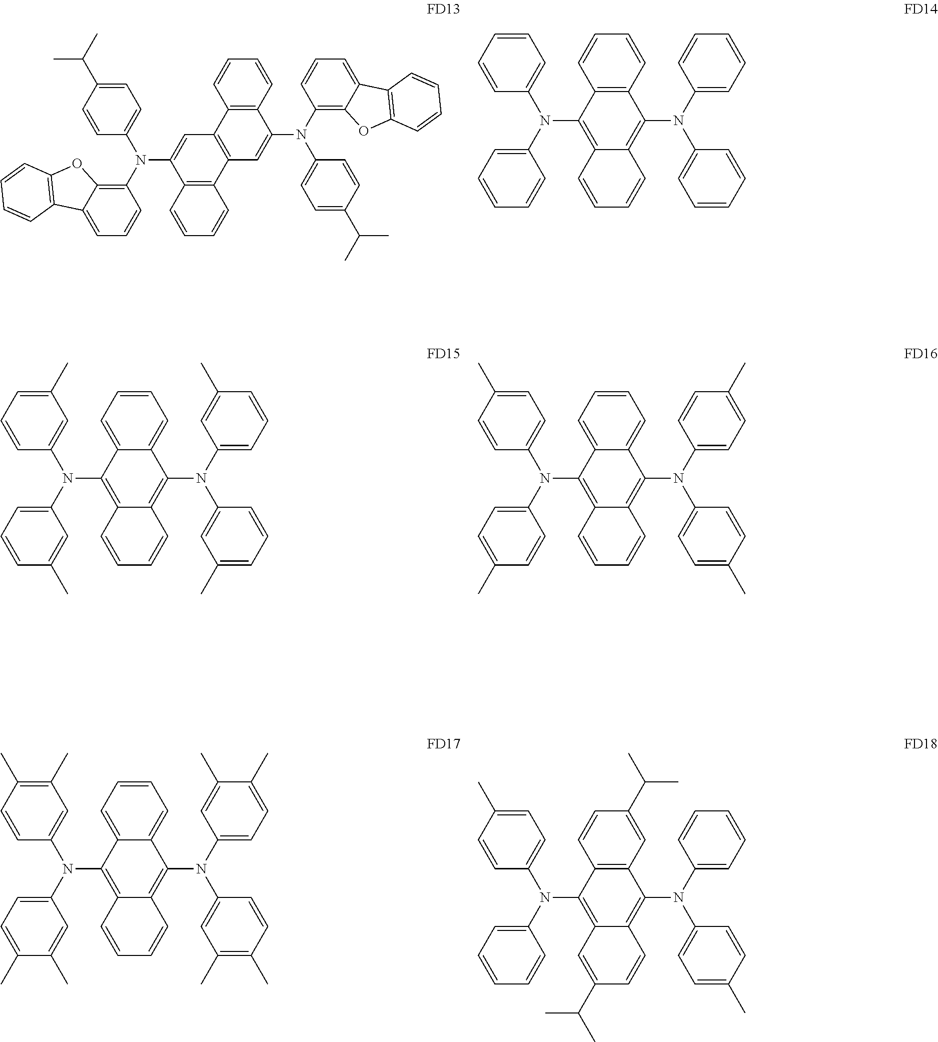

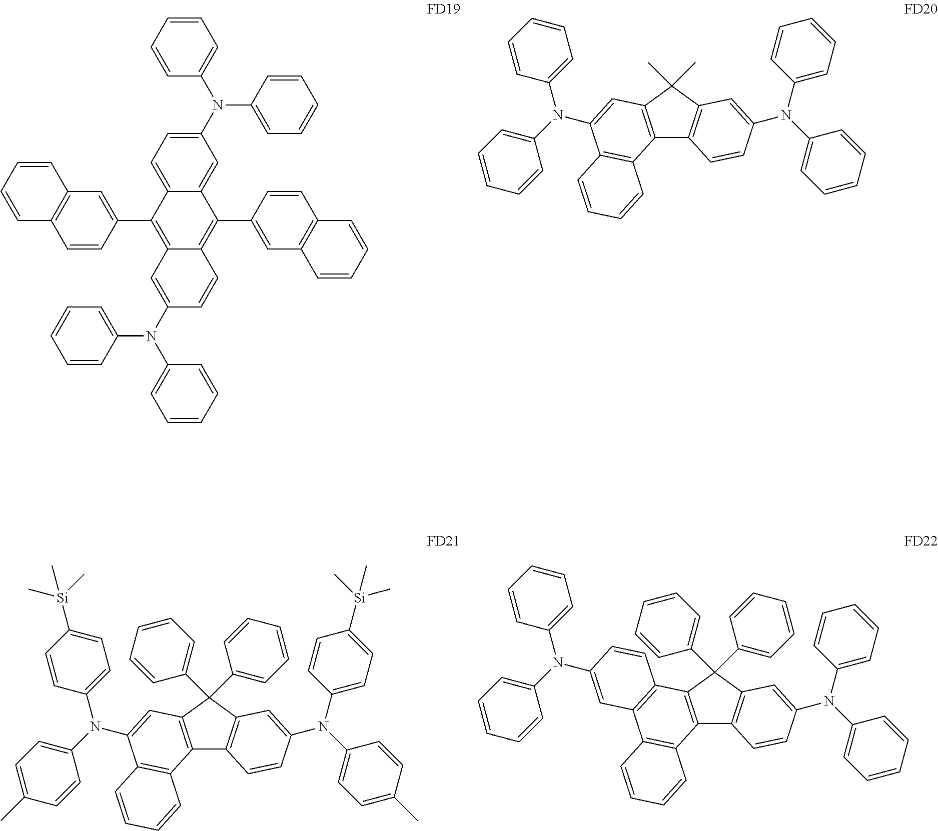

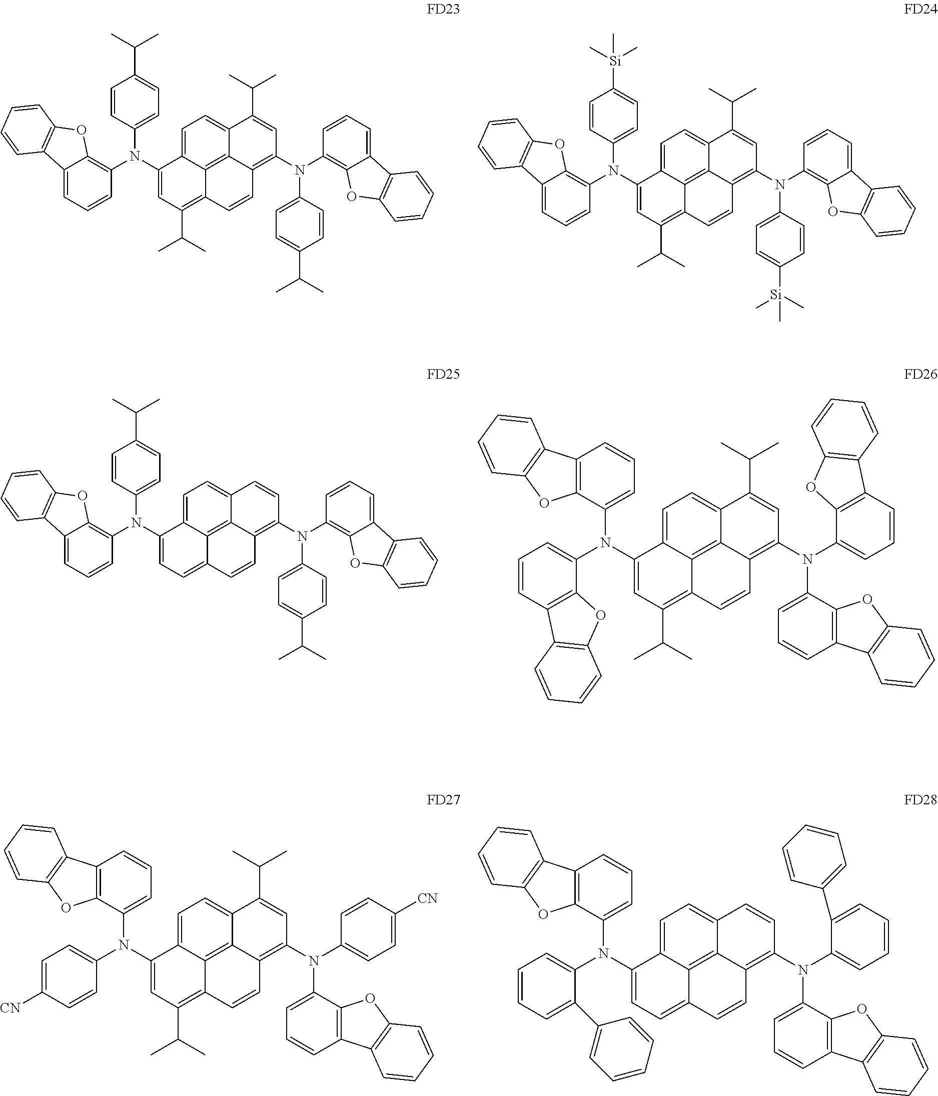

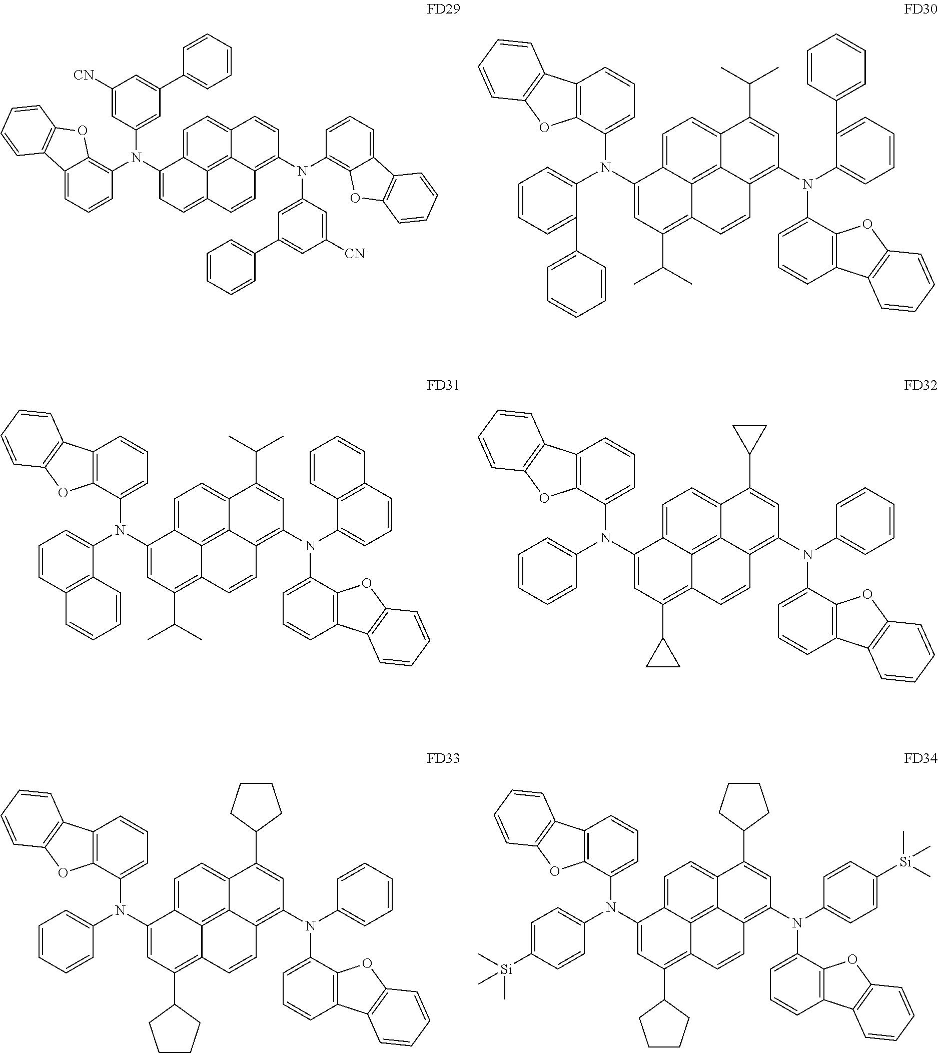

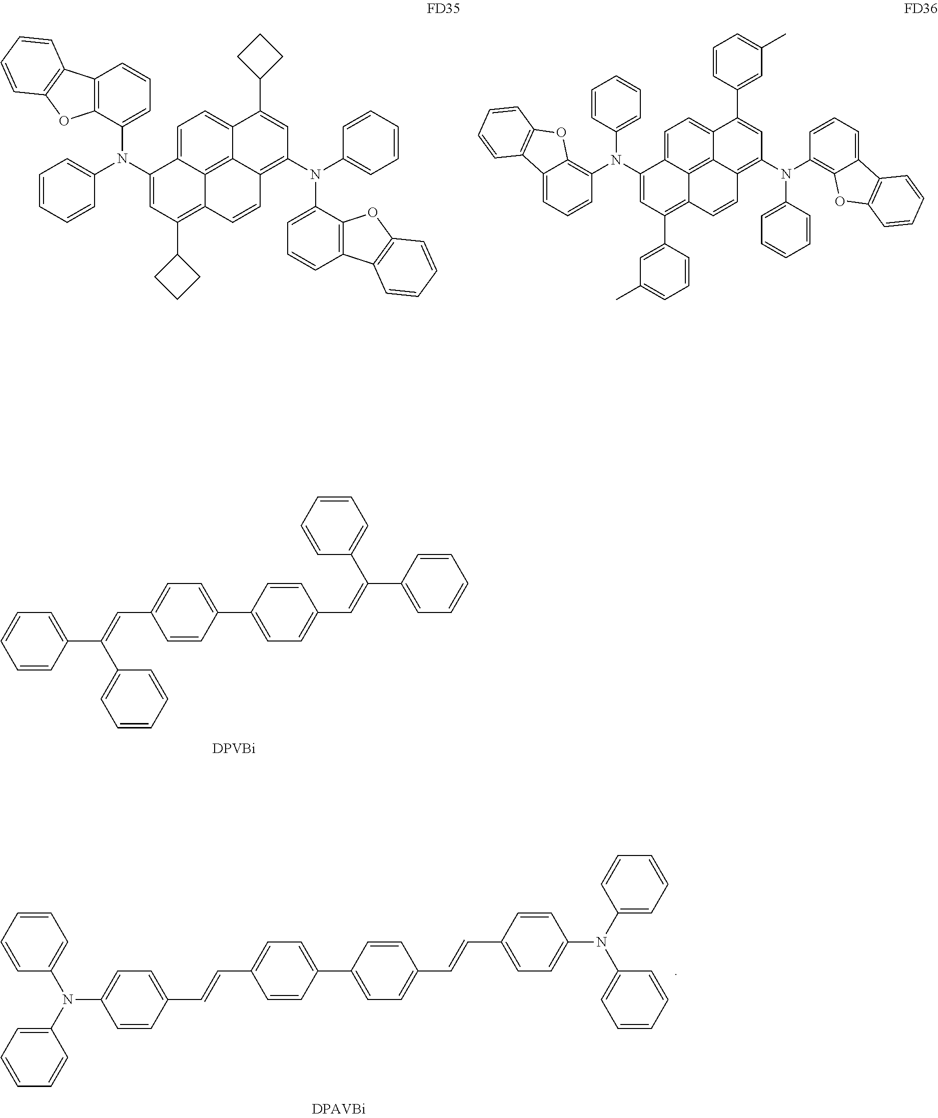

### Delayed Fluorescence Material

The emission layer may further include a delayed fluorescence material.

Herein, the delayed fluorescence material may be selected from among compounds capable of emitting delayed fluorescence based on a delayed fluorescence emission mechanism.

The delayed fluorescence material included in the emission layer may act or serve as a host or a dopant depending on the type (kind) of other materials included in the emission layer.

In one or more embodiments, a difference (e.g., an absolute value of the difference) between a triplet energy level (eV) of the delayed fluorescence material and the singlet energy level (eV) of the delayed fluorescence material may be at least about 0 eV and not more than about 0.5 eV. If (e.g., when) the difference between the triplet energy level (eV) of the delayed fluorescence material and the singlet energy level (eV) of the delayed fluorescence material is within the foregoing range, up-conversion from a triplet state to a singlet state of the delayed fluorescence material may effectively or suitably occur, and thus, the organic light-emitting device 10 may have improved or enhanced luminescence efficiency.

In one or more embodiments, the delayed fluorescence material may include: i) a material including at least one electron donor (for example, a π electron-rich C₃-C₆₀ cyclic group, such as a carbazole group) and at least one electron acceptor (for example, a sulfoxide group, a cyano group, a π electron-deficient nitrogen-containing C₁-C₆₀ heterocyclic group, and/or the like), ii) a material including a C₈-C₆₀ polycyclic group including at least two cyclic groups that are condensed with each other while sharing boron (B) (e.g., one being a first ring and the other being a second ring).

Non-limiting examples of the delayed fluorescence material may include at least one (or may be any one) selected from among Compounds DF1 to DF9:

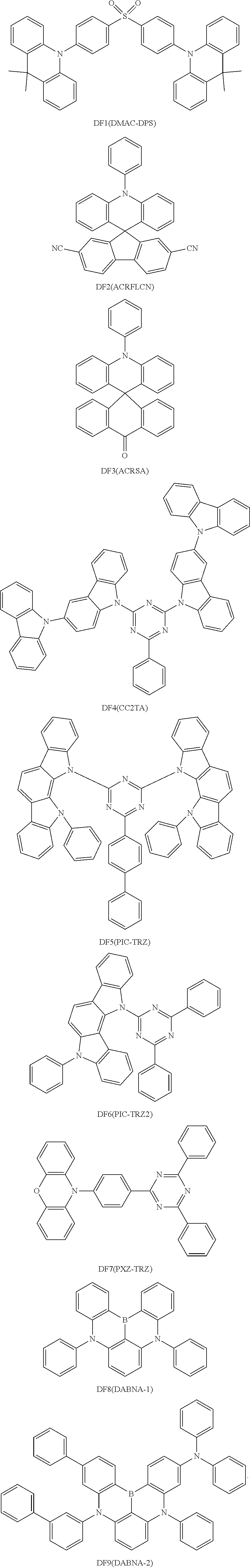

### Quantum Dot

The emission layer may include a quantum dot.

The term “quantum dot” as used herein refers to a crystal of a semiconductor compound. Quantum dots may be to emit light of one or more suitable emission wavelengths according to the size of the crystal. Quantum dots may be to emit light of one or more suitable emission wavelengths by adjusting the element ratio in the quantum dot compound.

A diameter of the quantum dot may be, for example, in a range of about 1 nm to about 10 nm.

In the present disclosure, if (e.g., when) dot, dots, or dot particles are spherical (e.g., substantially spherical), “diameter” indicates a particle diameter or an average particle diameter, and if (e.g., when) the particles are non-spherical, the “diameter” indicates a major axis length or an average major axis length. The diameter of the particles may be measured utilizing a scanning electron microscope and/or a particle size analyzer. As the particle size analyzer, for example, HORIBA, LA-950 laser particle size analyzer, may be utilized. If (e.g., when) the size of the particles is measured by utilizing a particle size analyzer, the average particle diameter is referred to as D50. D50 refers to the average diameter of particles whose cumulative volume corresponds to 50 vol % in the particle size distribution (e.g., cumulative distribution) and refers to the value of the particle size corresponding to 50% from the smallest particle if (e.g., when) the total number of particles is 100% in the distribution curve accumulated in the order of the smallest particle size to the largest particle size.

The quantum dot may be synthesized by a wet chemical process, a metal organic chemical vapor deposition process, a molecular beam epitaxy process, and/or any process similar thereto.

The wet chemical process may be a method including mixing a precursor material of a quantum dot with an organic solvent and then growing a quantum dot particle crystal. As the crystal grows, the organic solvent may act or serve as a dispersant that is naturally distributed on the quantum dot crystal surface and control the growth of the crystal. Therefore, wet chemical processes may control the growth of quantum dot particles through more feasible and lower-cost processes compared to vapor deposition methods, such as metal organic chemical vapor deposition (MOCVD) and molecular beam epitaxy (MBE).

The quantum dot may include a Group III-VI semiconductor compound; a Group II-VI semiconductor compound; a Group III-V semiconductor compound; a Group III-VI semiconductor compound; a Group I—III-VI semiconductor compound; a Group IV-VI semiconductor compound; a Group IV element or compound; or a (e.g., any suitable) combination thereof.

Non-limiting examples of the Group II-VI semiconductor compound may be a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or a (e.g., any suitable) combination thereof.

Non-limiting examples of the Group III-V semiconductor compound may be: a binary compound, such as GaN, GaP, GaAs, GaSb, AlN, AlP, AlAs, AISb, InN, InP, InAs, InSb, and/or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, GaAlNP, and/or the like; a quaternary compound, such as GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and/or the like; or a (e.g., any suitable) combination thereof. In one or more embodiments, the Group III-V semiconductor compound may further include a Group II element. Non-limiting examples of the Group III-V semiconductor compound further including a Group II element may be InZnP, InGaZnP, InAlZnP, and/or the like.

Non-limiting examples of the Group III-VI semiconductor compound may be: a binary compound, such as GaS, Ga₂S₃, GaSe, Ga₂Se₃, GaTe, InS, InSe, In₂Se₃, InTe, and/or the like; a ternary compound, such as InGaS₃, InGaSe₃, and/or the like; or a (e.g., any suitable) combination thereof.

Non-limiting examples of the Group I-III-VI semiconductor compound may be: a ternary compound, such as AgInS, AgInS₂, AgInSe₂, AgGaS, AgGaS₂, AgGaSe₂, CuInS, CuInS₂, CuInSe₂, CuGaS₂, CuGaSe₂, CuGaO₂, AgGaO₂, AgAlO₂, and/or the like; a ternary compound, such as AgInGaS, AgInGaS₂, AgInGaSe, AgInGaSe₂, CuInGaS, CuInGaS₂, and/or the like; or a (e.g., any suitable) combination thereof.

Non-limiting examples of the Group IV-VI semiconductor compound may be: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or a (e.g., any suitable) combination thereof.

The Group IV element or compound may include: a single element compound, such as Si and/or Ge; a binary compound, such as SiC and/or SiGe; or a (e.g., any suitable) combination thereof.

Each element included in a multi-element compound, such as the binary compound, the ternary compound, and the quaternary compound, may be present at a substantially uniform concentration or substantially non-uniform concentration in a particle. The foregoing formulae refer to the types (kinds) of elements included in each compound, and the element ratios in the compounds may be different from each other. For example, AgInGaS₂ may indicate AgInₓGa₁₋ₓS₂ (where x is a real number satisfying 0<x<1).

In one or more embodiments, the quantum dot may have a single structure in which the concentration of each element in the quantum dot is substantially uniform or a core-shell dual structure. For example, the material included in the core and the material included in the shell may be different from each other.

The shell of the quantum dot may act or serve as a protective layer that prevents chemical degeneration (or that reduces a degree or occurrence of chemical degeneration) of the core to maintain or provide semiconductor characteristics, and/or as a charging layer that imparts electrophoretic characteristics to the quantum dot. The shell may be a single layer or a multi-layer. The interface between the core and the shell may have a concentration gradient in which the concentration of an element that exists in the shell decreases toward the center of the core.

Non-limiting examples of the shell of the quantum dot may be an oxide of a metal or non-metal, a semiconductor compound, and a (e.g., any suitable) combination thereof. Non-limiting examples of the oxide of a metal or non-metal may be: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound, such as MgAl₂O₄, CoFe₂O₄, NiFe₂O₄, and/or CoMn₂O₄; and a (e.g., any suitable) combination thereof. Non-limiting examples of the semiconductor compound may be: a Group III-VI semiconductor compound; a Group II-VI semiconductor compound; a Group III-V semiconductor compound; a Group III-VI semiconductor compound; a Group I—III-VI semiconductor compound; a Group IV-VI semiconductor compound; or a (e.g., any suitable) combination thereof, as described in one or more embodiments. For example, the semiconductor compound suitable as a shell may include CdS, CdSe, CdTe, ZnS, ZnSe, ZnTe, ZnSeS, ZnSTe, GaAs, GaP, GaS, GaSe, AgGaS, AgGaS₂, GaSb, HgS, HgSe, HgTe, InAs, InP, InGaP, InSb, AlAs, AlP, AISb, or a (e.g., any suitable) combination thereof.

Each element included in a multi-element compound, such as the binary compound and the ternary compound, may be present at a substantially uniform concentration or substantially non-uniform concentration in a particle. The foregoing formulae refer to the types (kinds) of elements included in each compound, and the element ratios in these compounds may be different from each other.

A full width at half maximum (FWHM) of the emission wavelength spectrum of the quantum dot may be about 45 nm or less, for example, about 40 nm or less, for example, about 30 nm or less, and within the foregoing ranges, color purity or color reproducibility of the quantum dot may be increased or enhanced. In one or more embodiments, because the light emitted through the quantum dot is emitted in all directions, the wide viewing angle may be improved or enhanced.

In one or more embodiments, the quantum dot may be in the form of a spherical (e.g., substantially spherical) particle, a pyramidal (e.g., substantially pyramidal) particle, a multi-arm (e.g., substantially multi-arm) particle, a cubic (e.g., substantially cubic) nanoparticle, a nanotube (e.g., substantially nanotube) particle, a nanowire (e.g., substantially nanowire) particle, a nanofiber (e.g., substantially nanofiber) particle, or a nanoplate (e.g., substantially nanoplate) particle.

By adjusting the size of the quantum dots, the energy band gap of the quantum dot may be adjusted, and thus, light of one or more suitable wavelengths may be obtained in a quantum dot emission layer. Thus, by using quantum dots as described in one or more embodiments (by using quantum dots of different sizes or by varying the ratio of elements in a quantum dot compound), a light-emitting device that emits light of one or more suitable wavelengths may be realized. In one or more embodiments, the size of the quantum dots or the ratio of elements in the quantum dot compound may be selected so that red light, green light, and/or blue light may be emitted. In one or more embodiments, the quantum dots may be configured to emit white light by combination of light of one or more suitable colors.

### Electron Transport Region in Interlayer 130

The electron transport region may have: i) a single-layered structure including (e.g., consisting of) a single layer including (e.g., consisting of) a single material, ii) a single-layered structure including (e.g., consisting of) a single layer including multiple different materials, or iii) a multilayer structure including multiple layers including multiple different materials.

The electron transport region may include a buffer layer, a hole-blocking layer, an electron control layer, an electron transport layer, an electron injection layer, or a (e.g., any suitable) combination thereof.

For example, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, wherein the layers in each structure are sequentially stacked from the emission layer.

The electron transport region (e.g., the buffer layer, the hole-blocking layer, the electron control layer, or the electron transport layer in the electron transport region) may include a metal-free compound including at least one IT electron-deficient nitrogen-containing C₁-C₆₀ heterocyclic group.

In one or more embodiments, the electron transport region may include a compound represented by Formula 601.

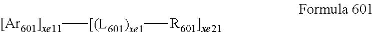

In Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₆₀₁)(Q₆₀₂)(Q₆₀₃), —C(═O) (Q₆₀₁), —S(═O)₂ (Q₆₀₁), or —P(═O)(Q₆₀₁)(Q₆₀₂),
Q₆₀₁ to Q₆₀₃ may each be the same as defined with respect to Q₁,
xe21 may be 1, 2, 3, 4, or 5, and
at least one selected from among Ar₆₀₁, L₆₀₁, and R₆₀₁ may each independently be a π electron-deficient nitrogen-containing C₁-C₆₀ heterocyclic group unsubstituted or substituted with at least one R₁₀ₐ.

In one or more embodiments, if (e.g., when) xe11 in Formula 601 is 2 or greater, two or more selected from among Ar₆₀₁ (s) may be linked together via a single bond (e.g., a single covalent bond).

In one or more embodiments, Ar₆₀₁ in Formula 601 may be a substituted or unsubstituted anthracene group.

In one or more embodiments, the electron transport region may include a compound represented by Formula 601-1:

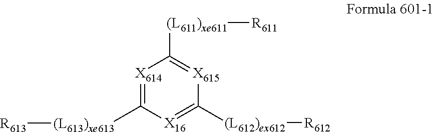

wherein, in Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each be the same as defined with respect to L₆₀₁,
xe611 to xe613 may each be the same as defined with respect to xe1,
R₆₁₁ to R₆₁₃ may each be the same as defined with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group that is unsubstituted or substituted with at least one R₁₀ₐ, or a C₁-C₆₀ heterocyclic group that is unsubstituted or substituted with at least one R₁₀ₐ.

In one or more embodiments, xe1 and xe611 to xe613 in Formulae 601 and 601-1 may each independently be 0, 1, or 2.

The electron transport region may include one selected from among Compounds ET1 to ET45, 2,9-dimethyl-4,7-diphenyl-1,10-phenanthroline (BCP), 4,7-diphenyl-1,10-phenanthroline (Bphen), Alq3, BAIq, TAZ, NTAZ, TSPO1, TPBI, or a (e.g., any suitable) combination thereof:

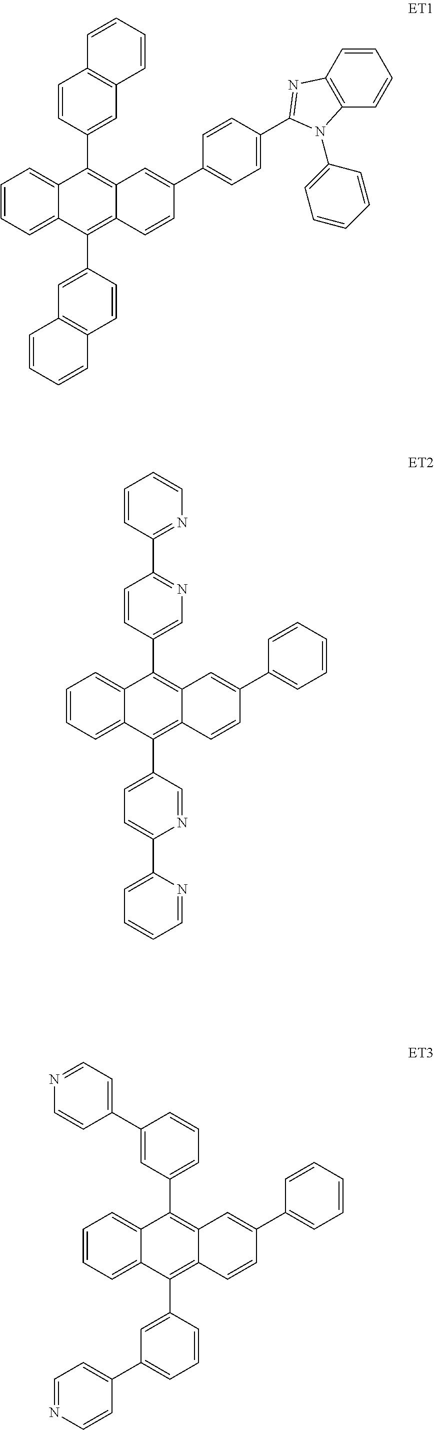 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

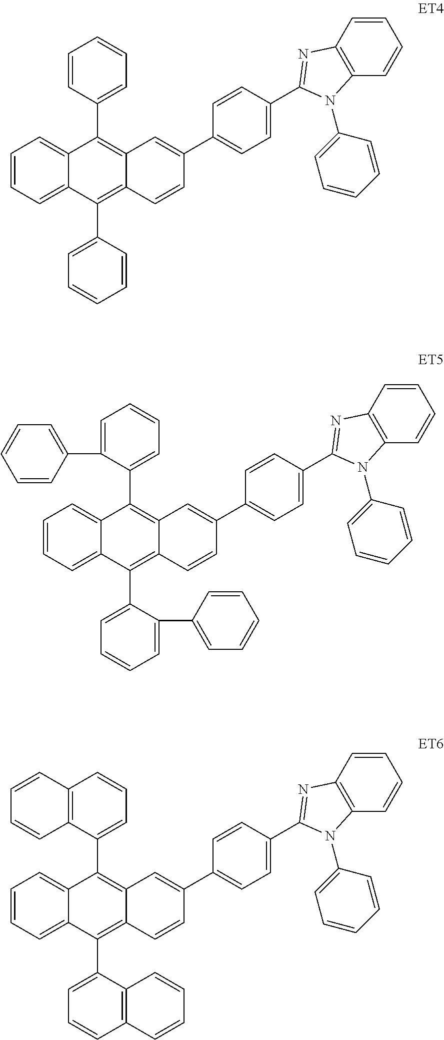 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

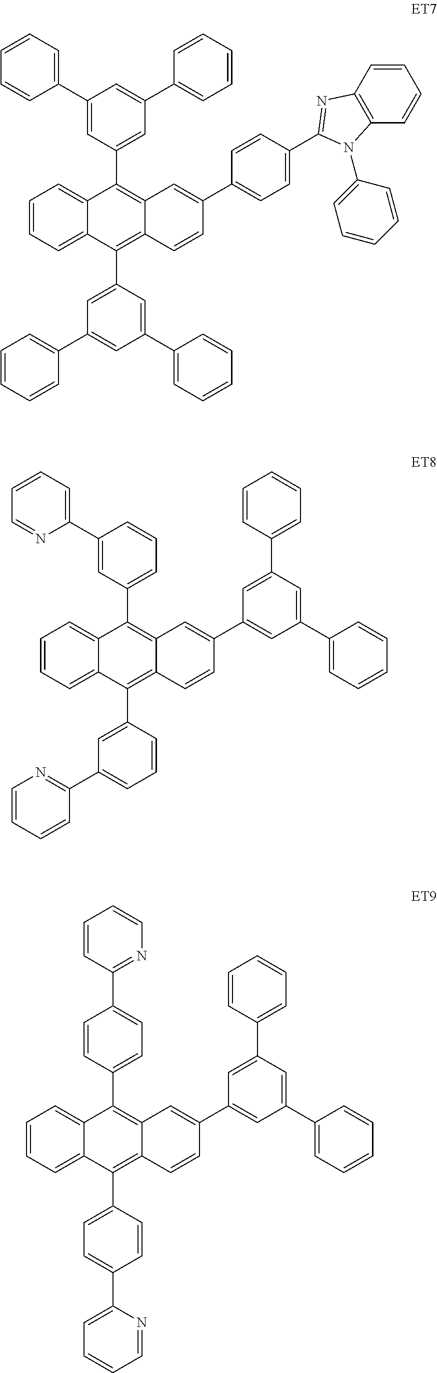 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

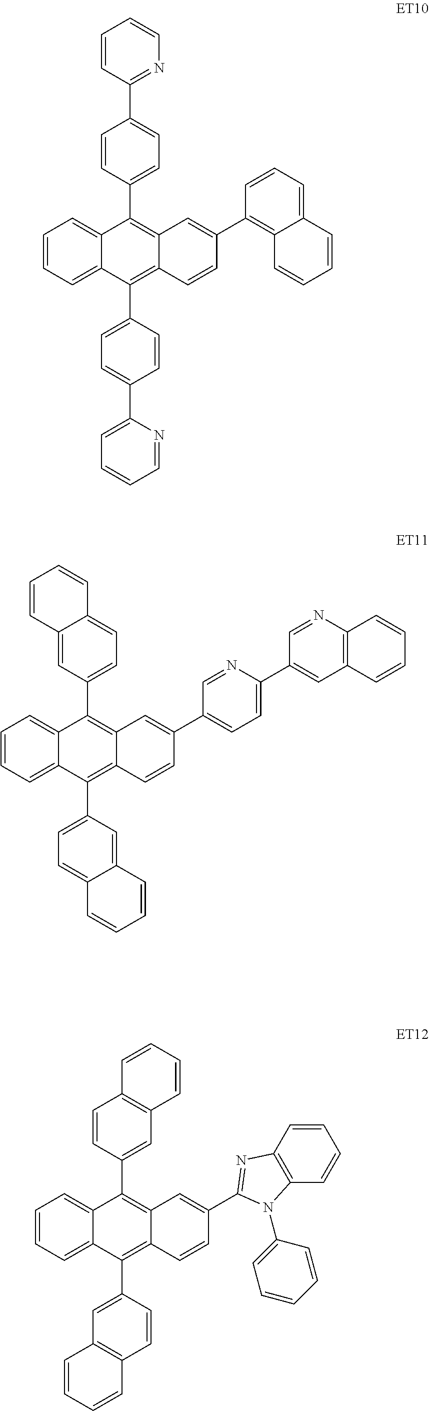 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

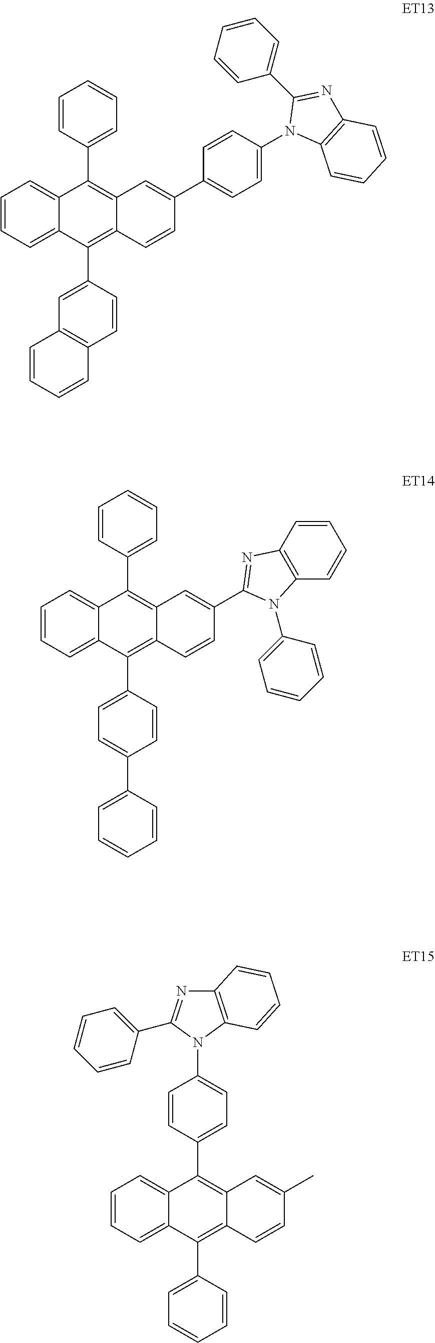 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

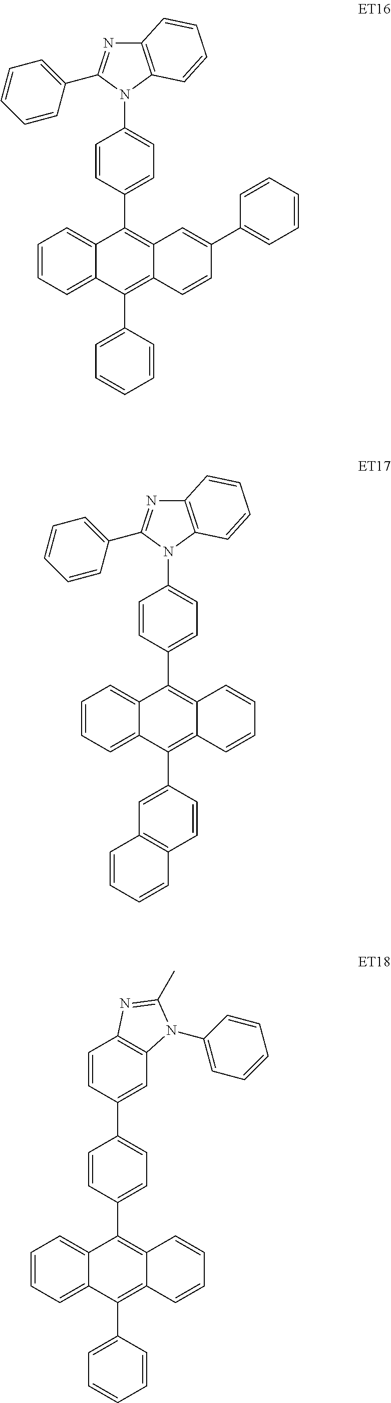 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

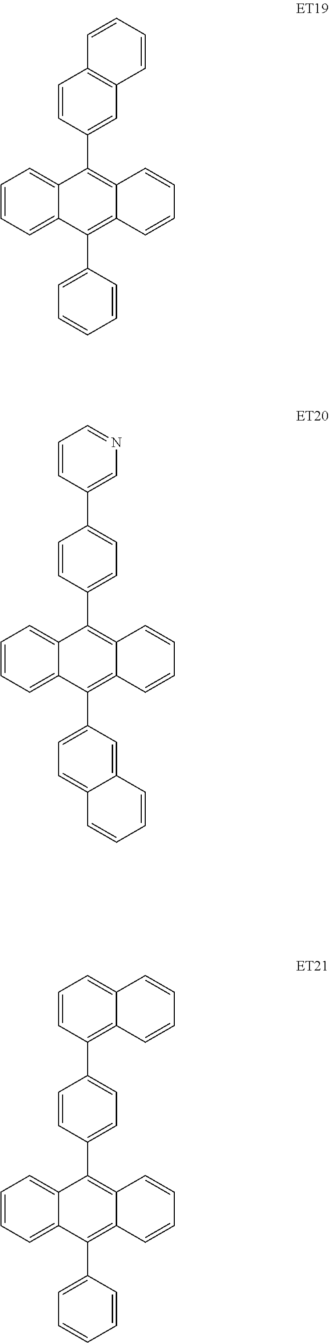

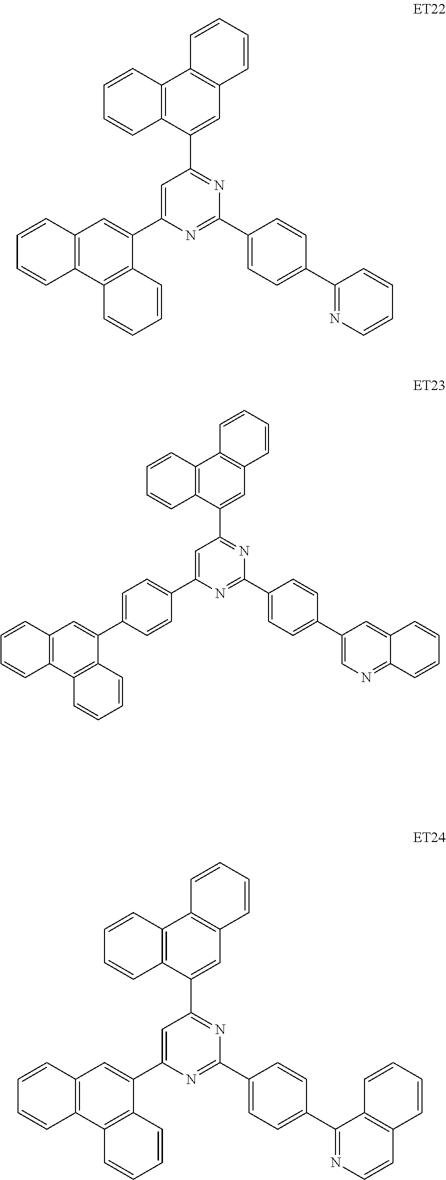 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

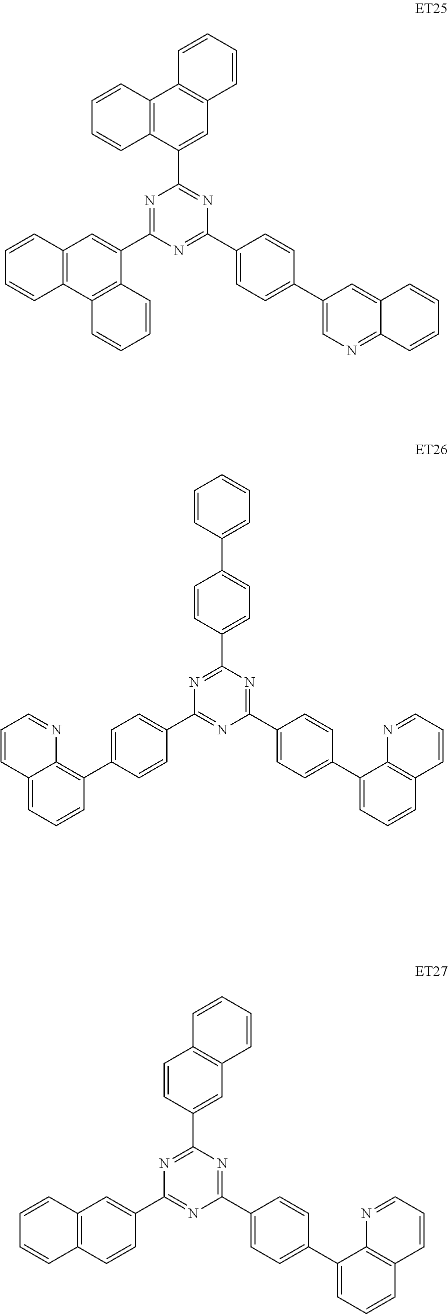 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

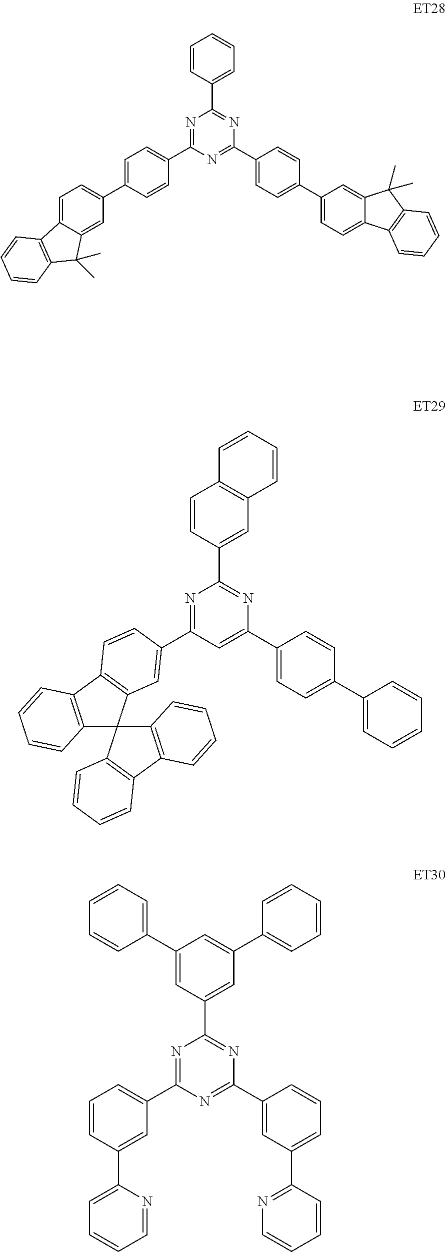 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

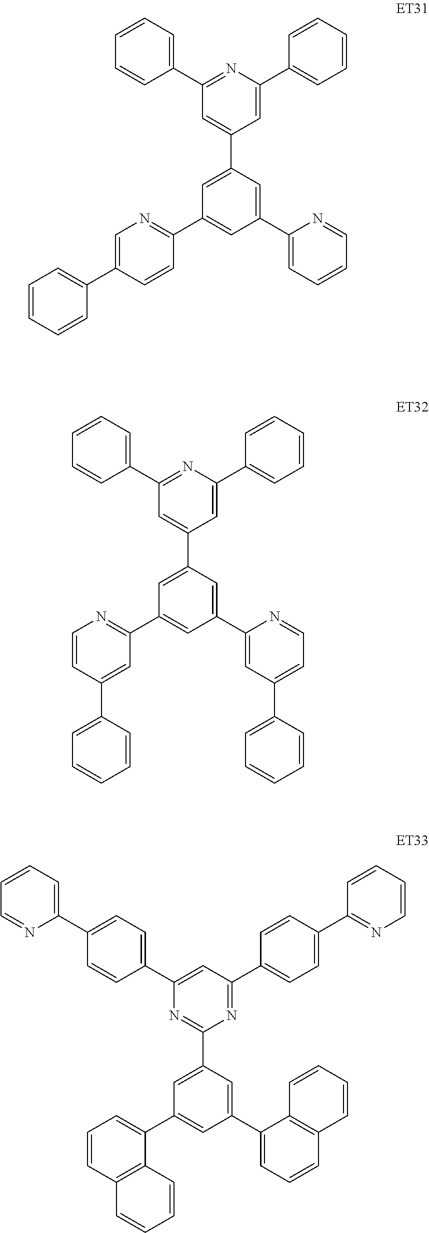 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

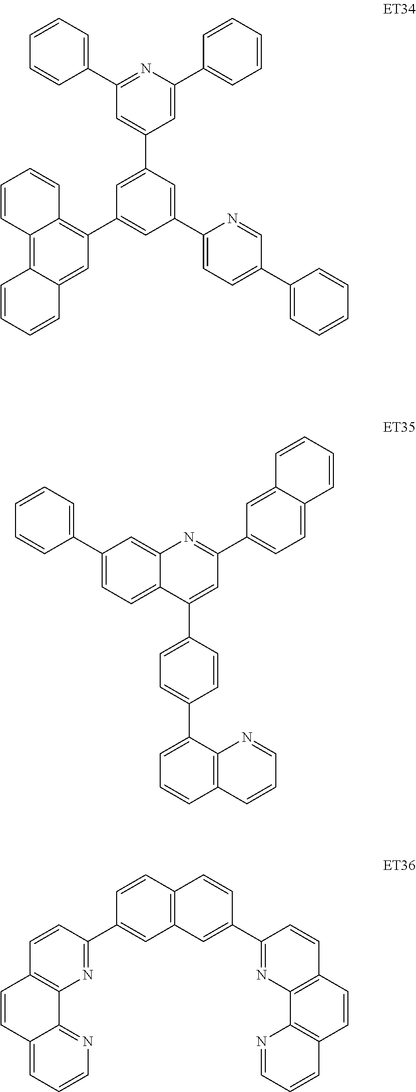 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

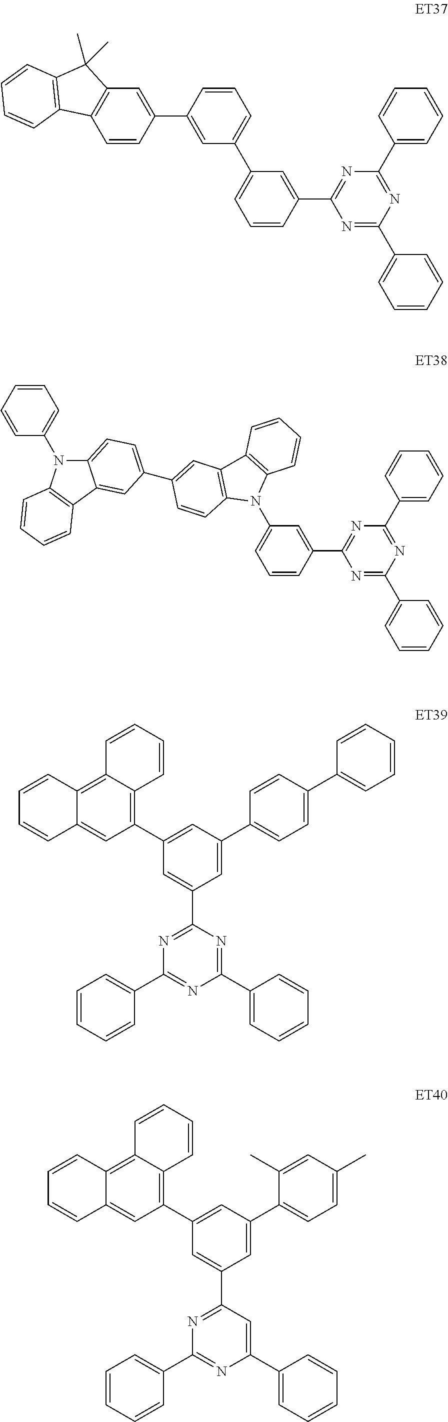 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

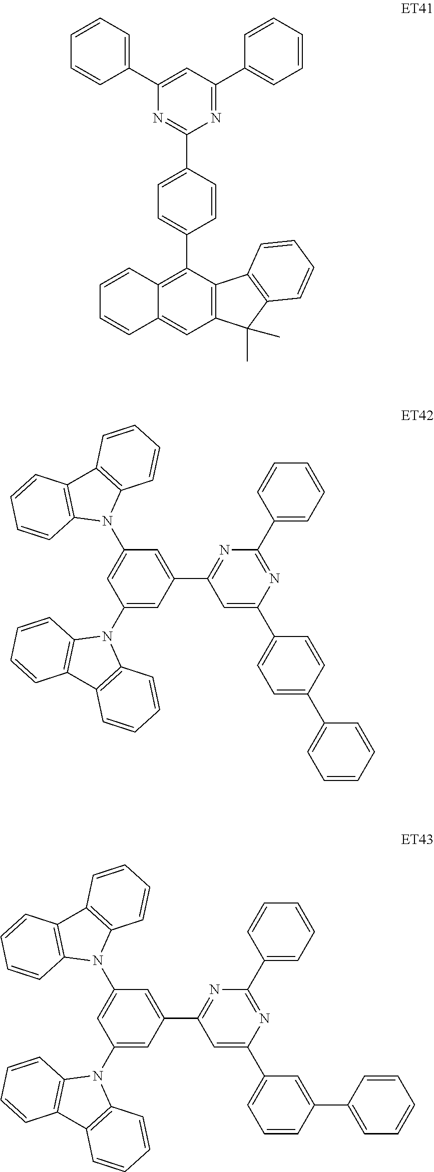 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

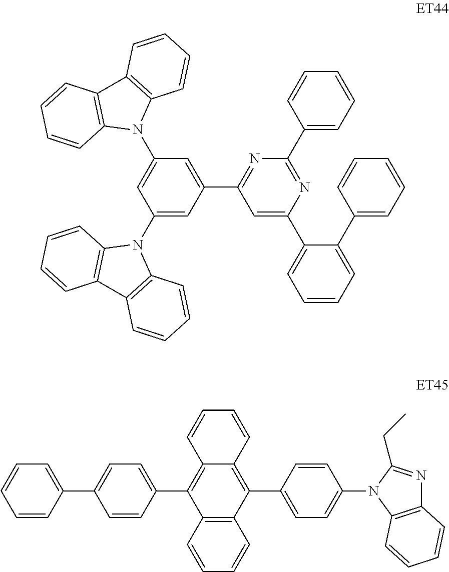 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

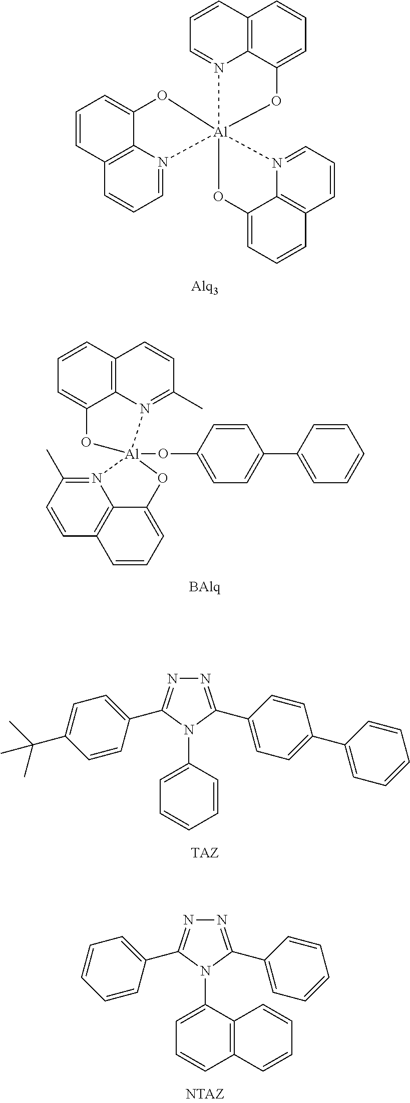

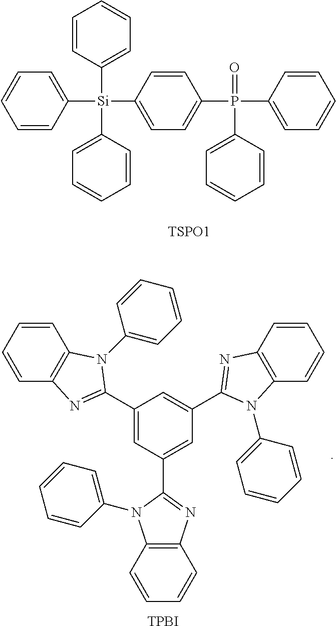

The thickness of the electron transport region may be about 100 Å to about 5,000 Å, for example, about 160 Å to about 4,000 Å. If (e.g., when) the electron transport region includes a buffer layer, a hole-blocking layer, an electron control layer, an electron transport layer, or a (e.g., any suitable) combination thereof, a thickness of the buffer layer, the hole-blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. If (e.g., when) the thickness of the buffer layer, the hole-blocking layer, the electron control layer, the electron transport layer, and/or the electron transport layer are within the foregoing ranges, satisfactory or desired electron transporting characteristics may be obtained without a substantial increase in driving voltage.

The electron transport region (for example, the electron transport layer in the electron transport region) may further include, in addition to the materials as described in one or more embodiments, a metal-containing material.

The metal-containing material may include an alkali metal complex, an alkaline earth metal complex, or a (e.g., any suitable) combination thereof. A metal ion of the alkali metal complex may be a Li ion, a Na ion, a K ion, a Rb ion, and/or a Cs ion, and a metal ion of the alkaline earth metal complex may be a Be ion, a Mg ion, a Ca ion, a Sr ion, and/or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or a (e.g., any suitable) combination thereof.

In one or more embodiments, the metal-containing material may include a Li complex. The Li complex may include, for example, Compound ET-D1 (LiQ) or ET-D2:

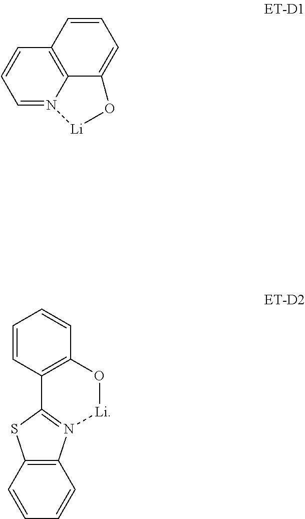

The electron transport region may include an electron injection layer that facilitates the injection of electrons from the second electrode 150. The electron injection layer may directly contact the second electrode 150.

The electron injection layer may have: i) a single-layered structure including (e.g., consisting of) a single layer including (e.g., consisting of) a single material, ii) a single-layered structure including (e.g., consisting of) a single layer including multiple different materials, or iii) a multilayer structure including multiple layers including multiple different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a (e.g., any suitable) combination thereof.

The alkali metal may include Li, Na, K, Rb, Cs, or a (e.g., any suitable) combination thereof. The alkaline earth metal may include Mg, Ca, Sr, Ba, or a (e.g., any suitable) combination thereof. The rare earth metal may include Sc, Y, Ce, Tb, Yb, Gd, or a (e.g., any suitable) combination thereof.

The alkali metal-containing compound, the alkaline earth metal-containing compound, and the rare earth metal-containing compound may include oxides, halides (for example, fluorides, chlorides, bromides, iodides, and/or the like), and/or tellurides of the alkali metal, the alkaline earth metal, and the rare earth metal, or a (e.g., any suitable) combination thereof.

The alkali metal-containing compound may include: alkali metal oxides, such as Li₂O, Cs₂O, and/or K₂O; alkali metal halides, such as LiF, NaF, CsF, KF, LiI, NaI, CsI, and/or KI; or a (e.g., any suitable) combination thereof. The alkaline earth metal-containing compound may include an alkaline earth metal oxide, such as BaO, SrO, CaO, BaₓSr₁₋ₓO (x is a real number satisfying 0<x<1), and/or BaₓCa₁₋ₓO (x is a real number satisfying 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or a (e.g., any suitable) combination thereof. In one or more embodiments, the rare earth metal-containing compound may include lanthanide metal telluride. Non-limiting examples of the lanthanide metal telluride may be LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂ Te₃, Gd₂Te₃, Tb₂Te₃, Dy₂Te₃, Ho₂Te₃, Er₂Te₃, Tm₂Te₃, Yb₂Te₃, and/or Lu₂ Te₃.

The alkali metal complex, the alkaline earth-metal complex, and the rare earth metal complex may include i) one of ions selected from among the alkali metal, the alkaline earth metal, and the rare earth metal and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or a (e.g., any suitable) combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a (e.g., any suitable) combination thereof, as described in one or more embodiments. In one or more embodiments, the electron injection layer may further include an organic material (for example, a compound represented by Formula 601).

In one or more embodiments, the electron injection layer may include (e.g., consist of) i) an alkali metal-containing compound (for example, alkali metal halide), ii) a) an alkali metal-containing compound (for example, alkali metal halide); and b) an alkali metal, an alkaline earth metal, a rare earth metal, or a (e.g., any suitable) combination thereof. For example, the electron injection layer may be a KI:Yb co-deposited layer, an RbI:Yb co-deposited layer, and/or the like.

If (e.g., when) the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or a (e.g., any suitable) combination thereof may be uniformly (e.g., substantially uniformly) or non-uniformly (e.g., substantially non-uniformly) dispersed in a matrix including the organic material.

The thickness of the electron injection layer may be about 1 Å to about 100 Å, and, for example, about 3 Å to about 90 Å. If (e.g., when) the thickness of the electron injection layer is within the foregoing ranges as described in one or more embodiments, satisfactory or desired electron injection characteristics may be obtained without a substantial increase in driving voltage.

### Second Electrode 150

The second electrode 150 may be on the interlayer 130. The second electrode 150 may be a cathode, which is an electron injection electrode, and as a material to form or provide the second electrode 150, a metal, an alloy, an electrically conductive compound, or a (e.g., any suitable) combination thereof, each having a low-work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or a (e.g., any suitable) combination thereof. The second electrode 150 may be a transmissive electrode, a transflective electrode, or a reflective electrode.

The second electrode 150 may have a single-layer structure or a multilayer structure including a plurality of layers.

### Capping Layer

A first capping layer may be outside the first electrode 110, and/or a second capping layer may be outside the second electrode 150. For example, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward the outside through the first electrode 110 which is a transflective electrode or a transmissive electrode, and the first capping layer. Light generated in the emission layer of the interlayer 130 of the light-emitting device 10 may be extracted toward the outside through the second electrode 150 which is a transflective electrode or a transmissive electrode, and the second capping layer.

The first capping layer and the second capping layer may increase or enhance the external emission efficiency according to the principle of constructive interference. Accordingly, the light extraction efficiency of the light-emitting device 10 may be increased or enhanced, such that the luminescence efficiency of the light-emitting device 10 may be increased.

Each of the first capping layer and the second capping layer may include a material having a refractive index of about 1.6 or higher (at 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from among the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or a (e.g., any suitable) combination thereof. The carbocyclic compound, the heterocyclic compound, and the amine group-containing compound may each optionally be substituted with a substituent including O, N, S, Se, Si, F, Cl, Br, I, or a (e.g., any suitable) combination thereof. In one or more embodiments, at least one selected from among the first capping layer and the second capping layer may each independently include an amine group-containing compound.

In one or more embodiments, at least one selected from among the first capping layer and the second capping layer may each independently include a compound represented by Formula 201, a compound represented by Formula 202, or a (e.g., any suitable) combination thereof.

In one or more embodiments, at least one selected from among the first capping layer and the second capping layer may each independently include one selected from among Compounds HT28 to HT33, one selected from among Compounds CP1 to CP6, β-NPB, or a (e.g., any suitable) combination thereof:

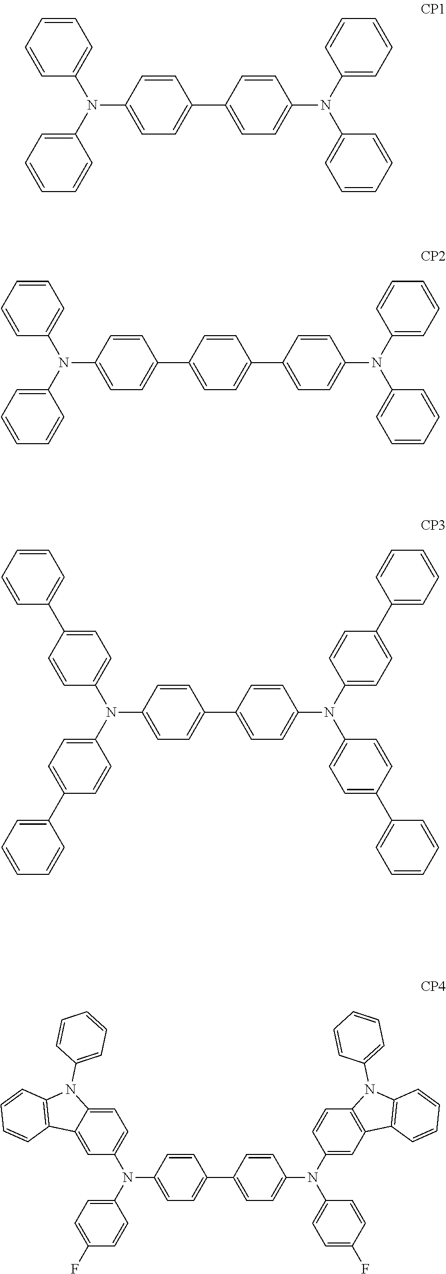 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

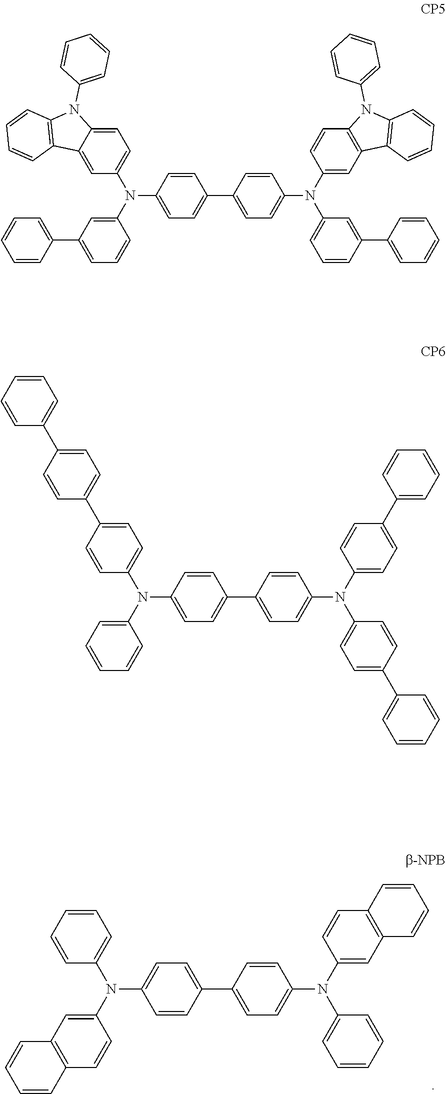 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The heterocyclic compound represented by Formula 1 may be included in one or more suitable films.

Thus, in one or more embodiments, a film including the heterocyclic compound represented by Formula 1 may be provided. The film may be, for example, an optical member (or a light control means) (e.g., a color filter, a color conversion member, a capping layer, a light extraction efficiency enhancement layer, a selective light absorbing layer, a polarizing layer, a quantum dot-containing layer, and/or the like), a light-blocking member (e.g., a light reflective layer, a light absorbing layer, and/or the like), a protective member (e.g., an insulating layer, a dielectric layer, and/or the like), and/or the like.

### Electronic Apparatus

The light-emitting device may be included in one or more suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be in at least one traveling direction of light emitted from the light-emitting device. For example, the light emitted from the light-emitting device may be blue light or white light. A more detailed description of the light-emitting device is provided in one or more embodiments. In one or more embodiments, the color conversion layer may include quantum dots. The quantum dot may be, for example, a quantum dot as described in one or more embodiments.

The electronic apparatus may include a first substrate. The first substrate may include a plurality of subpixel areas, the color filter may include a plurality of color filter areas that respectively correspond to the subpixel areas, and the color conversion layer may include a plurality of color conversion areas that respectively correspond to the subpixel areas.

A pixel-defining film may be among the subpixel areas to define each of the subpixel areas.

The color filter may further include a plurality of color filter areas and light-shielding patterns among the color filter areas, and the color conversion layer may further include a plurality of color conversion areas and light-shielding patterns among the color conversion areas.

The plurality of color filter areas (or the plurality of color conversion areas) may include a first area to emit first color light, a second area to emit second color light, and/or a third area to emit third color light, wherein the first color light, the second color light, and/or the third color light may have different maximum emission wavelengths. In one or more embodiments, the first color light may be red light, the second color light may be green light, and the third color light may be blue light. In one or more embodiments, the plurality of color filter areas (or the plurality of color conversion areas) may include quantum dots. For example, the first area may include red quantum dots, the second area may include green quantum dots, and the third area may not include (e.g., may exclude) quantum dots. A more detailed description of the quantum dots is provided in one or more embodiments. The first area, the second area, and/or the third area may each further include a scatterer (e.g., a light scatterer).

In one or more embodiments, the light-emitting device may be to emit first light, the first area may be to absorb the first light to emit first-first color light, the second area may be to absorb the first light to emit second-first color light, and the third area may be to absorb the first light to emit third-first color light. In one or more embodiments, the first-first color light, the second-first color light, and the third-first color light may have different maximum emission wavelengths. In one or more embodiments, the first light may be blue light, the first-first color light may be red light, the second-first color light may be green light, and the third-first color light may be blue light.

The electronic apparatus may further include a thin-film transistor, in addition to the light-emitting device as described in one or more embodiments. The thin-film transistor may include a source electrode, a drain electrode, and an activation layer, wherein any one selected from among the source electrode and the drain electrode may be electrically connected to any one selected from among the first electrode and the second electrode of the light-emitting device.

The thin-film transistor may further include a gate electrode, a gate insulating film, and/or the like.

The activation layer may include crystalline silicon, amorphous (e.g., non-crystalline) silicon, an organic semiconductor, an oxide semiconductor, and/or the like.

The electronic apparatus may further include a sealing portion to seal the light-emitting device. The sealing portion may be between the color filter and/or the color conversion layer and the light-emitting device. The sealing portion may allow light from the light-emitting device to be extracted to the outside, and concurrently (e.g., simultaneously) prevents ambient air and/or moisture from penetrating into the light-emitting device (or reduces a degree to or occurrence of which ambient air and/or moisture penetrates into the light-emitting device). The sealing portion may be a sealing substrate including a transparent (e.g., substantially transparent) glass substrate and/or a plastic substrate. The sealing portion may be a thin-film encapsulation layer including at least one layer of an organic layer and/or an inorganic layer. If (e.g., when) the sealing portion is a thin film encapsulation layer, the electronic apparatus may be flexible.

One or more suitable functional layers may be additionally arranged or provided on the sealing portion, in addition to the color filter and/or the color conversion layer, according to the use of the electronic apparatus. Non-limiting examples of the functional layers may be a touch screen layer and a polarizing layer. The touch screen layer may be a pressure-sensitive touch screen layer, a capacitive touch screen layer, or an infrared touch screen layer.

The authentication apparatus may further include, in addition to the light-emitting device as described in one or more embodiments, a biometric information collector. The authentication apparatus may be, for example, a biometric authentication apparatus that authenticates an individual by using biometric information of a living body (for example, fingertips, pupils, and/or the like).

The electronic apparatus may be applied to one or more suitable displays, light sources, lighting, personal computers (for example, mobile personal computers), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, and/or endoscope displays), fish finders, one or more suitable measuring instruments, meters (for example, meters for a vehicle, an aircraft, and/or a vessel), projectors, and/or the like.

### Electronic Device

The light-emitting device may be included in one or more suitable electronic devices.

In one or more embodiments, the electronic device including the light-emitting device may be one selected from among a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3D display, a virtual or augmented-reality display, a vehicle, a video wall including multiple displays tiled together, a theater screen, a stadium screen, a phototherapy device, and a signboard.

Because the light-emitting device has excellent or suitable effects in terms of luminescence efficiency long lifespan, the electronic device including the light-emitting device may have characteristics with high luminance, high resolution, and/or low power consumption.

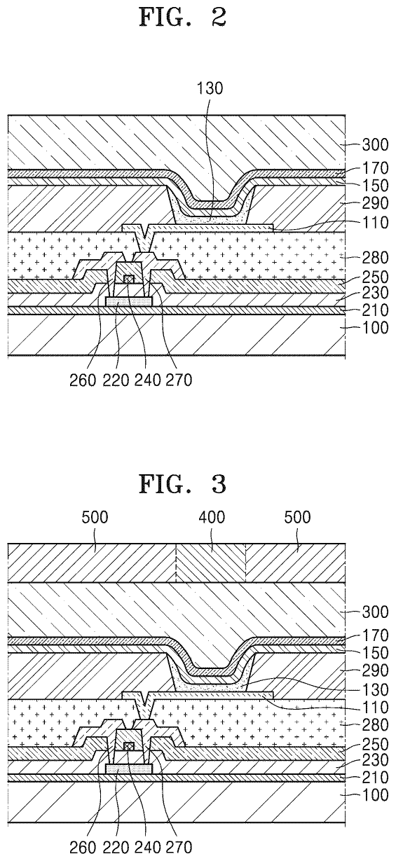

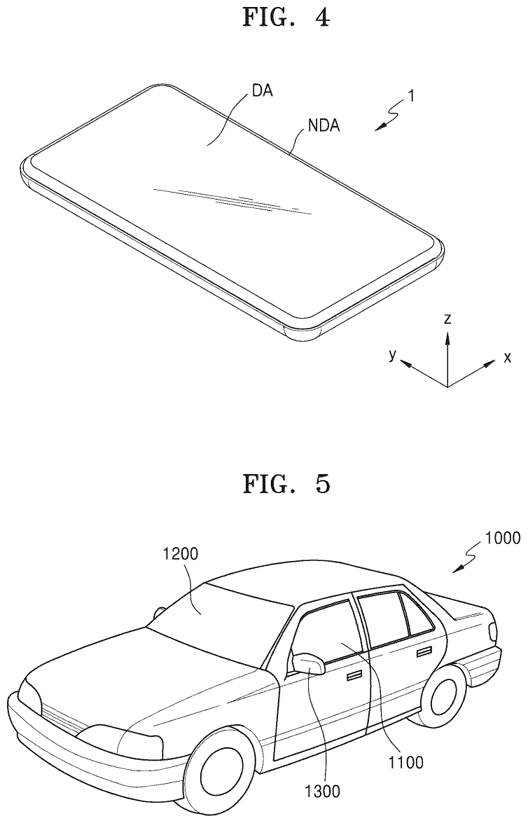

### Description of FIGS. 2 and 3

FIG. 2 is a cross-sectional view illustrating a light-emitting apparatus according to one or more embodiments.

The light-emitting apparatus of FIG. 2 may include a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be on the substrate 100. The buffer layer 210 may prevent penetration of impurities (or reduce a degree or occurrence of penetration fo impurities) through the substrate 100 and may provide a flat surface (e.g., a substantially flat surface) on the substrate 100.

A TFT may be on the buffer layer 210. The TFT may include an activation layer 220, a gate electrode 240, a source electrode 260, and a drain electrode 270.

The activation layer 220 may include an inorganic semiconductor, such as silicon and/or polysilicon, an organic semiconductor, and/or an oxide semiconductor, and may include a source region, a drain region, and a channel region.

A gate insulating film 230 to insulate the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270, to insulate these electrodes from one another.

The source electrode 260 and the drain electrode 270 may be on the interlayer insulating film 250. The interlayer insulating film 250 and the gate insulating film 230 may be formed or provided to expose the source region and the drain region of the activation layer 220, and the source electrode 260 and the drain electrode 270 may be arranged or provided to contact the exposed portions of the source region and the drain region of the activation layer 220.

The TFT may be electrically connected to a light-emitting device to drive the light-emitting device, and may be covered and protected by a passivation layer 280. The passivation layer 280 may include an inorganic insulating (e.g., electrically insulating) film, an organic insulating (e.g., electrically insulating) film, or a (e.g., any suitable) combination thereof. A light-emitting device may be provided on the passivation layer 280. The light-emitting device may include the first electrode 110, the interlayer 130, and the second electrode 150.

The first electrode 110 may be on the passivation layer 280. The passivation layer 280 may be arranged or provided to expose a portion of the drain electrode 270, not fully covering the drain electrode 270, and the first electrode 110 may be arranged or provided to be connected to the exposed portion of the drain electrode 270.

A pixel-defining film 290 including an insulating (e.g., electrically insulating) material may be on the first electrode 110. The pixel-defining film 290 may be to expose a certain region of the first electrode 110, and the interlayer 130 may be in the exposed region of the first electrode 110. The pixel-defining film 290 may be a polyimide-based organic film and/or a polyacrylic organic film. In one or more embodiments, at least one or more layers of the interlayer 130 may extend beyond the upper portion of the pixel-defining film 290 to be arranged or provided in the form of a common layer.

The second electrode 150 may be arranged or provided on the interlayer 130, and a capping layer 170 may be additionally formed or provided on the second electrode 150. The capping layer 170 may be formed or provided to cover the second electrode 150.

The encapsulation portion 300 may be on the capping layer 170. The encapsulation portion 300 may be on a light-emitting device to protect the light-emitting device from moisture and/or oxygen (or to reduce a degree to or occurrence of which moisture and/or oxygen penetrates into the light-emitting device). The encapsulation portion 300 may include: an inorganic film including silicon nitride (e.g., Si₃N₄ or SiNₓ, wherein 0<X≤2), silicon oxide (e.g., SiOₓ, wherein 0<X≤2; e.g., SiO₂), indium tin oxide, indium zinc oxide, or a (e.g., any suitable) combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or a (e.g., any suitable) combination thereof; and/or a (e.g., any suitable) combination of the inorganic film and the organic film.

FIG. 3 is a cross-sectional view illustrating a light-emitting apparatus according to one or more embodiments.

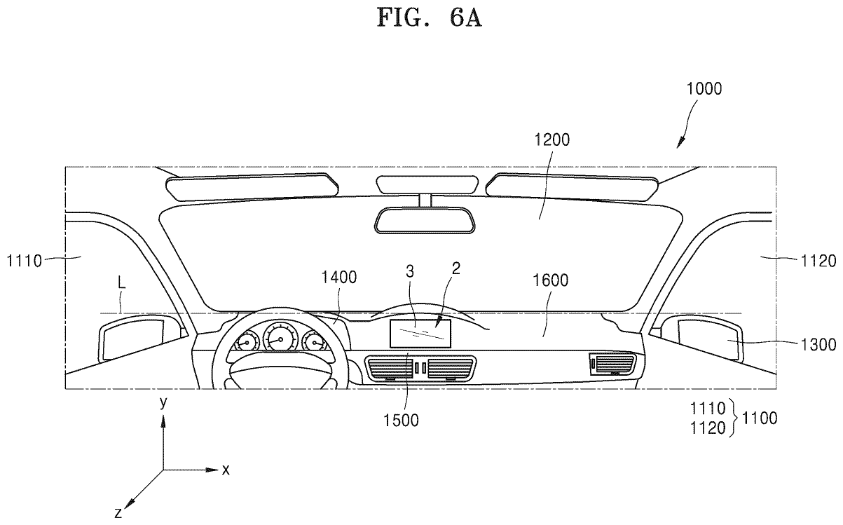

The light-emitting apparatus of FIG. 3 may be substantially the same as the light-emitting apparatus of FIG. 2, except that a light-shielding pattern 500 and a functional region 400 are additionally on the encapsulation portion 300. The functional region 400 may be i) a color filter area, ii) a color conversion area, or iii) a combination of the color filter area and the color conversion area. In one or more embodiments, a light-emitting device included in the light-emitting apparatus of FIG. 4 may be a tandem light-emitting device.

### Description of FIG. 4

FIG. 4 is a schematic perspective view of electronic device 1 including a light-emitting device according to one or more embodiments. The electronic device 1 may be, as an apparatus that displays a moving image and/or a still image, a portable electronic device, such as a mobile phone, a smartphone, a tablet personal computer (PC), a mobile communication terminal, an electronic notebook, an electronic book, a portable multimedia player (PMP), a navigation, or an ultra-mobile PC (UMPC), as well as one or more suitable products, such as a television, a laptop, a monitor, a billboard, or an Internet of things (IoT) device. The electronic device 1 may be such a product as described in one or more embodiments or a part thereof. In one or more embodiments, the electronic device 1 may be a wearable device, such as a smart watch, a watch phone, a glasses-type (kind) display, or a head mounted display (HMD), or a part of the wearable device. However, embodiments of the present disclosure are not limited thereto. In one or more embodiments, the electronic device 1 may be a dashboard of a vehicle, a center information display (CID) arranged on a center fascia or a dashboard of a vehicle, a room mirror display instead of a side-view mirror of a vehicle, an entertainment for the back seat of a vehicle, or a display arranged on the back of the front seat of a vehicle, a head up display (HUD) installed on the front of a vehicle or projected on a front window glass thereof, or a computer generated hologram augmented reality head up display (CGH AR HUD). FIG. 4 illustrates one or more embodiments in which the electronic device 1 is a smartphone for convenience of explanation.

The electronic device 1 may include a display area DA and a non-display area NDA outside (e.g., around) the display area DA. A display apparatus of the electronic device 1 may implement an image through an array of a plurality of pixels that are two-dimensionally arranged or provided in the display area DA.

The non-display area NDA may be an area that does not display an image, and may entirely be around (e.g., surround) the display area DA. On the non-display area NDA, a driver to provide electrical signals or power to display devices on the display area DA may be arranged or provided. On the non-display area NDA, a pad, which is an area to which an electronic element or a printed circuit board, may be electrically connected may be arranged or provided.

In the electronic device 1, the length in an x-axis direction and the length in a y-axis direction may be different from each other. In one or more embodiments, as shown in FIG. 4, the length in the x-axis direction may be less than the length in the y-axis direction. In one or more embodiments, the length in the x-axis direction may be substantially the same as the length in the y-axis direction. In one or more embodiments, the length in the x-axis direction may be greater than the length in the y-axis direction.

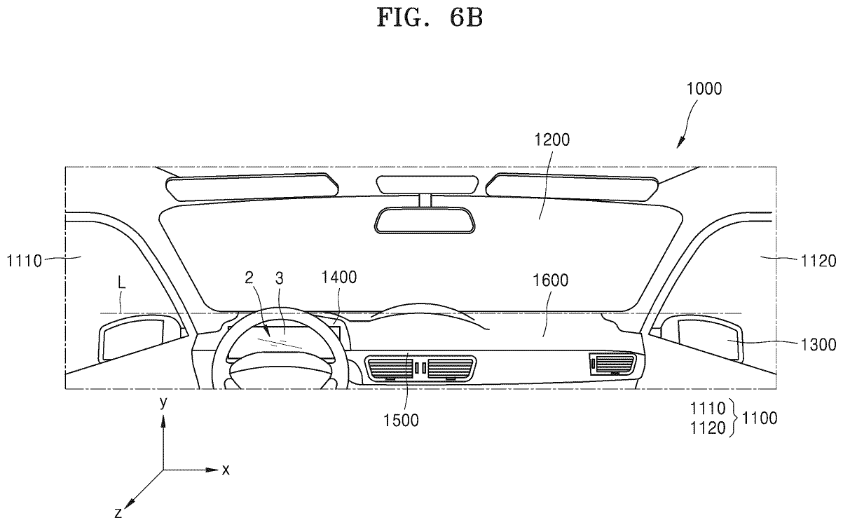

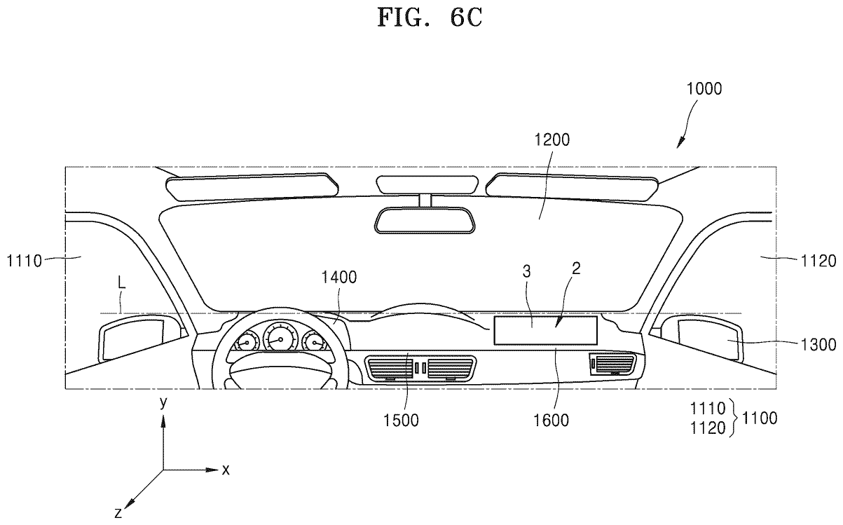

### Descriptions of FIGS. 5 and 6A to 6C

FIG. 5 is a schematic view of the exterior of a vehicle 1000 as electronic device including a light-emitting device, according to one or more embodiments. FIGS. 6A to 6C are each a schematic view of the interior of the vehicle 1000 according to one or more embodiments.

Referring to FIGS. 5, 6A, 6B, and 6C, the vehicle 1000 may refer to one or more suitable apparatuses to move a subject to be transported, such as a human, an object, and/or an animal, from a departure point to a destination point. The vehicle 1000 may include a vehicle that travels on a road or a track, a vessel that moves over the sea or a river, an airplane that flies in the sky using the action of air, and/or the like.

In one or more embodiments, the vehicle 1000 may travel on a road or a track. The vehicle 1000 may move in a certain direction according to rotation of at least one wheel. In one or more embodiments, the vehicle 1000 may include a three-wheeled vehicle, a four-wheeled vehicle, a construction machine, a two-wheeled vehicle, a prime mover device, a bicycle, and a train running on a track.

The vehicle 1000 may include a body having an interior and an exterior, and a chassis in which mechanical apparatuses necessary to drive may be installed as other parts except for the body of the vehicle 1000. The exterior of the body of the vehicle may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side-view mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face (e.g., opposite to) each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced and/or apart (e.g., spaced apart or separated) from each other in an x direction or a −x direction (e.g., the direction opposite the x-direction). In one or more embodiments, the first side window glass 1110 and the second side window glass 1120 may be spaced and/or apart (e.g., spaced apart or separated) from each other in the x direction or the −x direction (e.g., the direction opposite the x-direction). For example, an imaginary straight line L that connects the side window glasses 1100 may extend in the x direction or the −x direction (e.g., the direction opposite the x-direction). In one or more embodiments, an imaginary straight line L that connects the first side window glass 1110 and the second side window glass 1120 to each other may extend in the x direction or the −x direction (e.g., the direction opposite the x-direction).

The front window glass 1200 may be installed at the front of the vehicle 1000. The front window glass 1200 may be arranged between the side window glasses 1100 opposite to (e.g., facing) each other.

The side-view mirror 1300 may provide a rear view of the vehicle 1000. The side-view mirror 1300 may be installed on the exterior of the body of the vehicle. In one or more embodiments, a plurality of side-view mirrors 1300 may be provided. Any one of the plurality of side-view mirrors 1300 may be arranged outside the first side window glass 1110. Another of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge, a turn signal indicator, a high beam indicator, a warning light, a seat belt warning light, an odometer, a tachograph, an automatic shift selector indicator, a door open warning light, an engine oil warning light, and/or a low fuel warning light.

The center fascia 1500 may include a control panel on which a plurality of buttons to adjust an audio device, an air conditioning device, and/or a seat heater are arranged. The center fascia 1500 may be arranged on one side of the cluster 1400.

The passenger seat dashboard 1600 may be spaced and/or apart (e.g., spaced apart or separated) from the cluster 1400, and the center fascia 1500 may be arranged between the cluster 1400 and the passenger seat dashboard 1600. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may be to display an image. The display apparatus 2 may be arranged inside the vehicle 1000. In one or more embodiments, the display apparatus 2 may be arranged between the side window glasses 1100 opposite to (e.g., facing) each other. The display apparatus 2 may be arranged on at least one selected from among the cluster 1400, the center fascia 1500, and the passenger seat dashboard 1600.

The display apparatus 2 may include an organic light-emitting display, an inorganic electroluminescent display, a quantum dot display, and/or the like. Hereinafter, as the display apparatus 2 according to one or more embodiments, an organic light-emitting display apparatus including the light-emitting device will be described in more detail as an example, but one or more suitable types (kinds) of display apparatuses as described in one or more embodiments may be used in embodiments.

Referring to FIG. 6A, the display apparatus 2 may be arranged on the center fascia 1500. In one or more embodiments, the display apparatus 2 may be to display navigation information. In one or more embodiments, the display apparatus 2 may be to display information regarding audio settings, video setting, and/or vehicle settings.

Referring to FIG. 6B, the display apparatus 2 may be arranged on the cluster 1400. In one or more embodiments, the cluster 1400 may be to display driving information and/or the like through the display apparatus 2. For example, the cluster 1400 may be to digitally implement digital information and/or the like. The cluster 1400 may be to digitally display vehicle information and driving information as images. In one or more embodiments, a needle and a gauge of a tachometer and one or more suitable warning light icons may be displayed by a digital signal.

Referring to FIG. 6C, the display apparatus 2 may be arranged on the passenger seat dashboard 1600. The display apparatus 2 may be embedded in the passenger seat dashboard 1600 or arranged on the passenger seat dashboard 1600. In one or more embodiments, the display apparatus 2 on the passenger seat dashboard 1600 may be to display an image related to information displayed on the cluster 1400 and/or information displayed on the center fascia 1500. In one or more embodiments, the display apparatus 2 on the passenger seat dashboard 1600 may be to display information different from information displayed on the cluster 1400 and/or information displayed on the center fascia 1500.

### Manufacturing Method

The layers that constitute the hole transport region, the emission layer, and the layers that constitute the electron transport region may be each formed or arranged in a certain region by using one or more suitable methods, such as vacuum deposition, spin coating, casting, Langmuir-Blodgett (LB) deposition, ink-jet printing, laser-printing, laser-induced thermal imaging, and/or the like.

If (e.g., when) the layers that constitute the hole transport region, the emission layer, and the layers that constitute the electron transport region are each formed or arranged by vacuum deposition, the deposition may be performed at a deposition temperature in a range of about 100° C. to about 500° C., at a vacuum degree in a range of about 10⁻⁸ torr to about 10⁻³ torr, and at a deposition speed in a range of about 0.01 Å/sec to about 100 Å/sec, depending on a material to be included in a layer to be formed or arranged and the structure of a layer to be formed or arranged.

### DEFINITION OF TERMS

The term “C₃-C₆₀ carbocyclic group” as used herein refers to a cyclic group including (e.g., consisting of) carbon atoms as the only ring-forming atoms and having three to sixty carbon atoms, and the term “C₁-C₆₀ heterocyclic group” as used herein refers to a cyclic group that has one to sixty carbon atoms and further includes, in addition to carbon atom(s), a heteroatom as a ring-forming atom. The C₃-C₆₀ carbocyclic group and the C₁-C₆₀ heterocyclic group may each be a monocyclic group including (e.g., consisting of) one ring or a polycyclic group in which two or more rings are condensed with each other. In one or more embodiments, the number of ring-forming atoms of the C₁-C₆₀ heterocyclic group may be 3 to 61.

The term “cyclic group” as used herein may include both (e.g., simultaneously) the C₃-C₆₀ carbocyclic group and the C₁-C₆₀ heterocyclic group.

The term “π electron-rich C₃-C₆₀ cyclic group” as used herein refers to a cyclic group that has three to sixty carbon atoms and does not include *—N═*′ as a ring-forming moiety, and the term “π electron-deficient nitrogen-containing C₁-C₆₀ heterocyclic group” as used herein refers to a heterocyclic group that has one to sixty carbon atoms and includes *—N=*′ as a ring-forming moiety.

In one or more embodiments, the C₃-C₆₀ carbocyclic group may be i) Group T1 or ii) a condensed cyclic group in which two or more selected from among Group T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) Group T2, ii) a condensed cyclic group in which two or more selected from among Group T2 are condensed with each other, or iii) a condensed cyclic group in which at least one Group T2 and at least one Group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
the π electron-rich C₃-C₆₀ cyclic group may be i) Group T1, ii) a condensed cyclic group in which two or more selected from among Group T1 are condensed with each other, iii) Group T3, iv) a condensed cyclic group in which two or more selected from among Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like),
the π electron-deficient nitrogen-containing C₁-C₆₀ heterocyclic group may be i) Group T4, ii) a condensed cyclic group in which two or more selected from among Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T₄, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),

Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,

Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

Group T3 may be a furan group, a thiophene group, a 1H-pyrrole group, a silole group, or a borole group, and

Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ heterocyclic group” as used herein may refer to a group condensed to any cyclic group, a monovalent group, or a polyvalent group (for example, a divalent group, a trivalent group, a tetravalent group, and/or the like) according to the structure of a formula for which the corresponding term is used. In one or more embodiments, the “benzene group” may be a benzo group, a phenyl group, a phenylene group, and/or the like, which may be easily understood by those of ordinary skill in the art according to the structure of a formula including the “benzene group.”

Non-limiting examples of the monovalent C₃-C₆₀ carbocyclic group and monovalent C₁-C₆₀ heterocyclic group may be a C₅-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and non-limiting examples of the divalent C₃-C₆₀ carbocyclic group and the divalent C₁-C₆₀ heterocyclic group may be a C₃-C₁₀ cycloalkylene group, a C₁-C₁₀ heterocycloalkylene group, a C₅-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has one to sixty carbon atoms, and non-limiting examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl 1 group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond in the middle or at the terminus of a C₂-C₆₀ alkyl group, and non-limiting examples thereof include an ethenyl group, a propenyl group, and a butenyl group. The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of a C₂-C₆₀ alkyl group, and non-limiting examples thereof include an ethynyl group and a propynyl group. The term “C₂-C₆₀ alkynylene group” as used herein refers to a divalent group having substantially the same structure as a C₂-C₆₀ alkynyl group.

The term “C₁-C₆₀ alkoxy group” as used herein refers to a monovalent group represented by —OA₁₀₁ (wherein A₁₀₁ is a C₁-C₆₀ alkyl group), and non-limiting examples thereof include a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and non-limiting examples thereof may include a cyclopropyl group, a cyclobutyl group, a cyclopentyl group, a cyclohexyl group, a cycloheptyl group, a cyclooctyl group, an adamantanyl (or adamantyl) group, a norbornanyl (or norbornyl) group (or bicyclo[2.2.1]heptyl group), a bicyclo[1.1.1]pentyl group, a bicyclo[2.1.1]hexyl group, a bicyclo[2.2.2]octyl group, and/or the like. The term “C₃-C₁₀ cycloalkylene group” as used herein refers to a divalent group having substantially the same structure as the C₃-C₁₀ cycloalkyl group.

The term “C₁-C₁₀ heterocycloalkyl group” as used herein refers to a monovalent cyclic group that has one to ten carbon atoms and further includes, in addition to the carbon atoms, at least one heteroatom as a ring-forming atom, and non-limiting examples thereof include a 1,2,3,4-oxatriazolidinyl group, a tetrahydrofuranyl group, and a tetrahydrothiophenyl group. The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent cyclic group that has three to ten carbon atoms and at least one carbon-carbon double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having substantially the same structure as the C₃-C₁₀ cycloalkenyl group.

The term “C₁-C₁₀ heterocycloalkenyl group” as used herein refers to a monovalent cyclic group that has one to ten carbon atoms, further includes, in addition to the carbon atoms, at least one heteroatom as a ring-forming atom, and has at least one double bond in the ring thereof. Non-limiting examples of the C₁-C₁₀ heterocycloalkenyl group are a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having substantially the same structure as the C₁-C₁₀ heterocycloalkenyl group.

The term “C₆-C₆₀ aryl group” as used herein refers to a monovalent group having a carbocyclic aromatic system of six to sixty carbon atoms, and the term “C₆-C₆₀ arylene group” as used herein refers to a divalent group having a carbocyclic aromatic system of six to sixty carbon atoms. Non-limiting examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. If (e.g., when) the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having a heterocyclic aromatic system that has one to sixty carbon atoms and further includes, in addition to the carbon atoms, at least one heteroatom as a ring-forming atom. The term “C₁-C₆₀ heteroarylene group” as used herein refers to a divalent group having a heterocyclic aromatic system that has one to sixty carbon atoms and further includes, in addition to the carbon atoms, at least one heteroatom as a ring-forming atom. Non-limiting examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. If (e.g., when) the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group having two or more rings condensed with each other, only carbon atoms (for example, eight to sixty carbon atoms) as ring-forming atoms, and no aromaticity in its molecular structure if (e.g., when) considered as a whole. Non-limiting examples of the monovalent non-aromatic condensed polycyclic group are an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein refers to a divalent group having substantially the same structure as the monovalent non-aromatic condensed polycyclic group.

The term “monovalent non-aromatic condensed heteropolycyclic group” as used herein refers to a monovalent group that has two or more rings condensed with each other, further includes, in addition to carbon atoms (for example, one to sixty carbon atoms), at least one heteroatom as a ring-forming atom, and has no aromaticity in its molecular structure if (e.g., when) considered as a whole. Non-limiting examples of the monovalent non-aromatic condensed heteropolycyclic group are a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as used herein refers to a divalent group having substantially the same structure as the monovalent non-aromatic condensed heteropolycyclic group.

The term “C₆-C₆₀ aryloxy group” as used herein refers to —OA₁₀₂ (wherein A₁₀₂ is a C₆-C₆₀ aryl group), and the term “C₆-C₆₀ arylthio group” as used herein refers to —SA₁₀₃ (wherein Atos is a C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₅-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O) (Q₁₁), —S(═O)₂ (Q₁₁), —P(═O)(Q₁₁)(Q₁₂), or a (e.g., any suitable) combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, a C₁-C₆₀ heteroaryloxy group, or a C₁-C₆₀ heteroarylthio group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O) (Q₂₁), —S(═O)₂ (Q₂₁), —P(═O)(Q₂₁)(Q₂₂), or a (e.g., any suitable) combination thereof; or
—Si(Q₃₁)(Q₃₂)(Q₃₃), —N(Q₃₁)(Q₃₂), —B(Q₃₁)(Q₃₂), —C(═O) (Q₃₁), —S(═O)₂ (Q₃₁), or —P(═O)(Q₃₁)(Q₃₂).
Q₁ to Q₃, Q₁₁ to Q₁₃, Q₂₁ to Q₂₃, and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₅-C₆₀ carbocyclic group, a C₁-C₆₀ heterocyclic group, a C₇-C₆₀ arylalkyl group or a C₂-C₆₀ heteroarylalkyl group, each being unsubstituted or substituted with deuterium, —F, a cyano group, a C₁-C₆₀ alkyl group, a C₁-C₆₀ alkoxy group, a phenyl group, a biphenyl group, or a (e.g., any suitable) combination thereof;

The term “heteroatom” as used herein refers to an atom other than a carbon atom or a hydrogen atom. Non-limiting examples of the heteroatom are O, S, N, P, Si, B, Ge, Se, or a (e.g., any suitable) combination thereof.

The term “transition metal” as used herein includes hafnium (Hf), tantalum (Ta), tungsten (W), rhenium (Re), osmium (Os), iridium (Ir), platinum (Pt), gold (Au), and/or the like.

“Ph” as used herein refers to a phenyl group, “Me” as used herein refers to a methyl group, “Et” as used herein refers to an ethyl group, “tert-Bu” or “But” as used herein refers to a tert-butyl group, and “OMe” as used herein refers to a methoxy group. “D” represents deuterium,

The term “biphenyl group” as used herein refers to “a phenyl group that is substituted with a phenyl group.” For example, the “biphenyl group” may be a substituted phenyl group having a C₆-C₆₀ aryl group as a substituent.

The term “terphenyl group” as used herein refers to “a phenyl group substituted with a biphenyl group.” For example, the “terphenyl group” is a substituted phenyl group having, as a substituent, a C₆-C₆₀ aryl group substituted with a C₆-C₆₀ aryl group.
* and *′ as used herein, unless defined otherwise, each refer to a binding site to a neighboring atom in a corresponding formula or moiety.

The terms “x-axis”, “y-axis”, and “z-axis” as used herein are not limited to three axes in an orthogonal coordinate system, and may be interpreted in a broader sense than the aforementioned three axes in an orthogonal coordinate system. For example, the x-axis, y-axis, and z-axis may describe axes that are orthogonal to each other, or may describe axes that are in different directions that are not orthogonal to each other.

Hereinafter, the compounds according to one or more embodiments and the light-emitting devices according to one or more embodiments will be described in more detail with reference to the following Synthesis Examples and Examples. The wording “B was used instead of A” used in describing Synthesis Examples refers to that a substantially identical molar equivalent of B was used in place of A.

### EXAMPLES

### Synthesis Example 1: Synthesis of Compound 1

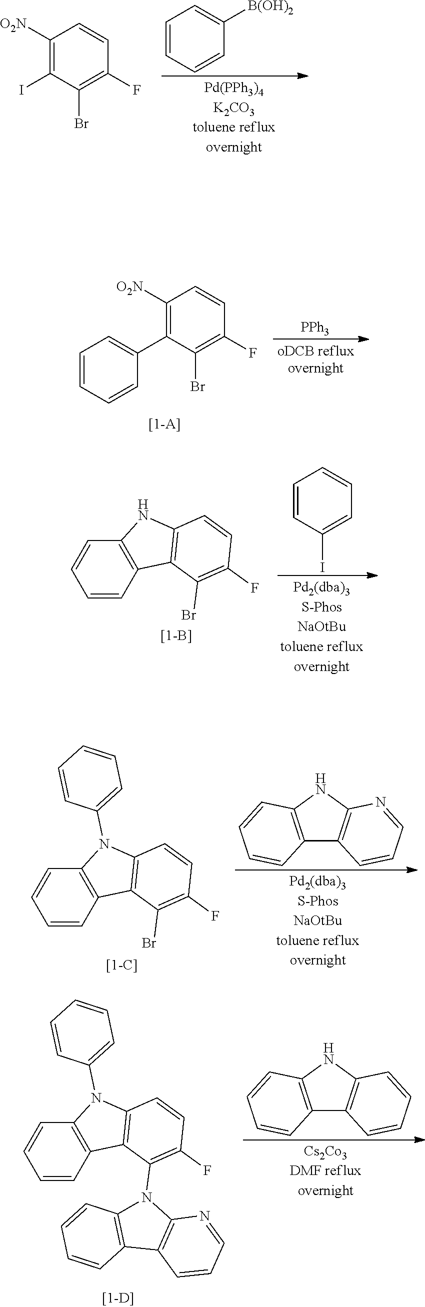

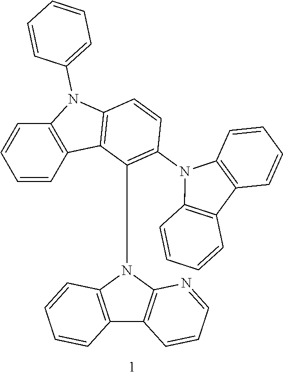 [SMILES: c1ccc(-n2c3ccccc3c3c(-n4c5ccccc5c5cccnc54)c(-n4c5ccccc5c5ccccc54)ccc32)cc1]

### 1) Synthesis of Intermediate 1-A

8.65 g (25.0 mmol) of 2-bromo-1-fluoro-3-iodo-4-nitrobenzene, 3.05 g (25.0 mmol) of phenylboronic acid, 1.44 g (1.25 mmol) of tetrakis(triphenylphosphine) palladium (0), and 10.4 g (75.0 mmol) of potassium carbonate were placed in a reaction vessel and suspended in 250 mL of toluene under reflux. The reaction temperature was raised to 110° C. and stirred overnight. After completion of the reaction, the mixture was cooled to room temperature and extracted with ethyl acetate. The extracted organic layer was washed with saturated sodium chloride solution and dried over sodium sulfate. The residue after removal of the solvent was separated by column chromatography to obtain 4.53 g (15.3 mmol) of the target compound.

### 2) Synthesis of Intermediate 1-B

4.53 g (15.3 mmol) of Intermediate 1-A and 8.03 g (30.6 mmol) of triphenylphosphine were placed in a reaction vessel and suspended in 150 mL of ortho-dichlorobenzene (oDCB; or 1,2-dichlorobenzene) under reflux. The reaction temperature was raised to 190° C. and stirred overnight. After completion of the reaction, the mixture was cooled to room temperature and extracted with ethyl acetate. The extracted organic layer was washed with saturated sodium chloride solution and dried over sodium sulfate. The residue after removal of the solvent was separated by column chromatography to obtain 1.37 g (5.20 mmol) of the target compound.

### 3) Synthesis of Intermediate 1-C

1.37 g (5.20 mmol) of Intermediate 1-B, 1.17 g (5.72 mmol) of iodobenzene, 240 mg (0.26 mmol) of tris(dibenzylideneacetone) dipalladium, 210 mg (0.52 mmol) of dicyclohexyl(2′,6′-dimethoxy[1,1′-biphenyl]-2-yl)phosphane (S-Phos), and 1.50 g (15.6 mmol) of sodium tert-butoxide were placed in a reaction vessel and suspended in 50 mL of toluene under reflux. The reaction temperature was raised to 110° C. and stirred overnight. After completion of the reaction, the mixture was cooled to room temperature and extracted with ethyl acetate. The extracted organic layer was washed with saturated sodium chloride solution and dried over sodium sulfate. The residue after removal of the solvent was separated by column chromatography to obtain 1.40 g (4.11 mmol) of the target compound.

### 4) Synthesis of Intermediate 1-D

1.40 g (4.11 mmol) of Intermediate 1-C, 760 mg (4.52 mmol) of 9H-pyrido[2,3-b]indole, 190 mg (0.21 mmol) of tris(dibenzylideneacetone) dipalladium, 170 mg (0.41 mmol) of S-Phos, and 1.18 g (12.3 mmol) of sodium tert-butoxide were placed in a reaction vessel and suspended in 40 mL of toluene under reflux. The reaction temperature was raised to 110° C. and stirred overnight. After completion of the reaction, the mixture was cooled to room temperature and extracted with ethyl acetate. The extracted organic layer was washed with saturated sodium chloride solution and dried over sodium sulfate. The residue after removal of the solvent was separated by column chromatography to obtain 1.27 g (2.96 mmol) of the target compound.

### 5) Synthesis of Compound 1

1.27 g (2.96 mmol) of Intermediate 1-D, 740 mg (4.44 mmol) of carbazole, and 2.89 g (8.88 mmol) of cesium carbonate were placed in a reaction vessel and suspended in 30 mL of N,N-dimethylmethanamide (DMF) under reflux. The reaction temperature was raised to 160° C. and stirred overnight. After completion of the reaction, the mixture was cooled to room temperature and extracted with ethyl acetate. The extracted organic layer was washed with saturated sodium chloride solution and 1 dried over sodium sulfate. The residue after removal of the solvent was separated by column chromatography to obtain 1.02 g (1.78 mmol) of the target compound.

### Synthesis Example 2: Synthesis of Compound 3

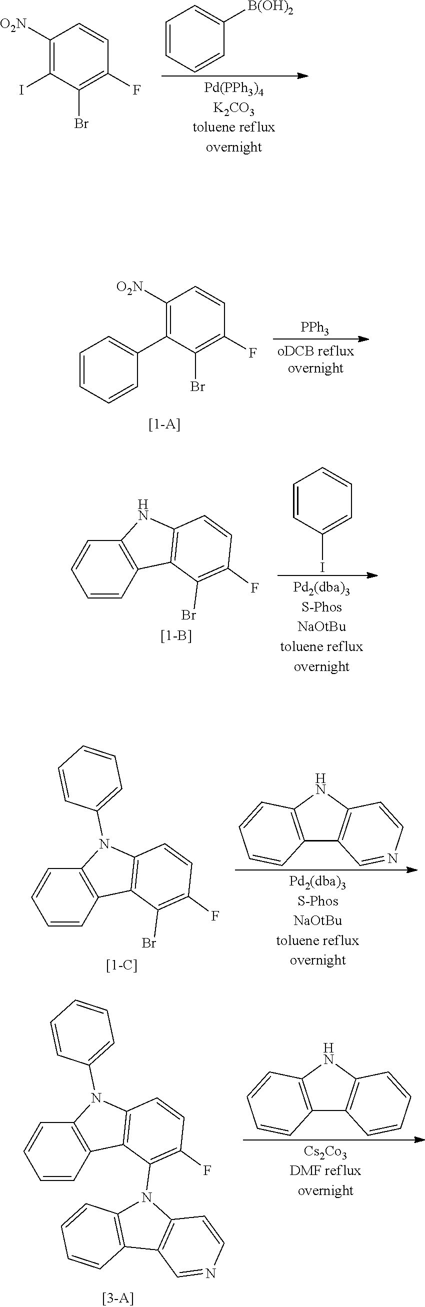

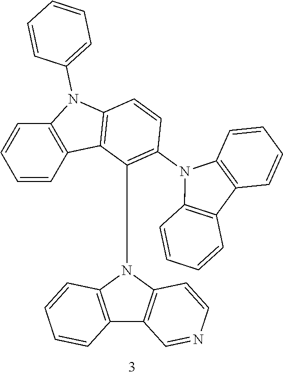 [SMILES: c1ccc(-n2c3ccccc3c3c(-n4c5ccccc5c5cnccc54)c(-n4c5ccccc5c5ccccc54)ccc32)cc1]

930 mg (1.61 mmol) of the target compound was obtained using substantially the same method as Synthesis Example 1, except that 5H-pyrido[4,3-b]indole was used instead of 9H-pyrido[2,3-b]indole.

### Synthesis Example 3: Synthesis of Compound 5

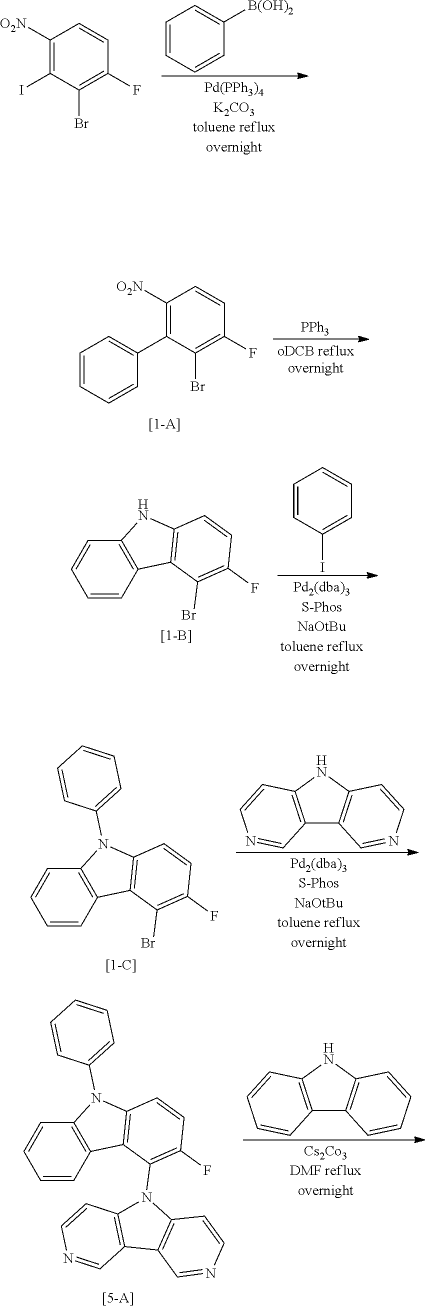

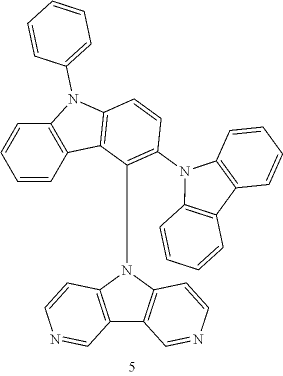 [SMILES: c1ccc(-n2c3ccccc3c3c(-n4c5ccncc5c5cnccc54)c(-n4c5ccccc5c5ccccc54)ccc32)cc1]

980 mg (1.70 mmol) of the target compound was obtained using substantially the same method as Synthesis Example 1, except that 5H-pyrrolo[3,2-c: 4,5-c′]dipyridine was used instead of 9H-pyrido[2,3-b]indole.9H-pyrido[2,3-b]indole.

### Synthesis Example 4: Synthesis of Compound 19

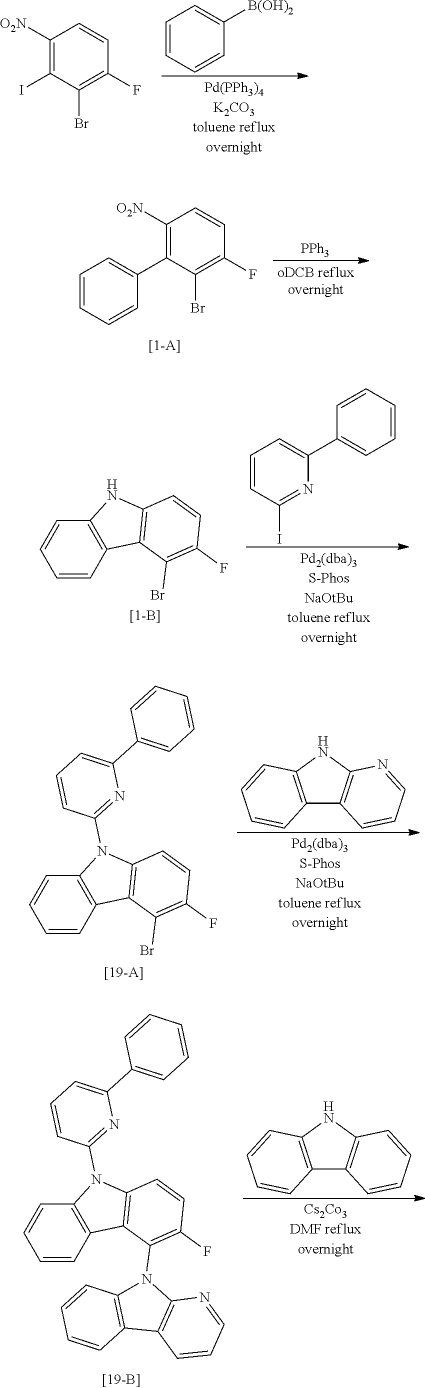

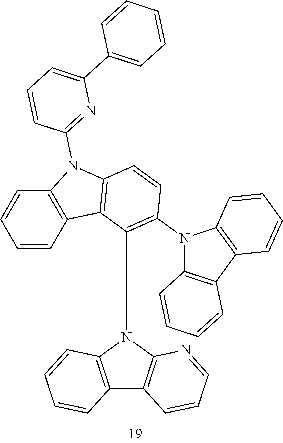 [SMILES: c1ccc(-c2cccc(-n3c4ccccc4c4c(-n5c6ccccc6c6cccnc65)c(-n5c6ccccc6c6ccccc65)ccc43)n2)cc1]

1.13 g (1.74 mmol) of the target compound was obtained using substantially the same method as Synthesis Example 1, except that 2-iodo-6-phenylpyridine was used instead of iodobenzene.

### Synthesis Example 5: Synthesis of Compound 26

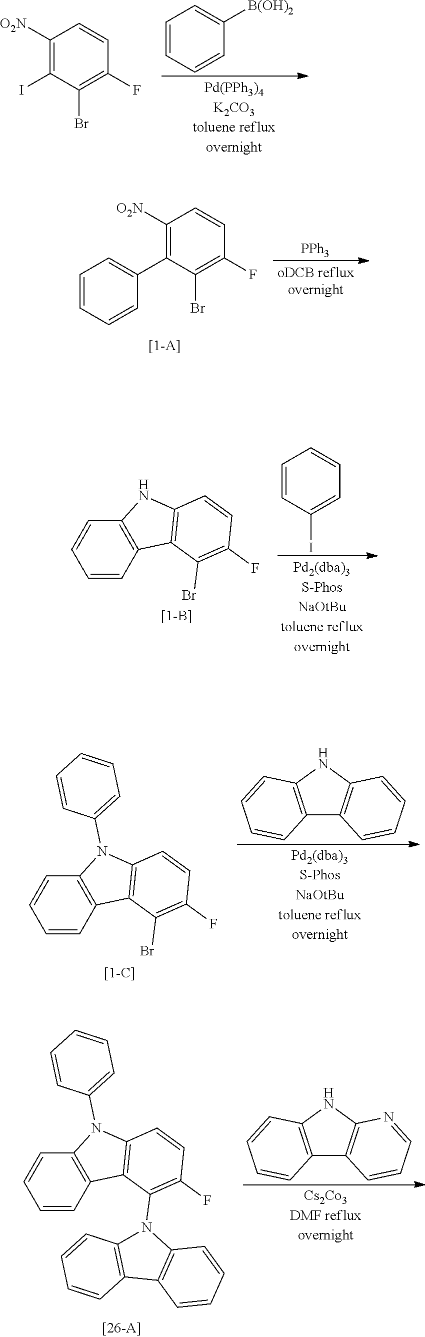

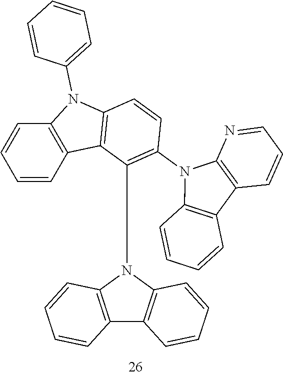 [SMILES: c1ccc(-n2c3ccccc3c3c(-n4c5ccccc5c5ccccc54)c(-n4c5ccccc5c5cccnc54)ccc32)cc1]

970 mg (1.69 mmol) of the target compound was obtained using substantially the same method as Synthesis Example 1, except that carbazole was used instead of 9H-pyrido[2,3-b]indole and 9H-pyrido[2,3-b]indole was used instead of carbazole.

### Synthesis Example 6: Synthesis of Compound 51

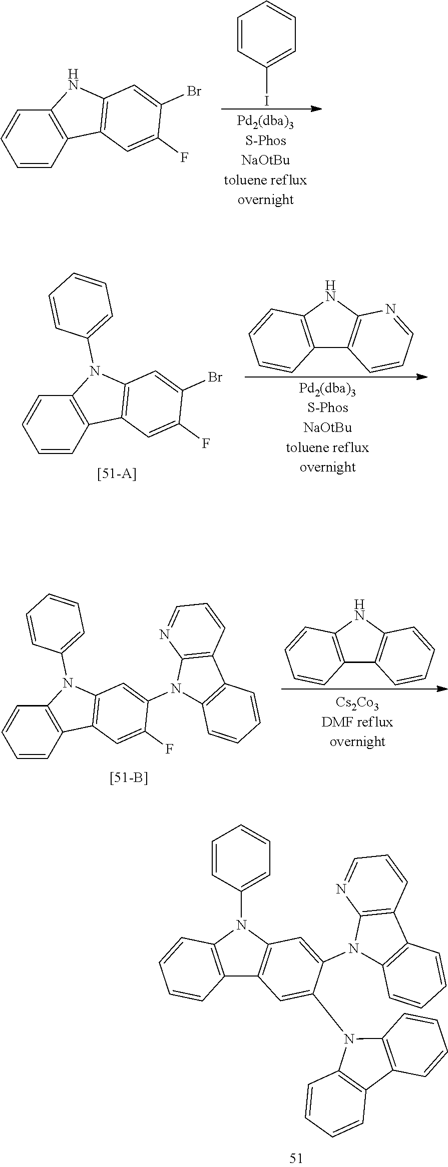

### 1) Synthesis of Intermediate 51-A

1.32 g (5.0 mmol) of 2-bromo-3-fluoro-9H-carbazole, 1.12 g (5.5 mmol) of iodobenzene, 230 mg (0.25 mmol) of tris(dibenzylideneacetone) dipalladium, 210 mg (0.50 mmol) of S-Phos, and 1.44 g (15.0 mmol) of sodium tert-butoxide were placed in a reaction vessel and suspended in 50 mL of toluene under reflux. The reaction temperature was raised to 110° C. and stirred overnight. After completion of the reaction, the mixture was cooled to room temperature and extracted with ethyl acetate. The extracted organic layer was washed with saturated sodium chloride solution and dried over sodium sulfate. The residue after removal of the solvent was separated by column chromatography to obtain 1.41 g (4.15 mmol) of the target compound.

### 2) Synthesis of Intermediate 51-B

1.41 g (4.15 mmol) of Intermediate 51-A, 770 mg (4.57 mmol) of 9H-pyrido[2,3-b]indole, 190 mg (0.21 mmol) of tris(dibenzylideneacetone) dipalladium, 180 mg (0.42 mmol) of S-Phos, and 1.20 g (12.5 mmol) of sodium tert-butoxide were placed in a reaction vessel and suspended in 40 mL of toluene under reflux. The reaction temperature was raised to 110° C. and stirred overnight. After completion of the reaction, the mixture was cooled to room temperature and extracted with ethyl acetate. The extracted organic layer was washed with saturated sodium chloride solution and dried over sodium sulfate. The residue after removal of the solvent was separated by column chromatography to obtain 1.39 g (3.24 mmol) of the target compound.

### 3) Synthesis of Compound 51

1.39 g (3.24 mmol) of Intermediate 51-B, 810 mg (4.86 mmol) of carbazole, and 3.16 g (9.72 mmol) of cesium carbonate were placed in a reaction vessel and suspended in 30 mL of DMF under reflux. The reaction temperature was raised to 160° C. and stirred overnight. After completion of the reaction, the mixture was cooled to room temperature and extracted with ethyl acetate. The extracted organic layer was washed with saturated sodium chloride solution and dried over sodium sulfate. The residue after removal of the solvent was separated by column chromatography to obtain 1.10 g (1.91 mmol) of the target compound.

### Synthesis Example 7: Synthesis of Compound 76

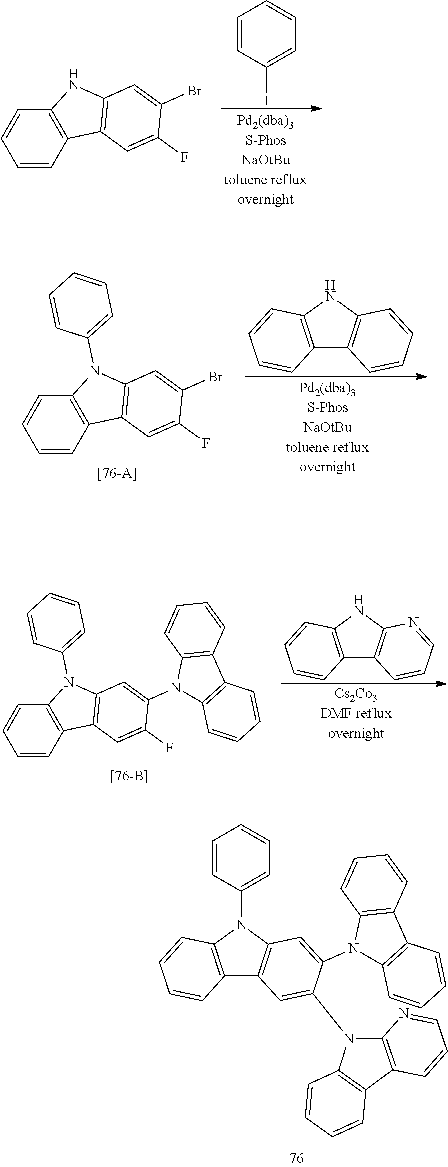

1.04 g (1.81 mmol) of the target compound was obtained using substantially the same method as Synthesis Example 8, except that carbazole was used instead of 9H-pyrido[2,3-b]indole and 9H-pyrido[2,3-b]indole was used instead of carbazole.

### Synthesis Example 8: Synthesis of Compound 162

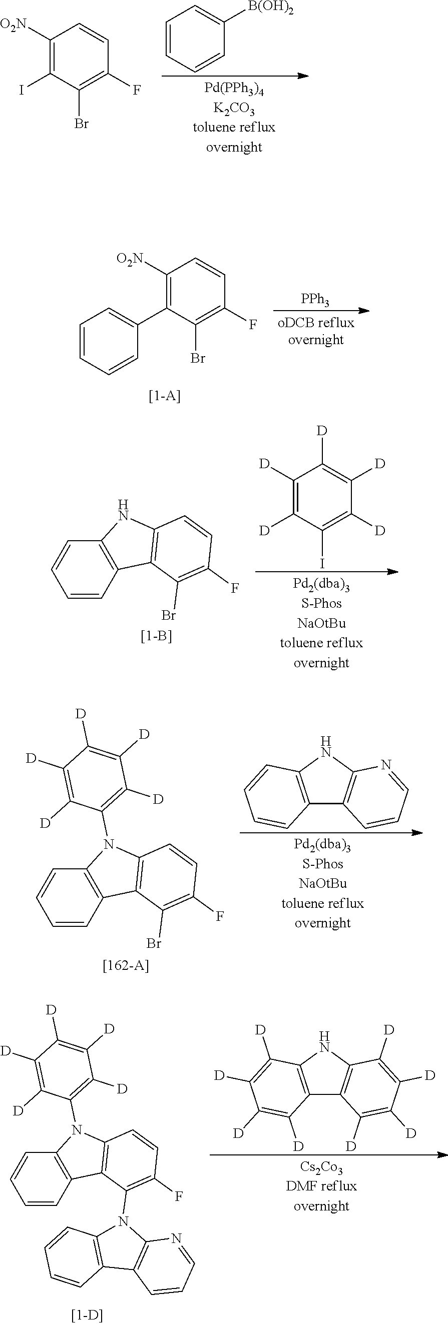

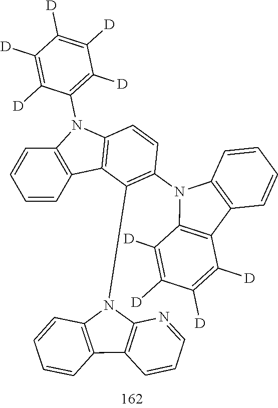 [SMILES: [2H]c1c([2H])c([2H])c(-n2c3ccccc3c3c(-n4c5ccccc5c5cccnc54)c(-n4c5ccccc5c5c([2H])c([2H])c([2H])c([2H])c54)ccc32)c([2H])c1[2H]]

1.13 g (1.92 mmol) of the target compound was obtained using substantially the same method as Synthesis Example 1, except that iodobenzene-D5 was used instead of iodobenzene and carbazole-D8 was used instead of carbazole.

TABLE 1

MS/FAB

| Compound No. | found | calc. |
|---|---|---|
| Compound 1 | 574.2155 | 574.2157 |
| Compound 3 | 574.2154 | 574.2157 |
| Compound 5 | 575.2114 | 575.2110 |
| Compound 19 | 651.2419 | 651.2423 |
| Compound 26 | 574.2153 | 574.2157 |
| Compound 51 | 574.2154 | 574.2157 |
| Compound 76 | 574.2152 | 574.2157 |
| Compound 162 | 587.2977 | 587.2973 |

### Example 1

A Corning 15 Ω/cm² (1200 Å) ITO glass substrate was cut to a size of 50 mm×50 mm×0.5 mm, ultrasonically cleaned with isopropyl alcohol and pure water for 5 minutes each, cleaned by ultraviolet (UV) irradiation and ozone exposure for 30 minutes, and then installed in a vacuum deposition apparatus.

On the substrate, HATCN was first deposited to form a hole injection layer having the thickness of 100 Å, followed by vacuum deposition of BCFN, which is a first hole transport material, to the thickness of 600 Å, and then SiCzCz, which is a second hole transport compound, was vacuum-deposited to the thickness of 50 Å, to form a hole transport layer.

On the hole transport layer, Compound 1 and SiTrzCz2 as hosts and PtON-TBBI as a phosphorescent dopant were co-deposited at a weight ratio of 60:27:13 to form an emission layer having the thickness of 350 Å.

Subsequently, mSiTrz was deposited on the emission layer to form a first electron transport layer having the thickness of 50 Å, followed by co-deposition of mSiTrz and lithium-8-hydroxyquinolinolate (LiQ) at a ratio of 1:1 to form a second electron transport layer having the thickness of 350 Å, to form an electron transport layer. Lithium fluoride (LiF), which is an alkali metal halide, was deposited to form an electron injection layer having the thickness of 15 Å on the electron transport layer, and Al was vacuum-deposited to form an electrode having the thickness of 80 Å, thereby completing the manufacture of an organic light-emitting device.

The materials used in the organic light-emitting device as described in one or more embodiments may be represented by the formulae:

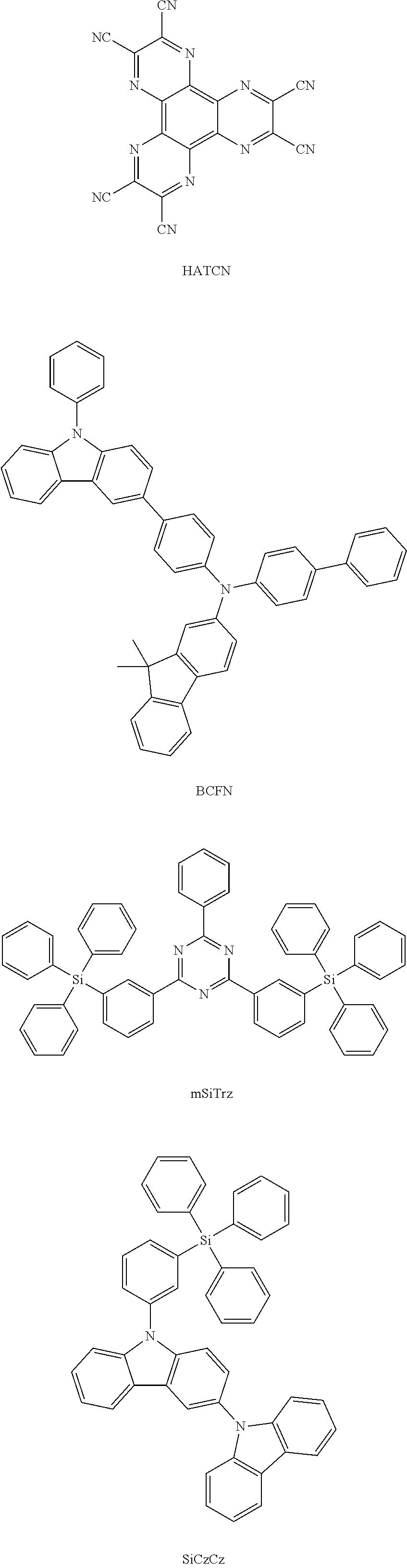

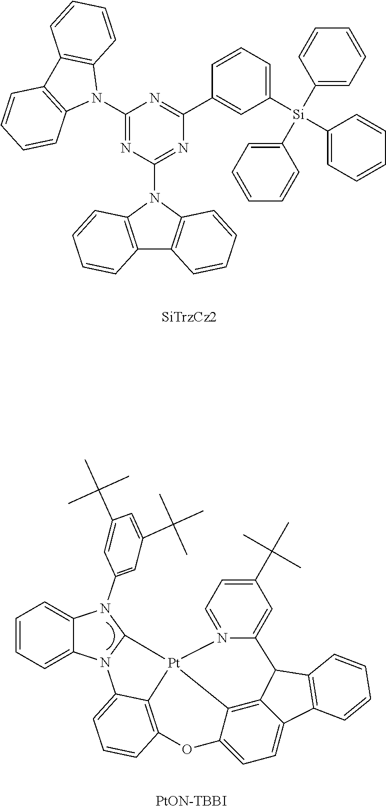

### Examples 2 to 8 and Comparative Examples 1 to 7

The organic light-emitting devices were manufactured in substantially the same manner as in Example 1, except that compounds shown in Table 1 were each used instead of Compound 1 in forming an emission layer.

### Evaluation Example 1

The driving voltage and maximum quantum efficiency at a current density of 10 mA/cm² and lifespan of the organic light-emitting devices according to Examples 1 to 8 and Comparative Examples 1 to 7 were measured. The driving voltage of the organic light-emitting devices was measured using a source meter (Keithley Instrument, 2400 series), and the maximum quantum efficiency was measured using an external quantum efficiency measurement device (Hamamatsu Photonics, C9920-2-12). In evaluating the maximum quantum efficiency, luminance/current density was measured using a luminance meter calibrated for wavelength sensitivity, and the maximum quantum efficiency was calculated assuming a Lambertian angular luminance distribution based on a perfect diffuse reflective surface. The values were then expressed as relative color conversion efficiency ratios, normalized to 100 with the color conversion efficiency of Comparative Example 1 set as the reference. The relative lifetime was expressed as the time required for the luminance to decrease to 95% of the initial luminance, normalized to 100 with the lifetime of Comparative Example 1 set as the reference. The characteristics evaluation results of organic light-emitting device are shown in Table 2.

TABLE 2

| | | | Color | | |
|---|---|---|---|---|---|
| | | Driving | conversion | | |
| | | voltage | efficiency | Lifespan | Emission |
| Classification | Host | (V) | ratio | ratio | color |
| Example 1 | Compound 1 | 4.8 | 120 | 112 | Blue |
| Example 2 | Compound 3 | 4.7 | 125 | 109 | Blue |
| Example 3 | Compound 5 | 4.8 | 114 | 107 | Blue |
| Example 4 | Compound 19 | 4.8 | 108 | 107 | Blue |
| Example 5 | Compound 26 | 4.6 | 111 | 108 | Blue |
| Example 6 | Compound 51 | 4.8 | 121 | 115 | Blue |
| Example 7 | Compound 76 | 4.6 | 109 | 111 | Blue |
| Example 8 | Compound 162 | 4.4 | 122 | 136 | Blue |
| Comparative | C1 | 5.0 | 100 | 100 | Blue |
| Example 1 | | | | | |
| Comparative | C2 | 5.2 | 87 | 72 | Blue |
| Example 2 | | | | | |
| Comparative | C3 | 5.1 | 106 | 68 | Blue |
| Example 3 | | | | | |
| Comparative | C4 | 5.3 | 110 | 75 | Blue |
| Example 4 | | | | | |
| Comparative | C5 | 5.1 | 115 | 82 | Blue |
| Example 5 | | | | | |
| Comparative | C6 | 4.8 | 79 | 49 | Blue |
| Example 6 | | | | | |
| Comparative | C7 | 5.3 | 81 | 37 | Blue |
| Example 7 | | | | | |

1 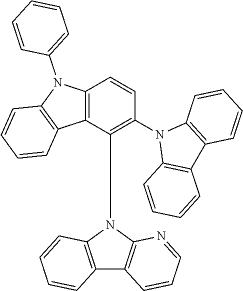 [SMILES: c1ccc(-n2c3ccccc3c3c(-n4c5ccccc5c5cccnc54)c(-n4c5ccccc5c5ccccc54)ccc32)cc1]
3 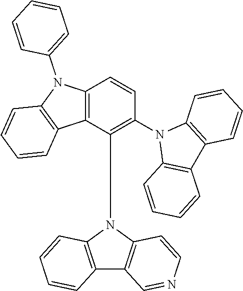 [SMILES: c1ccc(-n2c3ccccc3c3c(-n4c5ccccc5c5cnccc54)c(-n4c5ccccc5c5ccccc54)ccc32)cc1]
5 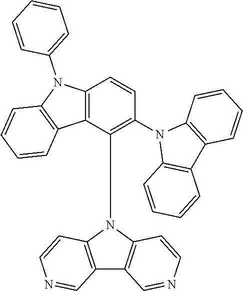 [SMILES: c1ccc(-n2c3ccccc3c3c(-n4c5ccncc5c5cnccc54)c(-n4c5ccccc5c5ccccc54)ccc32)cc1]
19 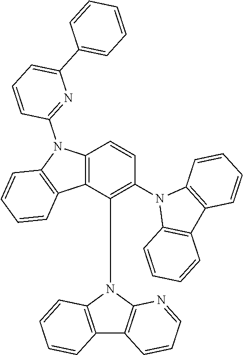 [SMILES: c1ccc(-c2cccc(-n3c4ccccc4c4c(-n5c6ccccc6c6cccnc65)c(-n5c6ccccc6c6ccccc65)ccc43)n2)cc1]
26 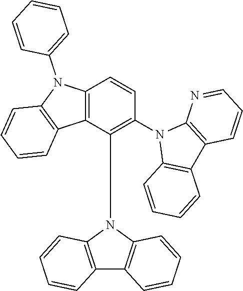 [SMILES: c1ccc(-n2c3ccccc3c3c(-n4c5ccccc5c5ccccc54)c(-n4c5ccccc5c5cccnc54)ccc32)cc1]
51 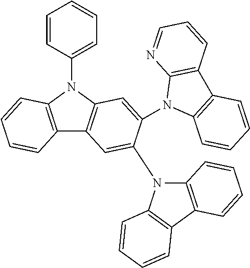 [SMILES: c1ccc(-n2c3ccccc3c3cc(-n4c5ccccc5c5ccccc54)c(-n4c5ccccc5c5cccnc54)cc32)cc1]
76 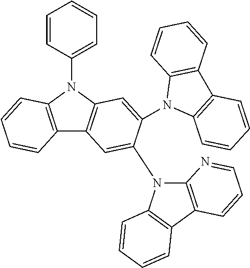 [SMILES: c1ccc(-n2c3ccccc3c3cc(-n4c5ccccc5c5cccnc54)c(-n4c5ccccc5c5ccccc54)cc32)cc1]
162 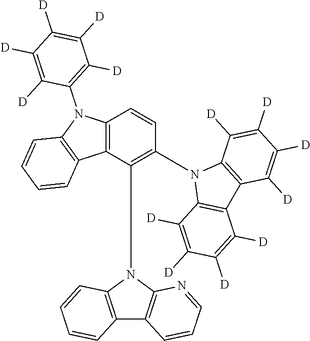 [SMILES: [2H]c1c([2H])c([2H])c(-n2c3ccccc3c3c(-n4c5ccccc5c5cccnc54)c(-n4c5c([2H])c([2H])c([2H])c([2H])c5c5c([2H])c([2H])c([2H])c([2H])c54)ccc32)c([2H])c1[2H]]
C1 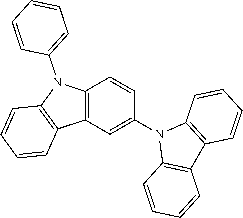 [SMILES: c1ccc(-n2c3ccccc3c3cc(-n4c5ccccc5c5ccccc54)ccc32)cc1]
C2 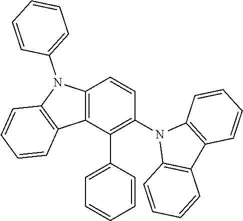 [SMILES: c1ccc(-c2c(-n3c4ccccc4c4ccccc43)ccc3c2c2ccccc2n3-c2ccccc2)cc1]
C3 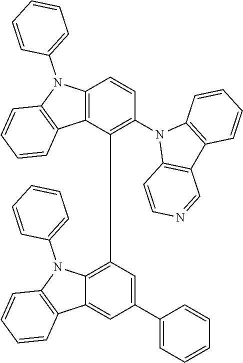 [SMILES: c1ccc(-c2cc(-c3c(-n4c5ccccc5c5cnccc54)ccc4c3c3ccccc3n4-c3ccccc3)c3c(c2)c2ccccc2n3-c2ccccc2)cc1]
C4 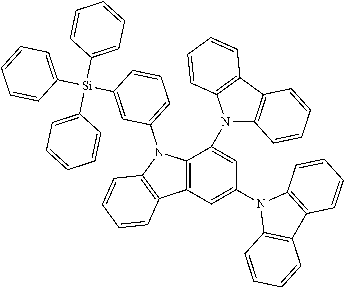 [SMILES: c1ccc([Si](c2ccccc2)(c2ccccc2)c2cccc(-n3c4ccccc4c4cc(-n5c6ccccc6c6ccccc65)cc(-n5c6ccccc6c6ccccc65)c43)c2)cc1]
C5 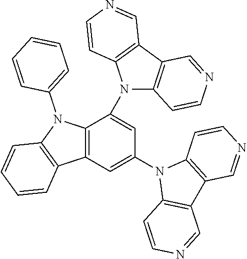 [SMILES: c1ccc(-n2c3ccccc3c3cc(-n4c5ccncc5c5cnccc54)cc(-n4c5ccncc5c5cnccc54)c32)cc1]
C6 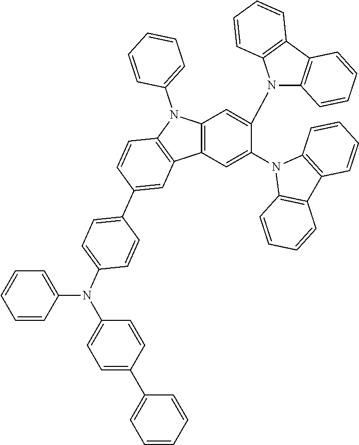 [SMILES: c1ccc(-c2ccc(N(c3ccccc3)c3ccc(-c4ccc5c(c4)c4cc(-n6c7ccccc7c7ccccc76)c(-n6c7ccccc7c7ccccc76)cc4n5-c4ccccc4)cc3)cc2)cc1]
C7

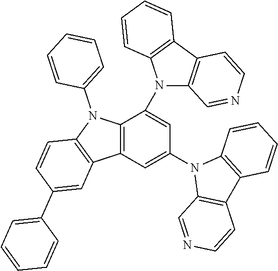 [SMILES: c1ccc(-c2ccc3c(c2)c2cc(-n4c5ccccc5c5ccncc54)cc(-n4c5ccccc5c5ccncc54)c2n3-c2ccccc2)cc1]

Referring to the results in Table 2, the organic light-emitting devices of Examples 1 to 8 each exhibits lower or equal driving voltage and superior lifespan compared to the organic light-emitting devices of Comparative Examples 1 to 7.

A light-emitting device including the heterocyclic compound represented by Formula 1 may have low driving voltage, high efficiency, and long lifespan. Furthermore, high-quality electronic apparatuses and consumer products may be manufactured by using the light-emitting device.

For example, the organic light-emitting devices of Examples 1 to 8 each exhibit lower driving voltage and superior lifespan compared to the organic light-emitting devices of Comparative Examples 1 to 7. Specifically, the driving voltage for the examples ranges from 4.4 V to 4.8 V, which is significantly lower than the 5.0 V to 5.3 V observed in the comparative examples. This reduction in driving voltage indicates higher energy efficiency, which is a critical factor for the performance and longevity of light-emitting devices.

Moreover, the lifespan ratios of the examples are notably higher, with Example 8 showing the highest lifespan ratio of 136 compared to the baseline of 100 set by Comparative Example 1. This improvement in lifespan may be attributed to the enhanced molecular structure of the heterocyclic compounds used in the examples, which enhances the stability and durability of the emission layer. The color conversion efficiency ratios are also superior, with values ranging from 108 to 125, indicating better performance in terms of light output and color quality.

A light-emitting device including the heterocyclic compound represented by Formula 1 may have low driving voltage, high efficiency, and long lifespan. Furthermore, high-quality electronic apparatuses and consumer products may be manufactured by using the light-emitting device. These advantages make the heterocyclic compounds described in the present disclosure highly suitable for advanced display technologies and other applications requiring efficient and durable light-emitting devices.

A light-emitting device, a display device, an electronic device, an electronic apparatus, a device for manufacturing substantially the same and/or any other relevant devices or components according to one or more embodiments of the present disclosure may be implemented by utilizing any suitable hardware, firmware (e.g., an application-specific integrated circuit), software, or a (e.g., any suitable) combination of software, firmware, and hardware. For example, the one or more components of the device may be provided on one integrated circuit (IC) chip or on separate IC chips. Further, the one or more components of the device may be implemented on a flexible printed circuit film, a tape carrier package (TCP), and/or a printed circuit board (PCB), or provided on one substrate. Further, the one or more components of the device may be a process or thread, running on one or more processors, in one or more computing devices, executing computer program instructions and interacting with other system components for performing the one or more functionalities described herein. The computer program instructions may be stored in a memory which may be implemented in a computing device using a standard memory device, such as, for example, a random access memory (RAM). The computer program instructions may also be stored in other non-transitory computer readable media, such as, for example, a CD-ROM, flash drive, and/or the like. Also, a person of skill in the art should recognize that the functionality of one or more computing devices may be combined or integrated into a single computing device, or the functionality of a particular computing device may be distributed across one or more other computing devices without departing from the scope of the present disclosure.

It should be understood that embodiments described herein should be considered in a descriptive sense only and not for purposes of limitation. Descriptions of features or aspects within each embodiment should typically be considered as available for other similar features or aspects in other embodiments. While the subject matter of the present disclosure has been described with reference to the drawings, it will be understood by those of ordinary skill in the art that one or more suitable changes in form and more details may be made therein without departing from the spirit and scope as defined by the appended claims and equivalents thereof.

What is claimed is:

1. A light-emitting device comprising:
a first electrode;
a second electrode opposite to the first electrode;
an interlayer between the first electrode and the second electrode and comprising an emission layer; and
a heterocyclic compound represented by Formula 1: 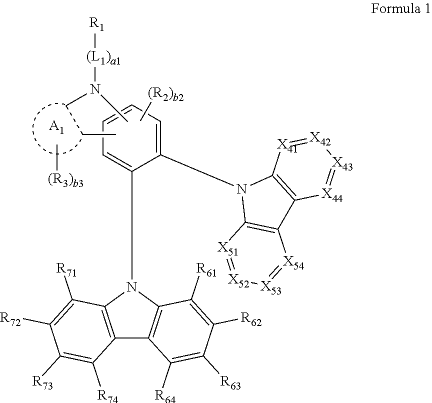
wherein, in Formula 1,
X₄₁ is N or C(R₄₁), X₄₂ is N or C(R₄₂), X₄₃ is N or C(R₄₃), X₄₄ is N or C(R₄₄), X₅₁ is N or C(R₅₁), X₅₂ is N or C(R₅₂), X₅₃ is N or C(R₅₃), and X₅₄ is N or C(R₅₄),
at least one selected from among X₄₁ to X₄₄ and X₅₁ to X₅₄ is N,
ring A₁ is a C₅-C₆₀ carbocyclic group or a C₁-C₆₀ heterocyclic group,
a1 is an integer of 0 to 5,
L₁ is a C₃-C₆₀ carbocyclic group that is unsubstituted or substituted with at least one R₁₀ₐ or a C₁-C₆₀ heterocyclic group that is unsubstituted or substituted with at least one R₁₀ₐ,
*-(L₁)ₐ₁-*′ is a single bond when a1 is 0,
b2 is an integer of 0 to 2,
b3 is an integer of 0 to 10,
R₁ to R₃, R₄₁ to R₄₄, R₅₁ to R₅₄, R₆₁ to R₆₄, and R₇₁ to R₇₄ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, a C₇-C₆₀ arylalkyl group unsubstituted or substituted with at least one R₁₀ₐ, —C(Q₁)(Q₂)(Q₃), —Si(Q₁)(Q₂)(Q₃), —B(Q₁)(Q₂), —N(Q₁)(Q₂), —P(Q₁)(Q₂), —C(═O) (Q₁), —S(═O) (Q₁), —S(═O)₂ (Q₁), —P(═O)(Q₁)(Q₂), or —P(═S)(Q₁)(Q₂),
neighboring two or more selected from among R₁ to R₃, R₄₁ to R₄₄, R₅₁ to R₅₄, R₆₁ to R₆₄, and R₇₁ to R₇₄ are optionally bonded together to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₅-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O) (Q₁₁), —S(═O)₂ (Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, or a C₁-C₆₀ heteroarylthio group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₂₁)(Q₂₂)(Q₂₃), —N (Q₂₁)(Q₂₂), —B(Q₂₁)(Q₂₂), —C(═O) (Q₂₁), —S(═O)₂ (Q₂₁), —P(═O)(Q₂₁)(Q₂₂), or any combination thereof; or
—O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O) (Q₃₁), —S(═O)₂ (Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each being unsubstituted or substituted with deuterium, —F, a cyano group, a C₁-C₆₀ alkyl group, a C₁-C₆₀ alkoxy group, a phenyl group, a biphenyl group, or any combination thereof.

2. The light-emitting device as claimed in claim 1, wherein
the interlayer further comprises a hole transport region between the first electrode and the emission layer and an electron transport region between the emission layer and the second electrode,
the hole transport region comprises a hole injection layer, a hole transport layer, an emission auxiliary layer, an electron-blocking layer, or any combination thereof, and
the electron transport region comprises a buffer layer, a hole-blocking layer, an electron control layer, an electron transport layer, an electron injection layer, or any combination thereof.

3. The light-emitting device as claimed in claim 1, wherein
the emission layer comprises the heterocyclic compound represented by Formula 1.

4. The light-emitting device as claimed in claim 1, wherein
the emission layer comprises a host and a dopant, and
the host comprises the heterocyclic compound represented by Formula 1.

5. The light-emitting device as claimed in claim 1, further comprising:
a first capping layer outside the first electrode and a second capping layer outside the second electrode,
wherein the first capping layer or the second capping layer comprises the heterocyclic compound represented by Formula 1.

6. The light-emitting device as claimed in claim 1, wherein
the emission layer emits blue light.

7. An electronic apparatus comprising a light-emitting device, wherein:
the light-emitting device comprises:
a first electrode;
a second electrode opposite to the first electrode;
an interlayer between the first electrode and the second electrode and comprising an emission layer; and
a heterocyclic compound represented by Formula 1: 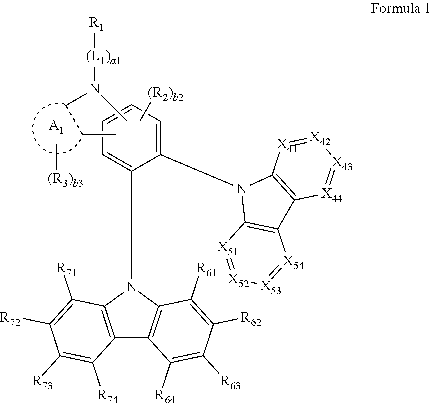
and
wherein, in Formula 1,
X₄₁ is N or C(R₄₁), X₄₂ is N or C(R₄₂), X₄₃ is N or C(R₄₃), X₄₄ is N or C(R₄₄), X₅₁ is N or C(R₅₁), X₅₂ is N or C(R₅₂), X₅₃ is N or C(R₅₃), and X₅₄ is N or C(R₅₄),
at least one selected from among X₄₁ to X₄₄ and X₅₁ to X₅₄ is N,
ring A₁ is a C₅-C₆₀ carbocyclic group or a C₁-C₆₀ heterocyclic group,
a1 is an integer of 0 to 5,
L₁ is a C₃-C₆₀ carbocyclic group that is unsubstituted or substituted with at least one R₁₀ₐ or a C₁-C₆₀ heterocyclic group that is unsubstituted or substituted with at least one R₁₀ₐ,
*-(L₁)ₐ₁-*′ is a single bond when a1 is 0,
b2 is an integer of 0 to 2,
b3 is an integer of 0 to 10,
R₁ to R₃, R₄₁ to R₄₄, R₅₁ to R₅₄, R₆₁ to R₆₄, and R₇₁ to R₇₄ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, a C₇-C₆₀ arylalkyl group unsubstituted or substituted with at least one R₁₀ₐ, —C(Q₁)(Q₂)(Q₃), —Si(Q₁)(Q₂)(Q₃), —B(Q₁)(Q₂), —N(Q₁)(Q₂), —P(Q₁)(Q₂), —C(═O) (Q₁), —S(═O) (Q₁), —S(═O)₂ (Q₁), —P(═O)(Q₁)(Q₂), or —P(═S)(Q₁)(Q₂),
neighboring two or more selected from among R₁ to R₃, R₄₁ to R₄₄, R₅₁ to R₅₄, R₆₁ to R₆₄, and R₇₁ to R₇₄ are optionally bonded together to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O) (Q₁₁), —S(═O)₂ (Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, or a C₁-C₆₀ heteroarylthio group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O) (Q₂₁), —S(═O)₂ (Q₂₁), —P(═O)(Q₂₁)(Q₂₂), or any combination thereof; or
—O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O) (Q₃₁), —S(═O)₂ (Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each being unsubstituted or substituted with deuterium, —F, a cyano group, a C₁-C₆₀ alkyl group, a C₁-C₆₀ alkoxy group, a phenyl group, a biphenyl group, or any combination thereof.

8. The electronic apparatus as claimed in claim 7, further comprising:
a thin-film transistor electrically connected to the light-emitting device; and
a color filter, a color conversion layer, a touch screen layer, a polarizing layer, or any combination thereof.

9. An electronic device comprising a light-emitting device, wherein:
the light-emitting device comprises:
a first electrode;
a second electrode opposite to the first electrode;
an interlayer between the first electrode and the second electrode and comprising an emission layer; and
a heterocyclic compound represented by Formula 1: 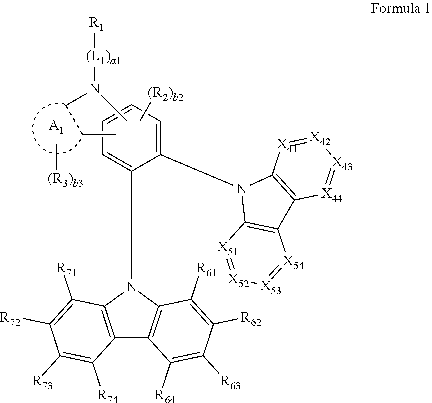
and
wherein, in Formula 1,
X₄₁ is N or C(R₄₁), X₄₂ is N or C(R₄₂), X₄₃ is N or C(R₄₃), X₄₄ is N or C(R₄₄), X₅₁ is N or C(R₅₁), X₅₂ is N or C(R₅₂), X₅₃ is N or C(R₅₃), and X₅₄ is N or C(R₅₄),
at least one selected from among X₄₁ to X₄₄ and X₅₁ to X₅₄ is N,
ring A₁ is a C₅-C₆₀ carbocyclic group or a C₁-C₆₀ heterocyclic group,
a1 is an integer of 0 to 5,
L₁ is a C₃-C₆₀ carbocyclic group that is unsubstituted or substituted with at least one R₁₀ₐ or a C₁-C₆₀ heterocyclic group that is unsubstituted or substituted with at least one R₁₀ₐ,
*-(L₁)ₐ₁-*′ is a single bond when a1 is 0,
b2 is an integer of 0 to 2,
b3 is an integer of 0 to 10,
R₁ to R₃, R₄₁ to R₄₄, R₅₁ to R₅₄, R₆₁ to R₆₄, and R₇₁ to R₇₄ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, a C₇-C₆₀ arylalkyl group unsubstituted or substituted with at least one R₁₀ₐ, —C(Q₁)(Q₂)(Q₃), —Si(Q₁)(Q₂)(Q₃), —B(Q₁)(Q₂), —N(Q₁)(Q₂), —P(Q₁)(Q₂), —C(═O) (Q₁), —S(═O) (Q₁), —S(═O)₂ (Q₁), —P(═O)(Q₁)(Q₂), or —P(═S)(Q₁)(Q₂),
neighboring two or more selected from among R₁ to R₃, R₄₁ to R₄₄, R₅₁ to R₅₄, R₆₁ to R₆₄, and R₇₁ to R₇₄ are optionally bonded together to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O) (Q₁₁), —S(═O)₂ (Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, or a C₁-C₆₀ heteroarylthio group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O) (Q₂₁), —S(═O)₂ (Q₂₁), —P(═O)(Q₂₁)(Q₂₂), or any combination thereof; or
—O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O) (Q₃₁), —S(═O)₂ (Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each being unsubstituted or substituted with deuterium, —F, a cyano group, a C₁-C₆₀ alkyl group, a C₁-C₆₀ alkoxy group, a phenyl group, a biphenyl group, or any combination thereof.

10. The electronic device as claimed in claim 9, wherein
the electronic device is at least one selected from among a flat panel display, a curved display, a computer monitor, a medical monitor, a television, a billboard, an indoor light, an outdoor light, a light for signal, a head-up display, a fully transparent display, a partially transparent display, a flexible display, a rollable display, a foldable display, a stretchable display, a laser printer, a telephone, a portable phone, a tablet personal computer, a phablet, a personal digital assistant, a wearable device, a laptop computer, a digital camera, a camcorder, a viewfinder, a micro display, a three-dimensional display, a virtual reality display, an augmented reality display, a vehicle, a video wall with multiple displays tiled together, a theater screen, a stadium screen, a phototherapy device, and a signboard.

11. A heterocyclic compound represented by Formula 1: 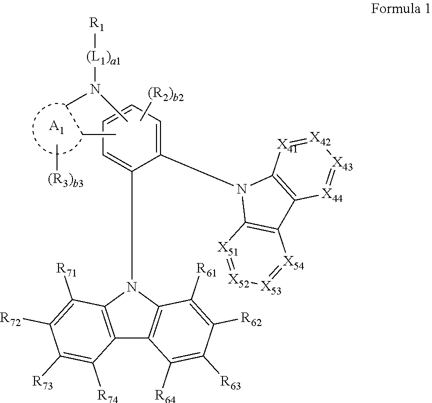
wherein, in Formula 1,
X₄₁ is N or C(R₄₁), X₄₂ is N or C(R₄₂), X₄₃ is N or C(R₄₃), X₄₄ is N or C(R₄₄), X₅₁ is N or C(R₅₁), X₅₂ is N or C(R₅₂), X₅₃ is N or C(R₅₃), and X₅₄ is N or C(R₅₄),
at least one selected from among X₄₁ to X₄₄ and X₅₁ to X₅₄ is N,
ring A₁ is a C₅-C₆₀ carbocyclic group or a C₁-C₆₀ heterocyclic group,
a1 is an integer of 0 to 5,
L₁ is a C₃-C₆₀ carbocyclic group that is unsubstituted or substituted with at least one R₁₀ₐ or a C₁-C₆₀ heterocyclic group that is unsubstituted or substituted with at least one R₁₀ₐ,
*-(L₁)ₐ₁-*′ is a single bond when a1 is 0,
b2 is an integer of 0 to 2,
b3 is an integer of 0 to 10,
R₁ to R₃, R₄₁ to R₄₄, R₅₁ to R₅₄, R₆₁ to R₆₄, and R₇₁ to R₇₄ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, a C₇-C₆₀ arylalkyl group unsubstituted or substituted with at least one R₁₀ₐ, —C(Q₁)(Q₂)(Q₃), —Si(Q₁)(Q₂)(Q₃), —B(Q₁)(Q₂), —N(Q₁)(Q₂), —P(Q₁)(Q₂), —C(═O) (Q₁), —S(═O) (Q₁), —S(═O)₂ (Q₁), —P(═O)(Q₁)(Q₂), or —P(═S)(Q₁)(Q₂),
neighboring two or more selected from among R₁ to R₃, R₄₁ to R₄₄, R₅₁ to R₅₄, R₆₁ to R₆₄, and R₇₁ to R₇₄ are optionally bonded together to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₁₁)(Q₁₂)(Q₁₃), —N(Q₁₁)(Q₁₂), —B(Q₁₁)(Q₁₂), —C(═O) (Q₁₁), —S(═O)₂ (Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, or a C₁-C₆₀ heteroarylthio group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, a C₁-C₆₀ heteroaryloxy group, a C₁-C₆₀ heteroarylthio group, —Si(Q₂₁)(Q₂₂)(Q₂₃), —N(Q₂₁)(Q₂₂), —B(Q₂₁)(Q₂₂), —C(═O) (Q₂₁), —S(═O)₂ (Q₂₁), —P(═O)(Q₂₁)(Q₂₂), or any combination thereof; or
—O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O) (Q₃₁), —S(═O)₂ (Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ group, or a C₂-C₆₀ heteroarylalkyl group, each being unsubstituted or substituted with
a C₃-C₆₀ carbocyclic group, a C₁-C₆₀ heterocyclic group, a C₇-C₆₀ arylalkyl group, or a C₂-C₆₀ heteroarylalkyl group, each being unsubstituted or substituted with deuterium, —F, a cyano group, a C₁-C₆₀ alkyl group, a C₁-C₆₀ alkoxy group, a phenyl group, a biphenyl group, or any combination thereof.

12. The heterocyclic compound as claimed in claim 11, wherein
ring A₁ is a benzene group, a naphthalene group, an anthracene group, a phenanthrene group, a triphenylene group, a pyrene group, a chrysene group, an acenaphthylene group, a perylene group, a benzopyrene group, a benzochrysene group, a benzotriphenylene group, a fluoranthene group, a coronene group, a cyclopentadiene group, a 1,2,3,4-tetrahydronaphthalene group, an acridine group, a thiophene group, a furan group, an indole group, a benzoborole group, a benzophosphole group, an indene group, a benzosilole group, a benzogermole group, a benzothiophene group, a benzoselenophene group, a benzofuran group, a benzotellurophene group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzotellurophene group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

13. The heterocyclic compound as claimed in claim 11, wherein
ring A₁ is a benzene group or a naphthalene group.

14. The heterocyclic compound as claimed in claim 11, wherein
L₁ is a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluoren-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each being unsubstituted or substituted with at least one R₁₀ₐ, and
R₁₀ₐ is the same as defined in Formula 1.

15. The heterocyclic compound as claimed in claim 11, wherein
L₁ is any one selected from among the groups represented by Formulae 3-1 to 3-28: 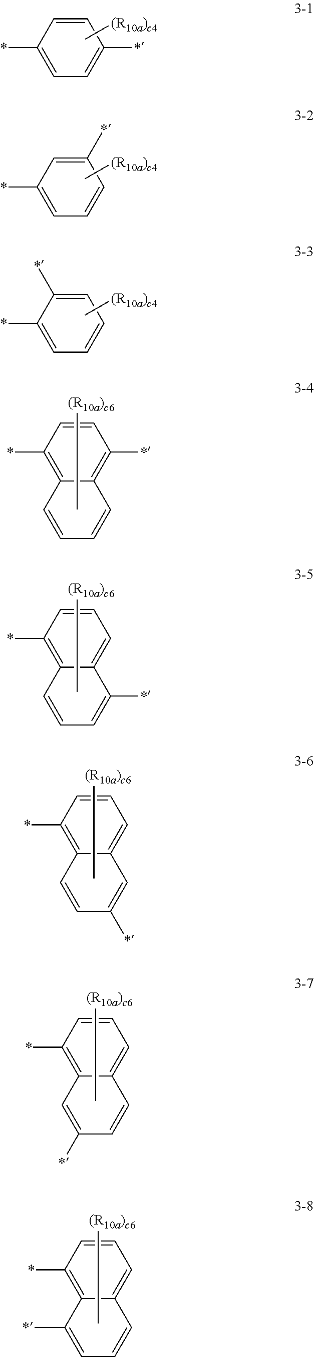 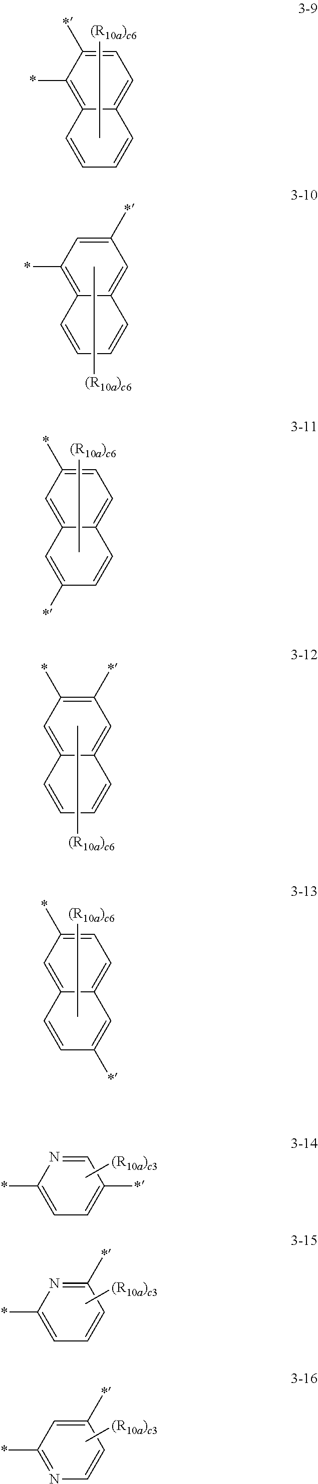 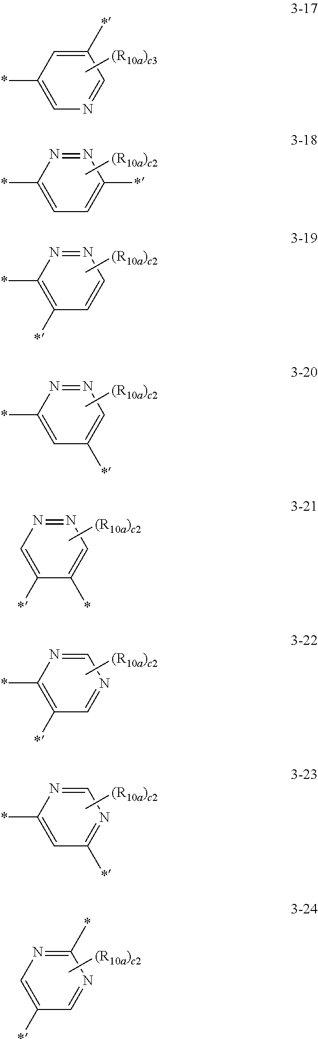 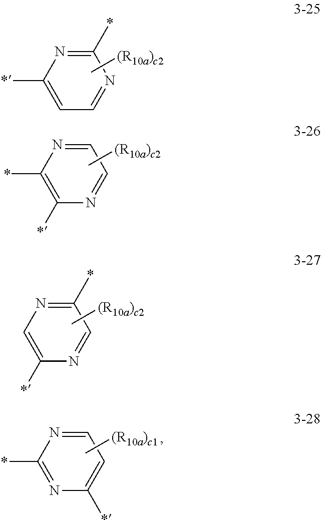
and
wherein, in Formulae 3-1 to 3-28,
R₁₀ₐ is the same as defined in Formula 1,
c1 is 0 or 1,
c2 is an integer of 0 to 2,
c3 is an integer of 0 to 3,
c4 is an integer of 0 to 4,
c6 is an integer of 0 to 6, and
* and * each indicate a binding site to a neighboring atom.

16. The heterocyclic compound as claimed in claim 15, wherein
L₁ is a group represented by any one selected from among Formulae 3-1 to 3-3 and 3-14 to 3-17.

17. The heterocyclic compound as claimed in claim 11, wherein
R₁ to R₃, R₄₁ to R₄₄, R₅₁ to R₅₄, R₆₁ to R₆₄, and R₇₁ to R₇₄ are each independently hydrogen, deuterium, —F, —Cl, —Br, —I, a hydroxyl group, a cyano group, a nitro group, a C₁-C₂₀ alkyl group, or a C₁-C₂₀ alkoxy group;
the C₁-C₂₀ alkyl group or the C₁-C₂₀ alkoxy group, each being unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a tetraphenylenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each being unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a achrysenyl group, a tetraphenylenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzosilolyl group, a dibenzocarbazolyl group, an imidazopyridinyl group, an imidazopyrimidinyl group, —O(Q₃₁), —S(Q₃₁), —Si(Q₃₁)(Q₃₂)(Q₃₃), —N(Q₃₁)(Q₃₂), —B(Q₃₁)(Q₃₂), —P(Q₃₁)(Q₃₂), —C(═O) (Q₃₁), —S(═O)₂ (Q₃₁), —P(═O)(Q₃₁)(Q₃₂), or any combination thereof; or
—C(Q₁)(Q₂)(Q₃), —Si(Q₁)(Q₂)(Q₃), —N(Q₁)(Q₂), —B(Q₁)(Q₂), —C(═O) (Q₁), —S(═O)₂ (Q₁), or —P(═O)(Q₁)(Q₂), and
Q₁ to Q₃ and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group; or a C₁-C₆₀ heterocyclic group, each being unsubstituted or substituted with deuterium, —F, a cyano group, a C₁-C₆₀ alkyl group, a C₁-C₆₀ alkoxy group, a phenyl group, a biphenyl group, or any combination thereof.

18. The heterocyclic compound as claimed in claim 11, wherein
the heterocyclic compound is a compound represented by Formula 1A: 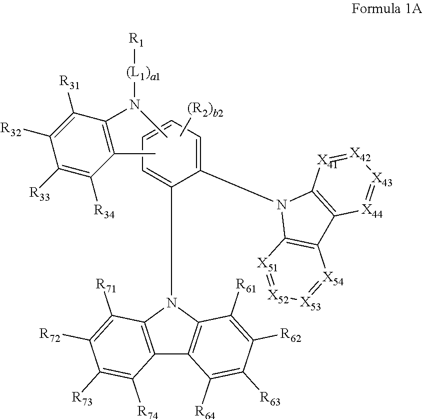
and
wherein, in Formula 1A,
X₄₁ to X₄₄, X₅₁ to X₅₄, a1, L₁, b2, R₁, R₂, R₄₁ to R₄₄, R₅₁ to R₅₄, R₆₁ to R₆₄, and R₇₁ to R₇₄ are the same as defined in Formula 1, and
R₃₁ to R₃₄ are each the same as defined with respect to R₃ in Formula 1.

19. The heterocyclic compound as claimed in claim 11, wherein
the heterocyclic compound is a compound represented by any one selected from among Formulae 1-1 to 1-6: 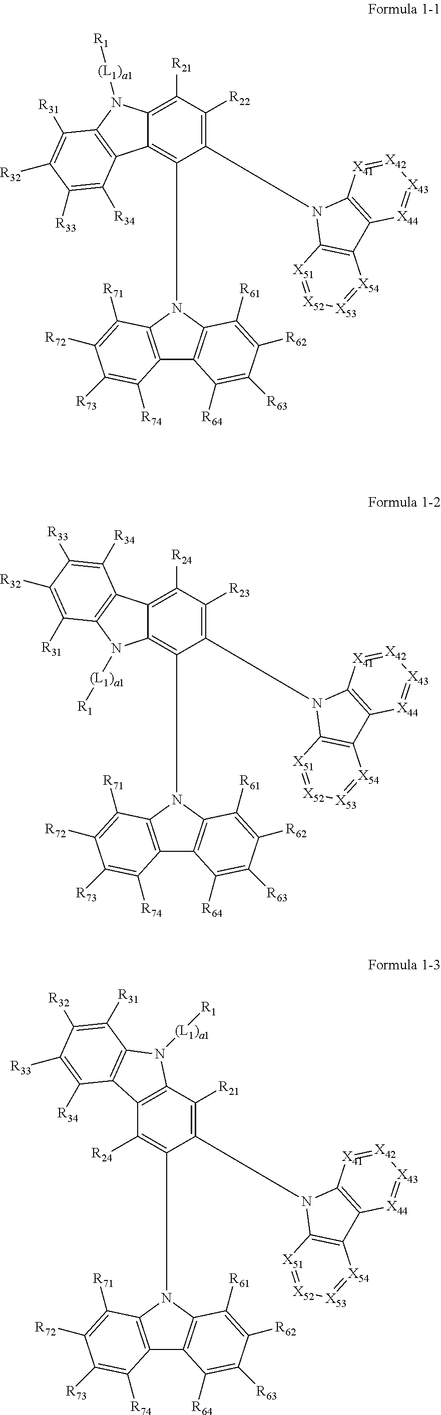 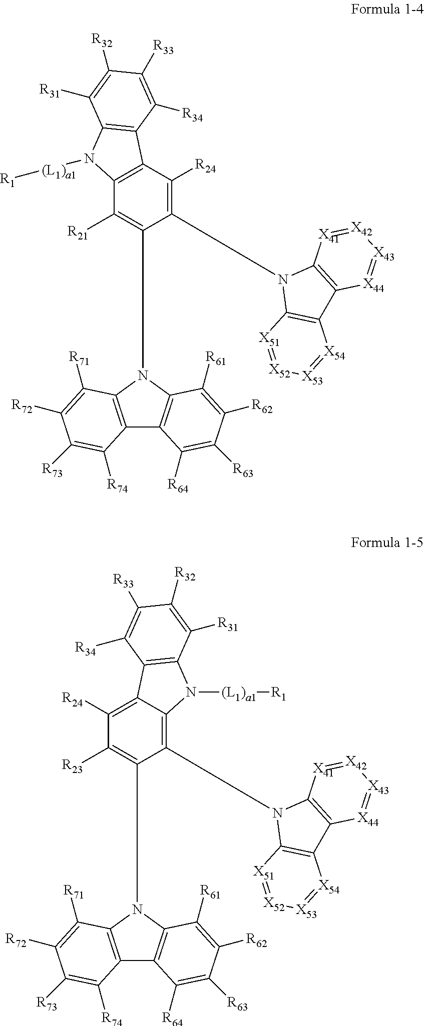 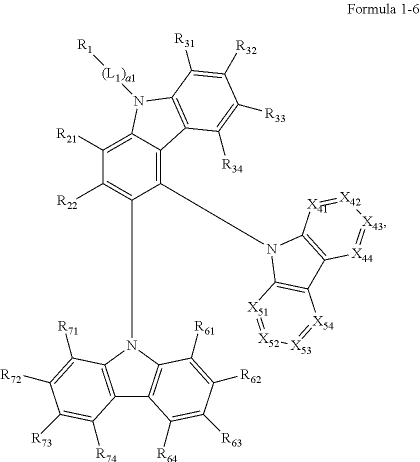
and
wherein, in Formulae 1-1 to 1-6,
X₄₁ to X₄₄, X₅₁ to X₅₄, a1, L₁, R₁, R₄₁ to R₄₄, R₅₁ to R₅₄, R₆₁ to R₆₄, and R₇₁ to R₇₄ are the same as defined in Formula 1,
R₂₁ to R₂₄ are each the same as defined with respect to R₂ in Formula 1, and
R₃₁ to R₃₄ are each the same as defined with respect to R₃ in Formula 1.

20. The heterocyclic compound as claimed in claim 11, wherein
the heterocyclic compound is represented by any one selected from among Compounds 1 to 170: 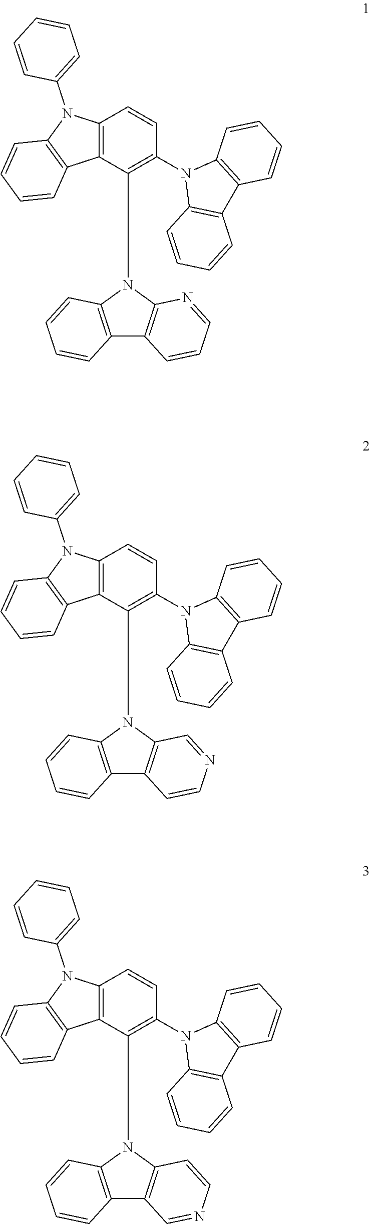 [SMILES: c1ccc(-n2c3ccccc3c3c(-n4c5ccccc5c5cccnc54)c(-n4c5ccccc5c5ccccc54)ccc32)cc1 ; c1ccc(-n2c3ccccc3c3c(-n4c5ccccc5c5ccncc54)c(-n4c5ccccc5c5ccccc54)ccc32)cc1 ; c1ccc(-n2c3ccccc3c3c(-n4c5ccccc5c5cnccc54)c(-n4c5ccccc5c5ccccc54)ccc32)cc1] 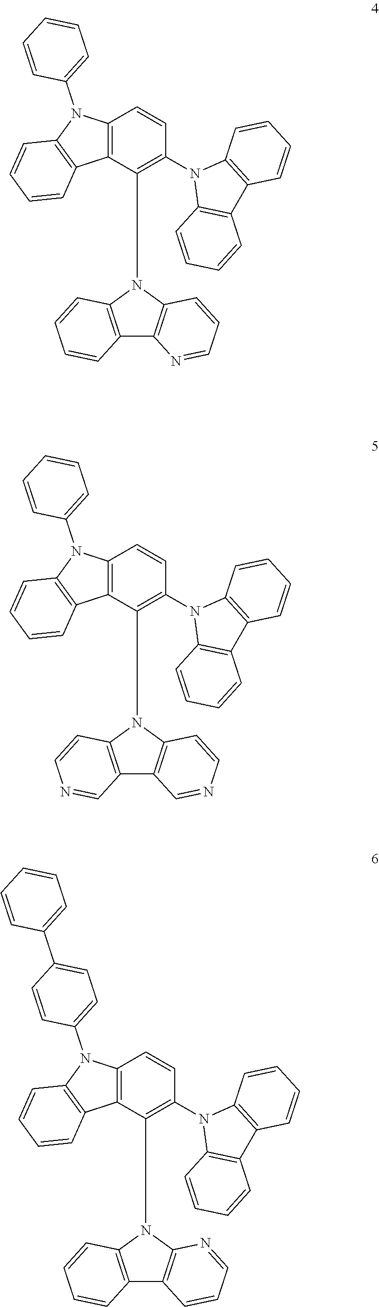 [SMILES: c1ccc(-n2c3ccccc3c3c(-n4c5ccccc5c5ncccc54)c(-n4c5ccccc5c5ccccc54)ccc32)cc1 ; c1ccc(-n2c3ccccc3c3c(-n4c5ccncc5c5cnccc54)c(-n4c5ccccc5c5ccccc54)ccc32)cc1 ; c1ccc(-c2ccc(-n3c4ccccc4c4c(-n5c6ccccc6c6cccnc65)c(-n5c6ccccc6c6ccccc65)ccc43)cc2)cc1] 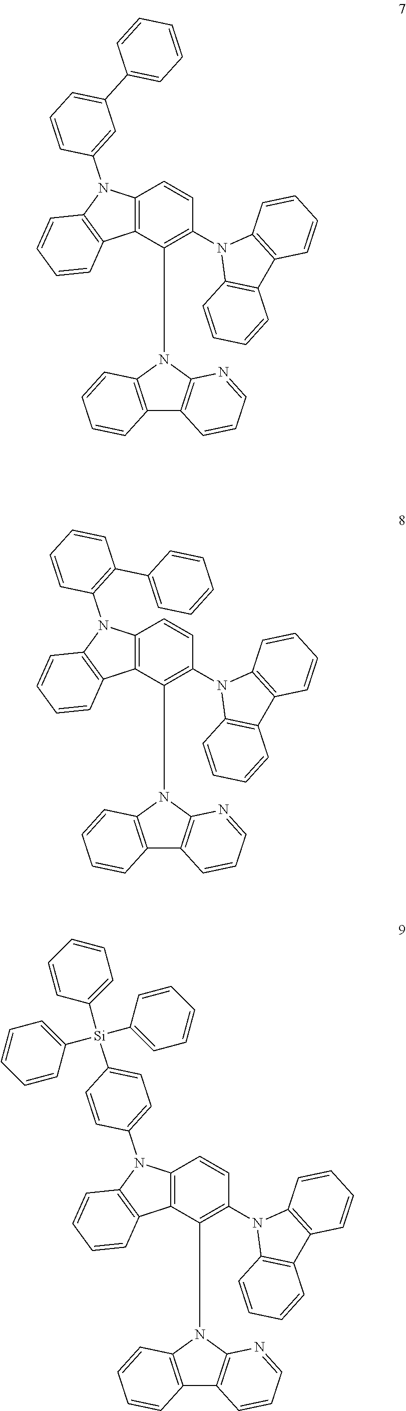 [SMILES: c1ccc(-c2cccc(-n3c4ccccc4c4c(-n5c6ccccc6c6cccnc65)c(-n5c6ccccc6c6ccccc65)ccc43)c2)cc1 ; c1ccc(-c2ccccc2-n2c3ccccc3c3c(-n4c5ccccc5c5cccnc54)c(-n4c5ccccc5c5ccccc54)ccc32)cc1 ; c1ccc([Si](c2ccccc2)(c2ccccc2)c2ccc(-n3c4ccccc4c4c(-n5c6ccccc6c6cccnc65)c(-n5c6ccccc6c6ccccc65)ccc43)cc2)cc1] 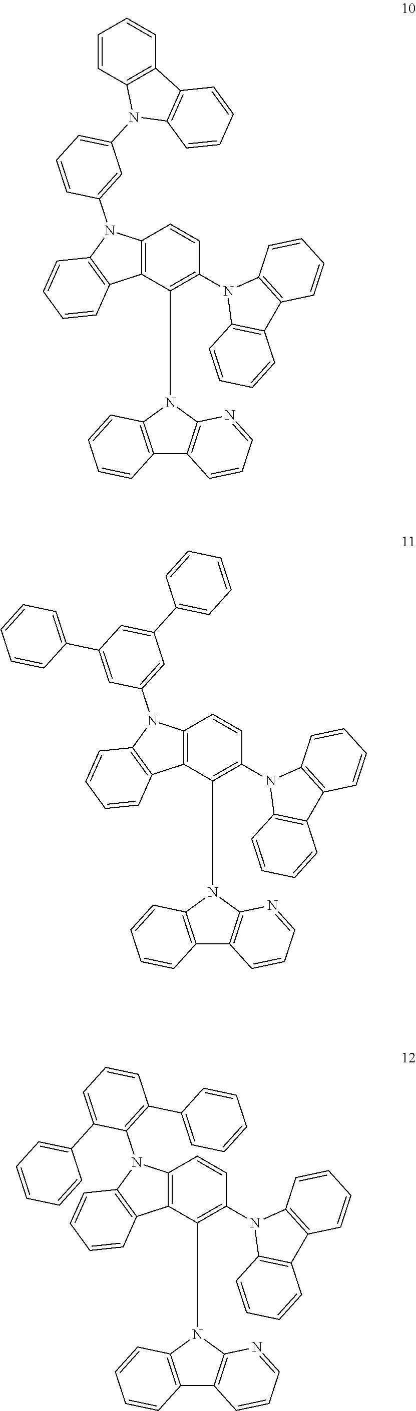 [SMILES: c1cc(-n2c3ccccc3c3ccccc32)cc(-n2c3ccccc3c3c(-n4c5ccccc5c5cccnc54)c(-n4c5ccccc5c5ccccc54)ccc32)c1 ; c1ccc(-c2cc(-c3ccccc3)cc(-n3c4ccccc4c4c(-n5c6ccccc6c6cccnc65)c(-n5c6ccccc6c6ccccc65)ccc43)c2)cc1 ; c1ccc(-c2cccc(-c3ccccc3)c2-n2c3ccccc3c3c(-n4c5ccccc5c5cccnc54)c(-n4c5ccccc5c5ccccc54)ccc32)cc1] 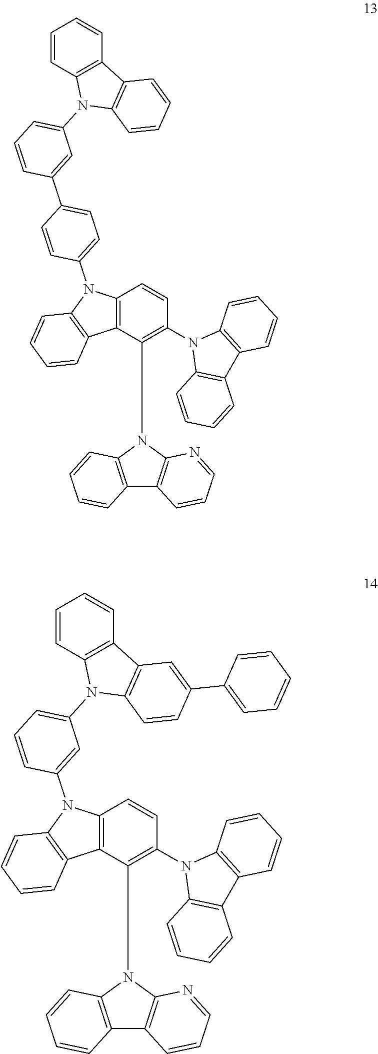 [SMILES: c1cc(-c2ccc(-n3c4ccccc4c4c(-n5c6ccccc6c6cccnc65)c(-n5c6ccccc6c6ccccc65)ccc43)cc2)cc(-n2c3ccccc3c3ccccc32)c1 ; c1ccc(-c2ccc3c(c2)c2ccccc2n3-c2cccc(-n3c4ccccc4c4c(-n5c6ccccc6c6cccnc65)c(-n5c6ccccc6c6ccccc65)ccc43)c2)cc1] 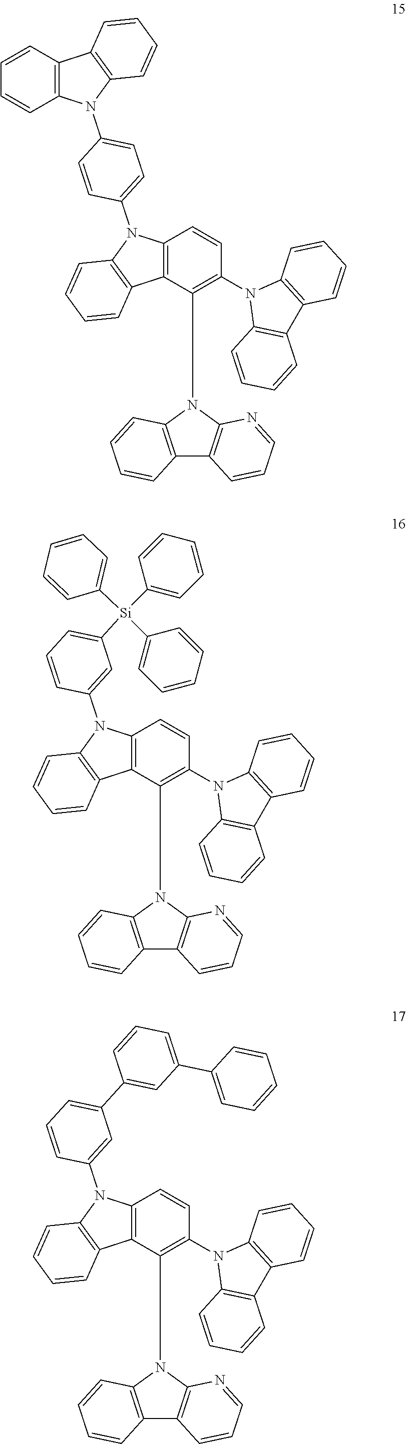 [SMILES: c1ccc2c(c1)c1ccccc1n2-c1ccc(-n2c3ccccc3c3c(-n4c5ccccc5c5cccnc54)c(-n4c5ccccc5c5ccccc54)ccc32)cc1 ; c1ccc([Si](c2ccccc2)(c2ccccc2)c2cccc(-n3c4ccccc4c4c(-n5c6ccccc6c6cccnc65)c(-n5c6ccccc6c6ccccc65)ccc43)c2)cc1 ; c1ccc(-c2cccc(-c3cccc(-n4c5ccccc5c5c(-n6c7ccccc7c7cccnc76)c(-n6c7ccccc7c7ccccc76)ccc54)c3)c2)cc1] 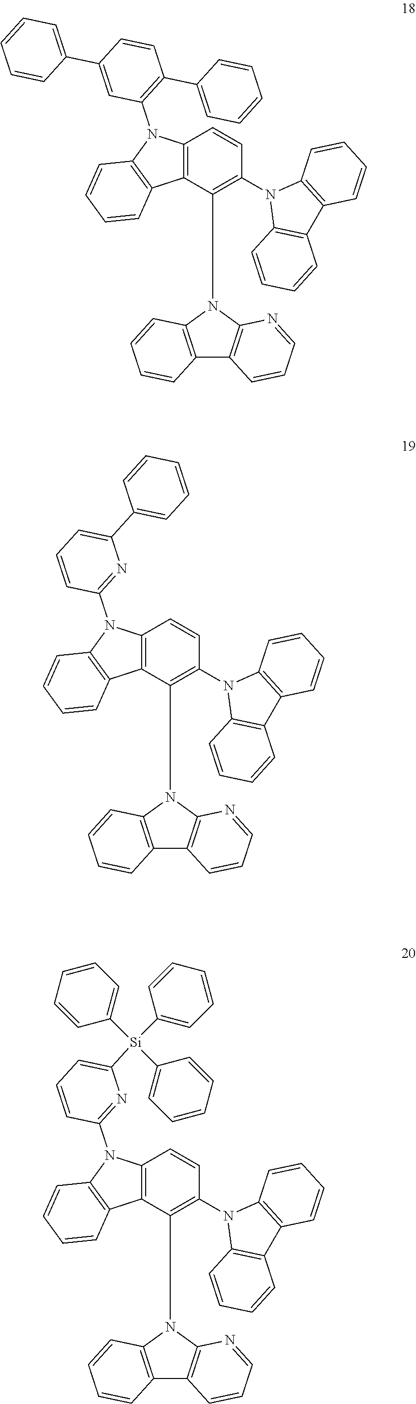 [SMILES: c1ccc(-c2ccc(-c3ccccc3)c(-n3c4ccccc4c4c(-n5c6ccccc6c6cccnc65)c(-n5c6ccccc6c6ccccc65)ccc43)c2)cc1 ; c1ccc(-c2cccc(-n3c4ccccc4c4c(-n5c6ccccc6c6cccnc65)c(-n5c6ccccc6c6ccccc65)ccc43)n2)cc1 ; c1ccc([Si](c2ccccc2)(c2ccccc2)c2cccc(-n3c4ccccc4c4c(-n5c6ccccc6c6cccnc65)c(-n5c6ccccc6c6ccccc65)ccc43)n2)cc1] 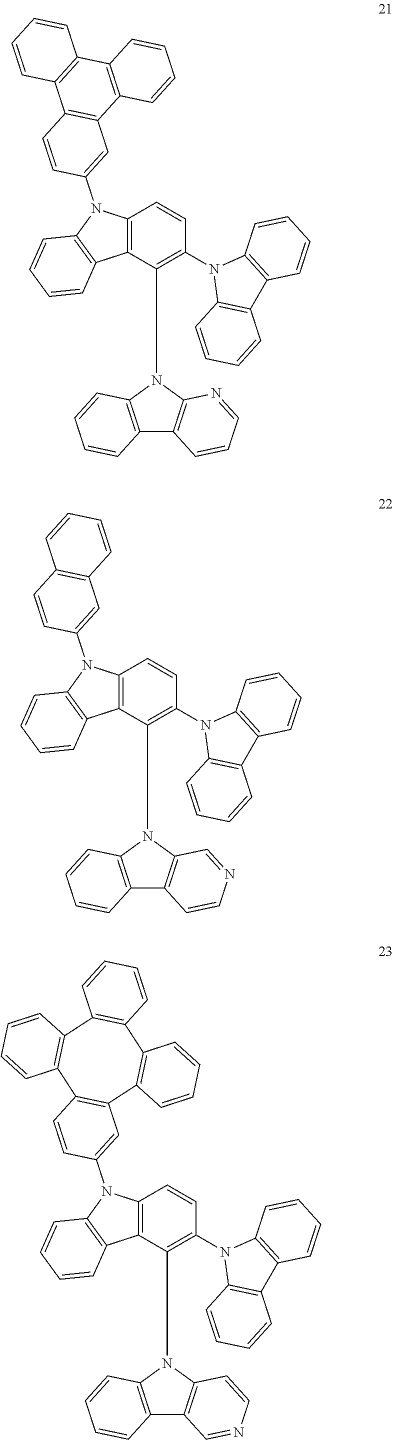 [SMILES: c1ccc2c(c1)c1ccccc1c1cc(-n3c4ccccc4c4c(-n5c6ccccc6c6cccnc65)c(-n5c6ccccc6c6ccccc65)ccc43)ccc21 ; c1ccc2cc(-n3c4ccccc4c4c(-n5c6ccccc6c6ccncc65)c(-n5c6ccccc6c6ccccc65)ccc43)ccc2c1 ; c1ccc2c(c1)-c1ccccc1-c1ccc(-n3c4ccccc4c4c(-n5c6ccccc6c6cnccc65)c(-n5c6ccccc6c6ccccc65)ccc43)cc1-c1ccccc1-2] 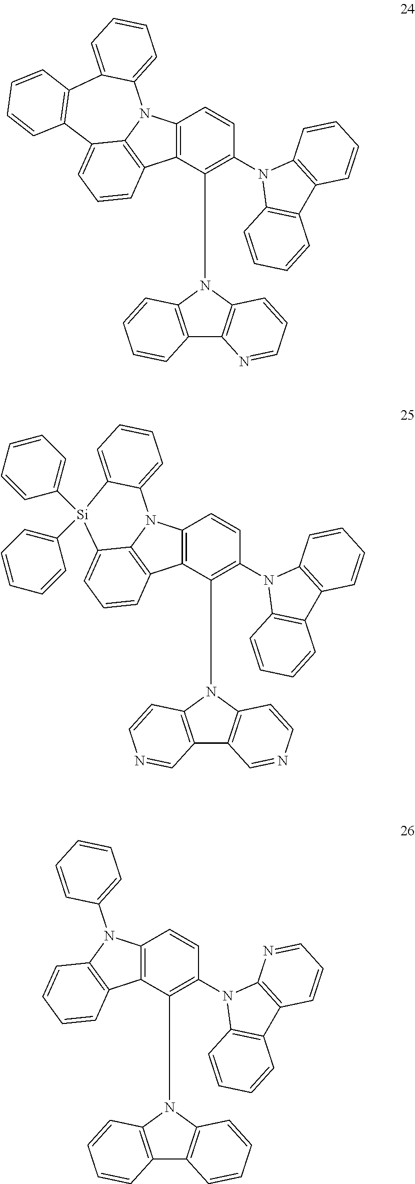 [SMILES: c1ccc2c(c1)-c1ccccc1-n1c3ccc(-n4c5ccccc5c5ccccc54)c(-n4c5ccccc5c5ncccc54)c3c3cccc-2c31 ; c1ccc([Si]2(c3ccccc3)c3ccccc3-n3c4ccc(-n5c6ccccc6c6ccccc65)c(-n5c6ccncc6c6cnccc65)c4c4cccc2c43)cc1 ; c1ccc(-n2c3ccccc3c3c(-n4c5ccccc5c5ccccc54)c(-n4c5ccccc5c5cccnc54)ccc32)cc1] 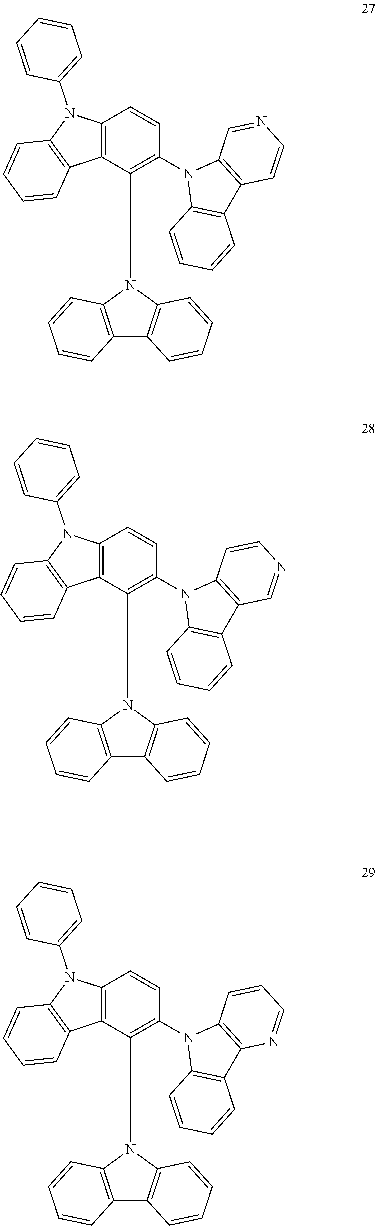 [SMILES: c1ccc(-n2c3ccccc3c3c(-n4c5ccccc5c5ccccc54)c(-n4c5ccccc5c5ccncc54)ccc32)cc1 ; c1ccc(-n2c3ccccc3c3c(-n4c5ccccc5c5ccccc54)c(-n4c5ccccc5c5cnccc54)ccc32)cc1 ; c1ccc(-n2c3ccccc3c3c(-n4c5ccccc5c5ccccc54)c(-n4c5ccccc5c5ncccc54)ccc32)cc1] 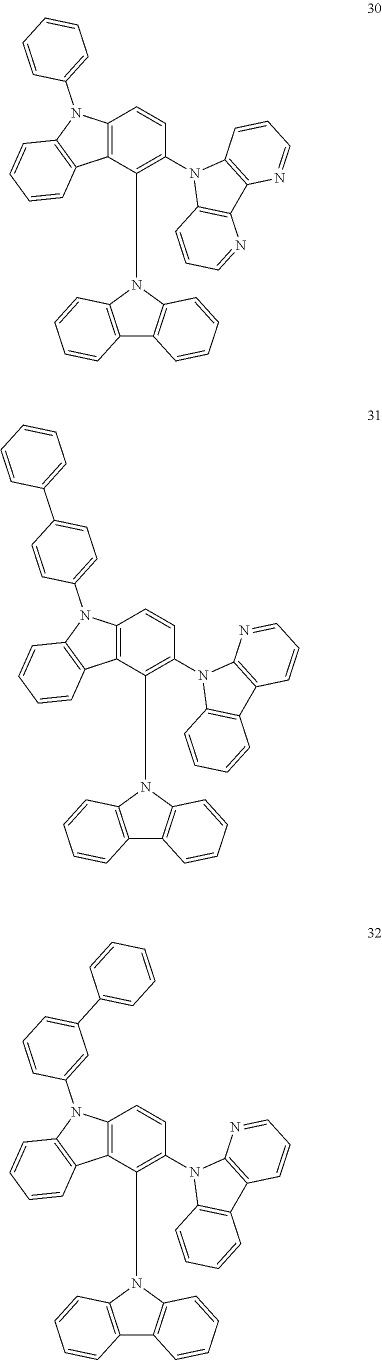 [SMILES: c1ccc(-n2c3ccccc3c3c(-n4c5ccccc5c5ccccc54)c(-n4c5cccnc5c5ncccc54)ccc32)cc1 ; c1ccc(-c2ccc(-n3c4ccccc4c4c(-n5c6ccccc6c6ccccc65)c(-n5c6ccccc6c6cccnc65)ccc43)cc2)cc1 ; c1ccc(-c2cccc(-n3c4ccccc4c4c(-n5c6ccccc6c6ccccc65)c(-n5c6ccccc6c6cccnc65)ccc43)c2)cc1] 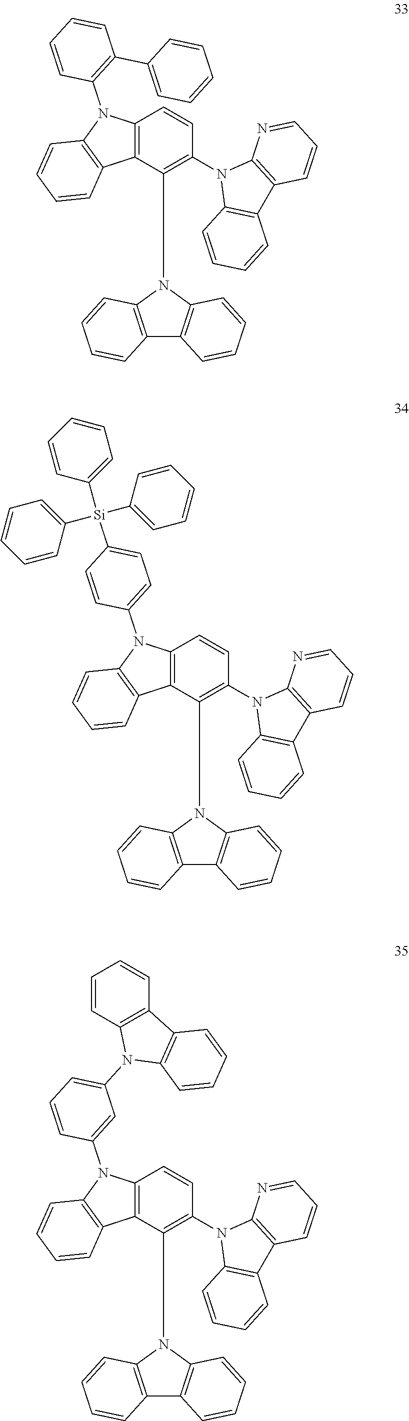 [SMILES: c1ccc(-c2ccccc2-n2c3ccccc3c3c(-n4c5ccccc5c5ccccc54)c(-n4c5ccccc5c5cccnc54)ccc32)cc1 ; c1ccc([Si](c2ccccc2)(c2ccccc2)c2ccc(-n3c4ccccc4c4c(-n5c6ccccc6c6ccccc65)c(-n5c6ccccc6c6cccnc65)ccc43)cc2)cc1 ; c1cc(-n2c3ccccc3c3ccccc32)cc(-n2c3ccccc3c3c(-n4c5ccccc5c5ccccc54)c(-n4c5ccccc5c5cccnc54)ccc32)c1] 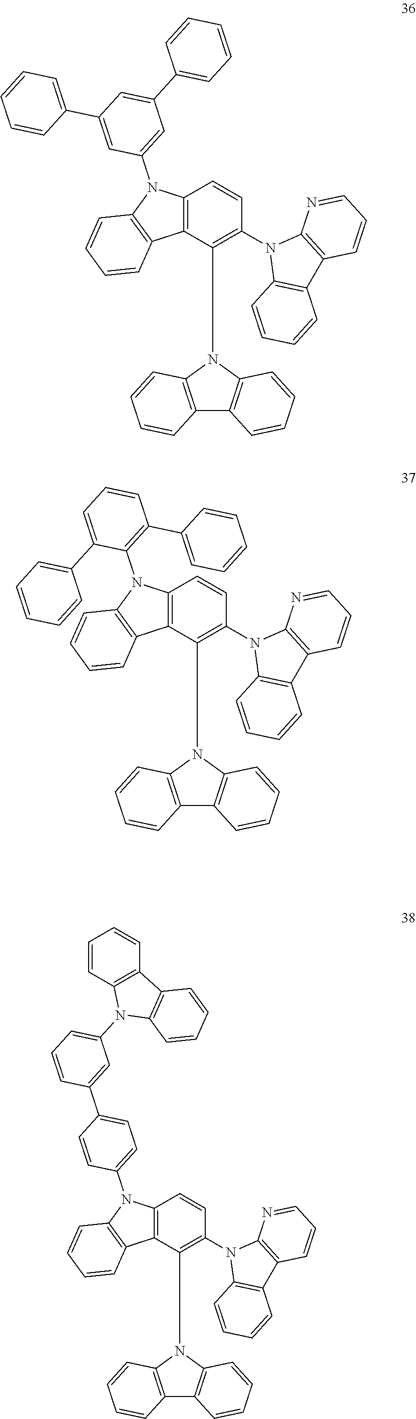 [SMILES: c1ccc(-c2cc(-c3ccccc3)cc(-n3c4ccccc4c4c(-n5c6ccccc6c6ccccc65)c(-n5c6ccccc6c6cccnc65)ccc43)c2)cc1 ; c1ccc(-c2cccc(-c3ccccc3)c2-n2c3ccccc3c3c(-n4c5ccccc5c5ccccc54)c(-n4c5ccccc5c5cccnc54)ccc32)cc1 ; c1cc(-c2ccc(-n3c4ccccc4c4c(-n5c6ccccc6c6ccccc65)c(-n5c6ccccc6c6cccnc65)ccc43)cc2)cc(-n2c3ccccc3c3ccccc32)c1] 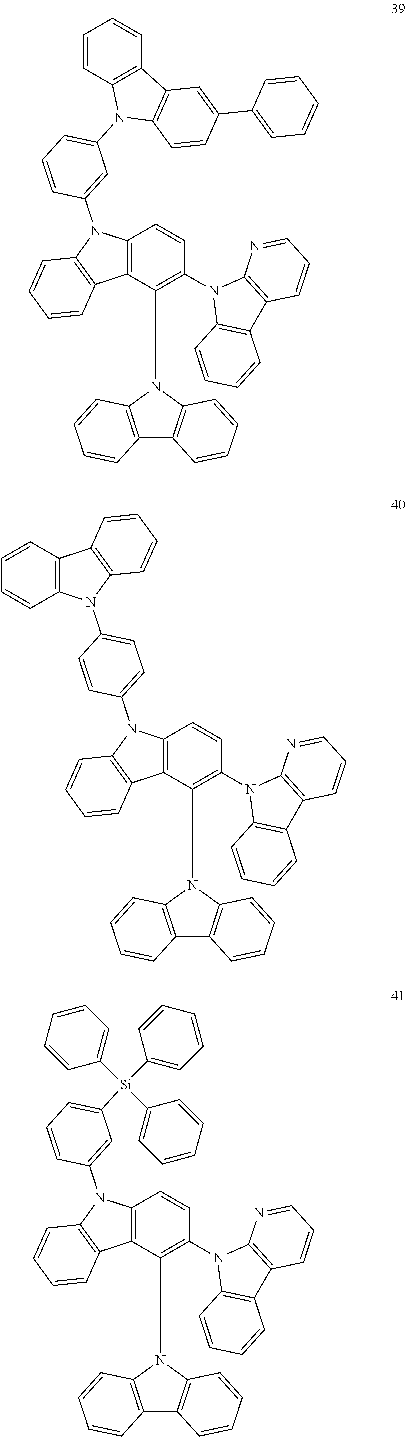 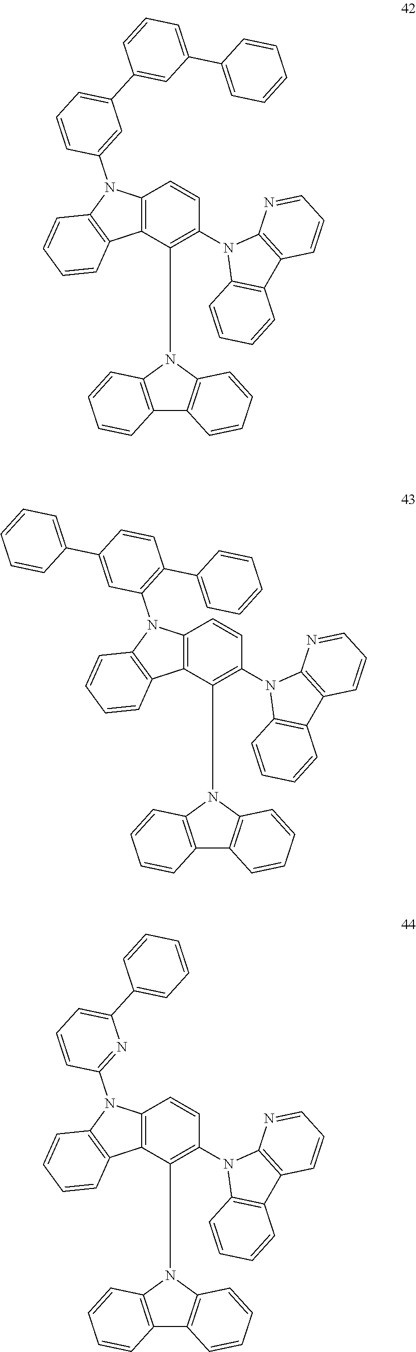 [SMILES: c1ccc(-c2cccc(-c3cccc(-n4c5ccccc5c5c(-n6c7ccccc7c7ccccc76)c(-n6c7ccccc7c7cccnc76)ccc54)c3)c2)cc1 ; c1ccc(-c2ccc(-c3ccccc3)c(-n3c4ccccc4c4c(-n5c6ccccc6c6ccccc65)c(-n5c6ccccc6c6cccnc65)ccc43)c2)cc1 ; c1ccc(-c2cccc(-n3c4ccccc4c4c(-n5c6ccccc6c6ccccc65)c(-n5c6ccccc6c6cccnc65)ccc43)n2)cc1] 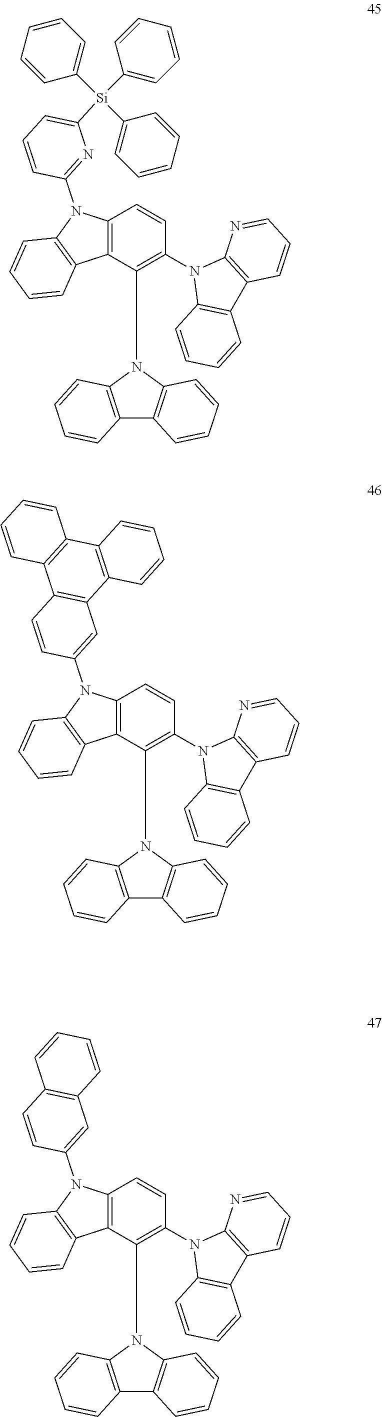 [SMILES: c1ccc([Si](c2ccccc2)(c2ccccc2)c2cccc(-n3c4ccccc4c4c(-n5c6ccccc6c6ccccc65)c(-n5c6ccccc6c6cccnc65)ccc43)n2)cc1 ; c1ccc2c(c1)c1ccccc1c1cc(-n3c4ccccc4c4c(-n5c6ccccc6c6ccccc65)c(-n5c6ccccc6c6cccnc65)ccc43)ccc21 ; c1ccc2cc(-n3c4ccccc4c4c(-n5c6ccccc6c6ccccc65)c(-n5c6ccccc6c6cccnc65)ccc43)ccc2c1] 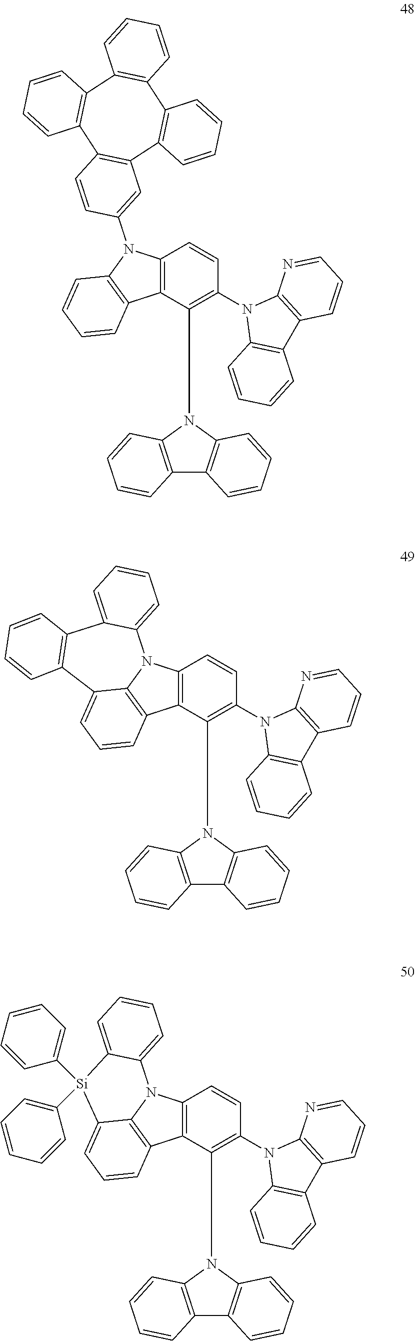 [SMILES: c1ccc2c(c1)-c1ccccc1-c1ccc(-n3c4ccccc4c4c(-n5c6ccccc6c6ccccc65)c(-n5c6ccccc6c6cccnc65)ccc43)cc1-c1ccccc1-2 ; c1ccc2c(c1)-c1ccccc1-n1c3ccc(-n4c5ccccc5c5cccnc54)c(-n4c5ccccc5c5ccccc54)c3c3cccc-2c31 ; c1ccc([Si]2(c3ccccc3)c3ccccc3-n3c4ccc(-n5c6ccccc6c6cccnc65)c(-n5c6ccccc6c6ccccc65)c4c4cccc2c43)cc1] 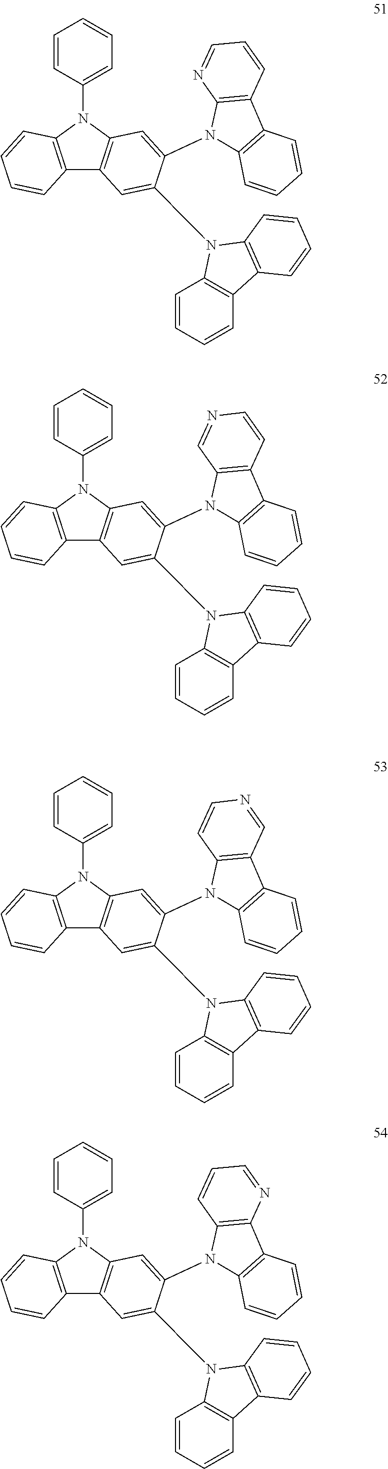 [SMILES: c1ccc(-n2c3ccccc3c3cc(-n4c5ccccc5c5ccccc54)c(-n4c5ccccc5c5cccnc54)cc32)cc1 ; c1ccc(-n2c3ccccc3c3cc(-n4c5ccccc5c5ccccc54)c(-n4c5ccccc5c5ccncc54)cc32)cc1 ; c1ccc(-n2c3ccccc3c3cc(-n4c5ccccc5c5ccccc54)c(-n4c5ccccc5c5cnccc54)cc32)cc1 ; c1ccc(-n2c3ccccc3c3cc(-n4c5ccccc5c5ccccc54)c(-n4c5ccccc5c5ncccc54)cc32)cc1] 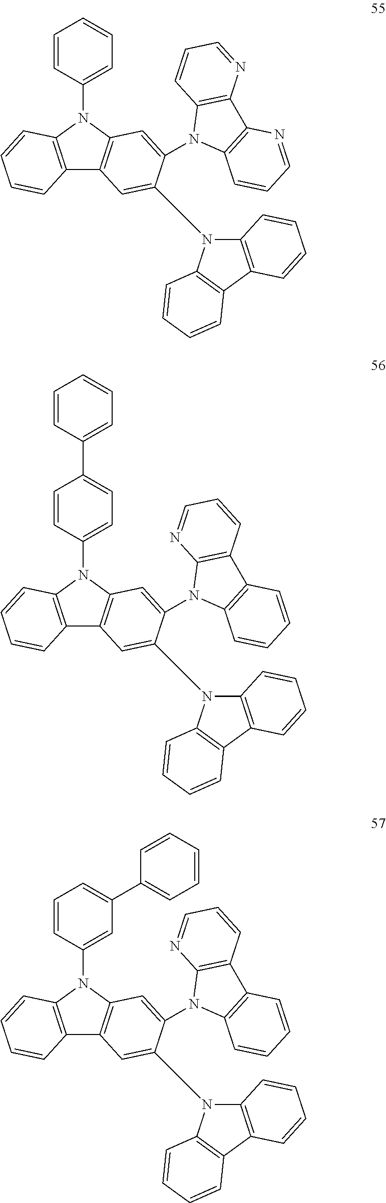 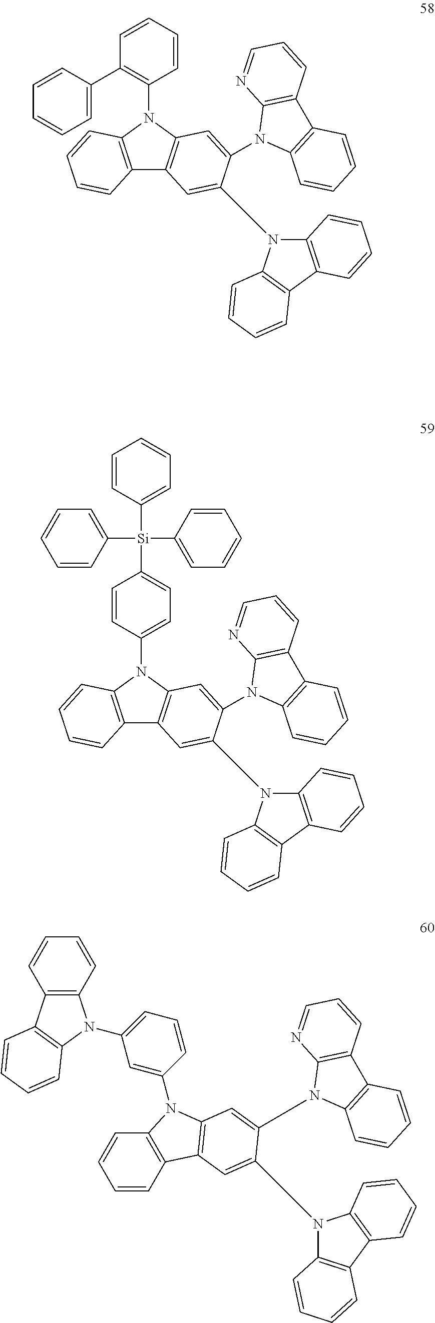 [SMILES: c1ccc(-c2ccccc2-n2c3ccccc3c3cc(-n4c5ccccc5c5ccccc54)c(-n4c5ccccc5c5cccnc54)cc32)cc1 ; c1ccc([Si](c2ccccc2)(c2ccccc2)c2ccc(-n3c4ccccc4c4cc(-n5c6ccccc6c6ccccc65)c(-n5c6ccccc6c6cccnc65)cc43)cc2)cc1 ; c1cc(-n2c3ccccc3c3ccccc32)cc(-n2c3ccccc3c3cc(-n4c5ccccc5c5ccccc54)c(-n4c5ccccc5c5cccnc54)cc32)c1] 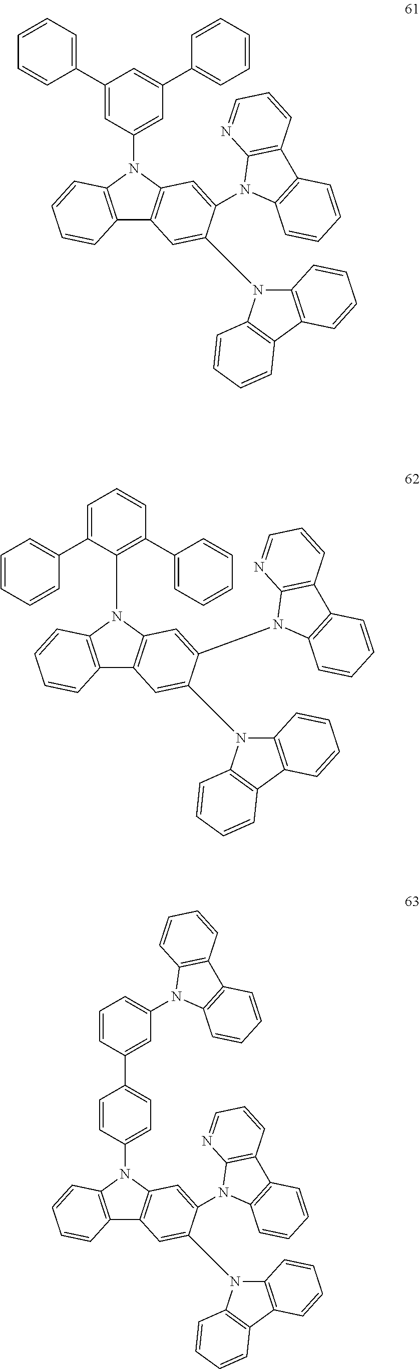 [SMILES: c1ccc(-c2cc(-c3ccccc3)cc(-n3c4ccccc4c4cc(-n5c6ccccc6c6ccccc65)c(-n5c6ccccc6c6cccnc65)cc43)c2)cc1 ; c1ccc(-c2cccc(-c3ccccc3)c2-n2c3ccccc3c3cc(-n4c5ccccc5c5ccccc54)c(-n4c5ccccc5c5cccnc54)cc32)cc1 ; c1cc(-c2ccc(-n3c4ccccc4c4cc(-n5c6ccccc6c6ccccc65)c(-n5c6ccccc6c6cccnc65)cc43)cc2)cc(-n2c3ccccc3c3ccccc32)c1] 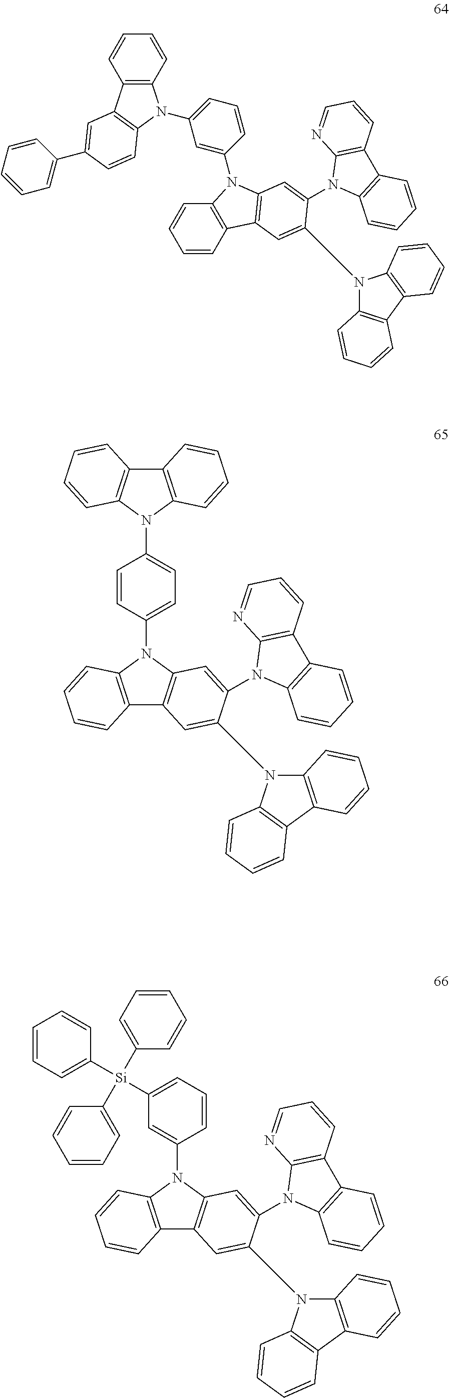 [SMILES: c1ccc(-c2ccc3c(c2)c2ccccc2n3-c2cccc(-n3c4ccccc4c4cc(-n5c6ccccc6c6ccccc65)c(-n5c6ccccc6c6cccnc65)cc43)c2)cc1 ; c1ccc2c(c1)c1ccccc1n2-c1ccc(-n2c3ccccc3c3cc(-n4c5ccccc5c5ccccc54)c(-n4c5ccccc5c5cccnc54)cc32)cc1 ; c1ccc([Si](c2ccccc2)(c2ccccc2)c2cccc(-n3c4ccccc4c4cc(-n5c6ccccc6c6ccccc65)c(-n5c6ccccc6c6cccnc65)cc43)c2)cc1] 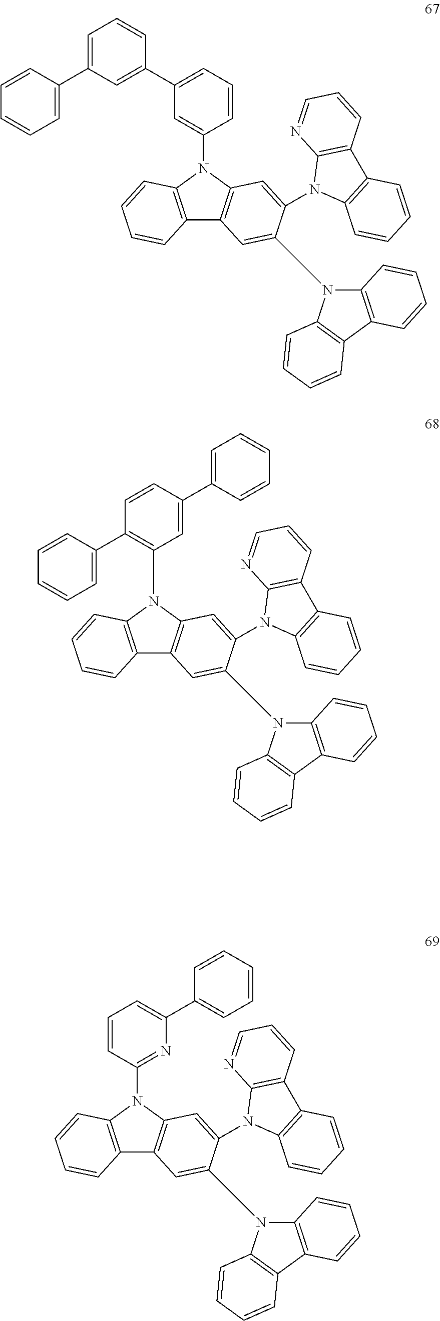 [SMILES: c1ccc(-c2cccc(-c3cccc(-n4c5ccccc5c5cc(-n6c7ccccc7c7ccccc76)c(-n6c7ccccc7c7cccnc76)cc54)c3)c2)cc1 ; c1ccc(-c2ccc(-c3ccccc3)c(-n3c4ccccc4c4cc(-n5c6ccccc6c6ccccc65)c(-n5c6ccccc6c6cccnc65)cc43)c2)cc1 ; c1ccc(-c2cccc(-n3c4ccccc4c4cc(-n5c6ccccc6c6ccccc65)c(-n5c6ccccc6c6cccnc65)cc43)n2)cc1] 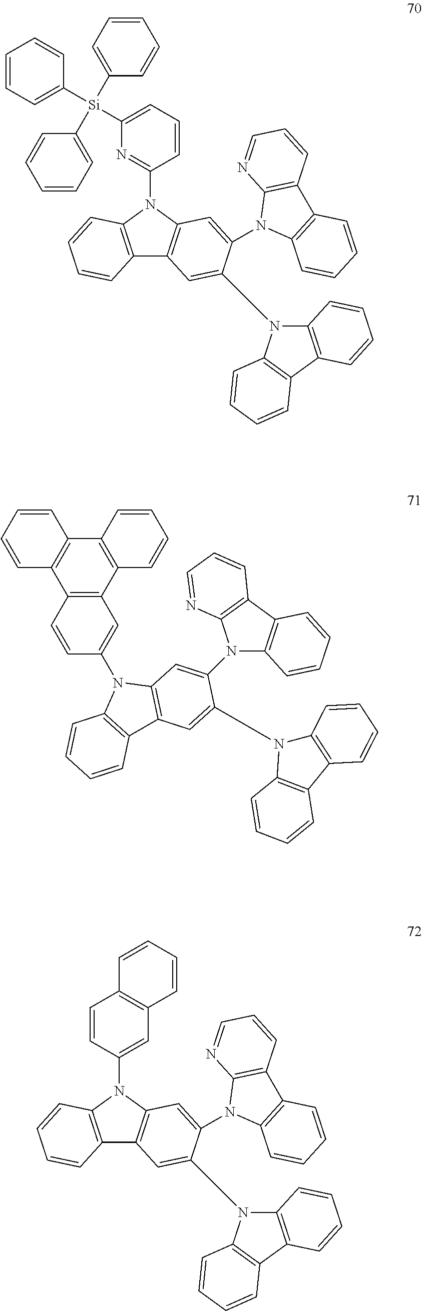 [SMILES: c1ccc([Si](c2ccccc2)(c2ccccc2)c2cccc(-n3c4ccccc4c4cc(-n5c6ccccc6c6ccccc65)c(-n5c6ccccc6c6cccnc65)cc43)n2)cc1 ; c1ccc2c(c1)c1ccccc1c1cc(-n3c4ccccc4c4cc(-n5c6ccccc6c6ccccc65)c(-n5c6ccccc6c6cccnc65)cc43)ccc21 ; c1ccc2cc(-n3c4ccccc4c4cc(-n5c6ccccc6c6ccccc65)c(-n5c6ccccc6c6cccnc65)cc43)ccc2c1] 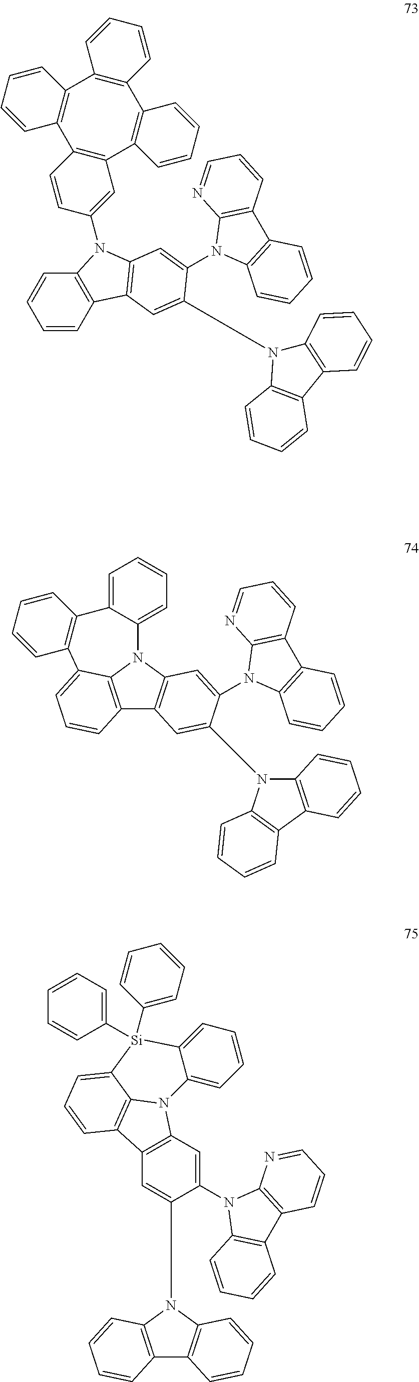 [SMILES: c1ccc2c(c1)-c1ccccc1-c1ccc(-n3c4ccccc4c4cc(-n5c6ccccc6c6ccccc65)c(-n5c6ccccc6c6cccnc65)cc43)cc1-c1ccccc1-2 ; c1ccc2c(c1)-c1ccccc1-n1c3cc(-n4c5ccccc5c5cccnc54)c(-n4c5ccccc5c5ccccc54)cc3c3cccc-2c31 ; c1ccc([Si]2(c3ccccc3)c3ccccc3-n3c4cc(-n5c6ccccc6c6cccnc65)c(-n5c6ccccc6c6ccccc65)cc4c4cccc2c43)cc1] 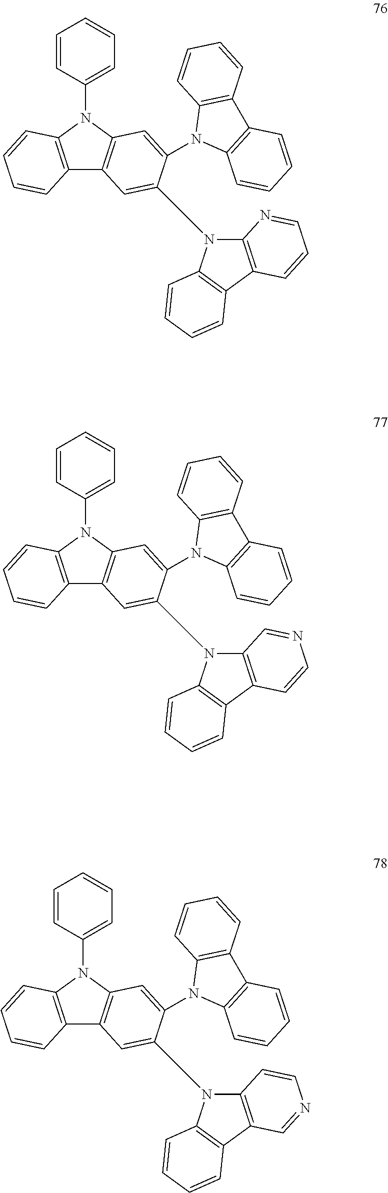 [SMILES: c1ccc(-n2c3ccccc3c3cc(-n4c5ccccc5c5cccnc54)c(-n4c5ccccc5c5ccccc54)cc32)cc1 ; c1ccc(-n2c3ccccc3c3cc(-n4c5ccccc5c5ccncc54)c(-n4c5ccccc5c5ccccc54)cc32)cc1 ; c1ccc(-n2c3ccccc3c3cc(-n4c5ccccc5c5cnccc54)c(-n4c5ccccc5c5ccccc54)cc32)cc1] 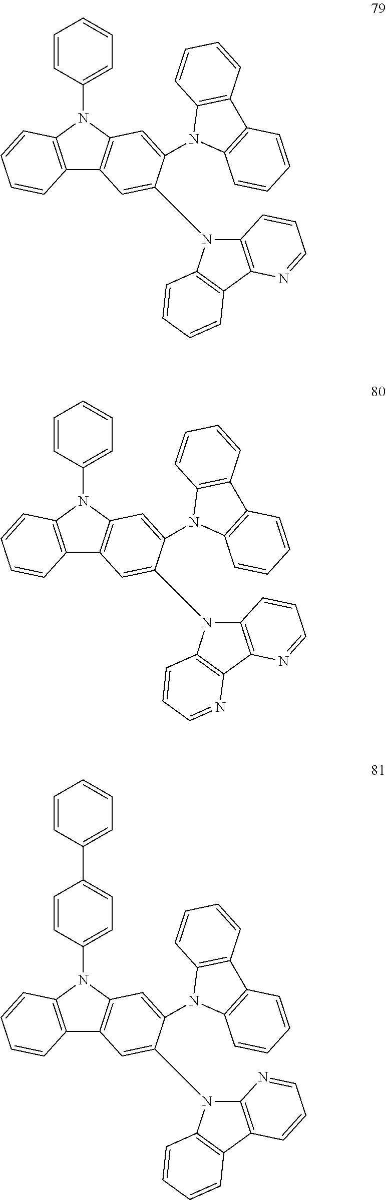 [SMILES: c1ccc(-n2c3ccccc3c3cc(-n4c5ccccc5c5ncccc54)c(-n4c5ccccc5c5ccccc54)cc32)cc1 ; c1ccc(-n2c3ccccc3c3cc(-n4c5cccnc5c5ncccc54)c(-n4c5ccccc5c5ccccc54)cc32)cc1 ; c1ccc(-c2ccc(-n3c4ccccc4c4cc(-n5c6ccccc6c6cccnc65)c(-n5c6ccccc6c6ccccc65)cc43)cc2)cc1] 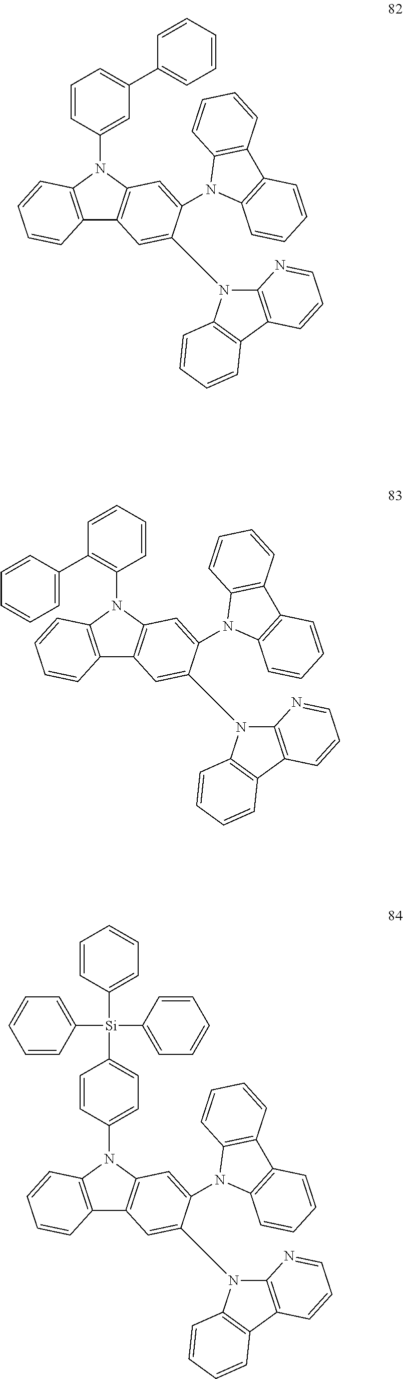 [SMILES: c1ccc(-c2cccc(-n3c4ccccc4c4cc(-n5c6ccccc6c6cccnc65)c(-n5c6ccccc6c6ccccc65)cc43)c2)cc1 ; c1ccc(-c2ccccc2-n2c3ccccc3c3cc(-n4c5ccccc5c5cccnc54)c(-n4c5ccccc5c5ccccc54)cc32)cc1 ; c1ccc([Si](c2ccccc2)(c2ccccc2)c2ccc(-n3c4ccccc4c4cc(-n5c6ccccc6c6cccnc65)c(-n5c6ccccc6c6ccccc65)cc43)cc2)cc1] 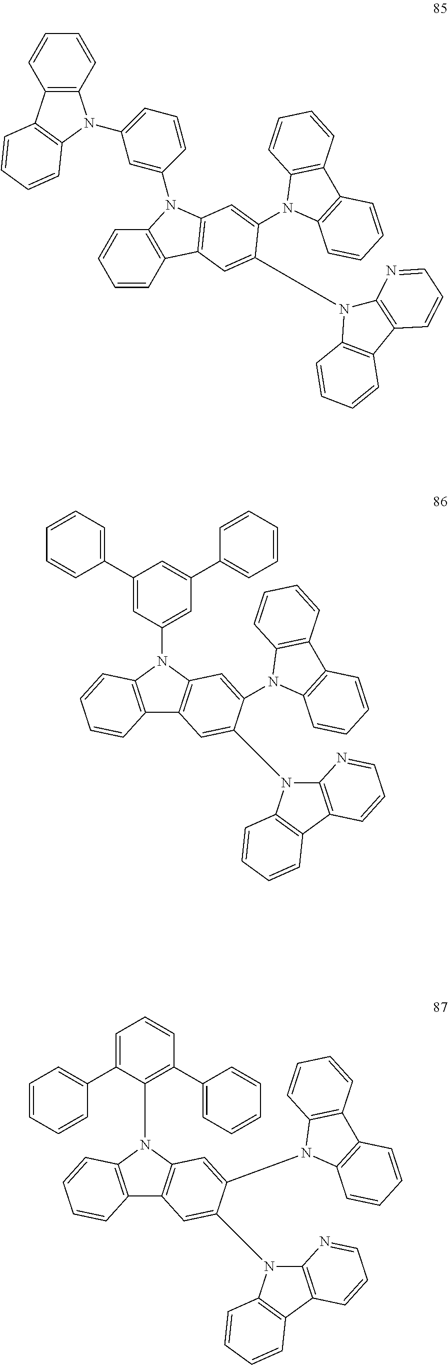 [SMILES: c1cc(-n2c3ccccc3c3ccccc32)cc(-n2c3ccccc3c3cc(-n4c5ccccc5c5cccnc54)c(-n4c5ccccc5c5ccccc54)cc32)c1 ; c1ccc(-c2cc(-c3ccccc3)cc(-n3c4ccccc4c4cc(-n5c6ccccc6c6cccnc65)c(-n5c6ccccc6c6ccccc65)cc43)c2)cc1 ; c1ccc(-c2cccc(-c3ccccc3)c2-n2c3ccccc3c3cc(-n4c5ccccc5c5cccnc54)c(-n4c5ccccc5c5ccccc54)cc32)cc1] 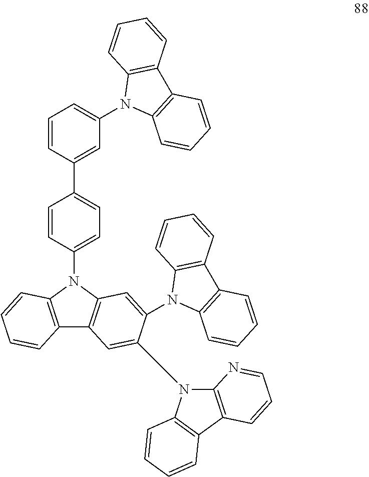 [SMILES: c1cc(-c2ccc(-n3c4ccccc4c4cc(-n5c6ccccc6c6cccnc65)c(-n5c6ccccc6c6ccccc65)cc43)cc2)cc(-n2c3ccccc3c3ccccc32)c1] 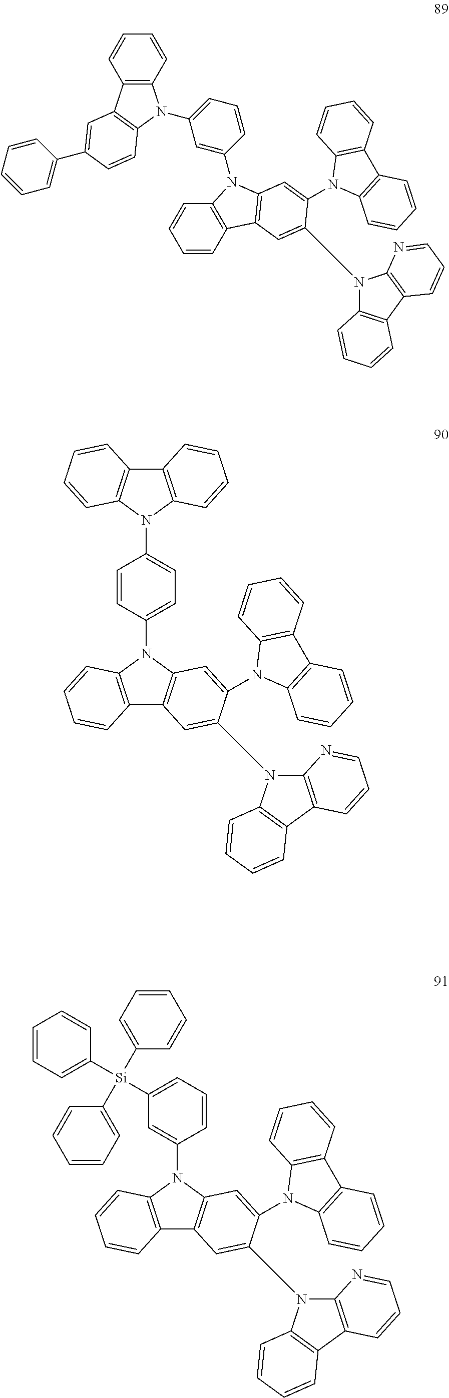 [SMILES: c1ccc(-c2ccc3c(c2)c2ccccc2n3-c2cccc(-n3c4ccccc4c4cc(-n5c6ccccc6c6cccnc65)c(-n5c6ccccc6c6ccccc65)cc43)c2)cc1 ; c1ccc2c(c1)c1ccccc1n2-c1ccc(-n2c3ccccc3c3cc(-n4c5ccccc5c5cccnc54)c(-n4c5ccccc5c5ccccc54)cc32)cc1 ; c1ccc([Si](c2ccccc2)(c2ccccc2)c2cccc(-n3c4ccccc4c4cc(-n5c6ccccc6c6cccnc65)c(-n5c6ccccc6c6ccccc65)cc43)c2)cc1] 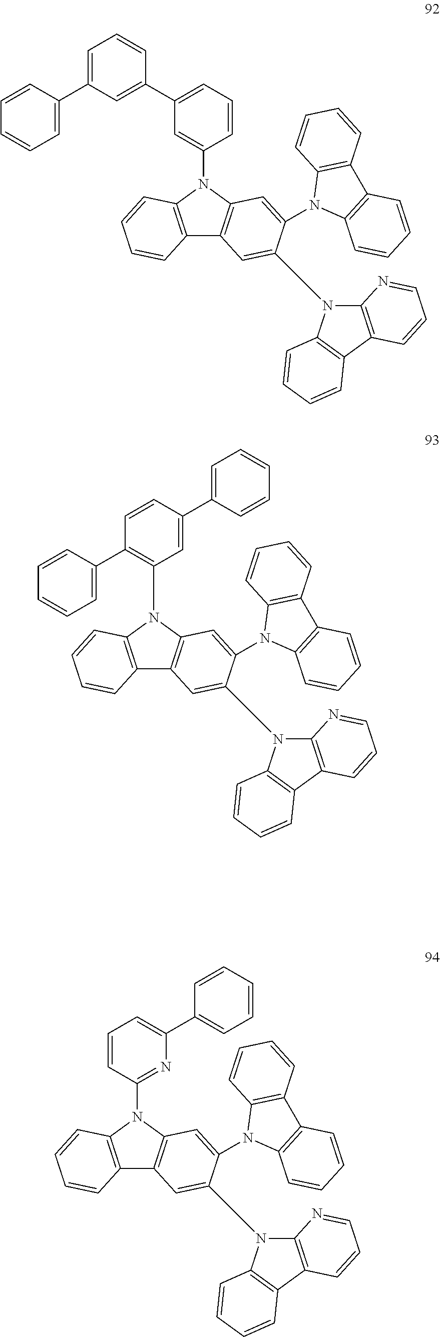 [SMILES: c1ccc(-c2cccc(-c3cccc(-n4c5ccccc5c5cc(-n6c7ccccc7c7cccnc76)c(-n6c7ccccc7c7ccccc76)cc54)c3)c2)cc1 ; c1ccc(-c2ccc(-c3ccccc3)c(-n3c4ccccc4c4cc(-n5c6ccccc6c6cccnc65)c(-n5c6ccccc6c6ccccc65)cc43)c2)cc1 ; c1ccc(-c2cccc(-n3c4ccccc4c4cc(-n5c6ccccc6c6cccnc65)c(-n5c6ccccc6c6ccccc65)cc43)n2)cc1] 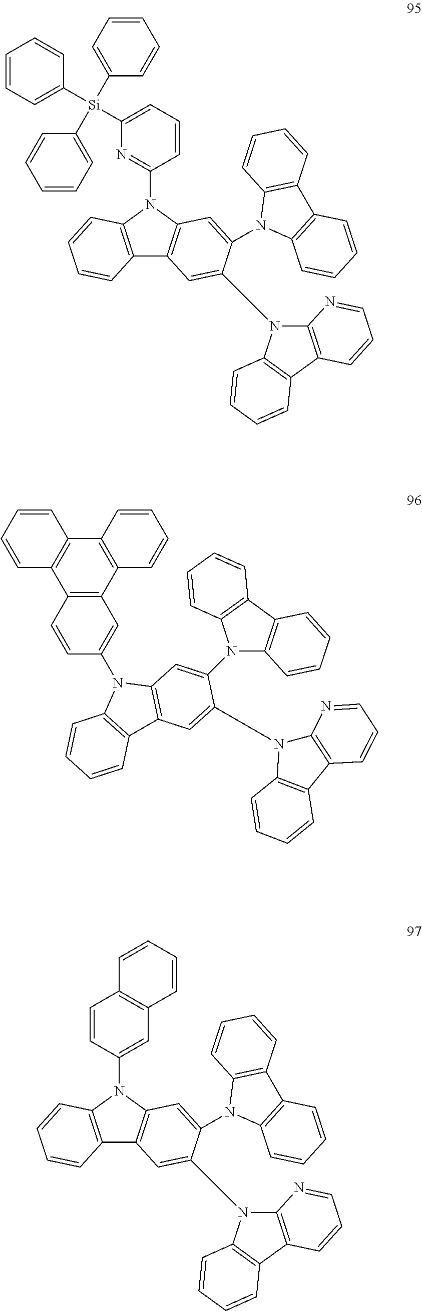 [SMILES: c1ccc([Si](c2ccccc2)(c2ccccc2)c2cccc(-n3c4ccccc4c4cc(-n5c6ccccc6c6cccnc65)c(-n5c6ccccc6c6ccccc65)cc43)n2)cc1 ; c1ccc2c(c1)c1ccccc1c1cc(-n3c4ccccc4c4cc(-n5c6ccccc6c6cccnc65)c(-n5c6ccccc6c6ccccc65)cc43)ccc21 ; c1ccc2cc(-n3c4ccccc4c4cc(-n5c6ccccc6c6cccnc65)c(-n5c6ccccc6c6ccccc65)cc43)ccc2c1] 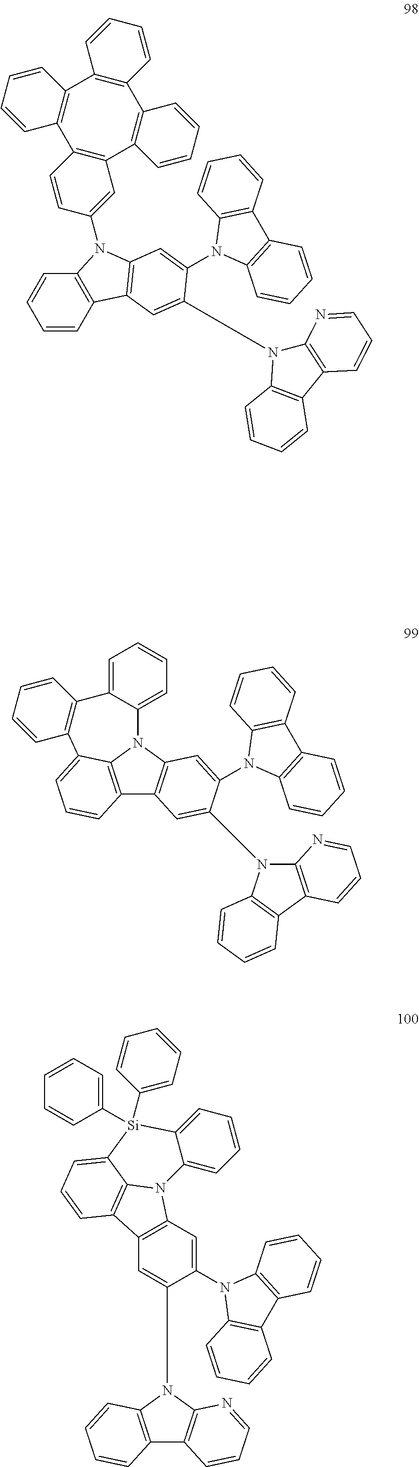 [SMILES: c1ccc2c(c1)-c1ccccc1-c1ccc(-n3c4ccccc4c4cc(-n5c6ccccc6c6cccnc65)c(-n5c6ccccc6c6ccccc65)cc43)cc1-c1ccccc1-2 ; c1ccc2c(c1)-c1ccccc1-n1c3cc(-n4c5ccccc5c5ccccc54)c(-n4c5ccccc5c5cccnc54)cc3c3cccc-2c31 ; c1ccc([Si]2(c3ccccc3)c3ccccc3-n3c4cc(-n5c6ccccc6c6ccccc65)c(-n5c6ccccc6c6cccnc65)cc4c4cccc2c43)cc1] 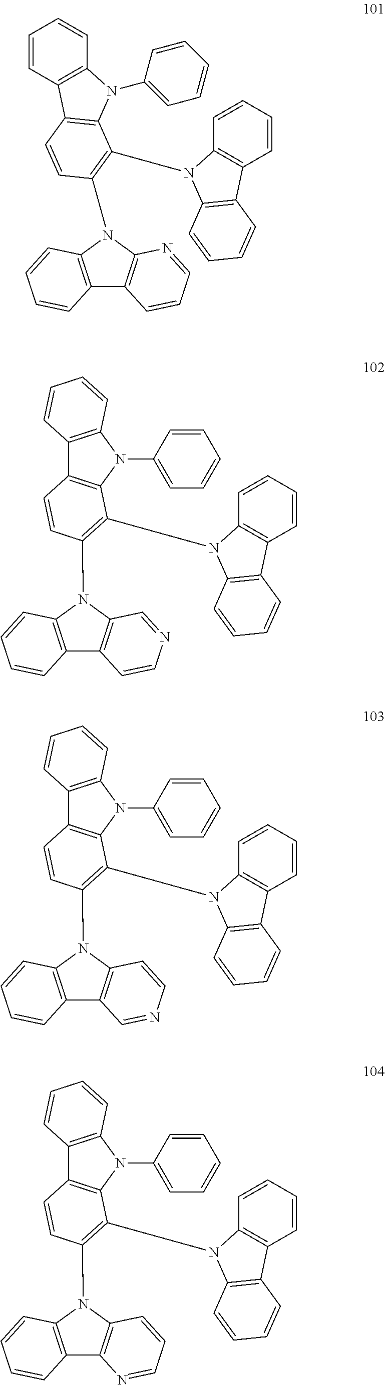 [SMILES: c1ccc(-n2c3ccccc3c3ccc(-n4c5ccccc5c5cccnc54)c(-n4c5ccccc5c5ccccc54)c32)cc1 ; c1ccc(-n2c3ccccc3c3ccc(-n4c5ccccc5c5ccncc54)c(-n4c5ccccc5c5ccccc54)c32)cc1 ; c1ccc(-n2c3ccccc3c3ccc(-n4c5ccccc5c5cnccc54)c(-n4c5ccccc5c5ccccc54)c32)cc1 ; c1ccc(-n2c3ccccc3c3ccc(-n4c5ccccc5c5ncccc54)c(-n4c5ccccc5c5ccccc54)c32)cc1] 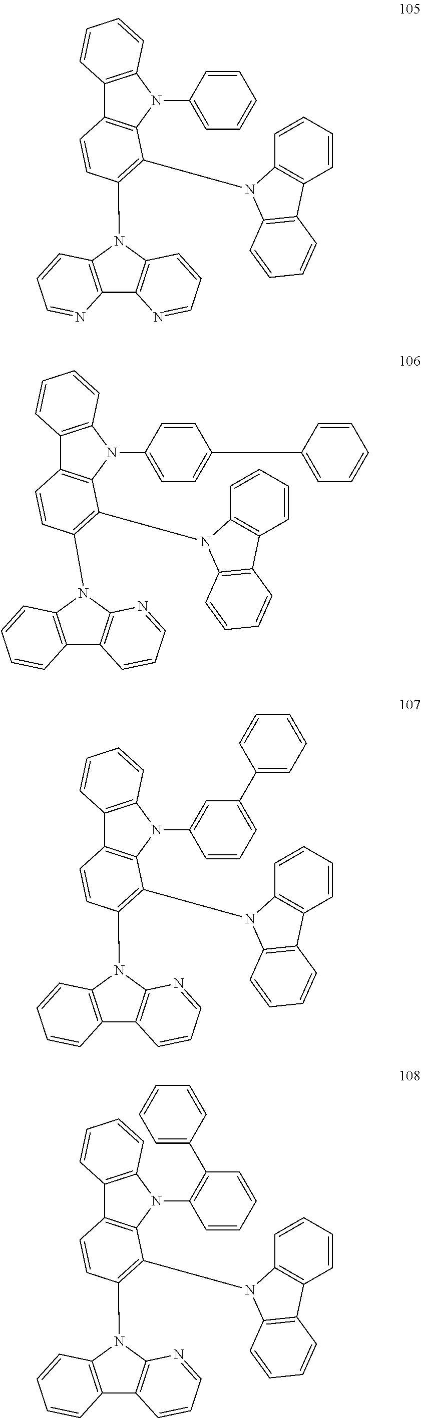 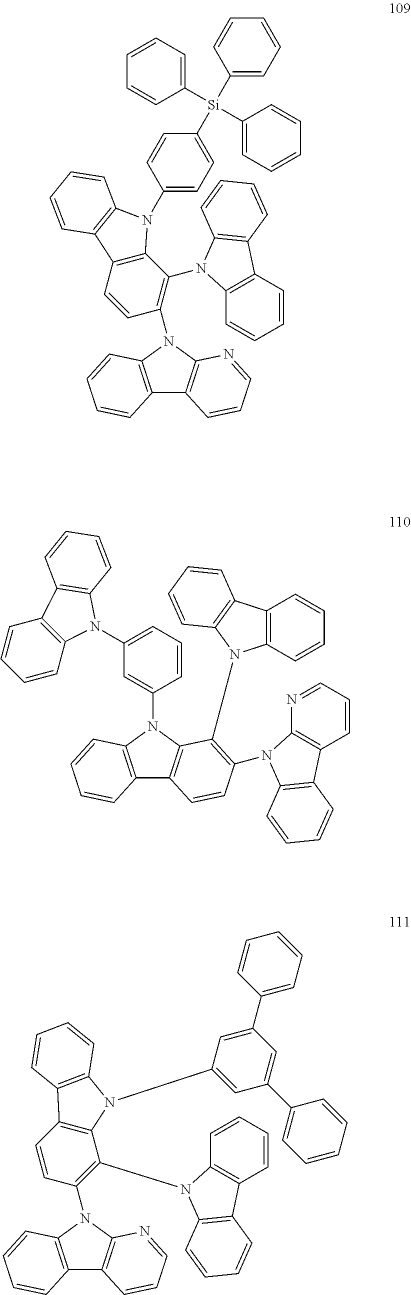 [SMILES: c1ccc([Si](c2ccccc2)(c2ccccc2)c2ccc(-n3c4ccccc4c4ccc(-n5c6ccccc6c6cccnc65)c(-n5c6ccccc6c6ccccc65)c43)cc2)cc1 ; c1cc(-n2c3ccccc3c3ccccc32)cc(-n2c3ccccc3c3ccc(-n4c5ccccc5c5cccnc54)c(-n4c5ccccc5c5ccccc54)c32)c1 ; c1ccc(-c2cc(-c3ccccc3)cc(-n3c4ccccc4c4ccc(-n5c6ccccc6c6cccnc65)c(-n5c6ccccc6c6ccccc65)c43)c2)cc1] 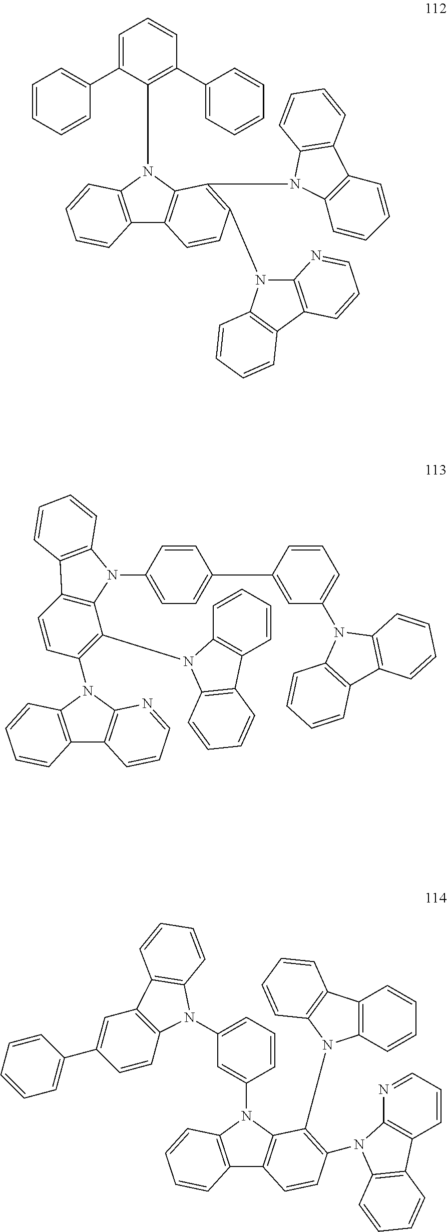 [SMILES: c1ccc(-c2cccc(-c3ccccc3)c2-n2c3ccccc3c3ccc(-n4c5ccccc5c5cccnc54)c(-n4c5ccccc5c5ccccc54)c32)cc1 ; c1cc(-c2ccc(-n3c4ccccc4c4ccc(-n5c6ccccc6c6cccnc65)c(-n5c6ccccc6c6ccccc65)c43)cc2)cc(-n2c3ccccc3c3ccccc32)c1 ; c1ccc(-c2ccc3c(c2)c2ccccc2n3-c2cccc(-n3c4ccccc4c4ccc(-n5c6ccccc6c6cccnc65)c(-n5c6ccccc6c6ccccc65)c43)c2)cc1] 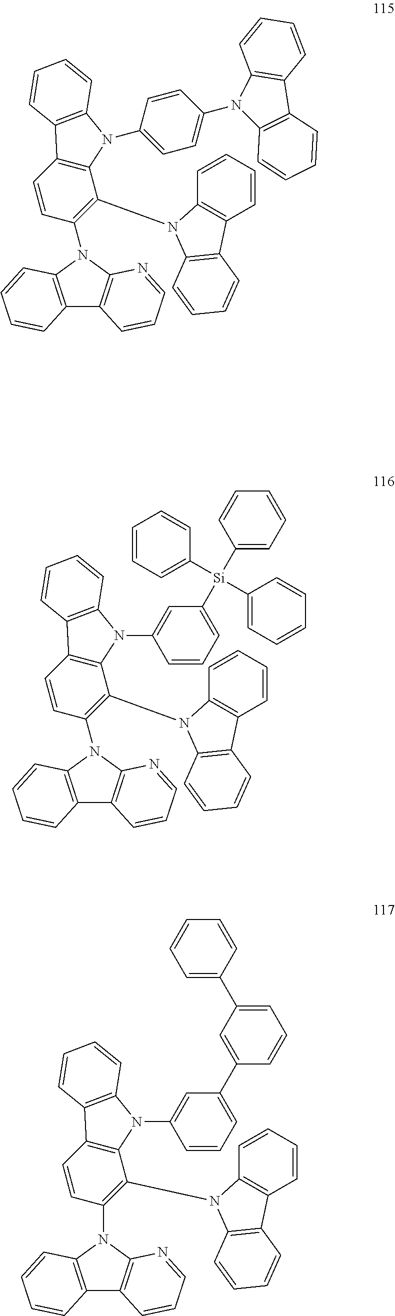 [SMILES: c1ccc2c(c1)c1ccccc1n2-c1ccc(-n2c3ccccc3c3ccc(-n4c5ccccc5c5cccnc54)c(-n4c5ccccc5c5ccccc54)c32)cc1 ; c1ccc([Si](c2ccccc2)(c2ccccc2)c2cccc(-n3c4ccccc4c4ccc(-n5c6ccccc6c6cccnc65)c(-n5c6ccccc6c6ccccc65)c43)c2)cc1 ; c1ccc(-c2cccc(-c3cccc(-n4c5ccccc5c5ccc(-n6c7ccccc7c7cccnc76)c(-n6c7ccccc7c7ccccc76)c54)c3)c2)cc1] 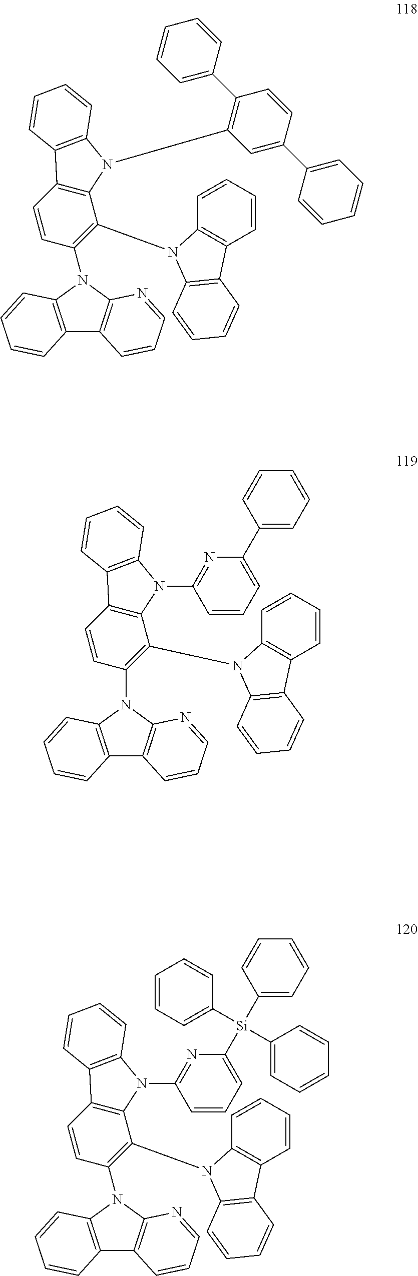 [SMILES: c1ccc(-c2ccc(-c3ccccc3)c(-n3c4ccccc4c4ccc(-n5c6ccccc6c6cccnc65)c(-n5c6ccccc6c6ccccc65)c43)c2)cc1 ; c1ccc(-c2cccc(-n3c4ccccc4c4ccc(-n5c6ccccc6c6cccnc65)c(-n5c6ccccc6c6ccccc65)c43)n2)cc1 ; c1ccc([Si](c2ccccc2)(c2ccccc2)c2cccc(-n3c4ccccc4c4ccc(-n5c6ccccc6c6cccnc65)c(-n5c6ccccc6c6ccccc65)c43)n2)cc1] 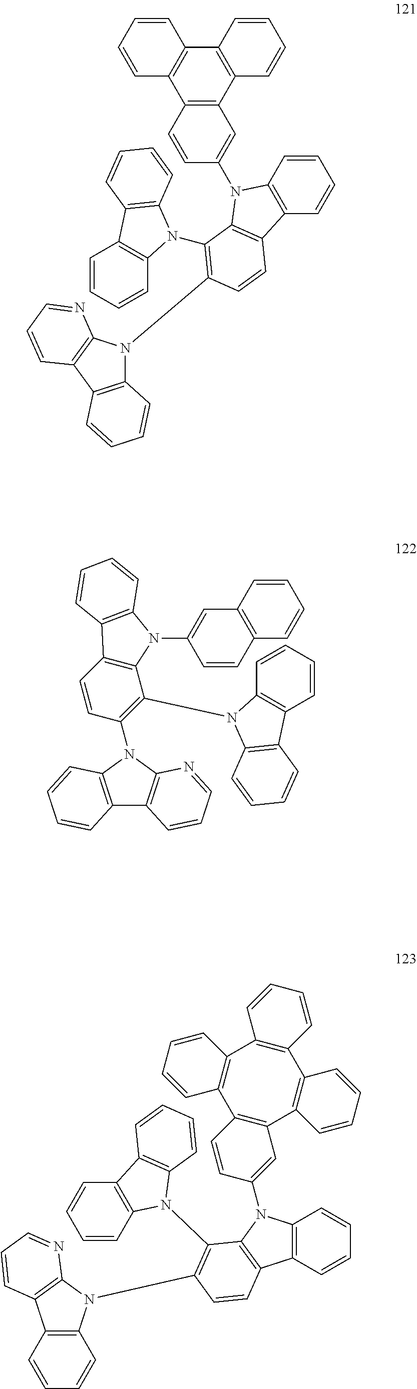 [SMILES: c1ccc2c(c1)c1ccccc1c1cc(-n3c4ccccc4c4ccc(-n5c6ccccc6c6cccnc65)c(-n5c6ccccc6c6ccccc65)c43)ccc21 ; c1ccc2cc(-n3c4ccccc4c4ccc(-n5c6ccccc6c6cccnc65)c(-n5c6ccccc6c6ccccc65)c43)ccc2c1 ; c1ccc2c(c1)-c1ccccc1-c1ccc(-n3c4ccccc4c4ccc(-n5c6ccccc6c6cccnc65)c(-n5c6ccccc6c6ccccc65)c43)cc1-c1ccccc1-2] 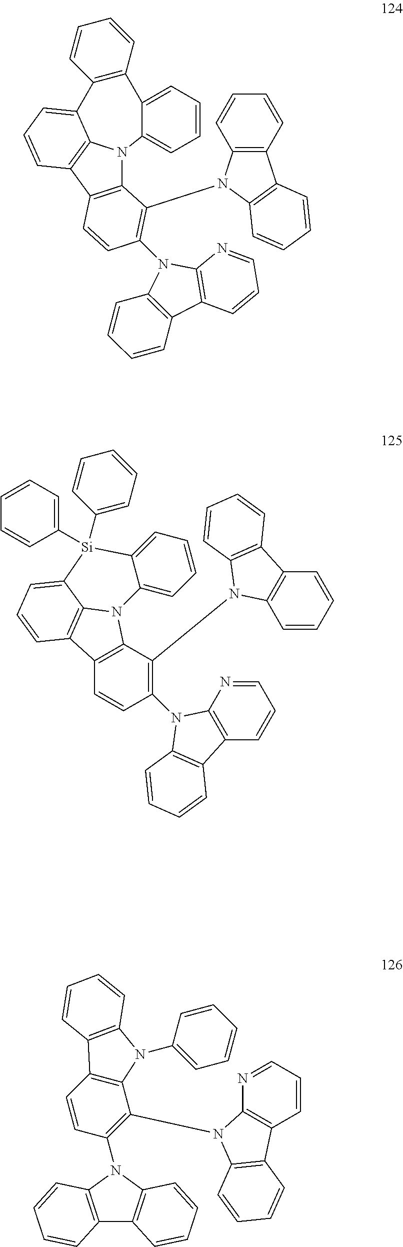 [SMILES: c1ccc2c(c1)-c1ccccc1-n1c3c-2cccc3c2ccc(-n3c4ccccc4c4cccnc43)c(-n3c4ccccc4c4ccccc43)c21 ; c1ccc([Si]2(c3ccccc3)c3ccccc3-n3c4c2cccc4c2ccc(-n4c5ccccc5c5cccnc54)c(-n4c5ccccc5c5ccccc54)c23)cc1 ; c1ccc(-n2c3ccccc3c3ccc(-n4c5ccccc5c5ccccc54)c(-n4c5ccccc5c5cccnc54)c32)cc1] 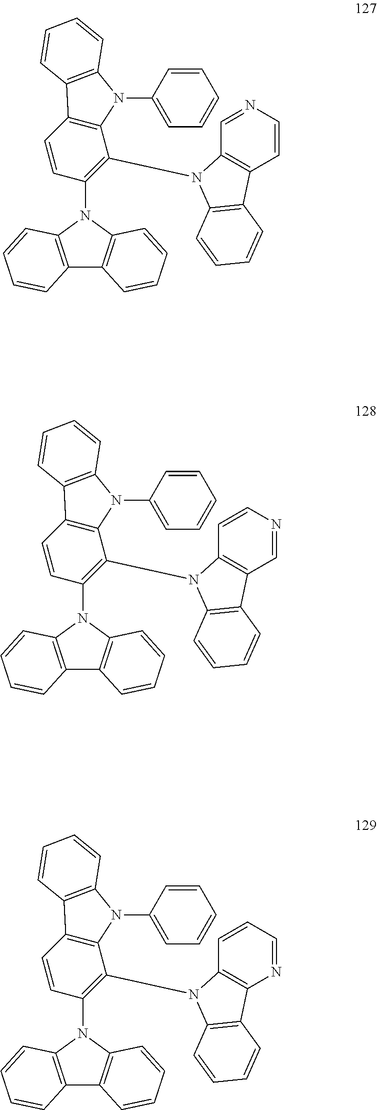 [SMILES: c1ccc(-n2c3ccccc3c3ccc(-n4c5ccccc5c5ccccc54)c(-n4c5ccccc5c5ccncc54)c32)cc1 ; c1ccc(-n2c3ccccc3c3ccc(-n4c5ccccc5c5ccccc54)c(-n4c5ccccc5c5cnccc54)c32)cc1 ; c1ccc(-n2c3ccccc3c3ccc(-n4c5ccccc5c5ccccc54)c(-n4c5ccccc5c5ncccc54)c32)cc1] 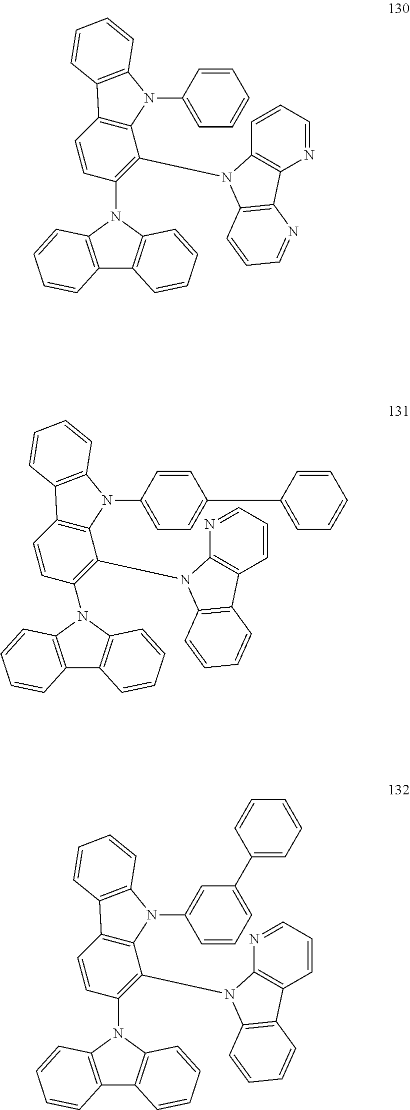 [SMILES: c1ccc(-n2c3ccccc3c3ccc(-n4c5ccccc5c5ccccc54)c(-n4c5cccnc5c5ncccc54)c32)cc1 ; c1ccc(-c2ccc(-n3c4ccccc4c4ccc(-n5c6ccccc6c6ccccc65)c(-n5c6ccccc6c6cccnc65)c43)cc2)cc1 ; c1ccc(-c2cccc(-n3c4ccccc4c4ccc(-n5c6ccccc6c6ccccc65)c(-n5c6ccccc6c6cccnc65)c43)c2)cc1] 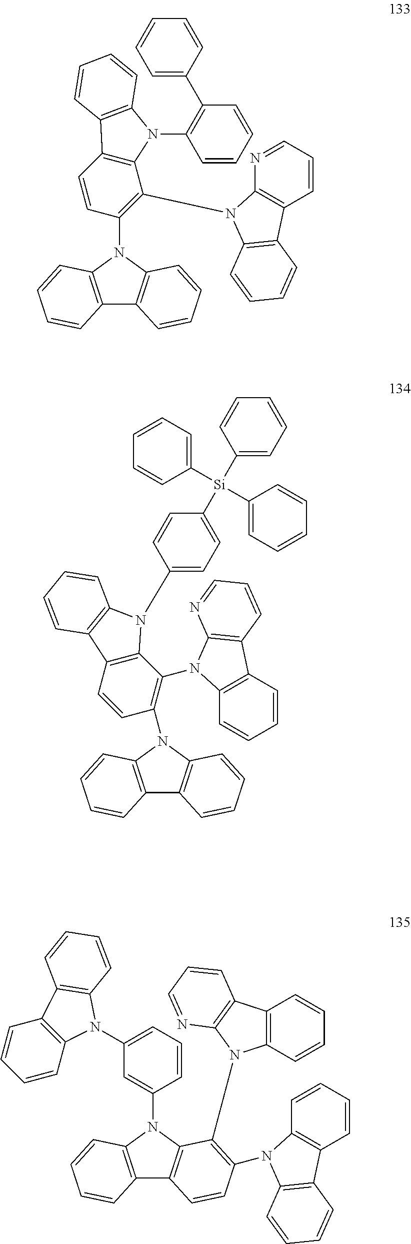 [SMILES: c1ccc(-c2ccccc2-n2c3ccccc3c3ccc(-n4c5ccccc5c5ccccc54)c(-n4c5ccccc5c5cccnc54)c32)cc1 ; c1ccc([Si](c2ccccc2)(c2ccccc2)c2ccc(-n3c4ccccc4c4ccc(-n5c6ccccc6c6ccccc65)c(-n5c6ccccc6c6cccnc65)c43)cc2)cc1 ; c1cc(-n2c3ccccc3c3ccccc32)cc(-n2c3ccccc3c3ccc(-n4c5ccccc5c5ccccc54)c(-n4c5ccccc5c5cccnc54)c32)c1] 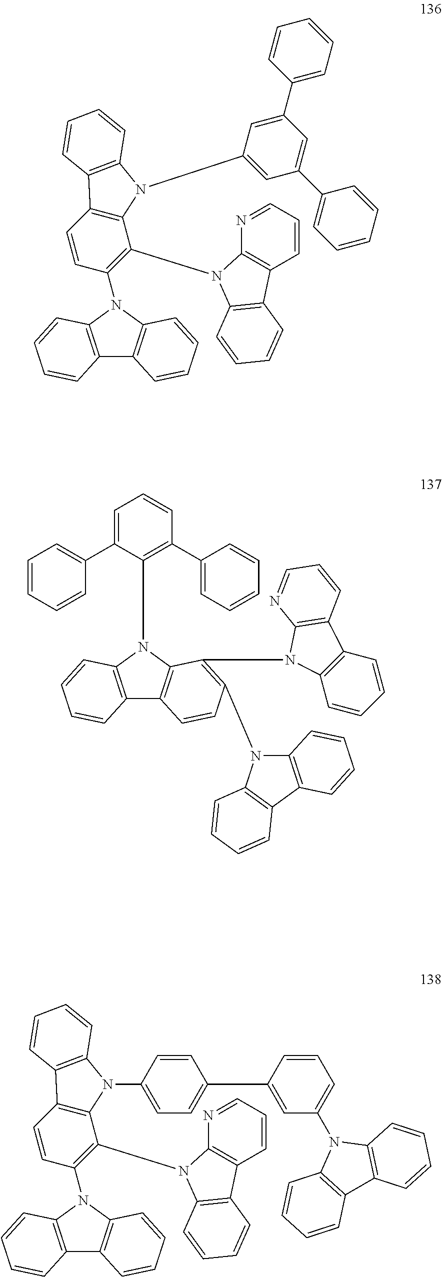 [SMILES: c1ccc(-c2cc(-c3ccccc3)cc(-n3c4ccccc4c4ccc(-n5c6ccccc6c6ccccc65)c(-n5c6ccccc6c6cccnc65)c43)c2)cc1 ; c1ccc(-c2cccc(-c3ccccc3)c2-n2c3ccccc3c3ccc(-n4c5ccccc5c5ccccc54)c(-n4c5ccccc5c5cccnc54)c32)cc1 ; c1cc(-c2ccc(-n3c4ccccc4c4ccc(-n5c6ccccc6c6ccccc65)c(-n5c6ccccc6c6cccnc65)c43)cc2)cc(-n2c3ccccc3c3ccccc32)c1] 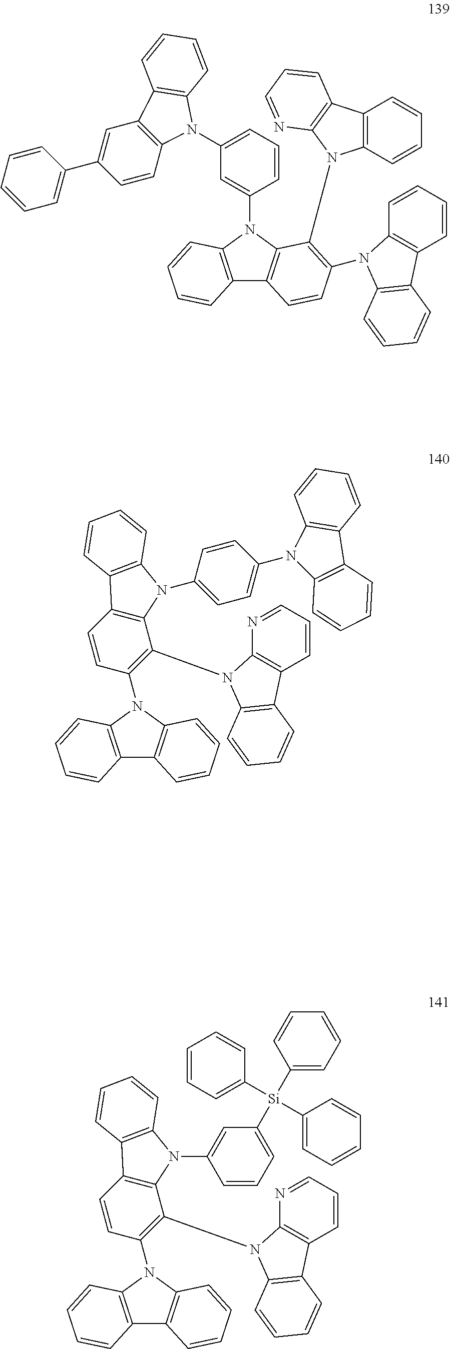 [SMILES: c1ccc(-c2ccc3c(c2)c2ccccc2n3-c2cccc(-n3c4ccccc4c4ccc(-n5c6ccccc6c6ccccc65)c(-n5c6ccccc6c6cccnc65)c43)c2)cc1 ; c1ccc2c(c1)c1ccccc1n2-c1ccc(-n2c3ccccc3c3ccc(-n4c5ccccc5c5ccccc54)c(-n4c5ccccc5c5cccnc54)c32)cc1 ; c1ccc([Si](c2ccccc2)(c2ccccc2)c2cccc(-n3c4ccccc4c4ccc(-n5c6ccccc6c6ccccc65)c(-n5c6ccccc6c6cccnc65)c43)c2)cc1] 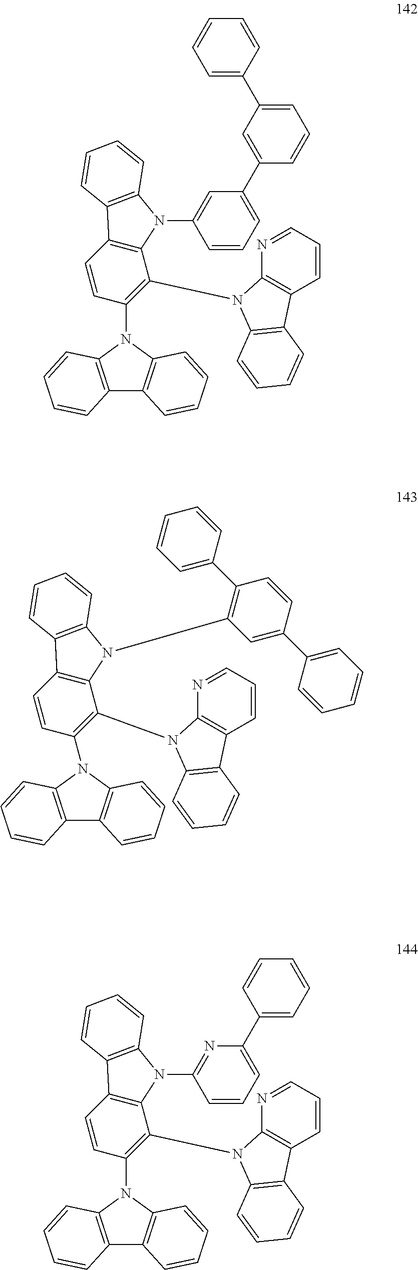 [SMILES: c1ccc(-c2cccc(-c3cccc(-n4c5ccccc5c5ccc(-n6c7ccccc7c7ccccc76)c(-n6c7ccccc7c7cccnc76)c54)c3)c2)cc1 ; c1ccc(-c2ccc(-c3ccccc3)c(-n3c4ccccc4c4ccc(-n5c6ccccc6c6ccccc65)c(-n5c6ccccc6c6cccnc65)c43)c2)cc1 ; c1ccc(-c2cccc(-n3c4ccccc4c4ccc(-n5c6ccccc6c6ccccc65)c(-n5c6ccccc6c6cccnc65)c43)n2)cc1] 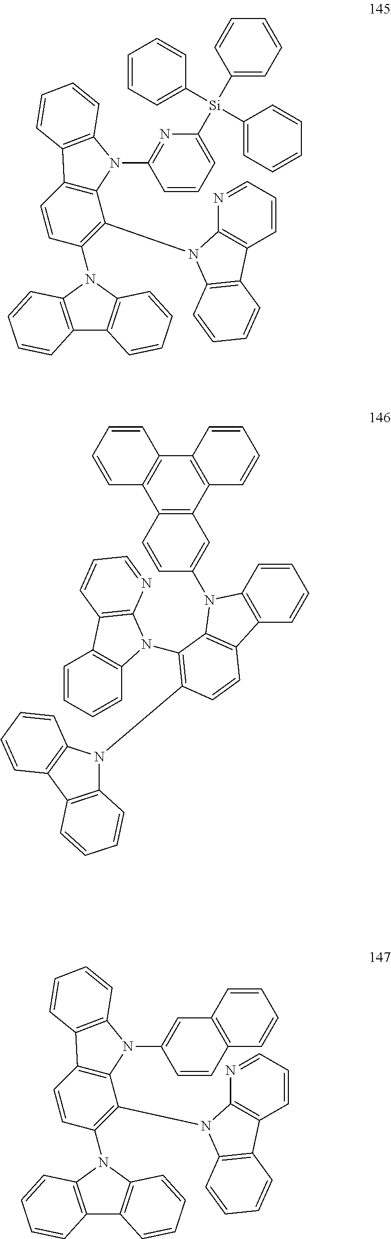 [SMILES: c1ccc([Si](c2ccccc2)(c2ccccc2)c2cccc(-n3c4ccccc4c4ccc(-n5c6ccccc6c6ccccc65)c(-n5c6ccccc6c6cccnc65)c43)n2)cc1 ; c1ccc2c(c1)c1ccccc1c1cc(-n3c4ccccc4c4ccc(-n5c6ccccc6c6ccccc65)c(-n5c6ccccc6c6cccnc65)c43)ccc21 ; c1ccc2cc(-n3c4ccccc4c4ccc(-n5c6ccccc6c6ccccc65)c(-n5c6ccccc6c6cccnc65)c43)ccc2c1] 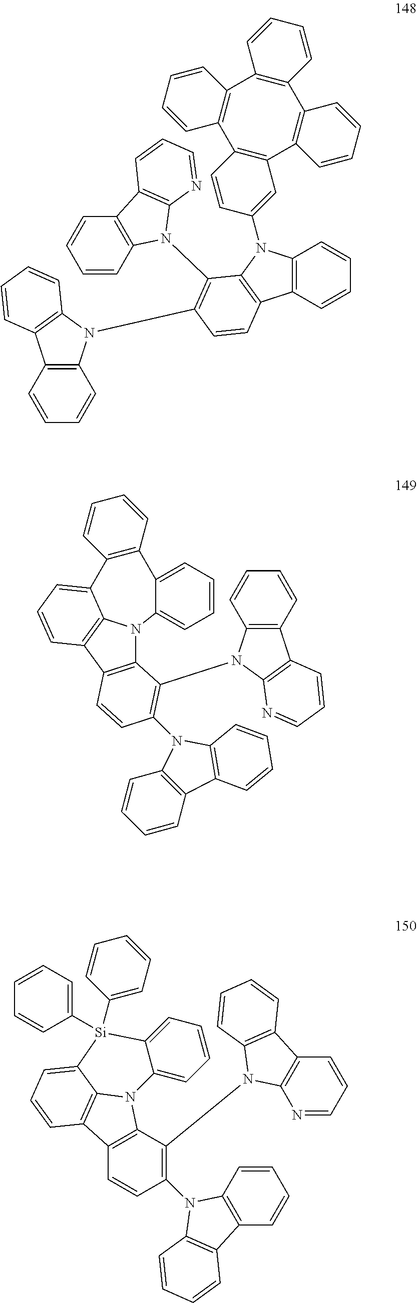 [SMILES: c1ccc2c(c1)-c1ccccc1-c1ccc(-n3c4ccccc4c4ccc(-n5c6ccccc6c6ccccc65)c(-n5c6ccccc6c6cccnc65)c43)cc1-c1ccccc1-2 ; c1ccc2c(c1)-c1ccccc1-n1c3c-2cccc3c2ccc(-n3c4ccccc4c4ccccc43)c(-n3c4ccccc4c4cccnc43)c21 ; c1ccc([Si]2(c3ccccc3)c3ccccc3-n3c4c2cccc4c2ccc(-n4c5ccccc5c5ccccc54)c(-n4c5ccccc5c5cccnc54)c23)cc1] 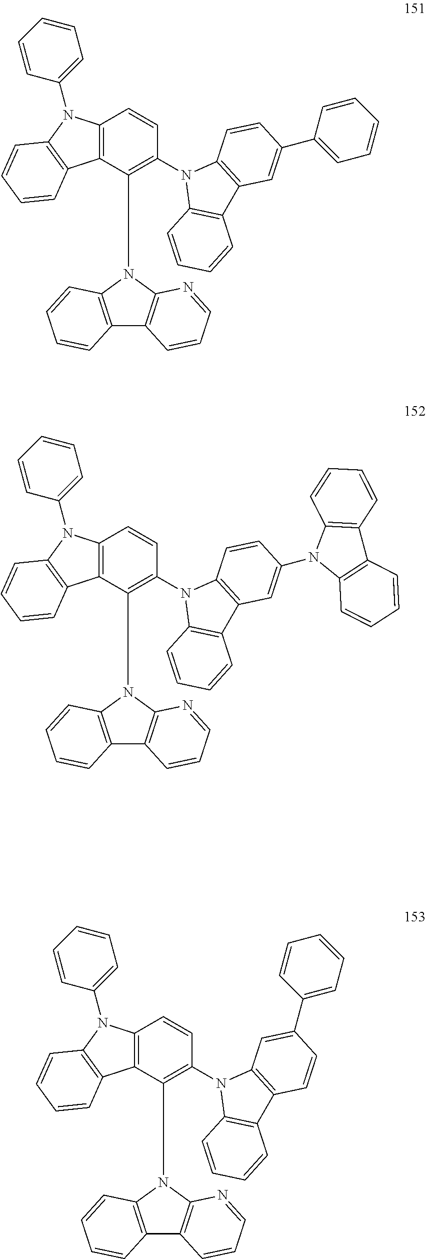 [SMILES: c1ccc(-c2ccc3c(c2)c2ccccc2n3-c2ccc3c(c2-n2c4ccccc4c4cccnc42)c2ccccc2n3-c2ccccc2)cc1 ; c1ccc(-n2c3ccccc3c3c(-n4c5ccccc5c5cccnc54)c(-n4c5ccccc5c5cc(-n6c7ccccc7c7ccccc76)ccc54)ccc32)cc1 ; c1ccc(-c2ccc3c4ccccc4n(-c4ccc5c(c4-n4c6ccccc6c6cccnc64)c4ccccc4n5-c4ccccc4)c3c2)cc1] 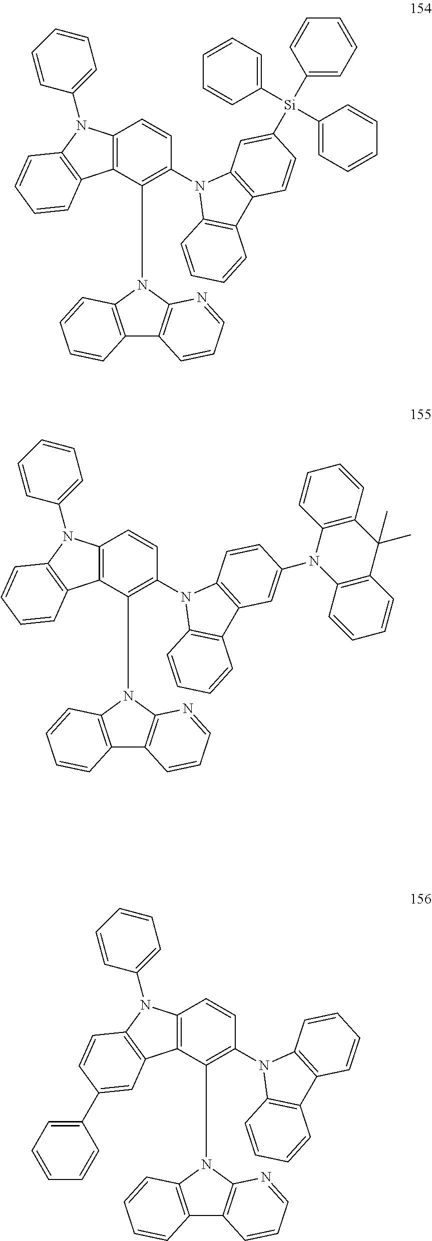 [SMILES: c1ccc(-n2c3ccccc3c3c(-n4c5ccccc5c5cccnc54)c(-n4c5ccccc5c5ccc([Si](c6ccccc6)(c6ccccc6)c6ccccc6)cc54)ccc32)cc1 ; CC1(C)c2ccccc2N(c2ccc3c(c2)c2ccccc2n3-c2ccc3c(c2-n2c4ccccc4c4cccnc42)c2ccccc2n3-c2ccccc2)c2ccccc21 ; c1ccc(-c2ccc3c(c2)c2c(-n4c5ccccc5c5cccnc54)c(-n4c5ccccc5c5ccccc54)ccc2n3-c2ccccc2)cc1] 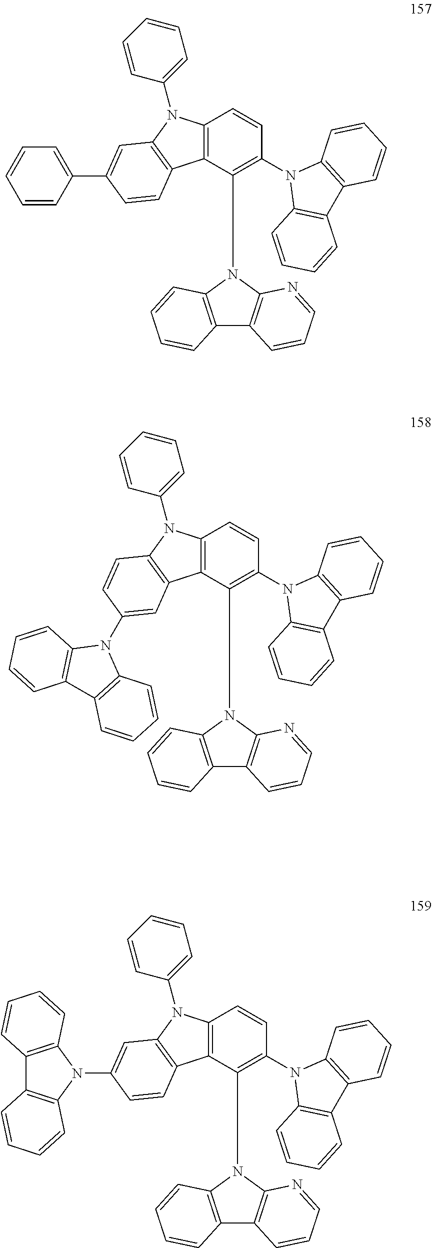 [SMILES: c1ccc(-c2ccc3c4c(-n5c6ccccc6c6cccnc65)c(-n5c6ccccc6c6ccccc65)ccc4n(-c4ccccc4)c3c2)cc1 ; c1ccc(-n2c3ccc(-n4c5ccccc5c5ccccc54)cc3c3c(-n4c5ccccc5c5cccnc54)c(-n4c5ccccc5c5ccccc54)ccc32)cc1 ; c1ccc(-n2c3cc(-n4c5ccccc5c5ccccc54)ccc3c3c(-n4c5ccccc5c5cccnc54)c(-n4c5ccccc5c5ccccc54)ccc32)cc1] 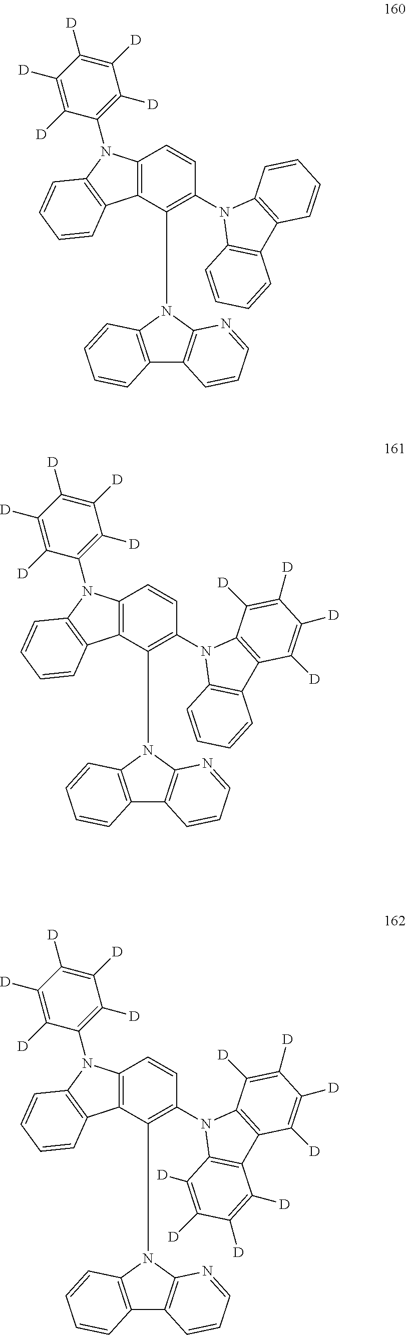 [SMILES: [2H]c1c([2H])c([2H])c(-n2c3ccccc3c3c(-n4c5ccccc5c5cccnc54)c(-n4c5ccccc5c5ccccc54)ccc32)c([2H])c1[2H] ; [2H]c1c([2H])c([2H])c(-n2c3ccccc3c3c(-n4c5ccccc5c5cccnc54)c(-n4c5ccccc5c5c([2H])c([2H])c([2H])c([2H])c54)ccc32)c([2H])c1[2H] ; [2H]c1c([2H])c([2H])c(-n2c3ccccc3c3c(-n4c5ccccc5c5cccnc54)c(-n4c5c([2H])c([2H])c([2H])c([2H])c5c5c([2H])c([2H])c([2H])c([2H])c54)ccc32)c([2H])c1[2H]] 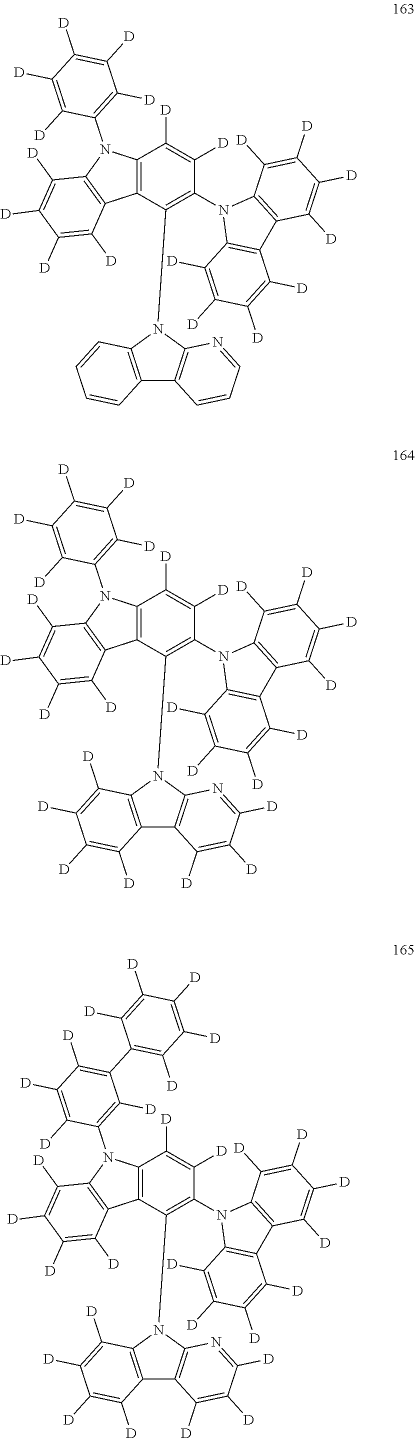 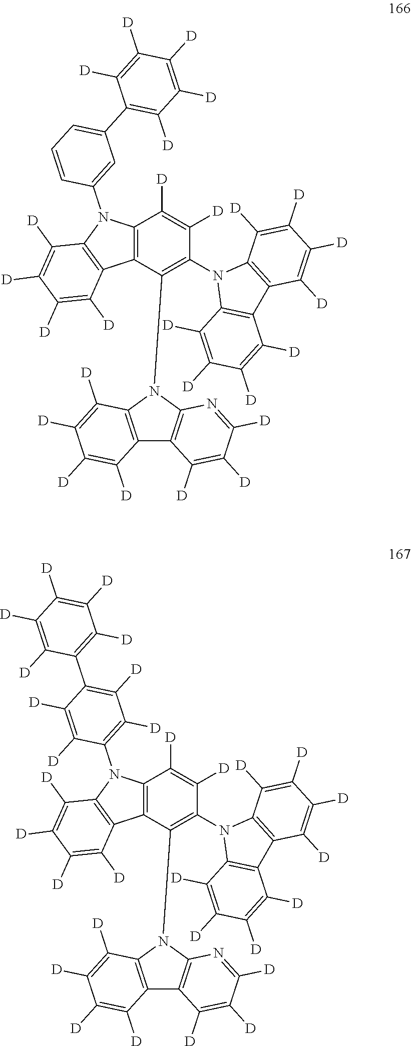 [SMILES: [2H]c1nc2c(c([2H])c1[2H])c1c([2H])c([2H])c([2H])c([2H])c1n2-c1c(-n2c3c([2H])c([2H])c([2H])c([2H])c3c3c([2H])c([2H])c([2H])c([2H])c32)c([2H])c([2H])c2c1c1c([2H])c([2H])c([2H])c([2H])c1n2-c1cccc(-c2c([2H])c([2H])c([2H])c([2H])c2[2H])c1 ; [2H]c1nc2c(c([2H])c1[2H])c1c([2H])c([2H])c([2H])c([2H])c1n2-c1c(-n2c3c([2H])c([2H])c([2H])c([2H])c3c3c([2H])c([2H])c([2H])c([2H])c32)c([2H])c([2H])c2c1c1c([2H])c([2H])c([2H])c([2H])c1n2-c1c([2H])c([2H])c(-c2c([2H])c([2H])c([2H])c([2H])c2[2H])c([2H])c1[2H]] 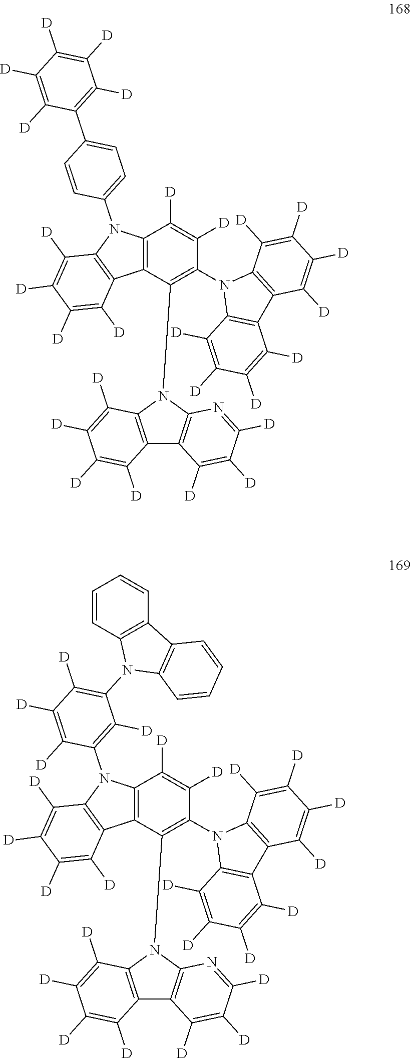 [SMILES: [2H]c1nc2c(c([2H])c1[2H])c1c([2H])c([2H])c([2H])c([2H])c1n2-c1c(-n2c3c([2H])c([2H])c([2H])c([2H])c3c3c([2H])c([2H])c([2H])c([2H])c32)c([2H])c([2H])c2c1c1c([2H])c([2H])c([2H])c([2H])c1n2-c1ccc(-c2c([2H])c([2H])c([2H])c([2H])c2[2H])cc1 ; [2H]c1nc2c(c([2H])c1[2H])c1c([2H])c([2H])c([2H])c([2H])c1n2-c1c(-n2c3c([2H])c([2H])c([2H])c([2H])c3c3c([2H])c([2H])c([2H])c([2H])c32)c([2H])c([2H])c2c1c1c([2H])c([2H])c([2H])c([2H])c1n2-c1c([2H])c([2H])c([2H])c(-n2c3ccccc3c3ccccc32)c1[2H]] [chemical structure]
